(19) United States
(12) Patent Application Publication
(10) Pub. No.: US 2024/0059706 A1
(43) Pub. Date: Feb. 22, 2024

(54) ORGANIC LUMINESCENT COMPOUND AND ORGANIC ELECTROLUMINESCENT DEVICE USING SAME

(71) Applicant: SOLUS ADVANCED MATERIALS CO., LTD., Iksan-si, Jeollabuk-do (KR)

(72) Inventors: Jeongkeun PARK, Yongin-si, Gyeonggi-do (KR); Minsik EUM, Yongin-si, Gyeonggi-do (KR); Jaeyi SIM, Yongin-si, Gyeonggi-do (KR); Doshik KIM, Yongin-si, Gyeonggi-do (KR)

(73) Assignee: SOLUS ADVANCED MATERIALS CO., LTD., Iksan-si, Jeollabuk-do (KR)

(21) Appl. No.: 18/266,975

(22) PCT Filed: Dec. 14, 2021
(86) PCT No.: PCT/KR2021/018963

§ 371 Date: Jun. 13, 2023

(30) Foreign Application Priority Data
Dec. 14, 2020 (KR) 10-2020-0174301

(51) Int. Cl. C07D 495/04; C07D 491/048; C07F 9/6561; H10K 50/11; H10K 50/15; H10K 50/16; H10K 50/17

(52) U.S. Cl. CPC C07D 495/04; C07D 491/048; C07F 9/6561; H10K 50/11; H10K 50/15; H10K 50/16; H10K 50/171

(57) ABSTRACT

A novel organic luminescent compound and an organic electroluminescent device using the novel organic luminescent compound are disclosed. The organic luminescent compound has excellent thermal stability, electrochemical stability, light emitting ability, and electron transport ability. An organic electroluminescent device includes one or more organic layers each including the organic luminescent compound and shows improved properties, for example, luminous efficiency, driving voltage, and service life characteristics.

### TECHNICAL FIELD

The present invention relates to a novel organic compound and an organic electroluminescent device using the same and, more particularly, to a compound having excellent electron transport ability; and to an organic electroluminescent device which includes one or more organic layers each including the compound and is thereby improved in terms of, for example, luminous efficiency, driving voltage, service life, and the like.

### BACKGROUND ART

Starting from Bernanose's observation of light emission from organic thin films in the 1950s, the study of organic electroluminescent (“EL”) devices led to blue electroluminescence using anthracene monocrystals in 1965, and Tang suggested in 1987 an organic EL device in a stack structure which may be divided into functional layers of hole layers and light emitting layers. Then, in order to develop high efficiency, long service life organic EL devices, organic layers each having distinctive characteristics have been introduced in the EL devices, leading to the development of specialized materials used therein.

In organic EL devices, upon application of voltage between two electrodes, holes are injected from an anode (e.g., positive electrode) to an organic layer and electrons are injected from a cathode (e.g., negative electrode) into the organic layer. Injected holes and electrons meet each other to form excitons, and light emission occurs when the excitons fall to a ground state. In such a case, materials used for the organic layer may be classified into, for example, luminescent materials, hole injection materials, hole transport materials, electron transport materials and electron injection materials depending on their function.

Luminescent materials may be classified into blue, green and red luminescent materials depending on their emission colors, and further into yellow and orange luminescent materials for realizing better natural colors. In addition, a host/dopant system may be employed in the luminescent material to increase color purity and luminous efficiency through energy transition.

Dopant materials may be classified into fluorescent dopants using organic materials and phosphorescent dopants using metal complex compounds which include heavy atoms such as Ir and Pt. In such a case, the developed phosphorescent materials may improve the luminous efficiency theoretically up to four times as compared to fluorescent materials, so attention is given to phosphorescent dopants as well as phosphorescent host materials.

To date, NPB, BCP and Alq₃, for example, are widely known as materials used in the hole injection layer, the hole transport layer, the hole blocking layer and the electron transport layer, and anthracene derivatives have been reported as luminescent materials. Particularly, metal complex compounds including Ir, such as Flrpic, Ir(ppy)₃, and (acac)Ir(btp)₂, which are known to have advantages in terms of efficiency improvement among luminescent materials, are used as blue, green and red phosphorescent dopant materials, and 4,4-dicarbazolybiphenyl (CBP) is used as a phosphorescent host material.

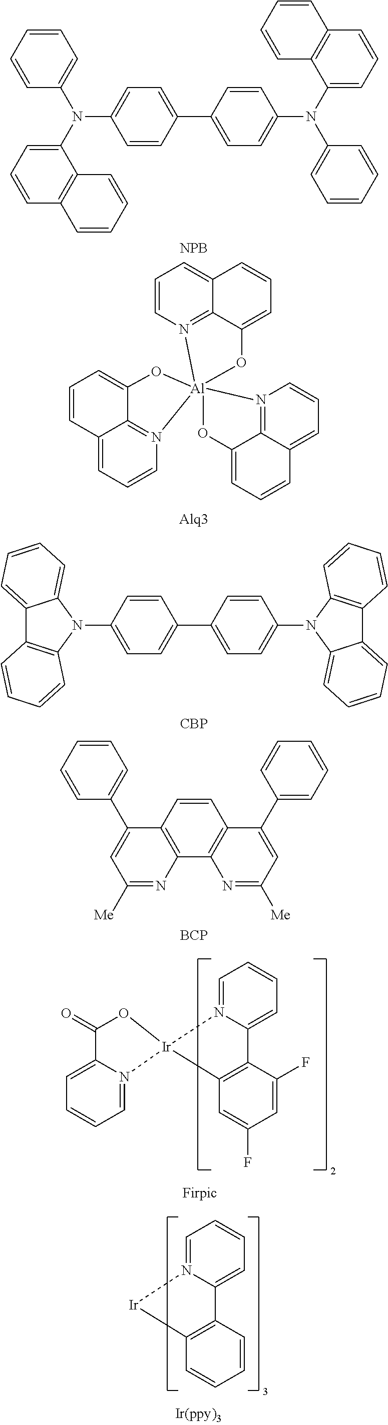

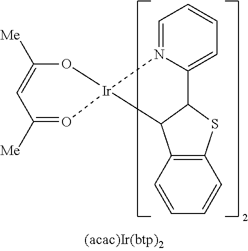

However, although conventional materials for organic layers are advantages in terms of luminescence properties, they have low glass transition temperatures, thus having poor thermal stability, and thus organic EL devices in which such conventional materials are used do not exhibit satisfactory service life characteristics. Accordingly, there is a demand for organic layer materials that are excellent in performance.

### PRIOR ART

Korean Patent Publication KR2015-7027517

Korean Patent Publication KR2019-0061314

### DETAILED DESCRIPTION OF THE INVENTION

### Technical Objectives

The present invention is directed to a novel compound applicable to an organic electroluminescent (“EL”) device having excellent characteristics such as electron injection and transport ability as well as thermal stability.

In addition, the present invention is also directed to an organic EL device including the aforementioned novel compound, thereby having low driving voltage, high luminous efficiency, and improved service life.

### Technical Means to Solve the Problem

To achieve the above objectives, the present invention provides a compound represented by the following Chemical Formula 1:

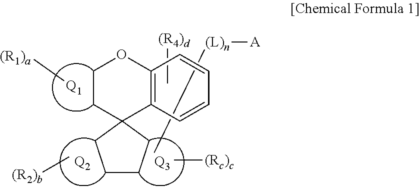

wherein in Chemical Formula 1,
Q₁ to Q₃ may be the same as or different from each other, each independently being a C₆ to C₁₀ aromatic ring;
L may be a single bond or may be selected from: a C₆ to C₁₈ arylene group and a heteroarylene group having 5 to 18 nuclear atoms;
R₁ to R₄ may be the same as or different from each other, each independently being selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms,

A may be a substituent represented by the following Chemical Formula a,

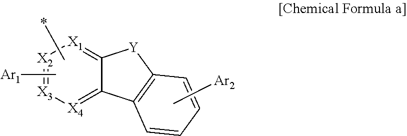

wherein in the above Chemical Formula a,
* may mean a site bonded to Chemical Formula 1,
Y may be S or O;
X₁ to X₄ may be the same as or different from each other, each independently being CH or N, provided that at least two of X₁ to X₄ are N,
Ar₁ and Ar₂ may be the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms,
each of a to d and n may be an integer in a range from 0 to 3, and
the arylene group and the heteroarylene group of L and the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphanyl group, the monoarylphosphinyl group, the diarylphosphinyl group, the arylamine group, the arylheteroarylamine group, and the heteroarylamine group of R₁ to R₄ and Ar₁ and Ar₂ may each independently be substitutable with one or more kinds of substituents selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, a C₆ to C₆₀ arylamine group, a C₅ to C₆₀ arylheteroarylamine group, and a heteroarylamine group having 5 to 60 nuclear atoms, and when the substituents are plural in number, the substituents may be the same as or different from each other.

In addition, the present invention provides an electroluminescent device including: an anode, a cathode, and one or more organic layers disposed between the anode and the cathode, wherein at least one of the one or more organic layers may include the compound represented by the above Chemical Formula 1.

### Effects of the Invention

A compound according to the present invention has excellent characteristics such as thermal stability, electron injecting/transport ability, and luminescent ability, thereby being usefully applicable as a material for an organic layer of an organic electroluminescent device.

In addition, an organic electroluminescent device of the present invention including the compound in an organic layer is significantly improved in aspects such as luminescent performance, driving voltage, service life, and efficiency, thereby being usefully applicable to, for example, a full color display panel.

### DETAILED DESCRIPTION OF THE INVENTION

Hereinafter, the present invention will be described in detail.

### <Novel Compounds>

According to the present invention, a novel compound represented by Chemical Formula 1 has a basic skeleton in which a structure in which a monocyclic or polycyclic aromatic ring such as a benzene ring or a naphthalene ring is condensed (e.g., fused) with a spiro structure (e.g., fluorene-xanthene) is used as a core, and a heterocyclic ring (e.g., BTP (benzo[4,5]thieno[3,2-d]pyrimidine), BFP (benzofuro[3,2-d]pyrimidine), etc.) as an electron withdrawing group (EWG) having high electron absorption is linked to the core directly or through various linkers L. The compound of Chemical Formula 1 has excellent electron injection and transport abilities, and accordingly, it may exhibit excellent properties as an electron transport layer material or an electron transport auxiliary layer material.

Specifically, the spiro structure is typically very excellent in electrochemical stability, has a high glass transition temperature (Tg), has excellent carrier transport ability, and particularly, has significantly excellent electron mobility, and thus the efficiency of blue light emission may be increased. In the case of a structure in which a monocyclic or polycyclic aromatic ring such as a benzene ring or a naphthalene ring is condensed with the spiro structure, a conjugation length may increase, while maintaining the original characteristics of the spiro structure, and the thermal stability of a device including the spiro structure may be improved to enhance the service life characteristics of the device. In addition, a compound having a core including the spiro structure may form more rings than compounds having fluorene as a core, thereby reducing packing of the core. Accordingly, when the compound is used as a light emitting layer, a refractive index may increase to improve the efficiency and service life of the device.

Furthermore, in the present invention, the heterocyclic ring (e.g., BTP (benzo[4,5]thieno[3,2-d]pyrimidine), BFP (benzofuro[3,2-d]pyrimidine), etc.) having an electron withdrawing (EWG) ability superior to that of the azine series is introduced to the core of the spiro structure, and accordingly, it is possible to improve the speed of electron transfer to achieve physicochemical properties more suitable for electron injection and transport.

Meanwhile, in Korean Patent Publication No. 2019-0061314, there is disclosed an organic compound used for an electron transport layer or a hole auxiliary layer, in which a core having a spiro structure is bonded to position 2 of a heterocyclic ring (e.g., BTP, BFP).

In the compound according to the present invention, the core containing the spiro structure is bonded to position 4 of the heterocyclic ring, and accordingly, the electron withdrawing property of the compound according to the present invention may be stronger than that of the compound of the prior art in which the core containing the spiro structure is bonded to position 2 of the heterocyclic ring, electron mobility may be improved, and ETL and aETL characteristics may thus be excellent.

When the compound of Chemical Formula 1 is applied as a material for an electron transport layer or an electron transport auxiliary layer, electrons may be well received from a cathode and electrons may be smoothly transported to a light emitting layer, thereby lowering a driving voltage of the device and improving efficiency and service life. Accordingly, such an organic electroluminescent (“EL”) device may maximize the performance of a full color organic EL panel.

Furthermore, HOMO and LUMO energy levels of the compound of Chemical Formula 1 may be easily adjusted according to a direction or position of a substituent, and thus electron mobility may be excellent. Accordingly, an organic EL device containing the compound may exhibit high efficiency.

As described above, since the compound represented by Chemical Formula 1 is excellent in terms of electron transport ability and luminescent characteristics, it may be used as a material for any one of a light emitting layer, an electron transport layer and an electron injection layer, which are organic layers of an organic EL device, and preferably, it may be used as a material for a blue phosphorescent light emitting layer or an electron transport layer. Accordingly, the compound represented by Chemical Formula 1 of the present invention may be used as an organic layer material of an organic EL device, preferably a light emitting layer material (green, red, and blue phosphorescent host material), an electron transport/injection layer material, a light emitting auxiliary layer material, an electron transport auxiliary layer material, and more preferably a light emitting layer material, an electron transport layer material, and an electron transport auxiliary layer material. The performance and service life characteristics of the organic EL device of the present invention including the compound of Chemical Formula 1 may be greatly improved, and the performance of a full-color organic EL panel to which the organic EL device is applied may also be maximized.

According to the present invention, the novel compound represented by Chemical Formula 1 has a basic skeleton in which a structure in which a monocyclic or polycyclic aromatic ring such as a benzene ring or a naphthalene ring is condensed (e.g., fused) with a spiro structure (e.g., fluorene-xanthene) is used as a core, and a heterocyclic ring (e.g., BTP (benzo[4,5]thieno[3,2-d]pyrimidine), BFP (benzofuro[3,2-d]pyrimidine), etc.) is linked to the core directly or through various linkers L.

In the compound represented by Chemical Formula 1, Q₁ to Q₃ are the same as or different from each other, each independently being a C₆ to C₁₀ aromatic ring. Specific examples of Q₁ to Q₃ may include a phenylene ring and a naphthalene ring. Specifically, Q₁ to Q₃ may all be C₆ aromatic rings, or any one of Q₁ to Q₃ may be a C₁₀ aromatic ring and the rest may be C₆ aromatic rings.

In the compound represented by Chemical Formula 1, a to d are integers in a range from 0 to 3.

In such a case, when each of a to d is 0, it means that each hydrogen is not substituted with substituents R₁ to R₄. In addition, when a to d is not 0, specifically, when a to d is an integer in a range from 1 to 3, the plurality of R₁ to R₄ may be the same as or different from each other, and may each independently be selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms.

In the compound represented by Chemical Formula 1, a substituent A, which is a heterocyclic ring bonded, directly or through a separate linker L, to the core in which Q₁ to Q₃ are condensed, is a kind of an electron withdrawing group (EWG) having excellent electron transport ability, and is represented by Chemical Formula a.

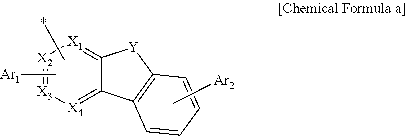

In Chemical Formula a, * means a site bonded to Chemical Formula 1, Y is S or O, X₁ to X₄ are the same as or different from each other and each independently is CH or N, provided that at least two of X₁ to X₄ are N. Since the substituent a shows EWG properties more strongly than the azine series, it exhibits excellent electron absorption characteristics and is advantageous for electron injection and transport.

In Chemical Formula a, Ar₁ and Ar₂ may be the same as or different from each other, and may each independently be selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms. Preferably, Ar₁ may be selected from: a C₆ to C₆₀ aryl group and a heteroaryl group having 5 to 60 nuclear atoms, and the aryl group and the heteroaryl group of Ar₁ may be substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents may be the same as or different from each other, and Ar₂ may be hydrogen.

In one embodiment of the substituent A, two of X₁ to X₄ may be N.

In another embodiment of the substituent A, the Chemical Formula a may be embodied as the following Chemical Formula a-1.

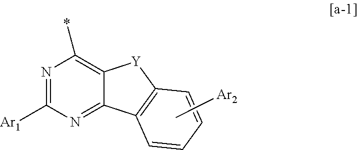

The aforementioned substituent A may be bonded to the core directly or through a separate linker L. As such, when a separate linker L is present between the substituent A and the core, a HOMO region may be expanded to give a benefit to a HOMO-LUMO distribution, and charge transfer efficiency may be increased through appropriate overlap of HOMO-LUMO.

This linker L may be a conventional divalent group linker known in the art. For example, L may be a single bond or may be selected from a C₆ to C₁₈ arylene group and a heteroarylene group having 5 to 18 nuclear atoms.

In the compound represented by Chemical Formula 1, n is an integer in a range from 0 to 3, specifically in a range from 0 to 2.

Here, when n is 0, it means that L is a single bond, and when n is an integer in a range from 1 to 3, L may be a divalent linker, and may be selected from: a C₆ to C₁₈ arylene group and a heteroarylene groups having 5 to 18 nuclear atoms.

In an example, L may be more further embodied as a linker of a single bond or selected from the following structural formulas:

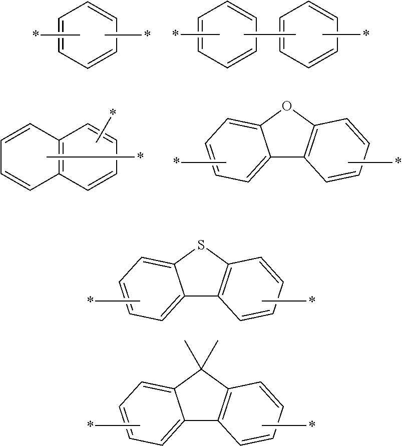

where * means a site bonded with Chemical Formula 1.

The arylene group and the heteroarylene group of L and the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphanyl group, the monoarylphosphinyl group, the diarylphosphinyl group, the arylamine group, the arylheteroarylamine group, and the heteroarylamine group of R₁ to R₄ and Ar₁ and Ar₂ may each independently be substitutable with one or more kinds of substituents selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, a C₆ to C₆₀ arylamine group, a C₅ to C₆₀ arylheteroarylamine group, and a heteroarylamine group having 5 to 60 nuclear atoms, and when the substituents are plural in number, the substituents may be the same as or different from each other.

In an example, the compound represented by Chemical Formula 1 may be further embodied as a compound represented by any one of the following Chemical Formulas 2 to 5 according to the kind and position of a ring condensed with the core. However, it is not limited thereto.

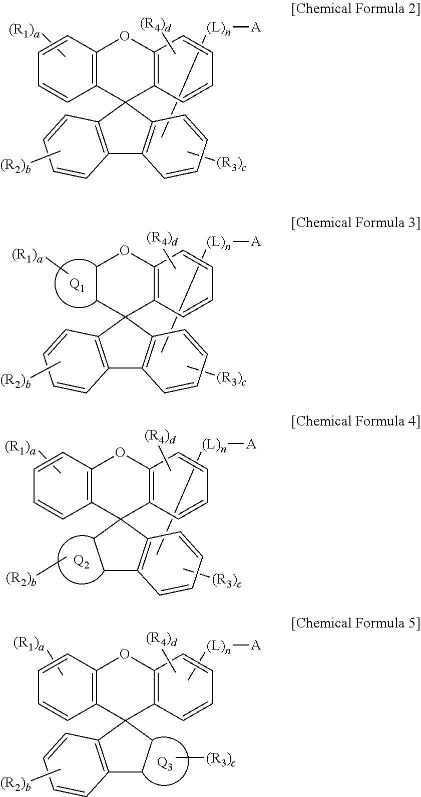

In Chemical Formulas 2 to 5,
each of Q₁ to Q₃ is a C₁₀ aromatic ring, and
L, R₁ to R₄, A, a to d, and n are as defined in Chemical Formula 1, respectively.

In an example, the compound represented by Chemical Formula 1 may be further embodied as a compound represented by any one of the following Chemical Formulas 6 to 12 according to the kind and position of the ring condensed with the core. However, it is not limited thereto.

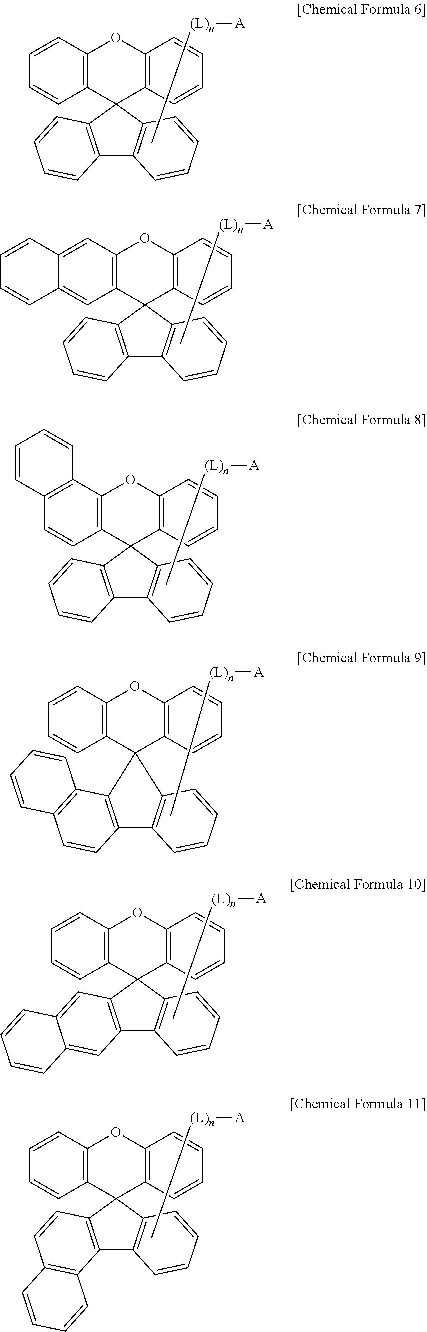

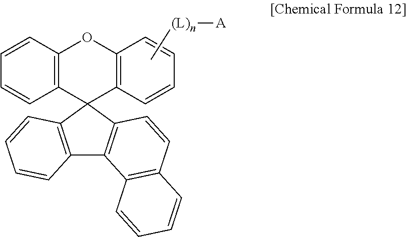

In Chemical Formulas 6 to 12, L, A and n are as defined in Chemical Formula 1, respectively.

In an example, the compound represented by Chemical Formula 1 may be further embodied as a compound represented by any one of the following Chemical Formulas 13 to 18 according to the position of the linker L bonded with the core. However, it is not limited thereto.

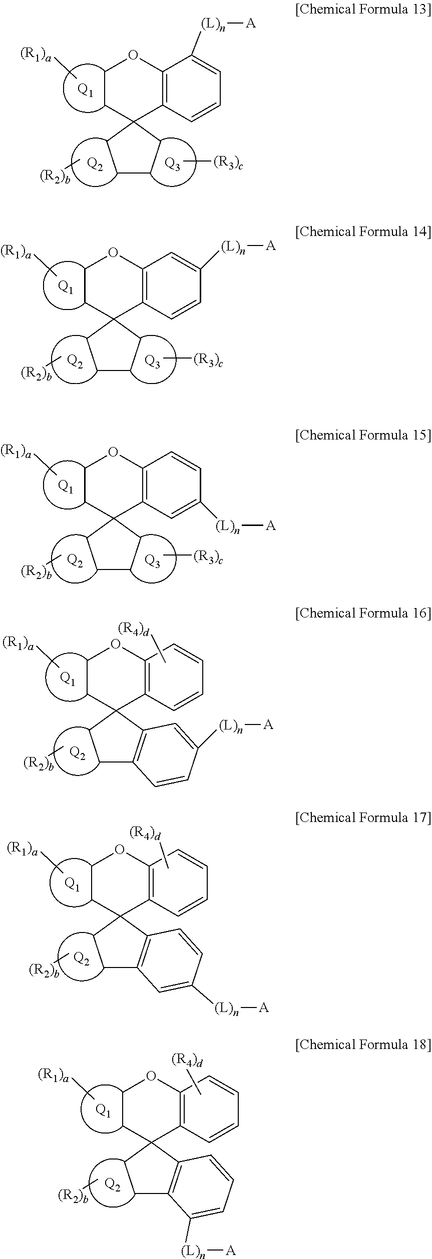

In Chemical Formulas 13 to 18, Q₁ to Q₃, L, R₁ to R₄, A, a to d, and n are as defined in Chemical Formula 1, respectively. In such a case, when the substituent A is bonded to a lower end of the spiro compound represented by Chemical Formula 1 (e.g., Formulas 16 to 18), electron mobility may be improved compared to the case where the substituent A is bonded to an upper end of the spiro compound.

The compound represented by Chemical Formula 1 according to the present invention described above may be further embodied in the following exemplary compounds, for example, Compound 1 to Compound 361. However, the compound represented by Chemical Formula 1 of the present invention is not limited to those exemplified below.

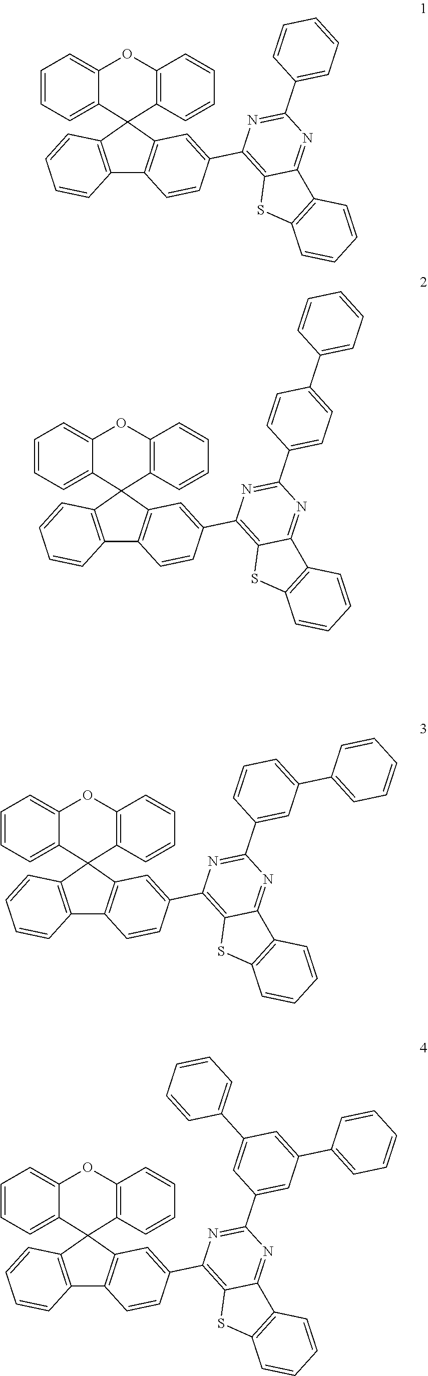

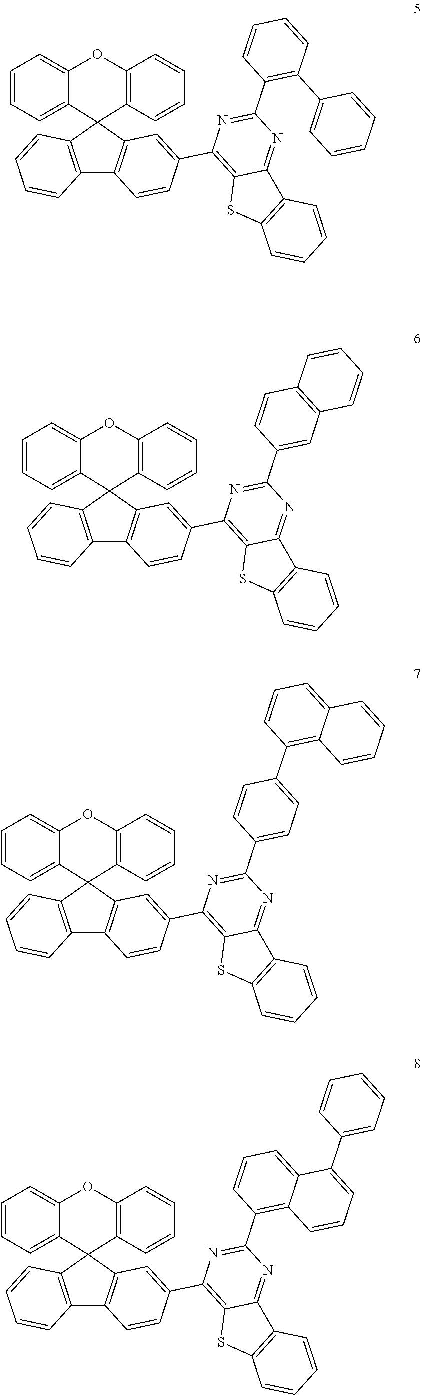 [SMILES: c1ccc(-c2ccccc2-c2nc(-c3ccc4c(c3)C3(c5ccccc5Oc5ccccc53)c3ccccc3-4)c3sc4ccccc4c3n2)cc1 ; c1ccc2c(c1)Oc1ccccc1C21c2ccccc2-c2ccc(-c3nc(-c4ccc5ccccc5c4)nc4c3sc3ccccc34)cc21 ; c1ccc2c(c1)Oc1ccccc1C21c2ccccc2-c2ccc(-c3nc(-c4ccc(-c5cccc6ccccc56)cc4)nc4c3sc3ccccc34)cc21 ; c1ccc(-c2cccc3c(-c4nc(-c5ccc6c(c5)C5(c7ccccc7Oc7ccccc75)c5ccccc5-6)c5sc6ccccc6c5n4)cccc23)cc1]

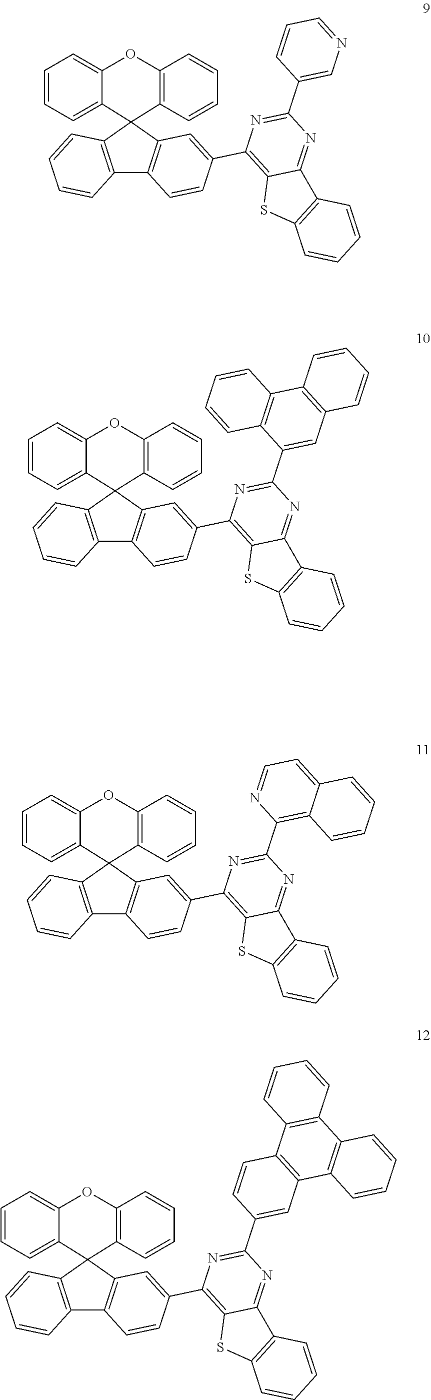 [SMILES: c1cncc(-c2nc(-c3ccc4c(c3)C3(c5ccccc5Oc5ccccc53)c3ccccc3-4)c3sc4ccccc4c3n2)c1 ; c1ccc2c(c1)Oc1ccccc1C21c2ccccc2-c2ccc(-c3nc(-c4cc5ccccc5c5ccccc45)nc4c3sc3ccccc34)cc21 ; c1ccc2c(c1)Oc1ccccc1C21c2ccccc2-c2ccc(-c3nc(-c4nccc5ccccc45)nc4c3sc3ccccc34)cc21 ; c1ccc2c(c1)Oc1ccccc1C21c2ccccc2-c2ccc(-c3nc(-c4ccc5c6ccccc6c6ccccc6c5c4)nc4c3sc3ccccc34)cc21]

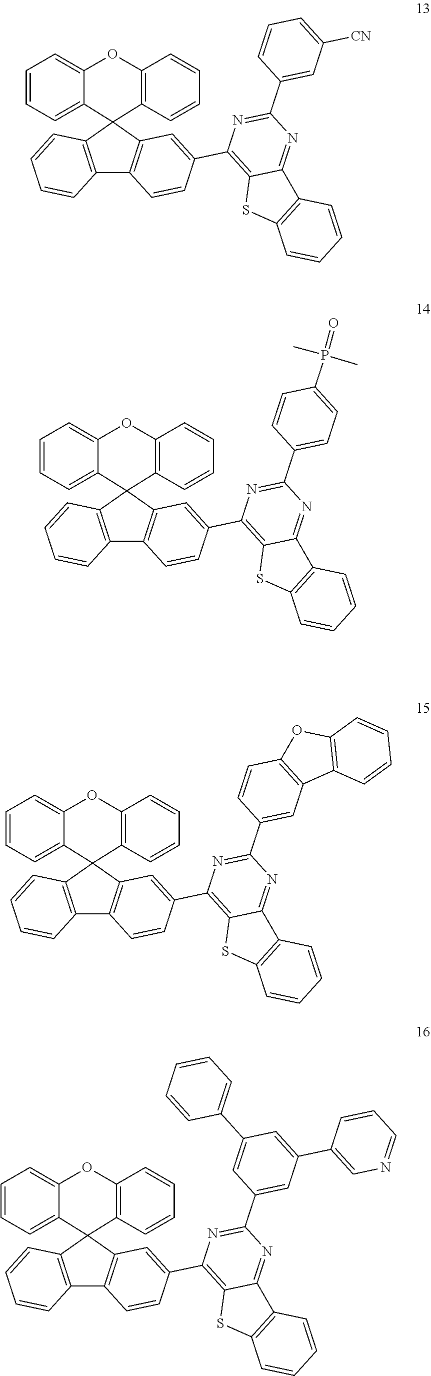 [SMILES: N#Cc1cccc(-c2nc(-c3ccc4c(c3)C3(c5ccccc5Oc5ccccc53)c3ccccc3-4)c3sc4ccccc4c3n2)c1 ; CP(C)(=O)c1ccc(-c2nc(-c3ccc4c(c3)C3(c5ccccc5Oc5ccccc53)c3ccccc3-4)c3sc4ccccc4c3n2)cc1 ; c1ccc2c(c1)Oc1ccccc1C21c2ccccc2-c2ccc(-c3nc(-c4ccc5oc6ccccc6c5c4)nc4c3sc3ccccc34)cc21 ; c1ccc(-c2cc(-c3cccnc3)cc(-c3nc(-c4ccc5c(c4)C4(c6ccccc6Oc6ccccc64)c4ccccc4-5)c4sc5ccccc5c4n3)c2)cc1]

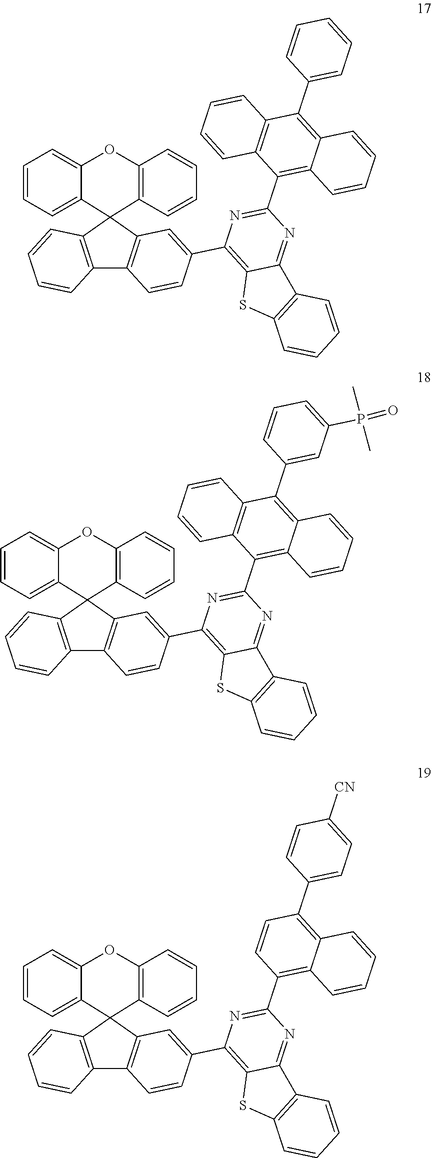

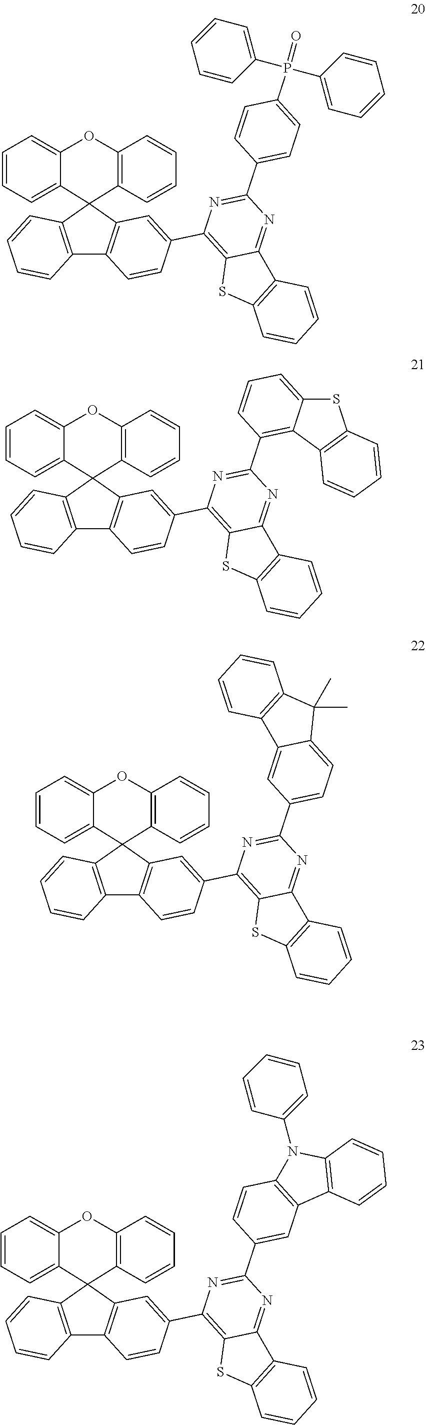

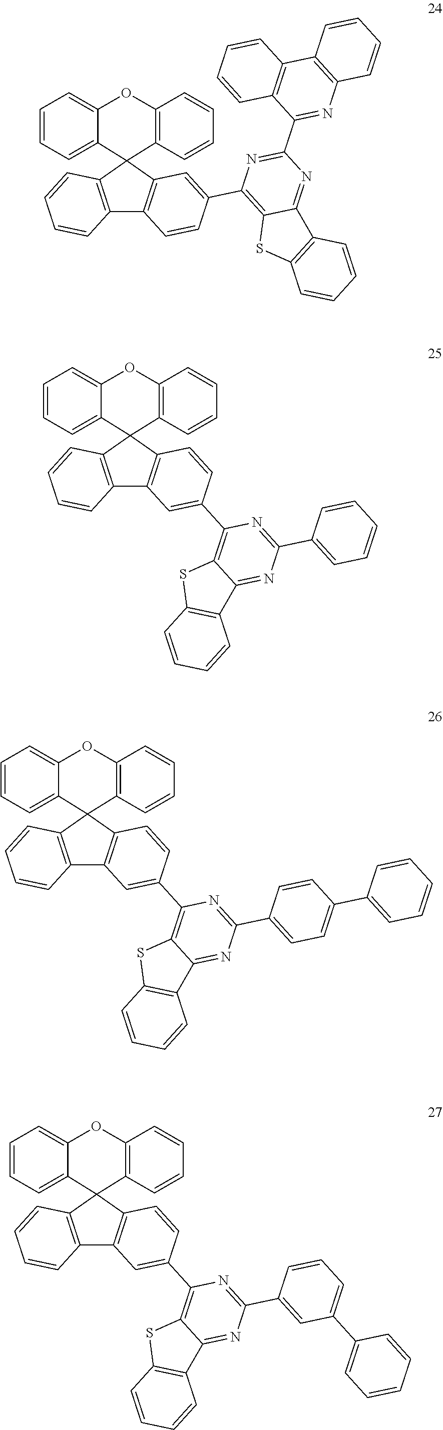 [SMILES: c1ccc2c(c1)Oc1ccccc1C21c2ccccc2-c2ccc(-c3nc(-c4nc5ccccc5c5ccccc45)nc4c3sc3ccccc34)cc21 ; c1ccc(-c2nc(-c3ccc4c(c3)-c3ccccc3C43c4ccccc4Oc4ccccc43)c3sc4ccccc4c3n2)cc1 ; c1ccc(-c2ccc(-c3nc(-c4ccc5c(c4)-c4ccccc4C54c5ccccc5Oc5ccccc54)c4sc5ccccc5c4n3)cc2)cc1 ; c1ccc(-c2cccc(-c3nc(-c4ccc5c(c4)-c4ccccc4C54c5ccccc5Oc5ccccc54)c4sc5ccccc5c4n3)c2)cc1]

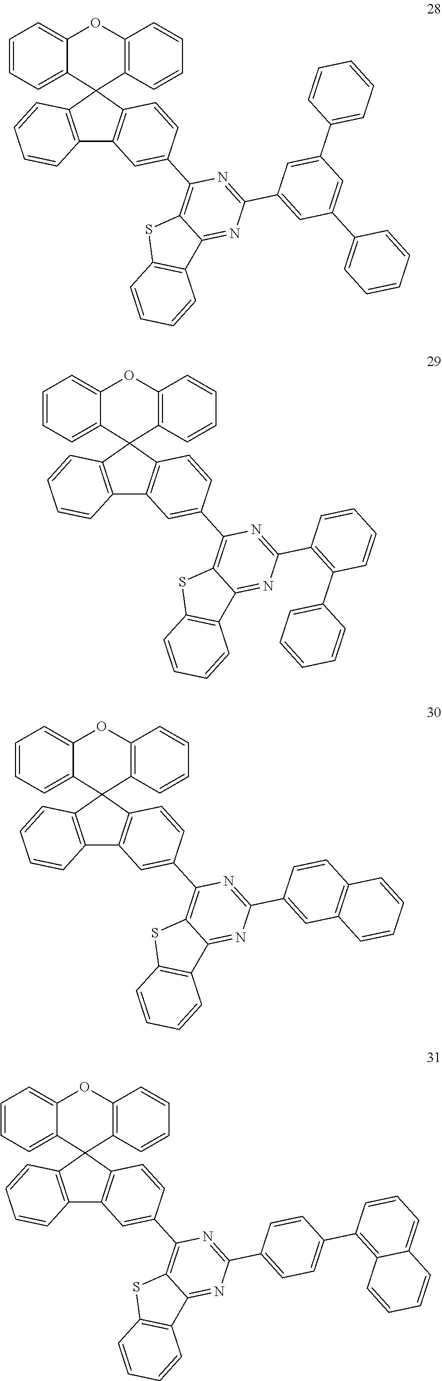 [SMILES: c1ccc(-c2cc(-c3ccccc3)cc(-c3nc(-c4ccc5c(c4)-c4ccccc4C54c5ccccc5Oc5ccccc54)c4sc5ccccc5c4n3)c2)cc1 ; c1ccc(-c2ccccc2-c2nc(-c3ccc4c(c3)-c3ccccc3C43c4ccccc4Oc4ccccc43)c3sc4ccccc4c3n2)cc1 ; c1ccc2c(c1)Oc1ccccc1C21c2ccccc2-c2cc(-c3nc(-c4ccc5ccccc5c4)nc4c3sc3ccccc34)ccc21 ; c1ccc2c(c1)Oc1ccccc1C21c2ccccc2-c2cc(-c3nc(-c4ccc(-c5cccc6ccccc56)cc4)nc4c3sc3ccccc34)ccc21]

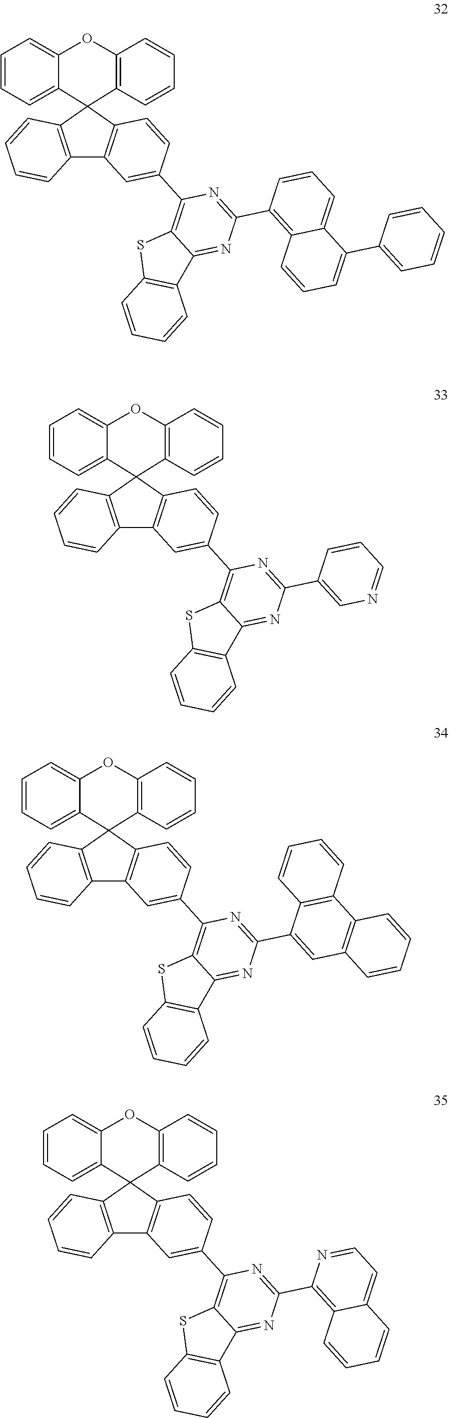 [SMILES: c1ccc(-c2cccc3c(-c4nc(-c5ccc6c(c5)-c5ccccc5C65c6ccccc6Oc6ccccc65)c5sc6ccccc6c5n4)cccc23)cc1 ; c1cncc(-c2nc(-c3ccc4c(c3)-c3ccccc3C43c4ccccc4Oc4ccccc43)c3sc4ccccc4c3n2)c1 ; c1ccc2c(c1)Oc1ccccc1C21c2ccccc2-c2cc(-c3nc(-c4cc5ccccc5c5ccccc45)nc4c3sc3ccccc34)ccc21 ; c1ccc2c(c1)Oc1ccccc1C21c2ccccc2-c2cc(-c3nc(-c4nccc5ccccc45)nc4c3sc3ccccc34)ccc21]

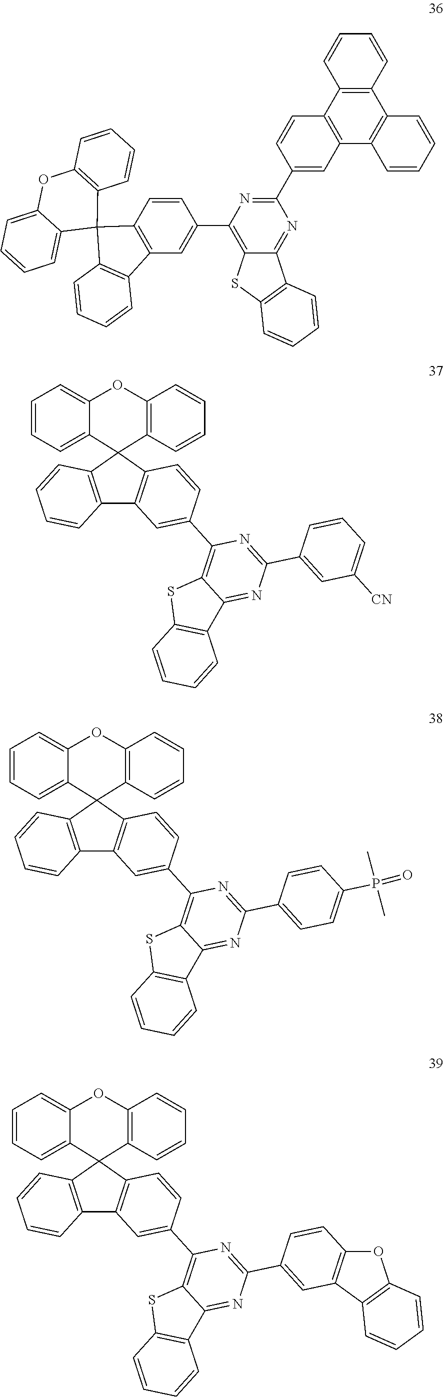 [SMILES: c1ccc2c(c1)Oc1ccccc1C21c2ccccc2-c2cc(-c3nc(-c4ccc5c6ccccc6c6ccccc6c5c4)nc4c3sc3ccccc34)ccc21 ; N#Cc1cccc(-c2nc(-c3ccc4c(c3)-c3ccccc3C43c4ccccc4Oc4ccccc43)c3sc4ccccc4c3n2)c1 ; CP(C)(=O)c1ccc(-c2nc(-c3ccc4c(c3)-c3ccccc3C43c4ccccc4Oc4ccccc43)c3sc4ccccc4c3n2)cc1 ; c1ccc2c(c1)Oc1ccccc1C21c2ccccc2-c2cc(-c3nc(-c4ccc5oc6ccccc6c5c4)nc4c3sc3ccccc34)ccc21]

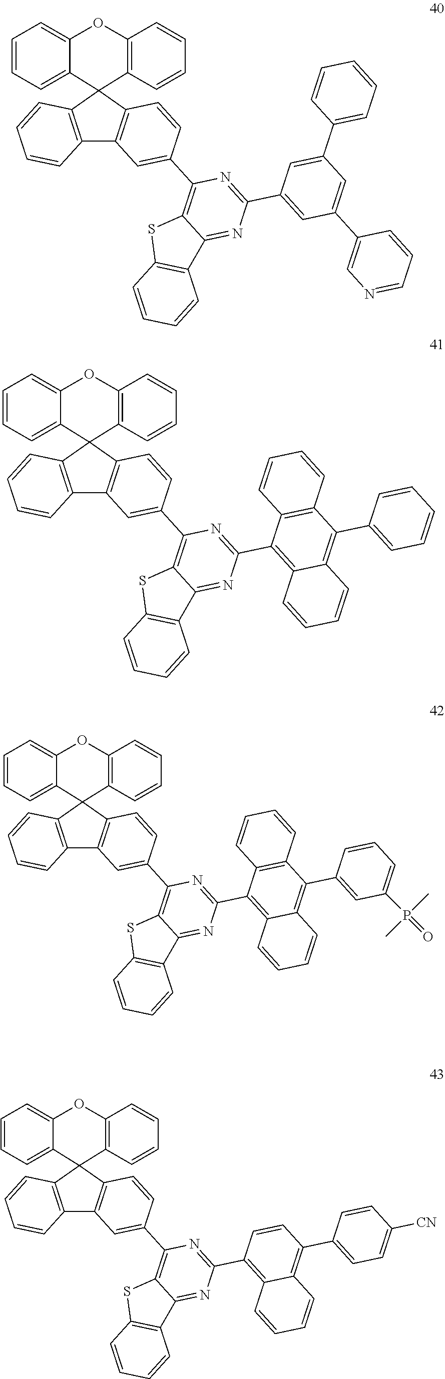 [SMILES: c1ccc(-c2cc(-c3cccnc3)cc(-c3nc(-c4ccc5c(c4)-c4ccccc4C54c5ccccc5Oc5ccccc54)c4sc5ccccc5c4n3)c2)cc1 ; c1ccc(-c2c3ccccc3c(-c3nc(-c4ccc5c(c4)-c4ccccc4C54c5ccccc5Oc5ccccc54)c4sc5ccccc5c4n3)c3ccccc23)cc1 ; CP(C)(=O)c1cccc(-c2c3ccccc3c(-c3nc(-c4ccc5c(c4)-c4ccccc4C54c5ccccc5Oc5ccccc54)c4sc5ccccc5c4n3)c3ccccc23)c1 ; N#Cc1ccc(-c2ccc(-c3nc(-c4ccc5c(c4)-c4ccccc4C54c5ccccc5Oc5ccccc54)c4sc5ccccc5c4n3)c3ccccc23)cc1]

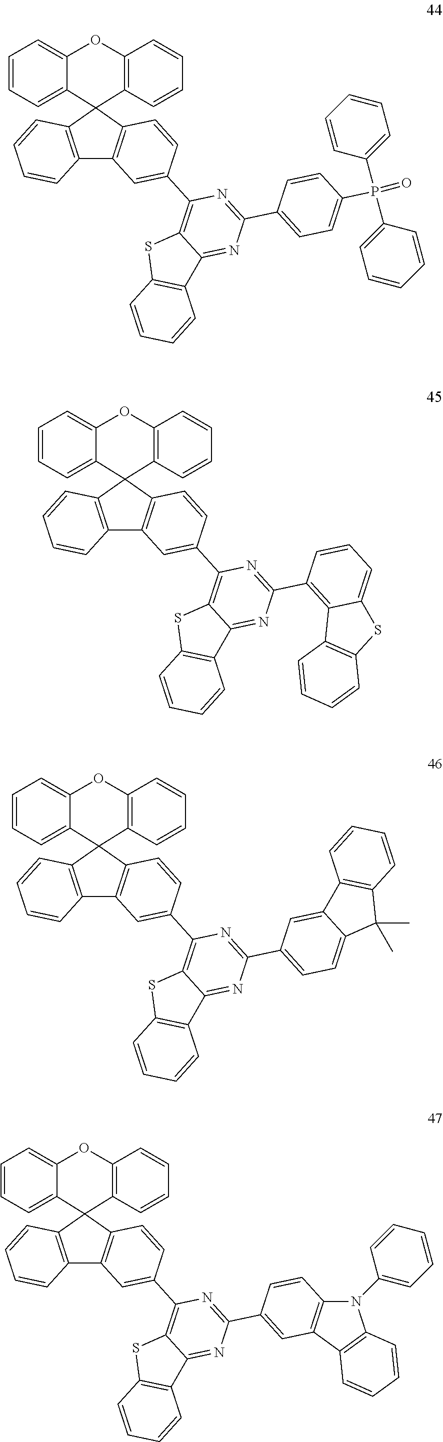 [SMILES: O=P(c1ccccc1)(c1ccccc1)c1ccc(-c2nc(-c3ccc4c(c3)-c3ccccc3C43c4ccccc4Oc4ccccc43)c3sc4ccccc4c3n2)cc1 ; c1ccc2c(c1)Oc1ccccc1C21c2ccccc2-c2cc(-c3nc(-c4cccc5sc6ccccc6c45)nc4c3sc3ccccc34)ccc21 ; CC1(C)c2ccccc2-c2cc(-c3nc(-c4ccc5c(c4)-c4ccccc4C54c5ccccc5Oc5ccccc54)c4sc5ccccc5c4n3)ccc21 ; c1ccc(-n2c3ccccc3c3cc(-c4nc(-c5ccc6c(c5)-c5ccccc5C65c6ccccc6Oc6ccccc65)c5sc6ccccc6c5n4)ccc32)cc1]

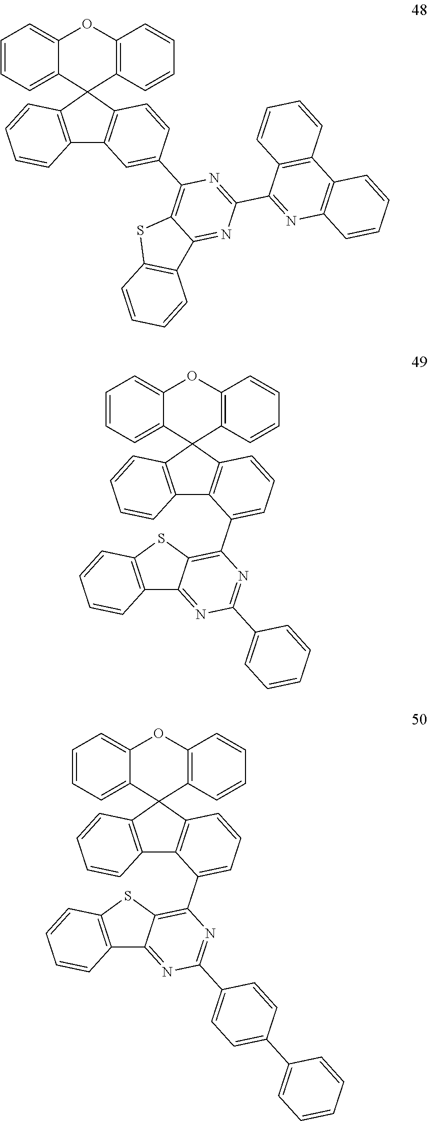 [SMILES: c1ccc2c(c1)Oc1ccccc1C21c2ccccc2-c2cc(-c3nc(-c4nc5ccccc5c5ccccc45)nc4c3sc3ccccc34)ccc21 ; c1ccc(-c2nc(-c3cccc4c3-c3ccccc3C43c4ccccc4Oc4ccccc43)c3sc4ccccc4c3n2)cc1 ; c1ccc(-c2ccc(-c3nc(-c4cccc5c4-c4ccccc4C54c5ccccc5Oc5ccccc54)c4sc5ccccc5c4n3)cc2)cc1]

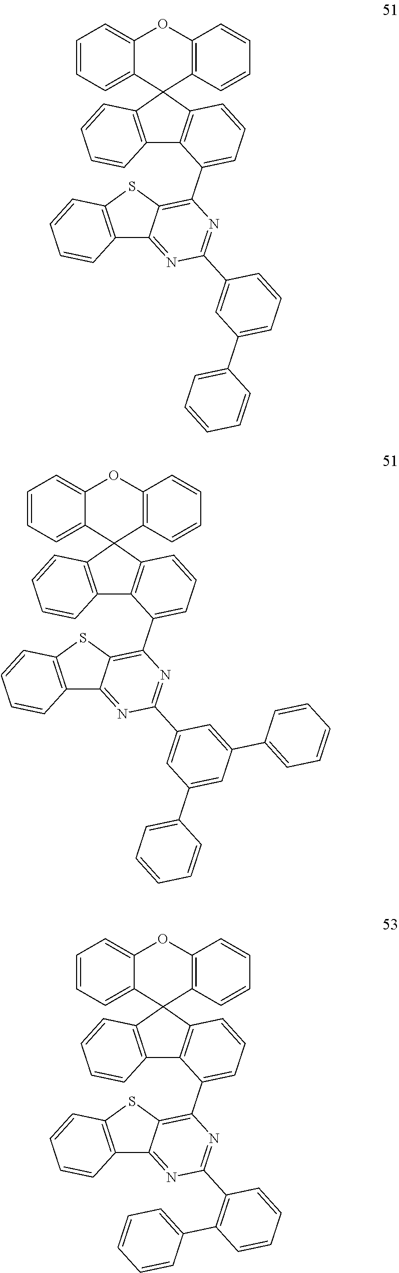 [SMILES: c1ccc(-c2cccc(-c3nc(-c4cccc5c4-c4ccccc4C54c5ccccc5Oc5ccccc54)c4sc5ccccc5c4n3)c2)cc1 ; c1ccc(-c2cc(-c3ccccc3)cc(-c3nc(-c4cccc5c4-c4ccccc4C54c5ccccc5Oc5ccccc54)c4sc5ccccc5c4n3)c2)cc1 ; c1ccc(-c2ccccc2-c2nc(-c3cccc4c3-c3ccccc3C43c4ccccc4Oc4ccccc43)c3sc4ccccc4c3n2)cc1]

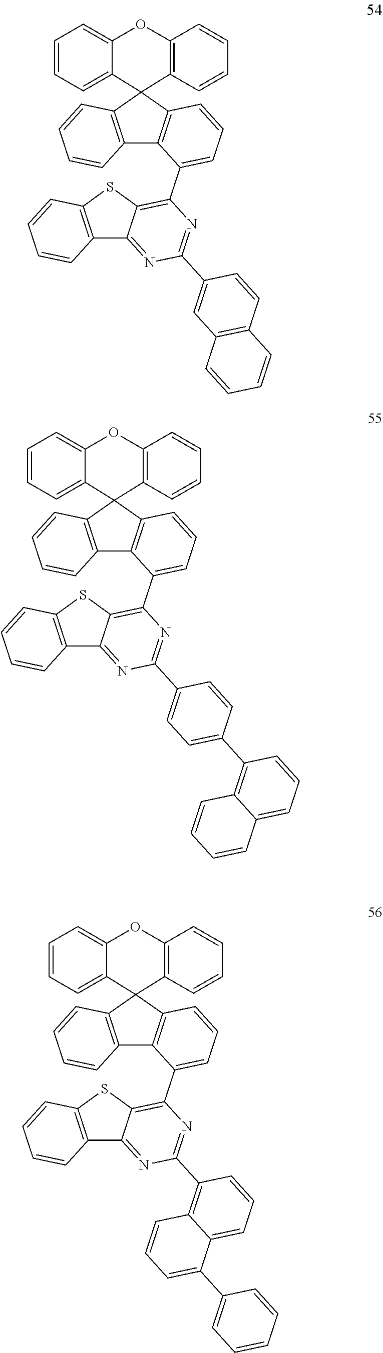 [SMILES: c1ccc2c(c1)Oc1ccccc1C21c2ccccc2-c2c(-c3nc(-c4ccc5ccccc5c4)nc4c3sc3ccccc34)cccc21 ; c1ccc2c(c1)Oc1ccccc1C21c2ccccc2-c2c(-c3nc(-c4ccc(-c5cccc6ccccc56)cc4)nc4c3sc3ccccc34)cccc21 ; c1ccc(-c2cccc3c(-c4nc(-c5cccc6c5-c5ccccc5C65c6ccccc6Oc6ccccc65)c5sc6ccccc6c5n4)cccc23)cc1]

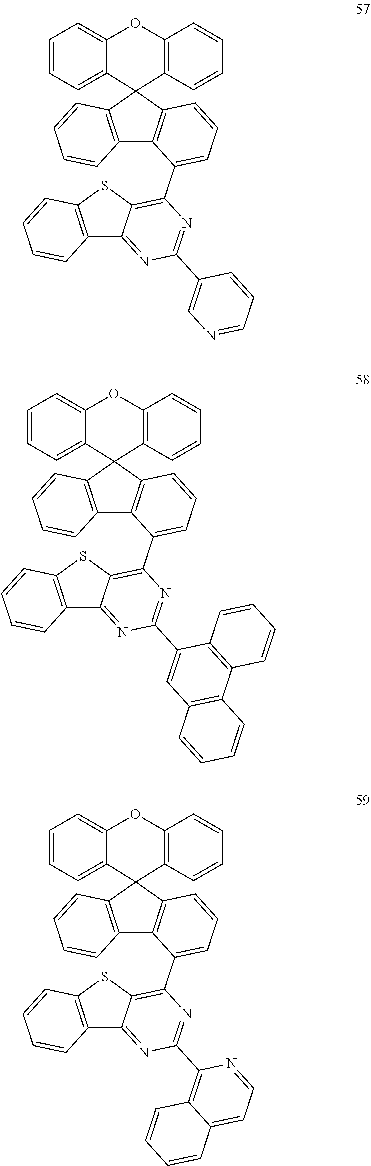 [SMILES: c1cncc(-c2nc(-c3cccc4c3-c3ccccc3C43c4ccccc4Oc4ccccc43)c3sc4ccccc4c3n2)c1 ; c1ccc2c(c1)Oc1ccccc1C21c2ccccc2-c2c(-c3nc(-c4cc5ccccc5c5ccccc45)nc4c3sc3ccccc34)cccc21 ; c1ccc2c(c1)Oc1ccccc1C21c2ccccc2-c2c(-c3nc(-c4nccc5ccccc45)nc4c3sc3ccccc34)cccc21]

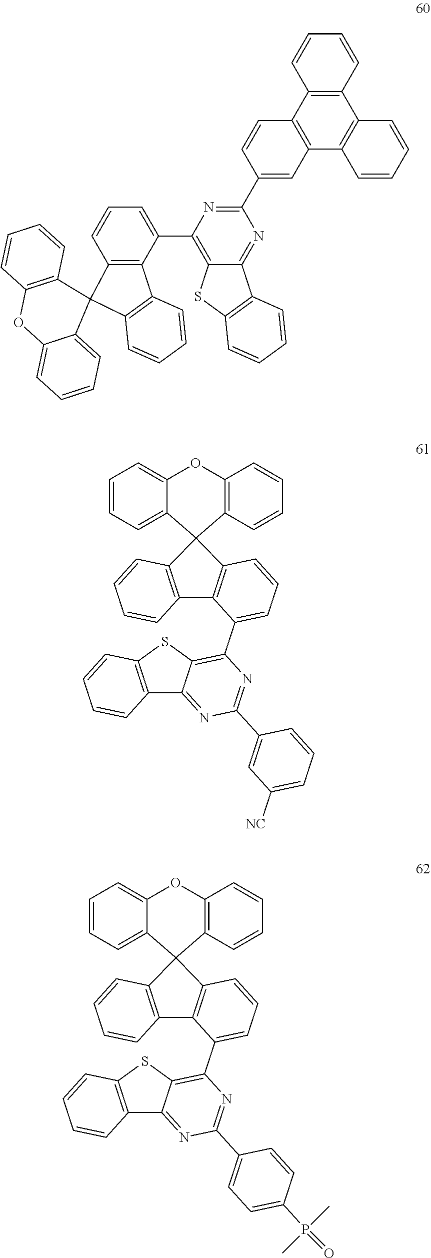 [SMILES: c1ccc2c(c1)Oc1ccccc1C21c2ccccc2-c2c(-c3nc(-c4ccc5c6ccccc6c6ccccc6c5c4)nc4c3sc3ccccc34)cccc21 ; N#Cc1cccc(-c2nc(-c3cccc4c3-c3ccccc3C43c4ccccc4Oc4ccccc43)c3sc4ccccc4c3n2)c1 ; CP(C)(=O)c1ccc(-c2nc(-c3cccc4c3-c3ccccc3C43c4ccccc4Oc4ccccc43)c3sc4ccccc4c3n2)cc1]

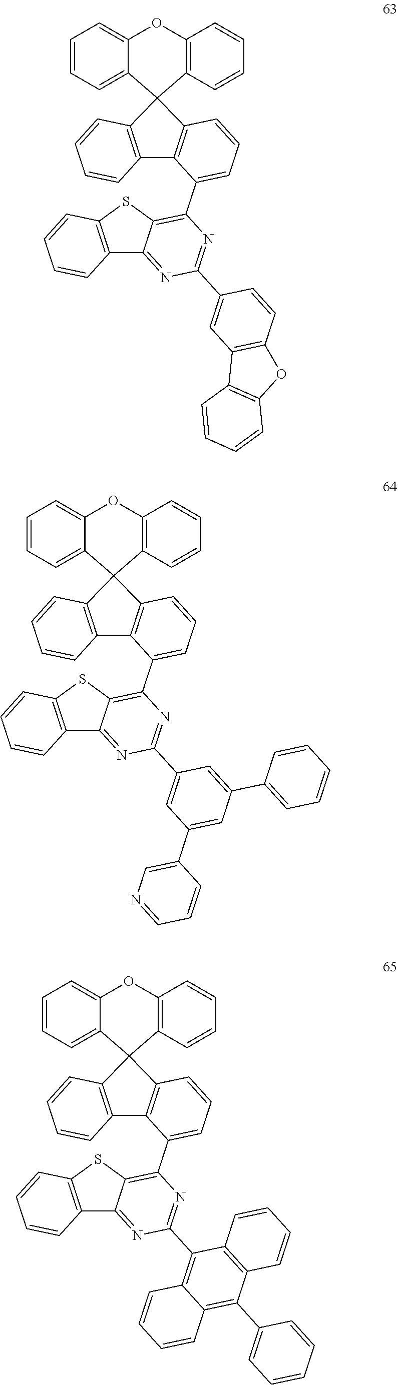 [SMILES: c1ccc2c(c1)Oc1ccccc1C21c2ccccc2-c2c(-c3nc(-c4ccc5oc6ccccc6c5c4)nc4c3sc3ccccc34)cccc21 ; c1ccc(-c2cc(-c3cccnc3)cc(-c3nc(-c4cccc5c4-c4ccccc4C54c5ccccc5Oc5ccccc54)c4sc5ccccc5c4n3)c2)cc1 ; c1ccc(-c2c3ccccc3c(-c3nc(-c4cccc5c4-c4ccccc4C54c5ccccc5Oc5ccccc54)c4sc5ccccc5c4n3)c3ccccc23)cc1]

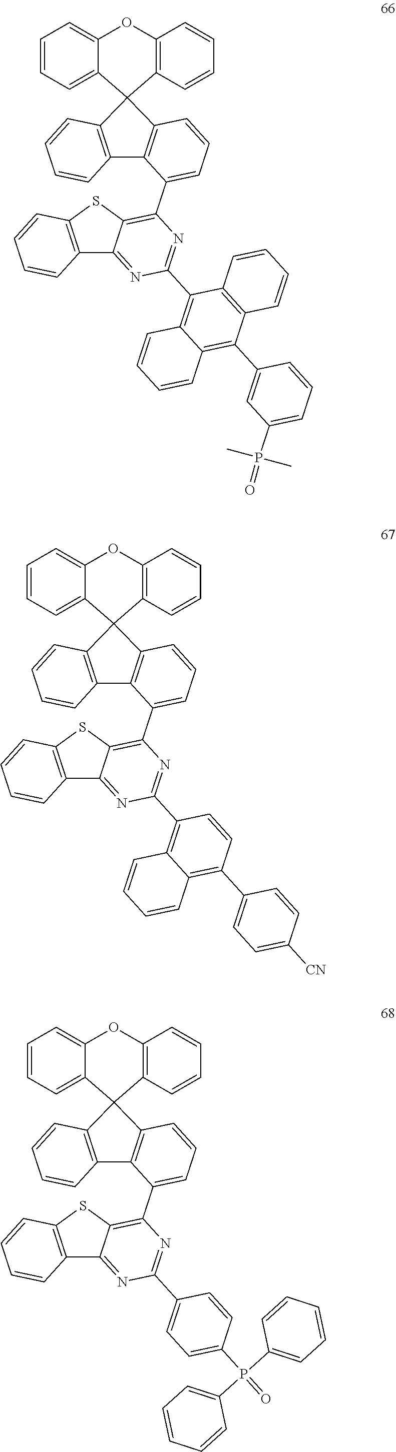 [SMILES: CP(C)(=O)c1cccc(-c2c3ccccc3c(-c3nc(-c4cccc5c4-c4ccccc4C54c5ccccc5Oc5ccccc54)c4sc5ccccc5c4n3)c3ccccc23)c1 ; N#Cc1ccc(-c2ccc(-c3nc(-c4cccc5c4-c4ccccc4C54c5ccccc5Oc5ccccc54)c4sc5ccccc5c4n3)c3ccccc23)cc1 ; O=P(c1ccccc1)(c1ccccc1)c1ccc(-c2nc(-c3cccc4c3-c3ccccc3C43c4ccccc4Oc4ccccc43)c3sc4ccccc4c3n2)cc1]

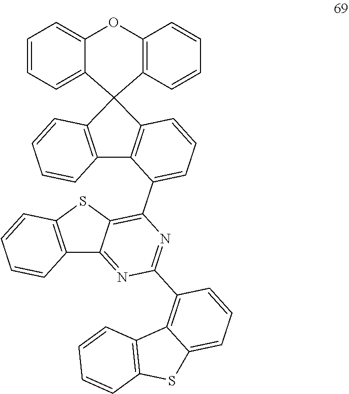 [SMILES: c1ccc2c(c1)Oc1ccccc1C21c2ccccc2-c2c(-c3nc(-c4cccc5sc6ccccc6c45)nc4c3sc3ccccc34)cccc21]

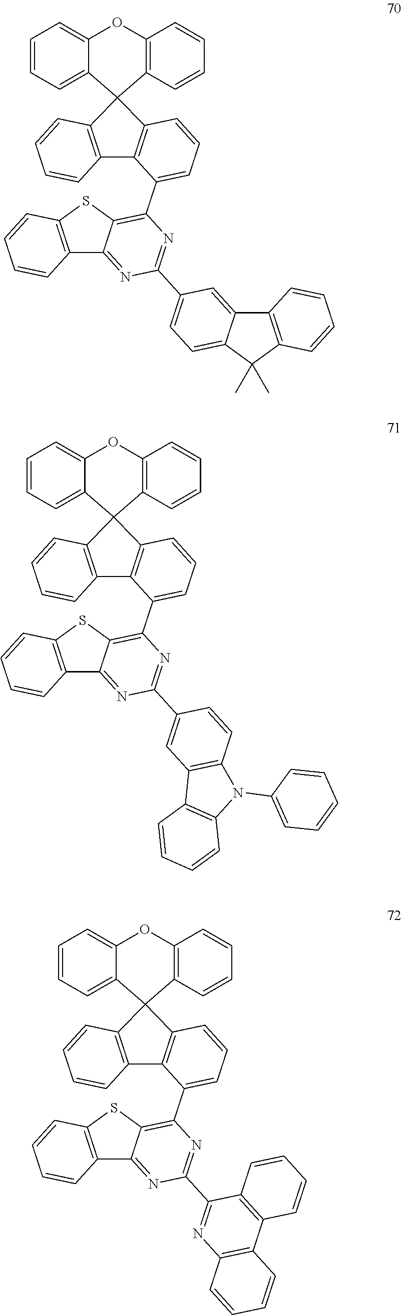 [SMILES: CC1(C)c2ccccc2-c2cc(-c3nc(-c4cccc5c4-c4ccccc4C54c5ccccc5Oc5ccccc54)c4sc5ccccc5c4n3)ccc21 ; c1ccc(-n2c3ccccc3c3cc(-c4nc(-c5cccc6c5-c5ccccc5C65c6ccccc6Oc6ccccc65)c5sc6ccccc6c5n4)ccc32)cc1 ; c1ccc2c(c1)Oc1ccccc1C21c2ccccc2-c2c(-c3nc(-c4nc5ccccc5c5ccccc45)nc4c3sc3ccccc34)cccc21]

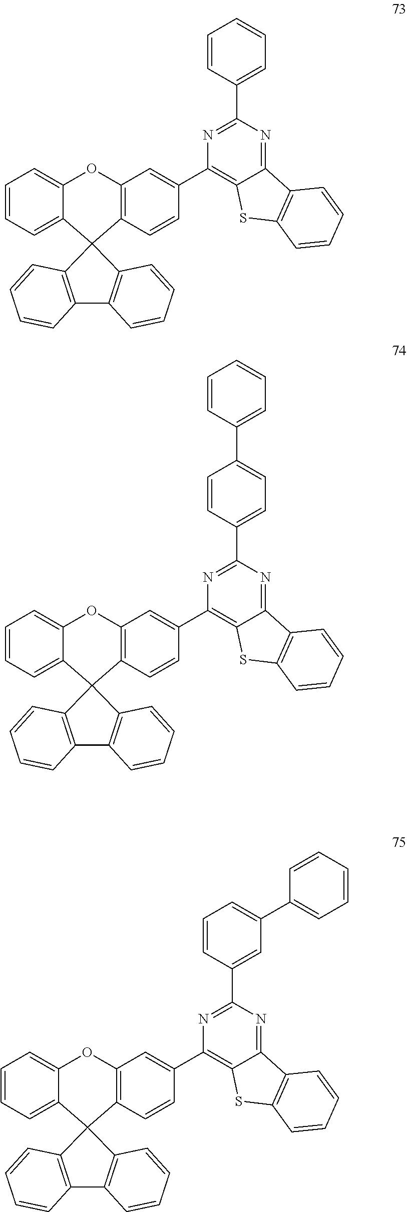 [SMILES: c1ccc(-c2nc(-c3ccc4c(c3)Oc3ccccc3C43c4ccccc4-c4ccccc43)c3sc4ccccc4c3n2)cc1 ; c1ccc(-c2ccc(-c3nc(-c4ccc5c(c4)Oc4ccccc4C54c5ccccc5-c5ccccc54)c4sc5ccccc5c4n3)cc2)cc1 ; c1ccc(-c2cccc(-c3nc(-c4ccc5c(c4)Oc4ccccc4C54c5ccccc5-c5ccccc54)c4sc5ccccc5c4n3)c2)cc1]

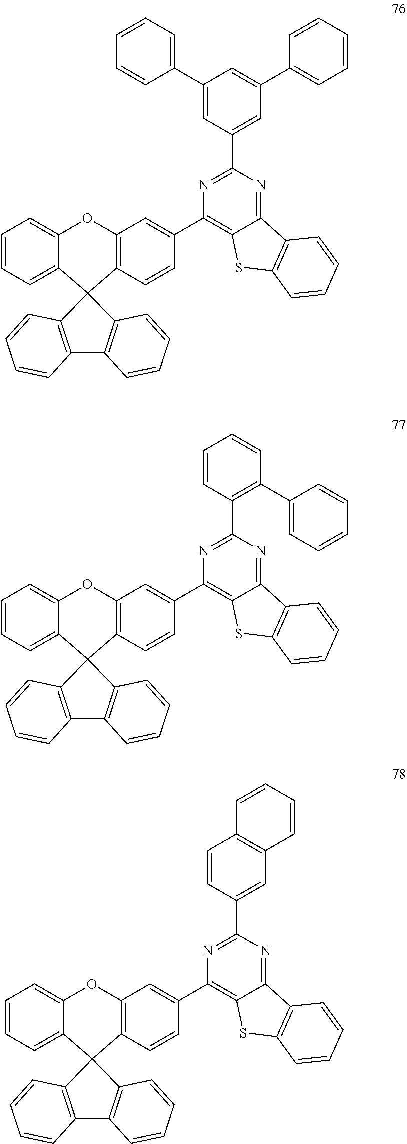 [SMILES: c1ccc(-c2cc(-c3ccccc3)cc(-c3nc(-c4ccc5c(c4)Oc4ccccc4C54c5ccccc5-c5ccccc54)c4sc5ccccc5c4n3)c2)cc1 ; c1ccc(-c2ccccc2-c2nc(-c3ccc4c(c3)Oc3ccccc3C43c4ccccc4-c4ccccc43)c3sc4ccccc4c3n2)cc1 ; c1ccc2c(c1)Oc1cc(-c3nc(-c4ccc5ccccc5c4)nc4c3sc3ccccc34)ccc1C21c2ccccc2-c2ccccc21]

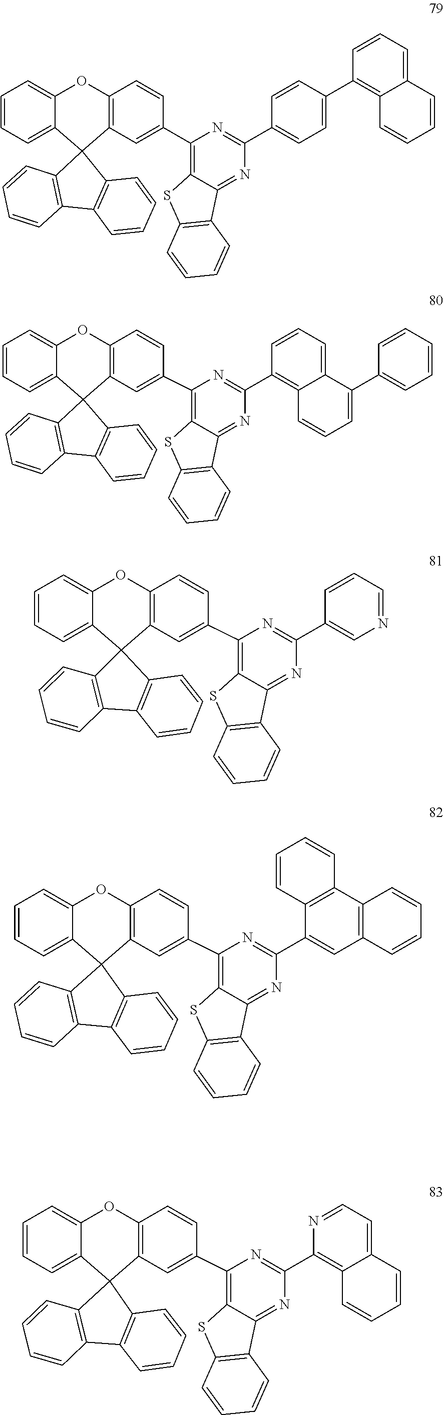 [SMILES: c1ccc2c(c1)Oc1ccc(-c3nc(-c4ccc(-c5cccc6ccccc56)cc4)nc4c3sc3ccccc34)cc1C21c2ccccc2-c2ccccc21 ; c1ccc(-c2cccc3c(-c4nc(-c5ccc6c(c5)C5(c7ccccc7O6)c6ccccc6-c6ccccc65)c5sc6ccccc6c5n4)cccc23)cc1 ; c1cncc(-c2nc(-c3ccc4c(c3)C3(c5ccccc5O4)c4ccccc4-c4ccccc43)c3sc4ccccc4c3n2)c1 ; c1ccc2c(c1)Oc1ccc(-c3nc(-c4cc5ccccc5c5ccccc45)nc4c3sc3ccccc34)cc1C21c2ccccc2-c2ccccc21 ; c1ccc2c(c1)Oc1ccc(-c3nc(-c4nccc5ccccc45)nc4c3sc3ccccc34)cc1C21c2ccccc2-c2ccccc21]

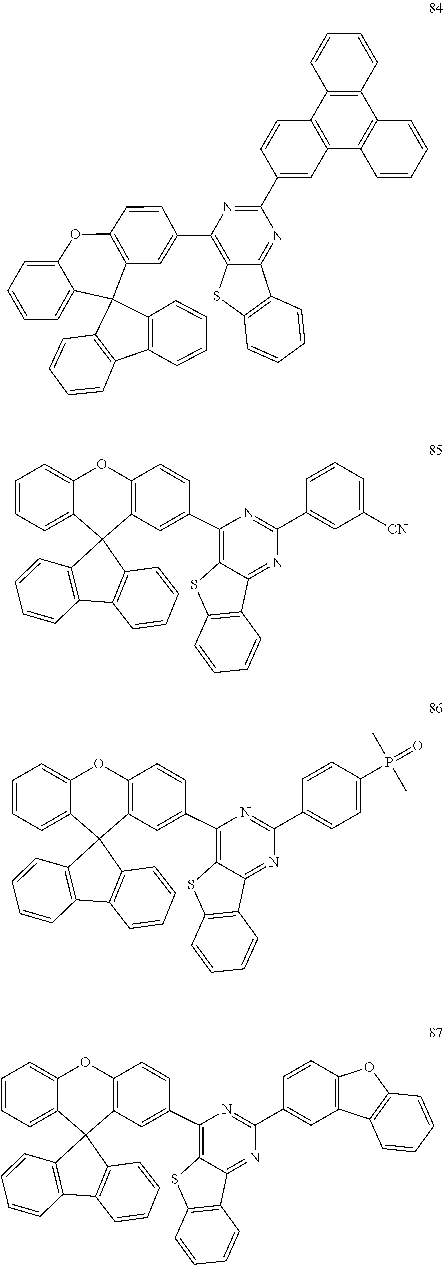 [SMILES: c1ccc2c(c1)Oc1ccc(-c3nc(-c4ccc5c6ccccc6c6ccccc6c5c4)nc4c3sc3ccccc34)cc1C21c2ccccc2-c2ccccc21 ; N#Cc1cccc(-c2nc(-c3ccc4c(c3)C3(c5ccccc5O4)c4ccccc4-c4ccccc43)c3sc4ccccc4c3n2)c1 ; CP(C)(=O)c1ccc(-c2nc(-c3ccc4c(c3)C3(c5ccccc5O4)c4ccccc4-c4ccccc43)c3sc4ccccc4c3n2)cc1 ; c1ccc2c(c1)Oc1ccc(-c3nc(-c4ccc5oc6ccccc6c5c4)nc4c3sc3ccccc34)cc1C21c2ccccc2-c2ccccc21]

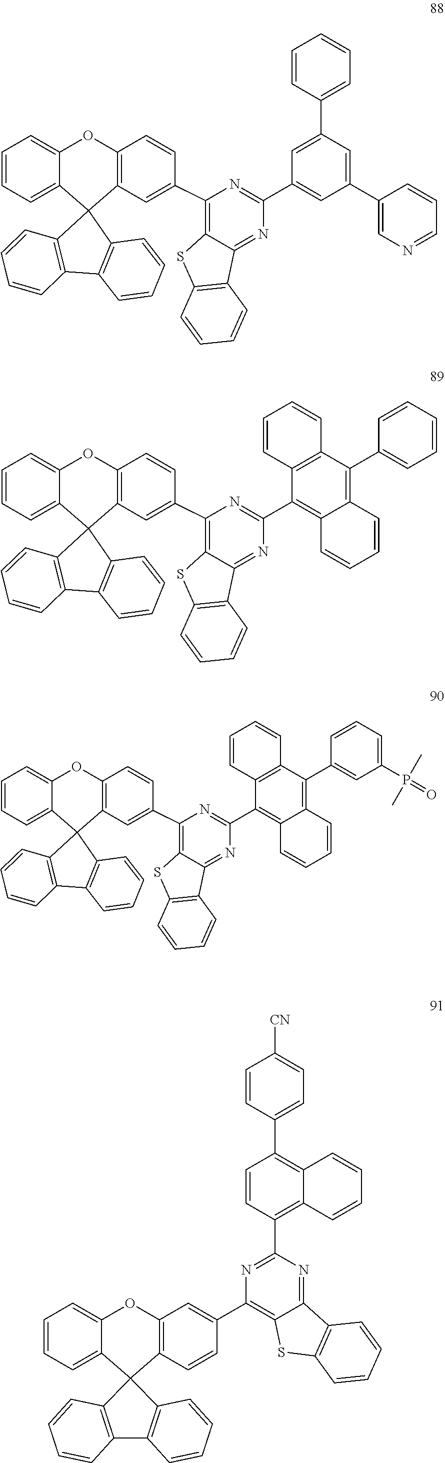 [SMILES: c1ccc(-c2cc(-c3cccnc3)cc(-c3nc(-c4ccc5c(c4)C4(c6ccccc6O5)c5ccccc5-c5ccccc54)c4sc5ccccc5c4n3)c2)cc1 ; c1ccc(-c2c3ccccc3c(-c3nc(-c4ccc5c(c4)C4(c6ccccc6O5)c5ccccc5-c5ccccc54)c4sc5ccccc5c4n3)c3ccccc23)cc1 ; CP(C)(=O)c1cccc(-c2c3ccccc3c(-c3nc(-c4ccc5c(c4)C4(c6ccccc6O5)c5ccccc5-c5ccccc54)c4sc5ccccc5c4n3)c3ccccc23)c1 ; N#Cc1ccc(-c2ccc(-c3nc(-c4ccc5c(c4)Oc4ccccc4C54c5ccccc5-c5ccccc54)c4sc5ccccc5c4n3)c3ccccc23)cc1]

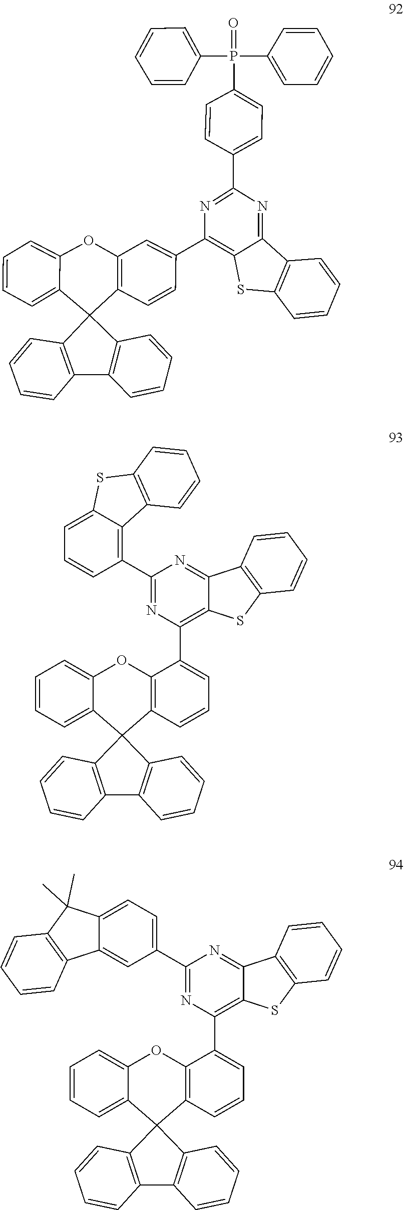 [SMILES: O=P(c1ccccc1)(c1ccccc1)c1ccc(-c2nc(-c3ccc4c(c3)Oc3ccccc3C43c4ccccc4-c4ccccc43)c3sc4ccccc4c3n2)cc1 ; c1ccc2c(c1)Oc1c(-c3nc(-c4cccc5sc6ccccc6c45)nc4c3sc3ccccc34)cccc1C21c2ccccc2-c2ccccc21 ; CC1(C)c2ccccc2-c2cc(-c3nc(-c4cccc5c4Oc4ccccc4C54c5ccccc5-c5ccccc54)c4sc5ccccc5c4n3)ccc21]

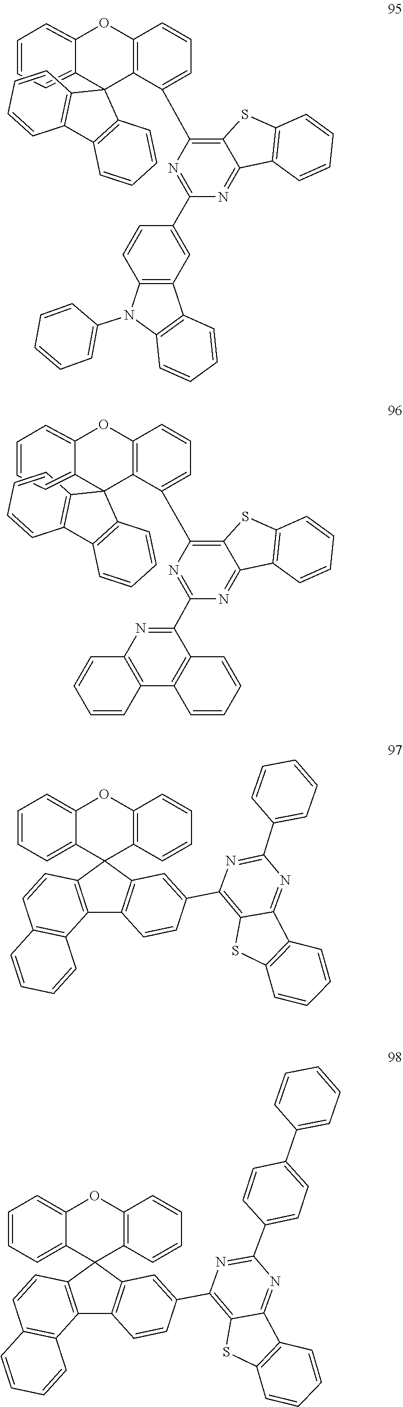 [SMILES: c1ccc(-n2c3ccccc3c3cc(-c4nc(-c5cccc6c5C5(c7ccccc7O6)c6ccccc6-c6ccccc65)c5sc6ccccc6c5n4)ccc32)cc1 ; c1ccc2c(c1)Oc1cccc(-c3nc(-c4nc5ccccc5c5ccccc45)nc4c3sc3ccccc34)c1C21c2ccccc2-c2ccccc21 ; c1ccc(-c2nc(-c3ccc4c(c3)C3(c5ccccc5Oc5ccccc53)c3ccc5ccccc5c3-4)c3sc4ccccc4c3n2)cc1 ; c1ccc(-c2ccc(-c3nc(-c4ccc5c(c4)C4(c6ccccc6Oc6ccccc64)c4ccc6ccccc6c4-5)c4sc5ccccc5c4n3)cc2)cc1]

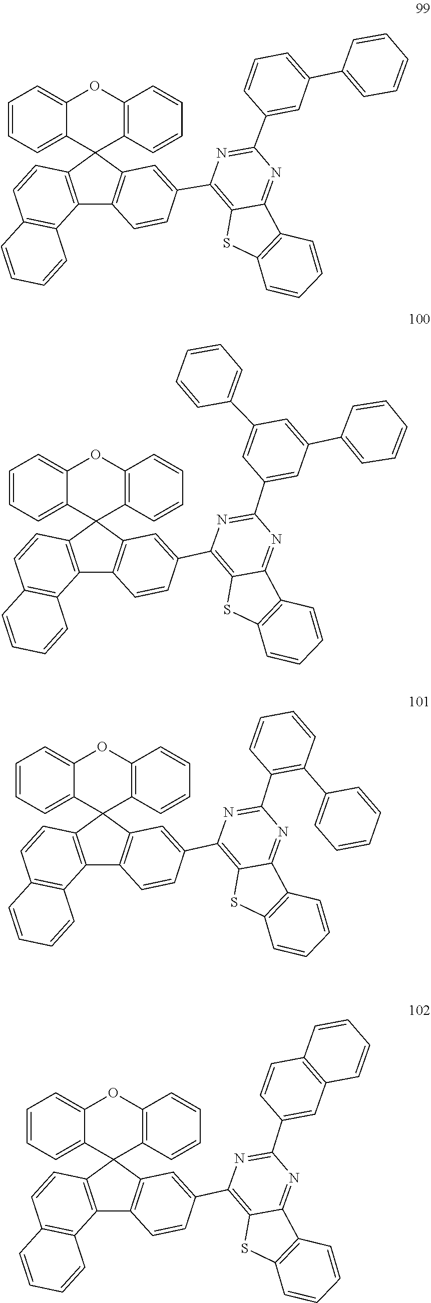 [SMILES: c1ccc(-c2cccc(-c3nc(-c4ccc5c(c4)C4(c6ccccc6Oc6ccccc64)c4ccc6ccccc6c4-5)c4sc5ccccc5c4n3)c2)cc1 ; c1ccc(-c2cc(-c3ccccc3)cc(-c3nc(-c4ccc5c(c4)C4(c6ccccc6Oc6ccccc64)c4ccc6ccccc6c4-5)c4sc5ccccc5c4n3)c2)cc1 ; c1ccc(-c2ccccc2-c2nc(-c3ccc4c(c3)C3(c5ccccc5Oc5ccccc53)c3ccc5ccccc5c3-4)c3sc4ccccc4c3n2)cc1 ; c1ccc2c(c1)Oc1ccccc1C21c2cc(-c3nc(-c4ccc5ccccc5c4)nc4c3sc3ccccc34)ccc2-c2c1ccc1ccccc21]

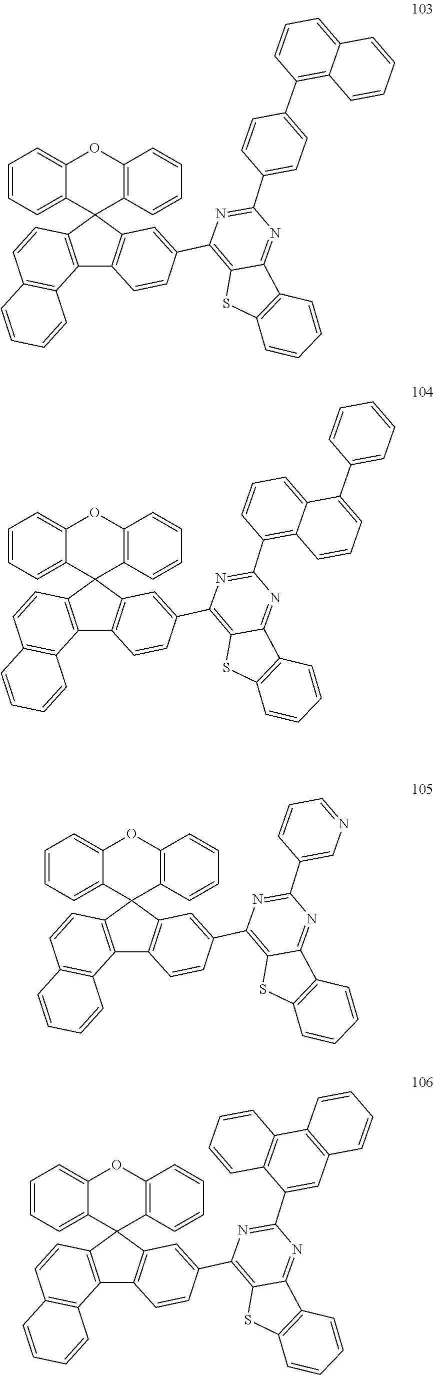 [SMILES: c1ccc2c(c1)Oc1ccccc1C21c2cc(-c3nc(-c4ccc(-c5cccc6ccccc56)cc4)nc4c3sc3ccccc34)ccc2-c2c1ccc1ccccc21 ; c1ccc(-c2cccc3c(-c4nc(-c5ccc6c(c5)C5(c7ccccc7Oc7ccccc75)c5ccc7ccccc7c5-6)c5sc6ccccc6c5n4)cccc23)cc1 ; c1cncc(-c2nc(-c3ccc4c(c3)C3(c5ccccc5Oc5ccccc53)c3ccc5ccccc5c3-4)c3sc4ccccc4c3n2)c1 ; c1ccc2c(c1)Oc1ccccc1C21c2cc(-c3nc(-c4cc5ccccc5c5ccccc45)nc4c3sc3ccccc34)ccc2-c2c1ccc1ccccc21]

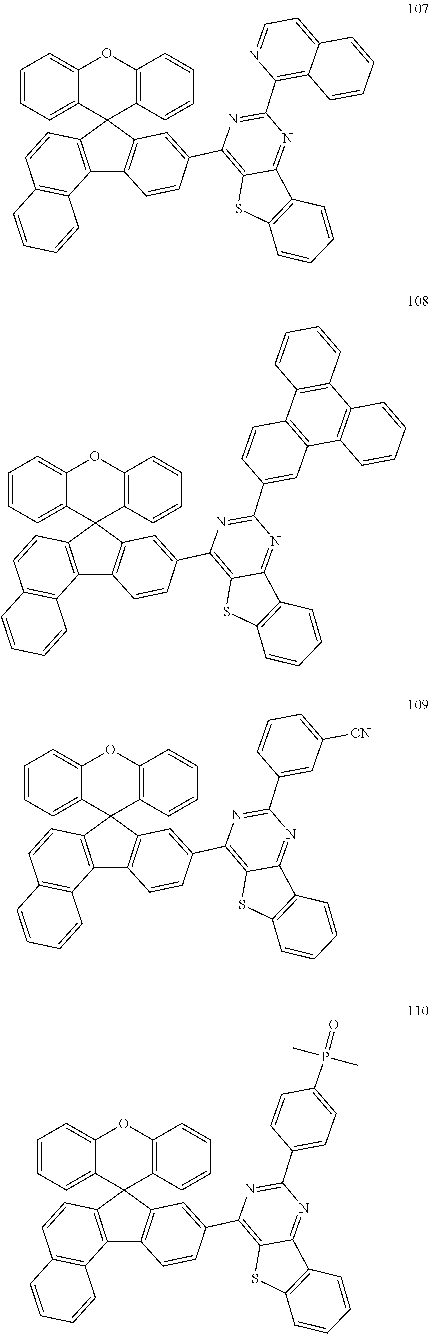 [SMILES: c1ccc2c(c1)Oc1ccccc1C21c2cc(-c3nc(-c4nccc5ccccc45)nc4c3sc3ccccc34)ccc2-c2c1ccc1ccccc21 ; c1ccc2c(c1)Oc1ccccc1C21c2cc(-c3nc(-c4ccc5c6ccccc6c6ccccc6c5c4)nc4c3sc3ccccc34)ccc2-c2c1ccc1ccccc21 ; N#Cc1cccc(-c2nc(-c3ccc4c(c3)C3(c5ccccc5Oc5ccccc53)c3ccc5ccccc5c3-4)c3sc4ccccc4c3n2)c1 ; CP(C)(=O)c1ccc(-c2nc(-c3ccc4c(c3)C3(c5ccccc5Oc5ccccc53)c3ccc5ccccc5c3-4)c3sc4ccccc4c3n2)cc1]

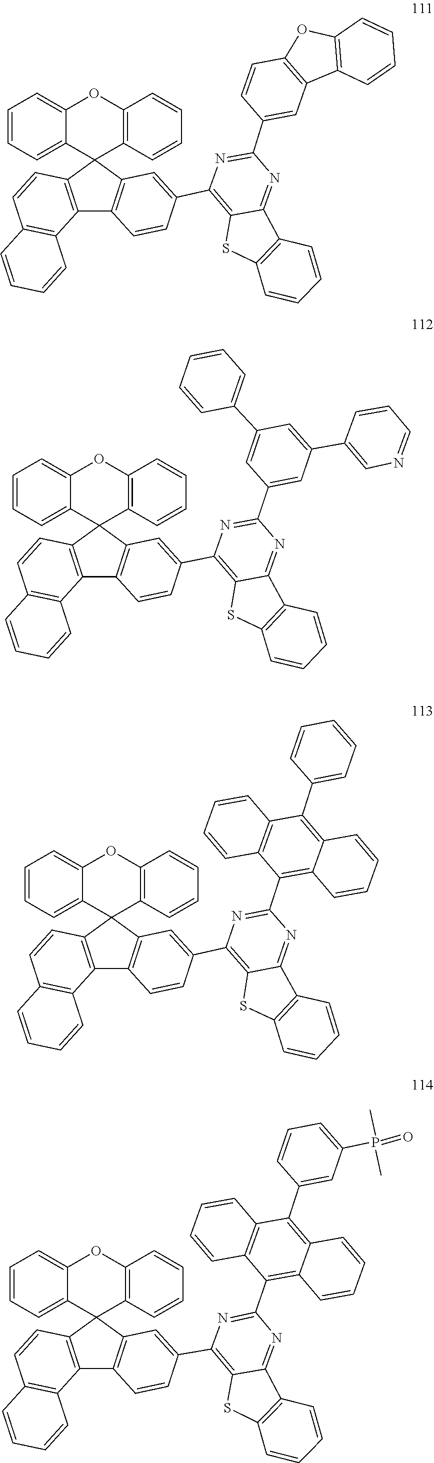 [SMILES: c1ccc2c(c1)Oc1ccccc1C21c2cc(-c3nc(-c4ccc5oc6ccccc6c5c4)nc4c3sc3ccccc34)ccc2-c2c1ccc1ccccc21 ; c1ccc(-c2cc(-c3cccnc3)cc(-c3nc(-c4ccc5c(c4)C4(c6ccccc6Oc6ccccc64)c4ccc6ccccc6c4-5)c4sc5ccccc5c4n3)c2)cc1 ; c1ccc(-c2c3ccccc3c(-c3nc(-c4ccc5c(c4)C4(c6ccccc6Oc6ccccc64)c4ccc6ccccc6c4-5)c4sc5ccccc5c4n3)c3ccccc23)cc1 ; CP(C)(=O)c1cccc(-c2c3ccccc3c(-c3nc(-c4ccc5c(c4)C4(c6ccccc6Oc6ccccc64)c4ccc6ccccc6c4-5)c4sc5ccccc5c4n3)c3ccccc23)c1]

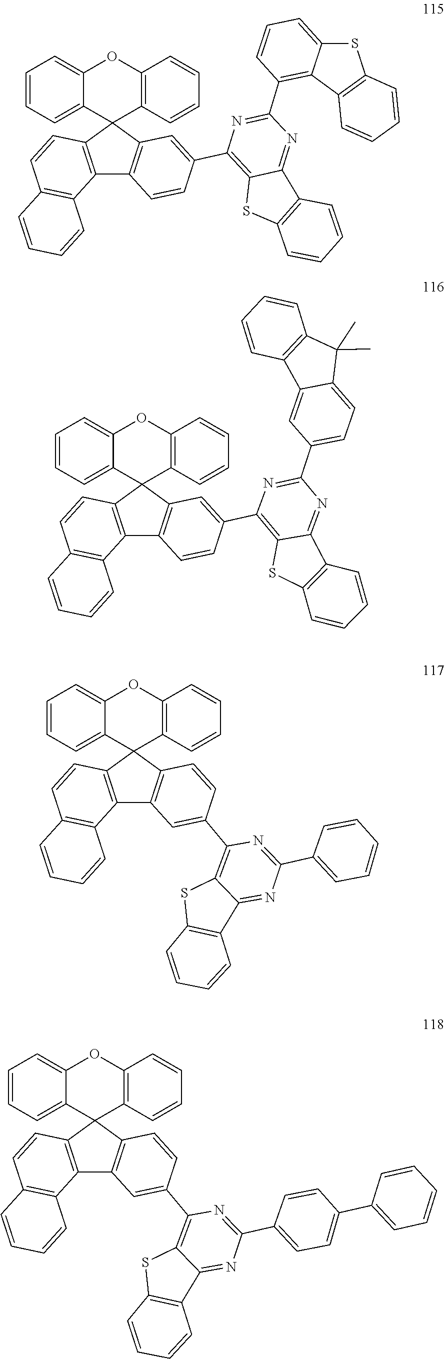 [SMILES: c1ccc2c(c1)Oc1ccccc1C21c2cc(-c3nc(-c4cccc5sc6ccccc6c45)nc4c3sc3ccccc34)ccc2-c2c1ccc1ccccc21 ; CC1(C)c2ccccc2-c2cc(-c3nc(-c4ccc5c(c4)C4(c6ccccc6Oc6ccccc64)c4ccc6ccccc6c4-5)c4sc5ccccc5c4n3)ccc21 ; c1ccc(-c2nc(-c3ccc4c(c3)-c3c(ccc5ccccc35)C43c4ccccc4Oc4ccccc43)c3sc4ccccc4c3n2)cc1 ; c1ccc(-c2ccc(-c3nc(-c4ccc5c(c4)-c4c(ccc6ccccc46)C54c5ccccc5Oc5ccccc54)c4sc5ccccc5c4n3)cc2)cc1]

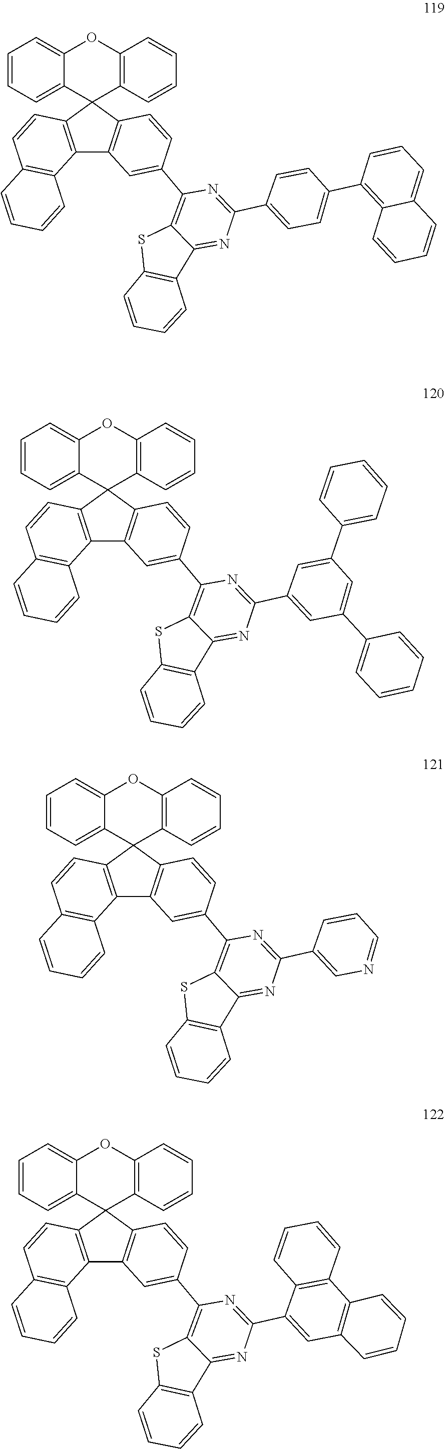 [SMILES: c1ccc2c(c1)Oc1ccccc1C21c2ccc(-c3nc(-c4ccc(-c5cccc6ccccc56)cc4)nc4c3sc3ccccc34)cc2-c2c1ccc1ccccc21 ; c1ccc(-c2cc(-c3ccccc3)cc(-c3nc(-c4ccc5c(c4)-c4c(ccc6ccccc46)C54c5ccccc5Oc5ccccc54)c4sc5ccccc5c4n3)c2)cc1 ; c1cncc(-c2nc(-c3ccc4c(c3)-c3c(ccc5ccccc35)C43c4ccccc4Oc4ccccc43)c3sc4ccccc4c3n2)c1 ; c1ccc2c(c1)Oc1ccccc1C21c2ccc(-c3nc(-c4cc5ccccc5c5ccccc45)nc4c3sc3ccccc34)cc2-c2c1ccc1ccccc21]

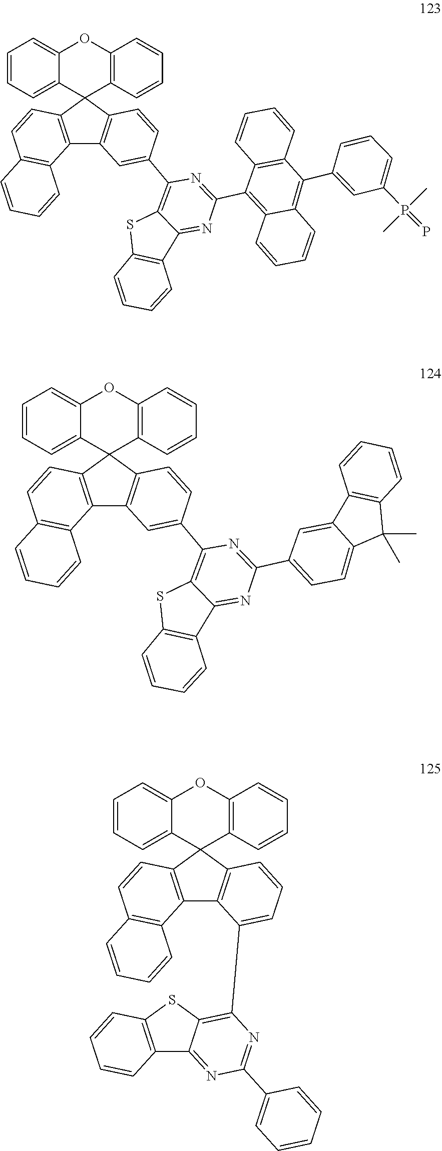

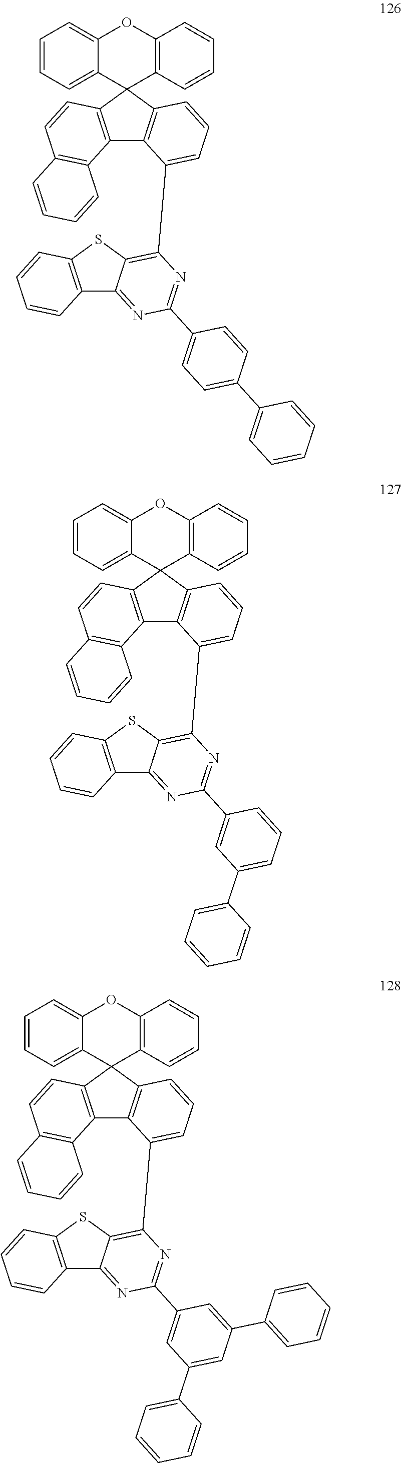 [SMILES: c1ccc(-c2ccc(-c3nc(-c4cccc5c4-c4c(ccc6ccccc46)C54c5ccccc5Oc5ccccc54)c4sc5ccccc5c4n3)cc2)cc1 ; c1ccc(-c2cccc(-c3nc(-c4cccc5c4-c4c(ccc6ccccc46)C54c5ccccc5Oc5ccccc54)c4sc5ccccc5c4n3)c2)cc1 ; c1ccc(-c2cc(-c3ccccc3)cc(-c3nc(-c4cccc5c4-c4c(ccc6ccccc46)C54c5ccccc5Oc5ccccc54)c4sc5ccccc5c4n3)c2)cc1]

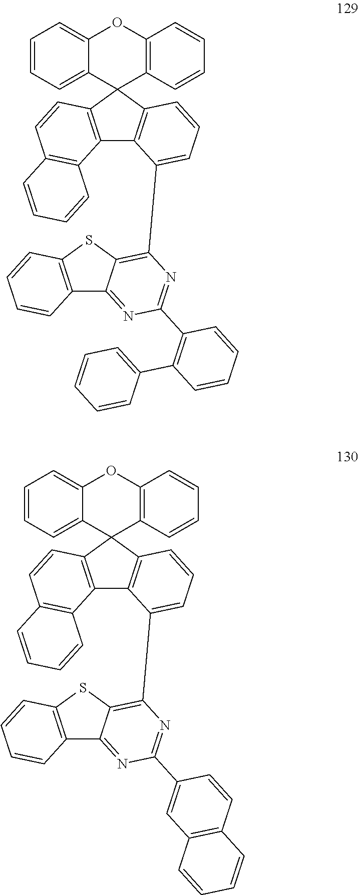 [SMILES: c1ccc(-c2ccccc2-c2nc(-c3cccc4c3-c3c(ccc5ccccc35)C43c4ccccc4Oc4ccccc43)c3sc4ccccc4c3n2)cc1 ; c1ccc2c(c1)Oc1ccccc1C21c2cccc(-c3nc(-c4ccc5ccccc5c4)nc4c3sc3ccccc34)c2-c2c1ccc1ccccc21]

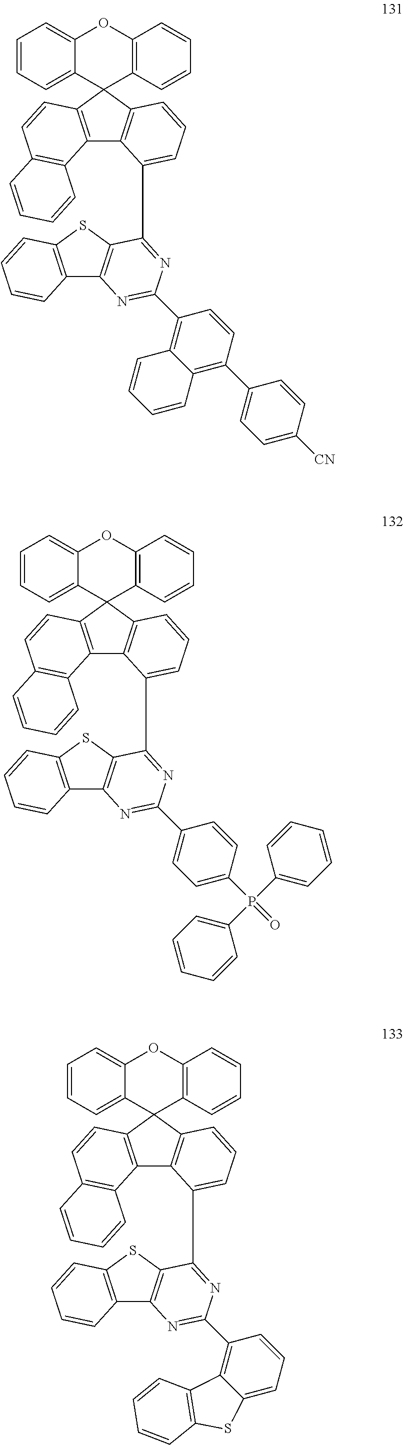 [SMILES: N#Cc1ccc(-c2ccc(-c3nc(-c4cccc5c4-c4c(ccc6ccccc46)C54c5ccccc5Oc5ccccc54)c4sc5ccccc5c4n3)c3ccccc23)cc1 ; O=P(c1ccccc1)(c1ccccc1)c1ccc(-c2nc(-c3cccc4c3-c3c(ccc5ccccc35)C43c4ccccc4Oc4ccccc43)c3sc4ccccc4c3n2)cc1 ; c1ccc2c(c1)Oc1ccccc1C21c2cccc(-c3nc(-c4cccc5sc6ccccc6c45)nc4c3sc3ccccc34)c2-c2c1ccc1ccccc21]

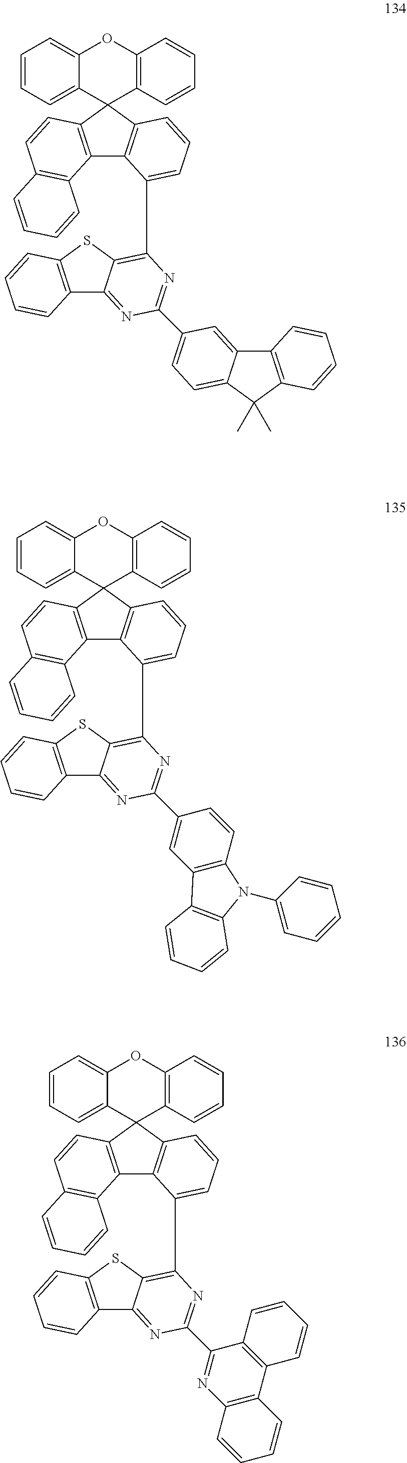 [SMILES: CC1(C)c2ccccc2-c2cc(-c3nc(-c4cccc5c4-c4c(ccc6ccccc46)C54c5ccccc5Oc5ccccc54)c4sc5ccccc5c4n3)ccc21 ; c1ccc(-n2c3ccccc3c3cc(-c4nc(-c5cccc6c5-c5c(ccc7ccccc57)C65c6ccccc6Oc6ccccc65)c5sc6ccccc6c5n4)ccc32)cc1 ; c1ccc2c(c1)Oc1ccccc1C21c2cccc(-c3nc(-c4nc5ccccc5c5ccccc45)nc4c3sc3ccccc34)c2-c2c1ccc1ccccc21]

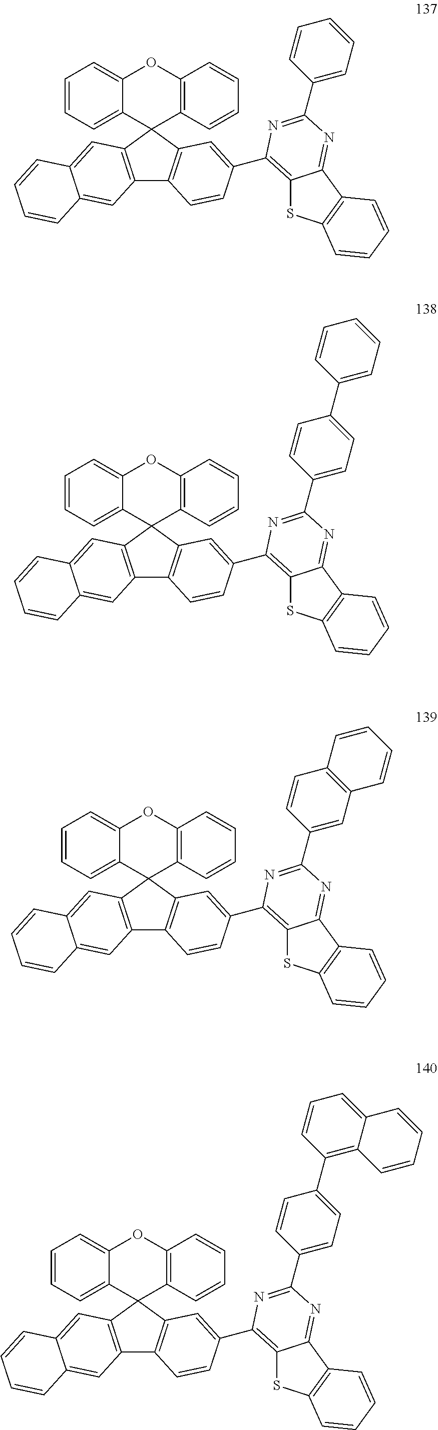 [SMILES: c1ccc(-c2nc(-c3ccc4c(c3)C3(c5ccccc5Oc5ccccc53)c3cc5ccccc5cc3-4)c3sc4ccccc4c3n2)cc1 ; c1ccc(-c2ccc(-c3nc(-c4ccc5c(c4)C4(c6ccccc6Oc6ccccc64)c4cc6ccccc6cc4-5)c4sc5ccccc5c4n3)cc2)cc1 ; c1ccc2c(c1)Oc1ccccc1C21c2cc(-c3nc(-c4ccc5ccccc5c4)nc4c3sc3ccccc34)ccc2-c2cc3ccccc3cc21 ; c1ccc2c(c1)Oc1ccccc1C21c2cc(-c3nc(-c4ccc(-c5cccc6ccccc56)cc4)nc4c3sc3ccccc34)ccc2-c2cc3ccccc3cc21]

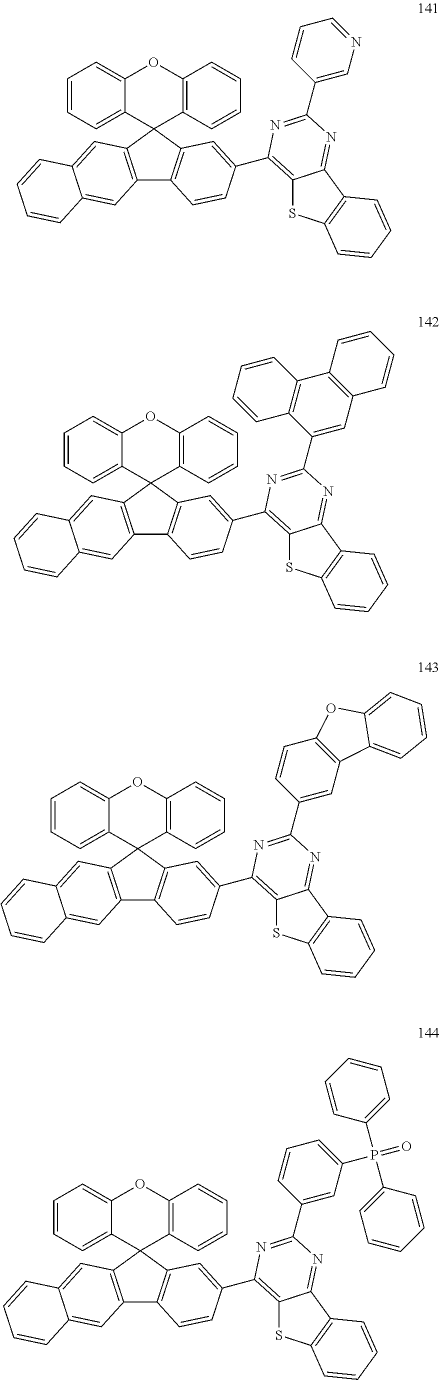 [SMILES: c1cncc(-c2nc(-c3ccc4c(c3)C3(c5ccccc5Oc5ccccc53)c3cc5ccccc5cc3-4)c3sc4ccccc4c3n2)c1 ; c1ccc2c(c1)Oc1ccccc1C21c2cc(-c3nc(-c4cc5ccccc5c5ccccc45)nc4c3sc3ccccc34)ccc2-c2cc3ccccc3cc21 ; c1ccc2c(c1)Oc1ccccc1C21c2cc(-c3nc(-c4ccc5oc6ccccc6c5c4)nc4c3sc3ccccc34)ccc2-c2cc3ccccc3cc21 ; O=P(c1ccccc1)(c1ccccc1)c1cccc(-c2nc(-c3ccc4c(c3)C3(c5ccccc5Oc5ccccc53)c3cc5ccccc5cc3-4)c3sc4ccccc4c3n2)c1]

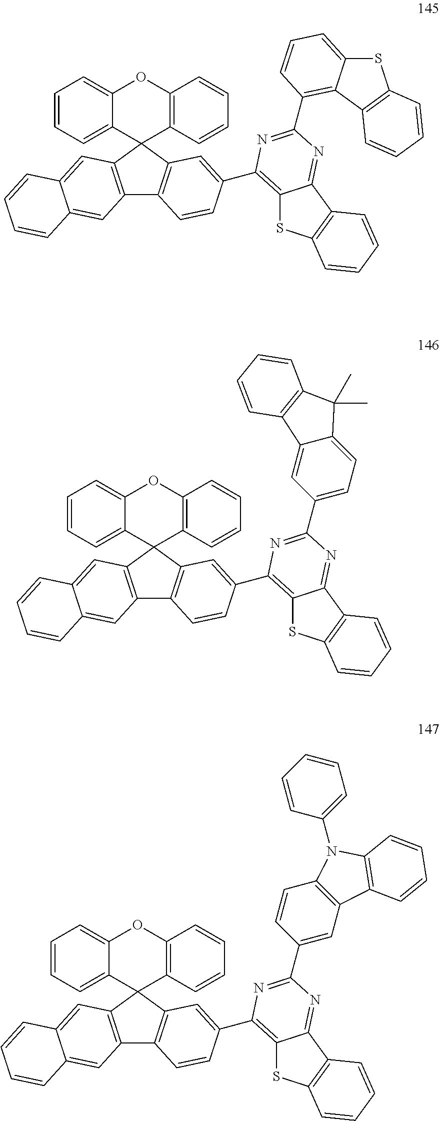 [SMILES: c1ccc2c(c1)Oc1ccccc1C21c2cc(-c3nc(-c4cccc5sc6ccccc6c45)nc4c3sc3ccccc34)ccc2-c2cc3ccccc3cc21 ; CC1(C)c2ccccc2-c2cc(-c3nc(-c4ccc5c(c4)C4(c6ccccc6Oc6ccccc64)c4cc6ccccc6cc4-5)c4sc5ccccc5c4n3)ccc21 ; c1ccc(-n2c3ccccc3c3cc(-c4nc(-c5ccc6c(c5)C5(c7ccccc7Oc7ccccc75)c5cc7ccccc7cc5-6)c5sc6ccccc6c5n4)ccc32)cc1]

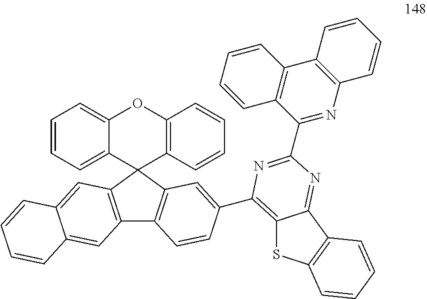 [SMILES: c1ccc2c(c1)Oc1ccccc1C21c2cc(-c3nc(-c4nc5ccccc5c5ccccc45)nc4c3sc3ccccc34)ccc2-c2cc3ccccc3cc21]

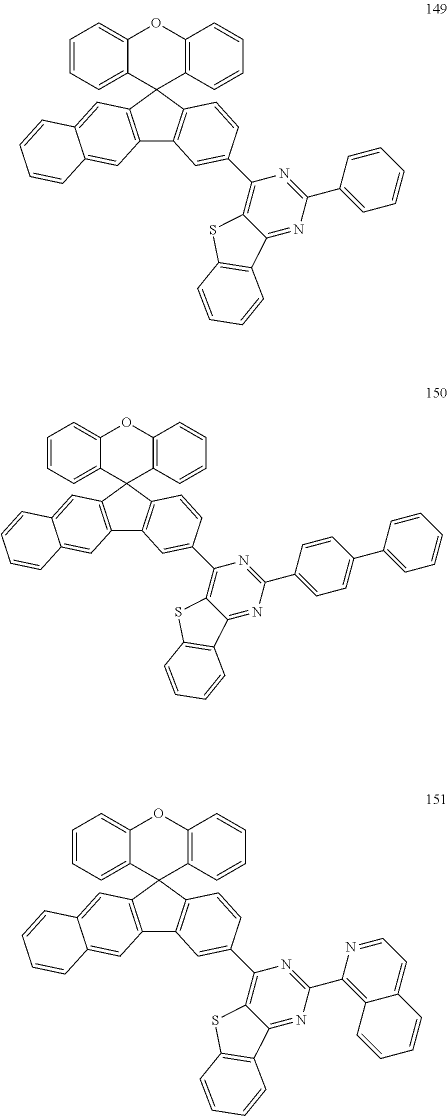 [SMILES: c1ccc(-c2nc(-c3ccc4c(c3)-c3cc5ccccc5cc3C43c4ccccc4Oc4ccccc43)c3sc4ccccc4c3n2)cc1 ; c1ccc(-c2ccc(-c3nc(-c4ccc5c(c4)-c4cc6ccccc6cc4C54c5ccccc5Oc5ccccc54)c4sc5ccccc5c4n3)cc2)cc1 ; c1ccc2c(c1)Oc1ccccc1C21c2ccc(-c3nc(-c4nccc5ccccc45)nc4c3sc3ccccc34)cc2-c2cc3ccccc3cc21]

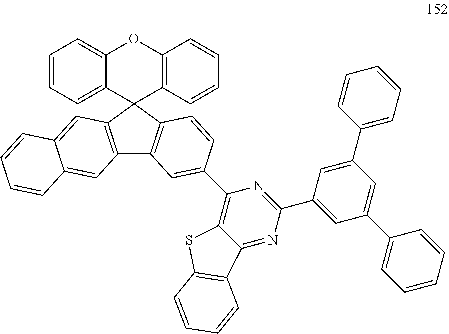 [SMILES: c1ccc(-c2cc(-c3ccccc3)cc(-c3nc(-c4ccc5c(c4)-c4cc6ccccc6cc4C54c5ccccc5Oc5ccccc54)c4sc5ccccc5c4n3)c2)cc1]

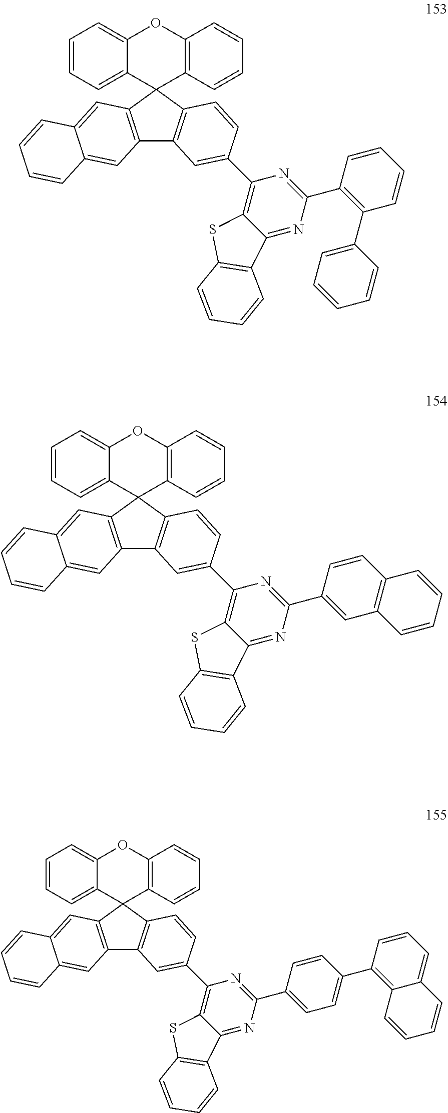 [SMILES: c1ccc(-c2ccccc2-c2nc(-c3ccc4c(c3)-c3cc5ccccc5cc3C43c4ccccc4Oc4ccccc43)c3sc4ccccc4c3n2)cc1 ; c1ccc2c(c1)Oc1ccccc1C21c2ccc(-c3nc(-c4ccc5ccccc5c4)nc4c3sc3ccccc34)cc2-c2cc3ccccc3cc21 ; c1ccc2c(c1)Oc1ccccc1C21c2ccc(-c3nc(-c4ccc(-c5cccc6ccccc56)cc4)nc4c3sc3ccccc34)cc2-c2cc3ccccc3cc21]

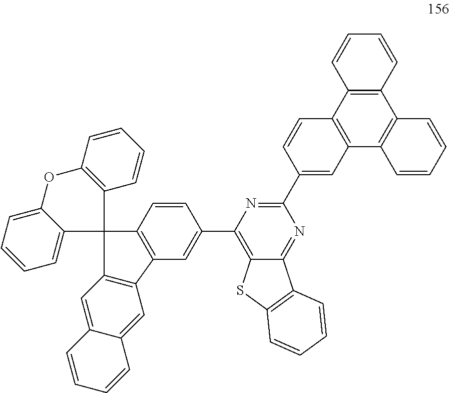 [SMILES: c1ccc2c(c1)Oc1ccccc1C21c2ccc(-c3nc(-c4ccc5c6ccccc6c6ccccc6c5c4)nc4c3sc3ccccc34)cc2-c2cc3ccccc3cc21]

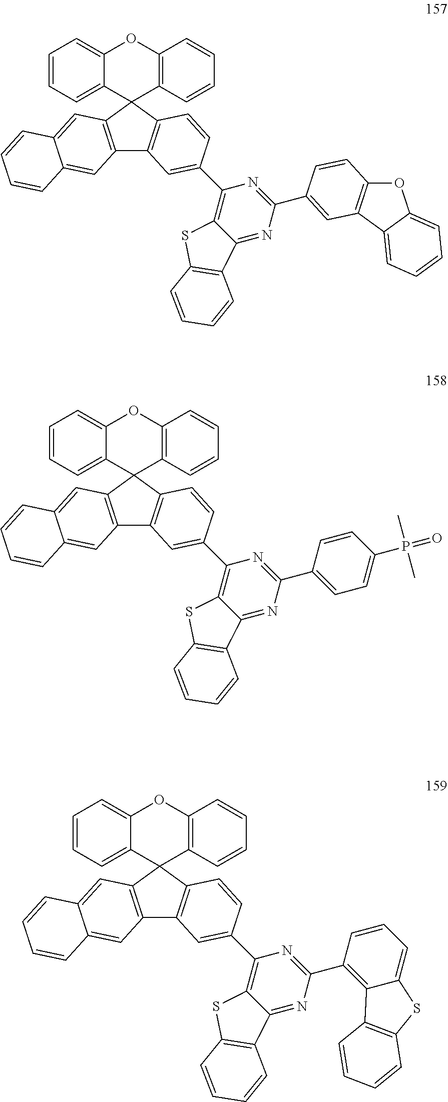 [SMILES: c1ccc2c(c1)Oc1ccccc1C21c2ccc(-c3nc(-c4ccc5oc6ccccc6c5c4)nc4c3sc3ccccc34)cc2-c2cc3ccccc3cc21 ; CP(C)(=O)c1ccc(-c2nc(-c3ccc4c(c3)-c3cc5ccccc5cc3C43c4ccccc4Oc4ccccc43)c3sc4ccccc4c3n2)cc1 ; c1ccc2c(c1)Oc1ccccc1C21c2ccc(-c3nc(-c4cccc5sc6ccccc6c45)nc4c3sc3ccccc34)cc2-c2cc3ccccc3cc21]

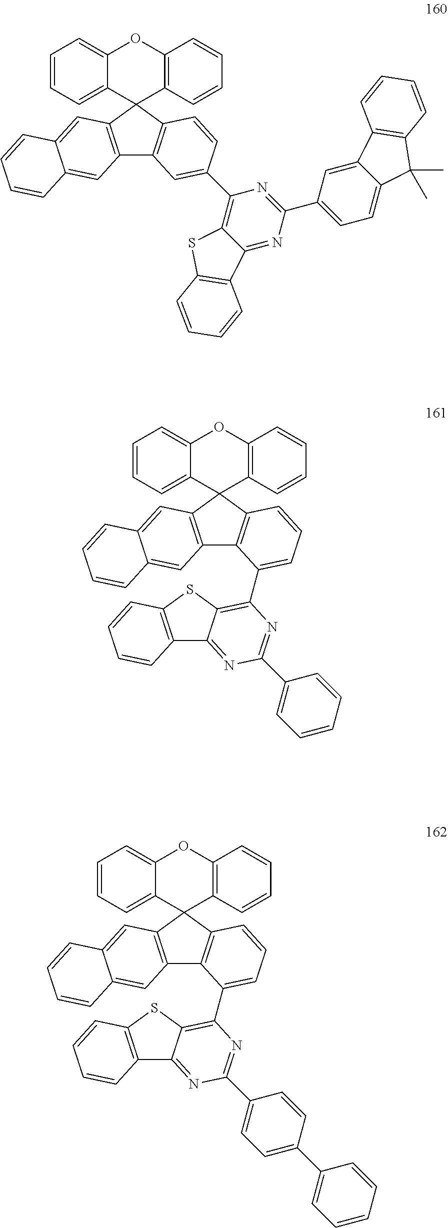 [SMILES: CC1(C)c2ccccc2-c2cc(-c3nc(-c4ccc5c(c4)-c4cc6ccccc6cc4C54c5ccccc5Oc5ccccc54)c4sc5ccccc5c4n3)ccc21 ; c1ccc(-c2nc(-c3cccc4c3-c3cc5ccccc5cc3C43c4ccccc4Oc4ccccc43)c3sc4ccccc4c3n2)cc1 ; c1ccc(-c2ccc(-c3nc(-c4cccc5c4-c4cc6ccccc6cc4C54c5ccccc5Oc5ccccc54)c4sc5ccccc5c4n3)cc2)cc1]

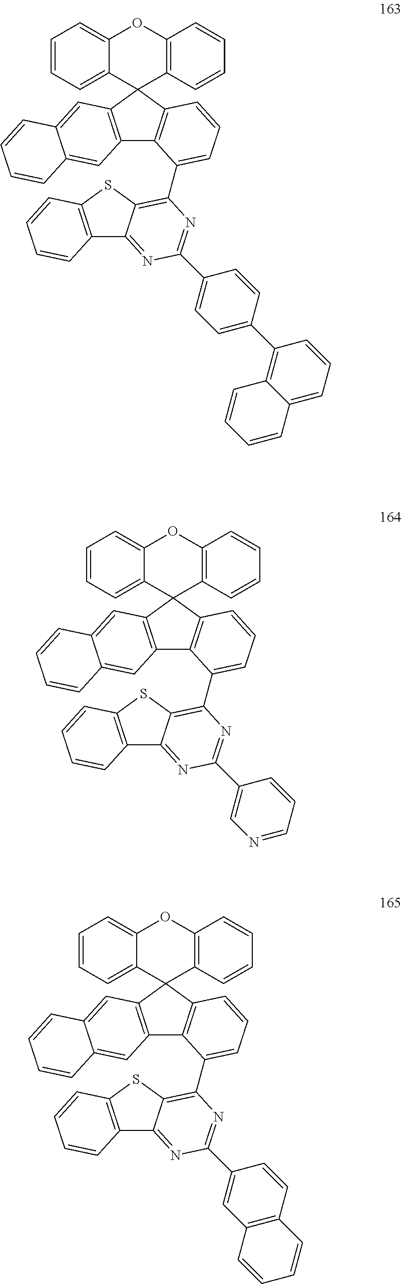 [SMILES: c1ccc2c(c1)Oc1ccccc1C21c2cc3ccccc3cc2-c2c(-c3nc(-c4ccc(-c5cccc6ccccc56)cc4)nc4c3sc3ccccc34)cccc21 ; c1cncc(-c2nc(-c3cccc4c3-c3cc5ccccc5cc3C43c4ccccc4Oc4ccccc43)c3sc4ccccc4c3n2)c1 ; c1ccc2c(c1)Oc1ccccc1C21c2cc3ccccc3cc2-c2c(-c3nc(-c4ccc5ccccc5c4)nc4c3sc3ccccc34)cccc21]

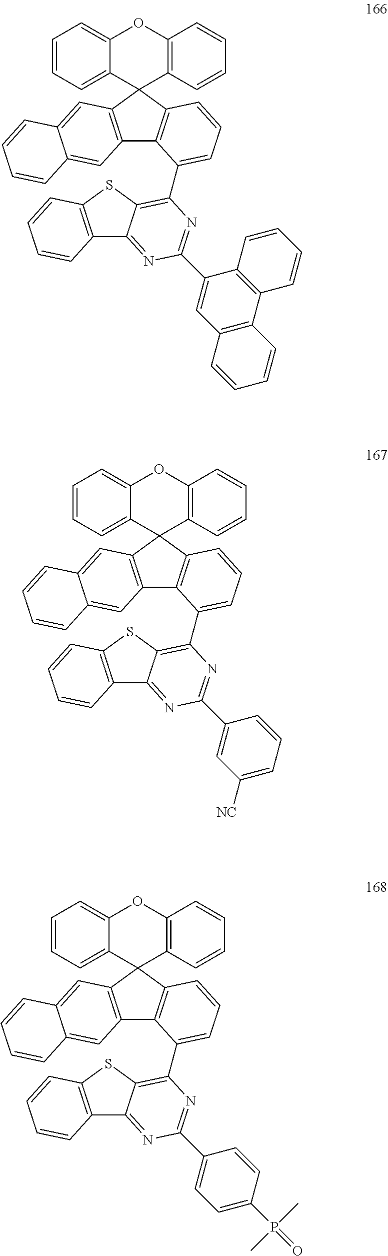 [SMILES: c1ccc2c(c1)Oc1ccccc1C21c2cc3ccccc3cc2-c2c(-c3nc(-c4cc5ccccc5c5ccccc45)nc4c3sc3ccccc34)cccc21 ; N#Cc1cccc(-c2nc(-c3cccc4c3-c3cc5ccccc5cc3C43c4ccccc4Oc4ccccc43)c3sc4ccccc4c3n2)c1 ; CP(C)(=O)c1ccc(-c2nc(-c3cccc4c3-c3cc5ccccc5cc3C43c4ccccc4Oc4ccccc43)c3sc4ccccc4c3n2)cc1]

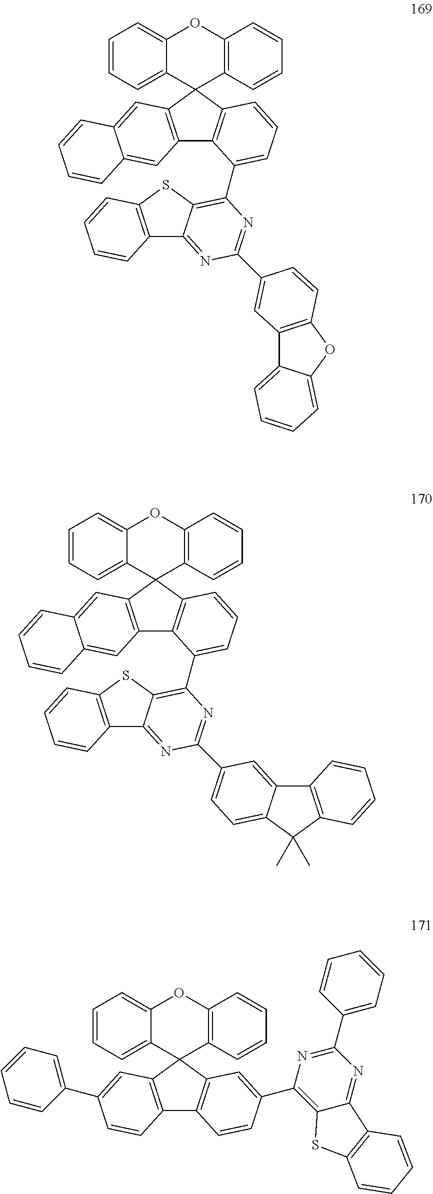 [SMILES: c1ccc2c(c1)Oc1ccccc1C21c2cc3ccccc3cc2-c2c(-c3nc(-c4ccc5oc6ccccc6c5c4)nc4c3sc3ccccc34)cccc21 ; CC1(C)c2ccccc2-c2cc(-c3nc(-c4cccc5c4-c4cc6ccccc6cc4C54c5ccccc5Oc5ccccc54)c4sc5ccccc5c4n3)ccc21 ; c1ccc(-c2ccc3c(c2)C2(c4ccccc4Oc4ccccc42)c2cc(-c4nc(-c5ccccc5)nc5c4sc4ccccc45)ccc2-3)cc1]

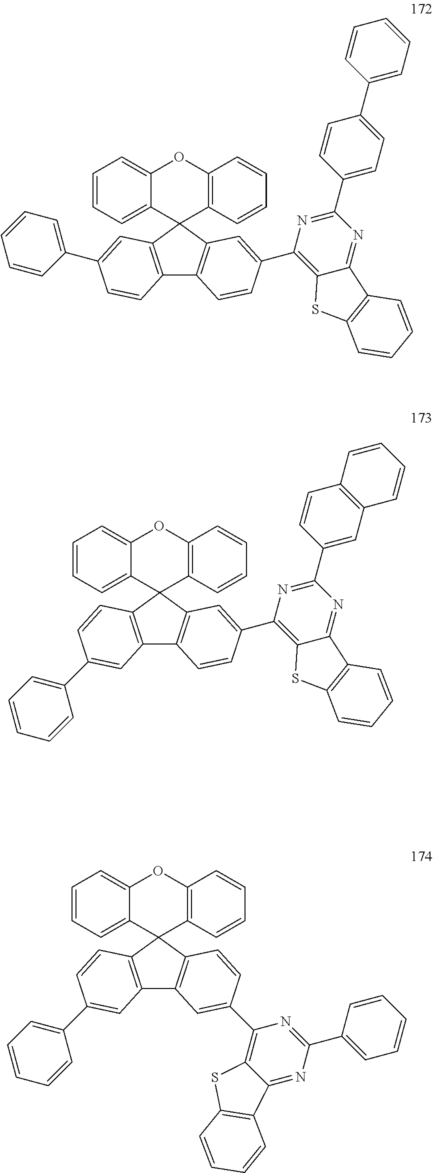 [SMILES: c1ccc(-c2ccc(-c3nc(-c4ccc5c(c4)C4(c6ccccc6Oc6ccccc64)c4cc(-c6ccccc6)ccc4-5)c4sc5ccccc5c4n3)cc2)cc1 ; c1ccc(-c2ccc3c(c2)-c2ccc(-c4nc(-c5ccc6ccccc6c5)nc5c4sc4ccccc45)cc2C32c3ccccc3Oc3ccccc32)cc1 ; c1ccc(-c2ccc3c(c2)-c2cc(-c4nc(-c5ccccc5)nc5c4sc4ccccc45)ccc2C32c3ccccc3Oc3ccccc32)cc1]

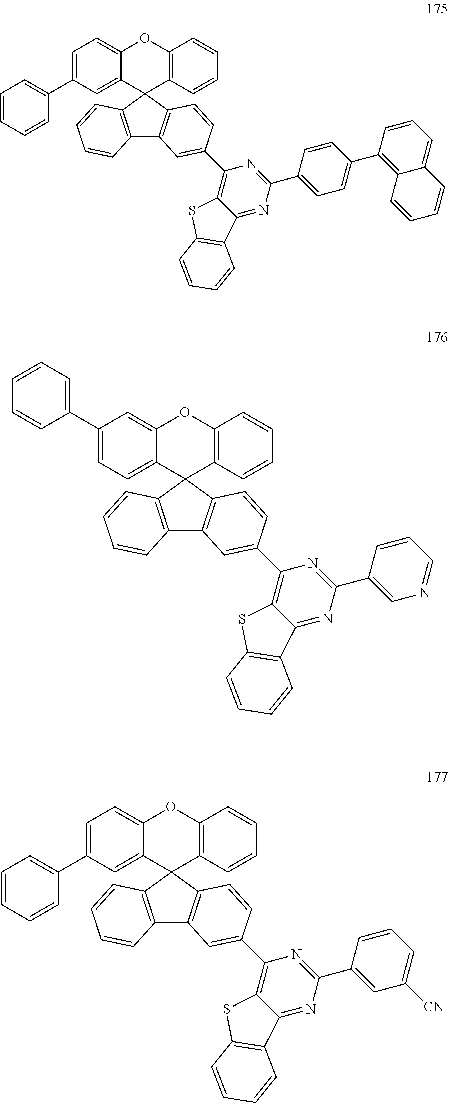 [SMILES: c1ccc(-c2ccc3c(c2)C2(c4ccccc4O3)c3ccccc3-c3cc(-c4nc(-c5ccc(-c6cccc7ccccc67)cc5)nc5c4sc4ccccc45)ccc32)cc1 ; c1ccc(-c2ccc3c(c2)Oc2ccccc2C32c3ccccc3-c3cc(-c4nc(-c5cccnc5)nc5c4sc4ccccc45)ccc32)cc1 ; N#Cc1cccc(-c2nc(-c3ccc4c(c3)-c3ccccc3C43c4ccccc4Oc4ccc(-c5ccccc5)cc43)c3sc4ccccc4c3n2)c1]

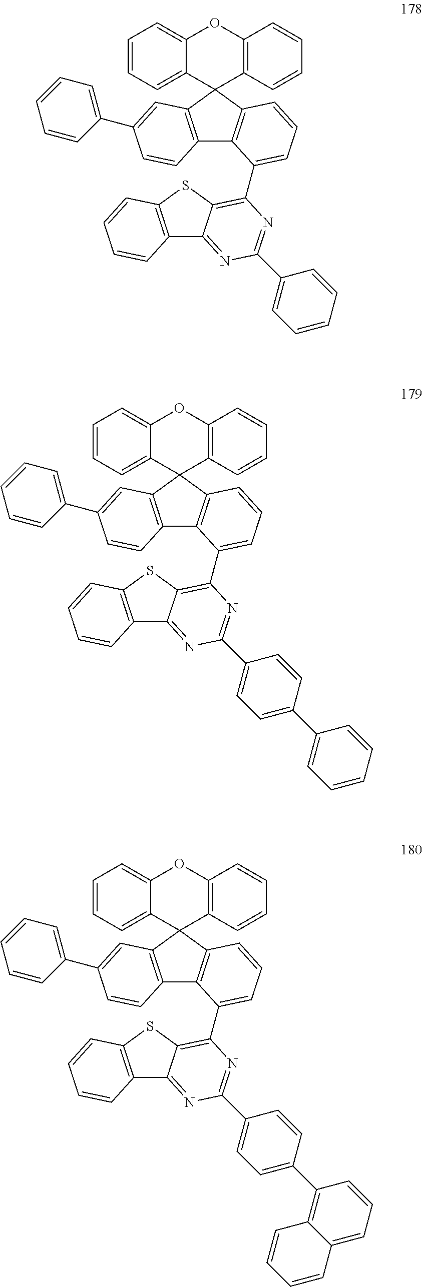 [SMILES: c1ccc(-c2ccc3c(c2)C2(c4ccccc4Oc4ccccc42)c2cccc(-c4nc(-c5ccccc5)nc5c4sc4ccccc45)c2-3)cc1 ; c1ccc(-c2ccc(-c3nc(-c4cccc5c4-c4ccc(-c6ccccc6)cc4C54c5ccccc5Oc5ccccc54)c4sc5ccccc5c4n3)cc2)cc1 ; c1ccc(-c2ccc3c(c2)C2(c4ccccc4Oc4ccccc42)c2cccc(-c4nc(-c5ccc(-c6cccc7ccccc67)cc5)nc5c4sc4ccccc45)c2-3)cc1]

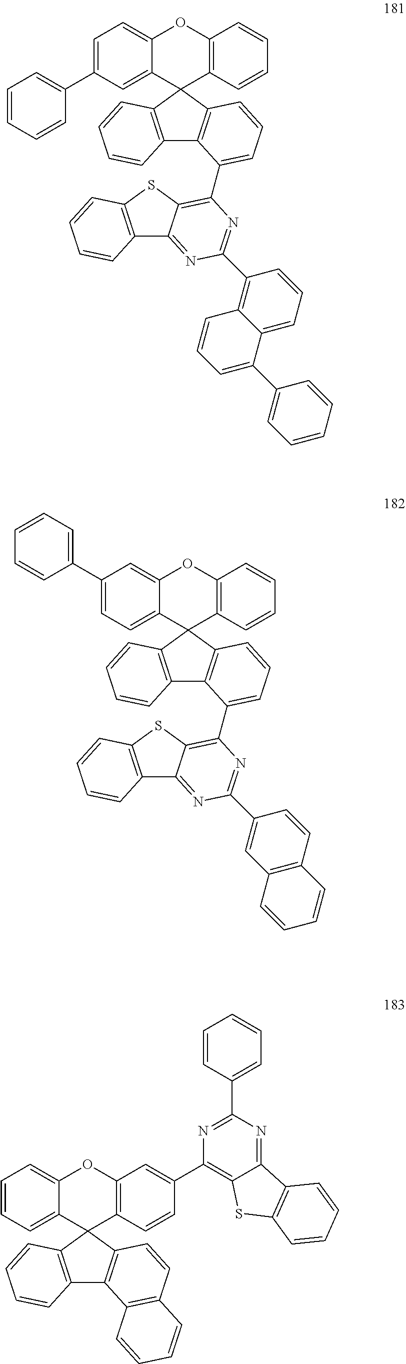 [SMILES: c1ccc(-c2ccc3c(c2)C2(c4ccccc4O3)c3ccccc3-c3c(-c4nc(-c5cccc6c(-c7ccccc7)cccc56)nc5c4sc4ccccc45)cccc32)cc1 ; c1ccc(-c2ccc3c(c2)Oc2ccccc2C32c3ccccc3-c3c(-c4nc(-c5ccc6ccccc6c5)nc5c4sc4ccccc45)cccc32)cc1 ; c1ccc(-c2nc(-c3ccc4c(c3)Oc3ccccc3C43c4ccccc4-c4c3ccc3ccccc43)c3sc4ccccc4c3n2)cc1]

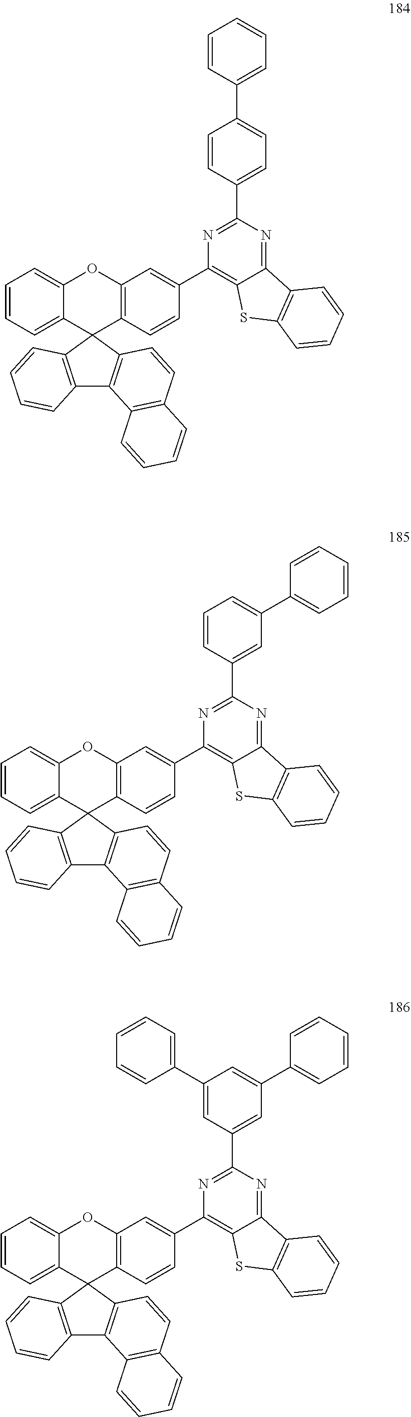 [SMILES: c1ccc(-c2ccc(-c3nc(-c4ccc5c(c4)Oc4ccccc4C54c5ccccc5-c5c4ccc4ccccc54)c4sc5ccccc5c4n3)cc2)cc1 ; c1ccc(-c2cccc(-c3nc(-c4ccc5c(c4)Oc4ccccc4C54c5ccccc5-c5c4ccc4ccccc54)c4sc5ccccc5c4n3)c2)cc1 ; c1ccc(-c2cc(-c3ccccc3)cc(-c3nc(-c4ccc5c(c4)Oc4ccccc4C54c5ccccc5-c5c4ccc4ccccc54)c4sc5ccccc5c4n3)c2)cc1]

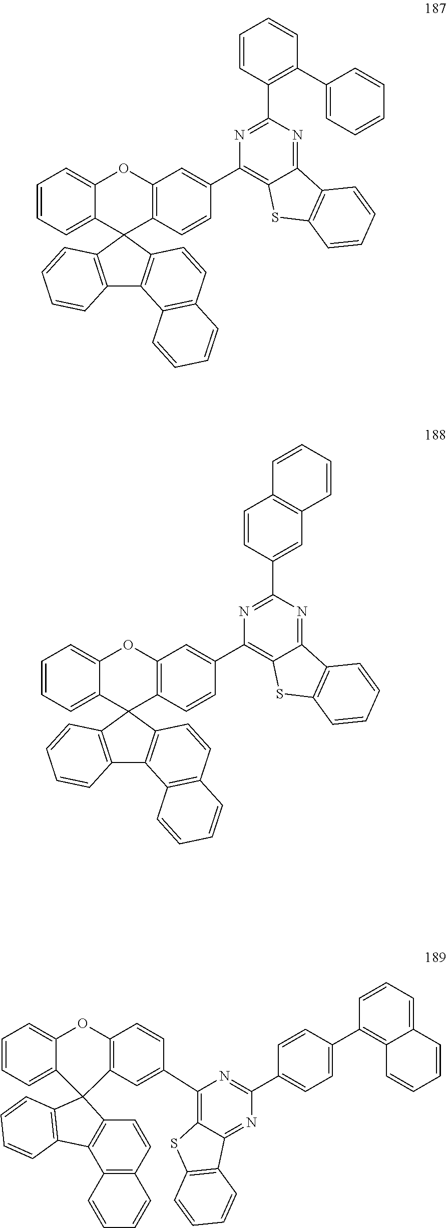 [SMILES: c1ccc(-c2ccccc2-c2nc(-c3ccc4c(c3)Oc3ccccc3C43c4ccccc4-c4c3ccc3ccccc43)c3sc4ccccc4c3n2)cc1 ; c1ccc2c(c1)Oc1cc(-c3nc(-c4ccc5ccccc5c4)nc4c3sc3ccccc34)ccc1C21c2ccccc2-c2c1ccc1ccccc21 ; c1ccc2c(c1)Oc1ccc(-c3nc(-c4ccc(-c5cccc6ccccc56)cc4)nc4c3sc3ccccc34)cc1C21c2ccccc2-c2c1ccc1ccccc21]

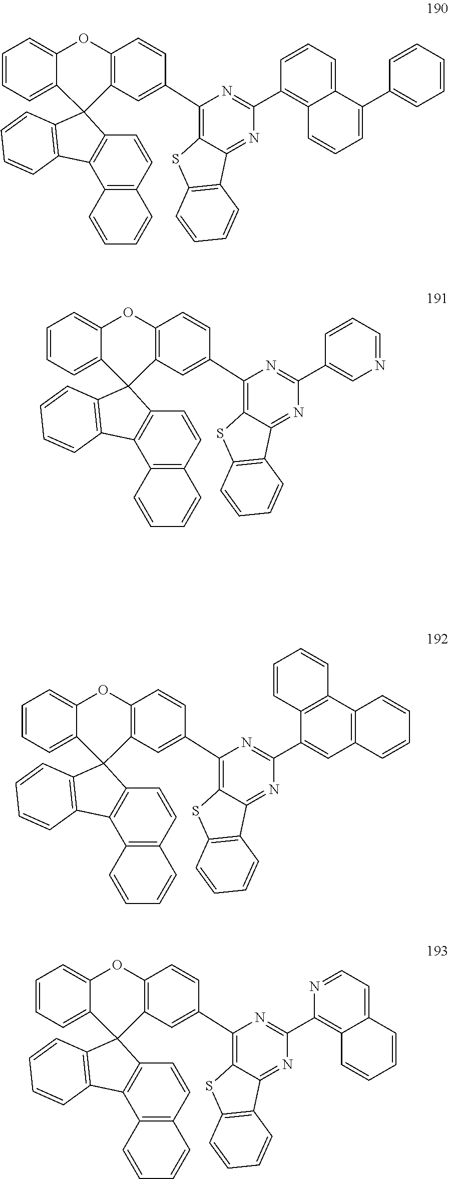 [SMILES: c1ccc(-c2cccc3c(-c4nc(-c5ccc6c(c5)C5(c7ccccc7O6)c6ccccc6-c6c5ccc5ccccc65)c5sc6ccccc6c5n4)cccc23)cc1 ; c1cncc(-c2nc(-c3ccc4c(c3)C3(c5ccccc5O4)c4ccccc4-c4c3ccc3ccccc43)c3sc4ccccc4c3n2)c1 ; c1ccc2c(c1)Oc1ccc(-c3nc(-c4cc5ccccc5c5ccccc45)nc4c3sc3ccccc34)cc1C21c2ccccc2-c2c1ccc1ccccc21 ; c1ccc2c(c1)Oc1ccc(-c3nc(-c4nccc5ccccc45)nc4c3sc3ccccc34)cc1C21c2ccccc2-c2c1ccc1ccccc21]

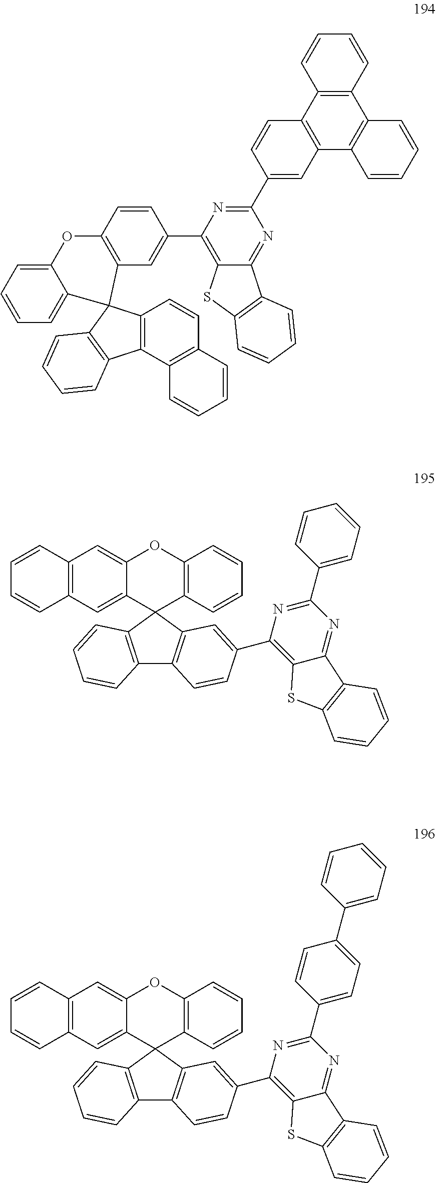 [SMILES: c1ccc2c(c1)Oc1ccc(-c3nc(-c4ccc5c6ccccc6c6ccccc6c5c4)nc4c3sc3ccccc34)cc1C21c2ccccc2-c2c1ccc1ccccc21 ; c1ccc(-c2nc(-c3ccc4c(c3)C3(c5ccccc5Oc5cc6ccccc6cc53)c3ccccc3-4)c3sc4ccccc4c3n2)cc1 ; c1ccc(-c2ccc(-c3nc(-c4ccc5c(c4)C4(c6ccccc6Oc6cc7ccccc7cc64)c4ccccc4-5)c4sc5ccccc5c4n3)cc2)cc1]

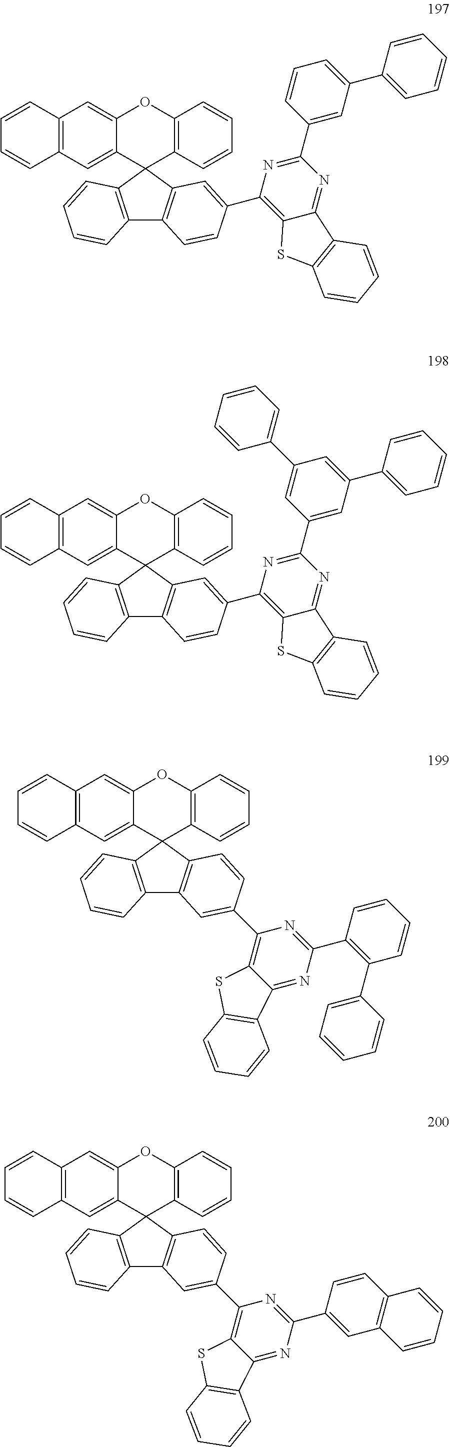 [SMILES: c1ccc(-c2cccc(-c3nc(-c4ccc5c(c4)C4(c6ccccc6Oc6cc7ccccc7cc64)c4ccccc4-5)c4sc5ccccc5c4n3)c2)cc1 ; c1ccc(-c2cc(-c3ccccc3)cc(-c3nc(-c4ccc5c(c4)C4(c6ccccc6Oc6cc7ccccc7cc64)c4ccccc4-5)c4sc5ccccc5c4n3)c2)cc1 ; c1ccc(-c2ccccc2-c2nc(-c3ccc4c(c3)-c3ccccc3C43c4ccccc4Oc4cc5ccccc5cc43)c3sc4ccccc4c3n2)cc1 ; c1ccc2c(c1)Oc1cc3ccccc3cc1C21c2ccccc2-c2cc(-c3nc(-c4ccc5ccccc5c4)nc4c3sc3ccccc34)ccc21]

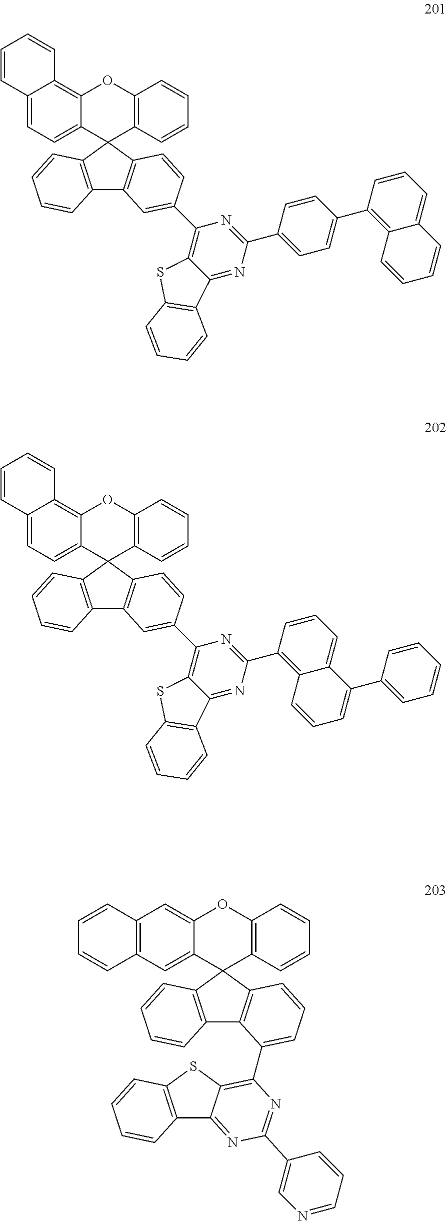 [SMILES: c1ccc2c(c1)Oc1c(ccc3ccccc13)C21c2ccccc2-c2cc(-c3nc(-c4ccc(-c5cccc6ccccc56)cc4)nc4c3sc3ccccc34)ccc21 ; c1ccc(-c2cccc3c(-c4nc(-c5ccc6c(c5)-c5ccccc5C65c6ccccc6Oc6c5ccc5ccccc65)c5sc6ccccc6c5n4)cccc23)cc1 ; c1cncc(-c2nc(-c3cccc4c3-c3ccccc3C43c4ccccc4Oc4cc5ccccc5cc43)c3sc4ccccc4c3n2)c1]

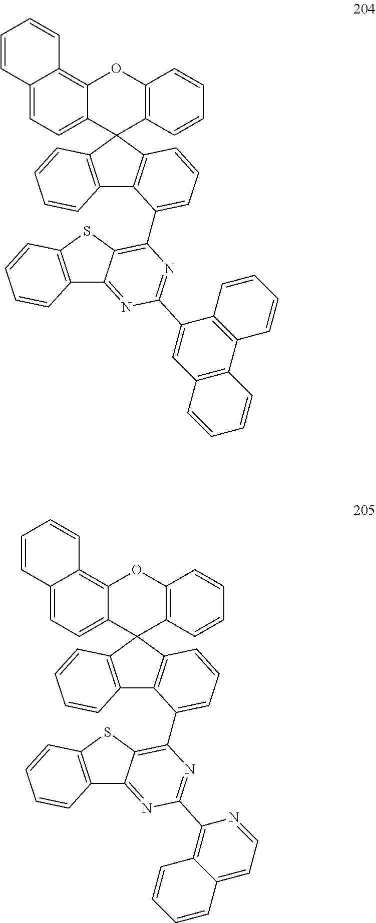 [SMILES: c1ccc2c(c1)Oc1c(ccc3ccccc13)C21c2ccccc2-c2c(-c3nc(-c4cc5ccccc5c5ccccc45)nc4c3sc3ccccc34)cccc21 ; c1ccc2c(c1)Oc1c(ccc3ccccc13)C21c2ccccc2-c2c(-c3nc(-c4nccc5ccccc45)nc4c3sc3ccccc34)cccc21]

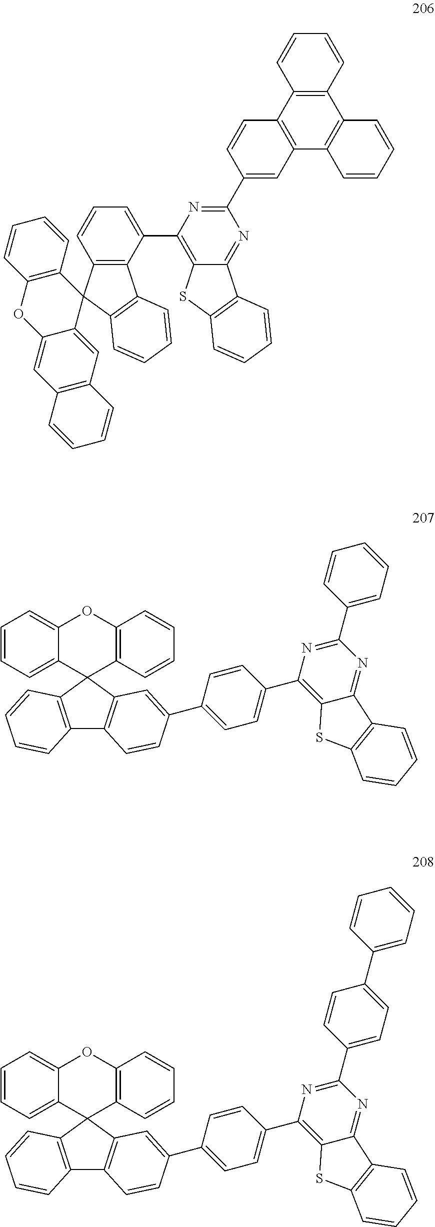 [SMILES: c1ccc2c(c1)Oc1cc3ccccc3cc1C21c2ccccc2-c2c(-c3nc(-c4ccc5c6ccccc6c6ccccc6c5c4)nc4c3sc3ccccc34)cccc21 ; c1ccc(-c2nc(-c3ccc(-c4ccc5c(c4)C4(c6ccccc6Oc6ccccc64)c4ccccc4-5)cc3)c3sc4ccccc4c3n2)cc1 ; c1ccc(-c2ccc(-c3nc(-c4ccc(-c5ccc6c(c5)C5(c7ccccc7Oc7ccccc75)c5ccccc5-6)cc4)c4sc5ccccc5c4n3)cc2)cc1]

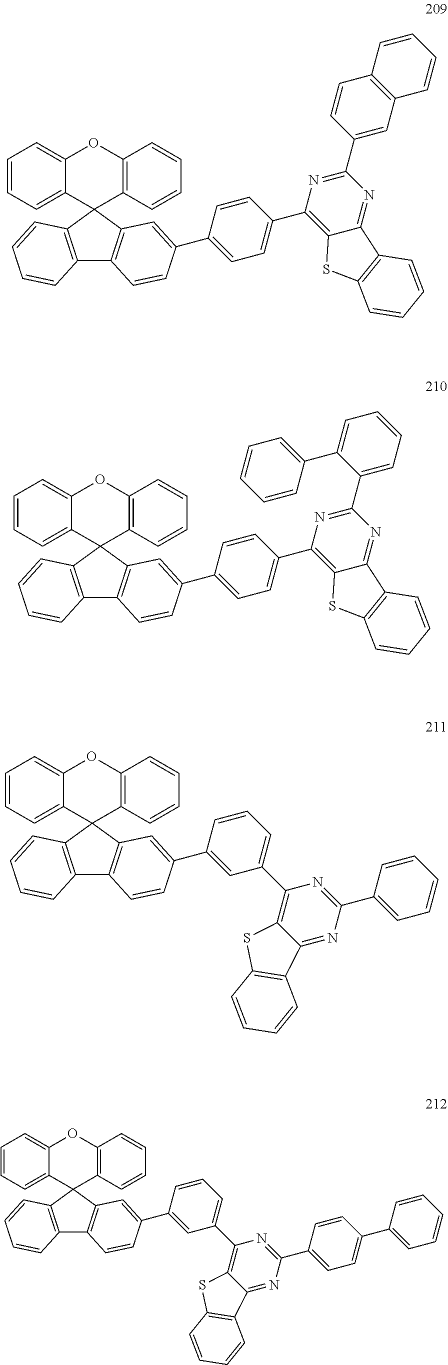 [SMILES: c1ccc2c(c1)Oc1ccccc1C21c2ccccc2-c2ccc(-c3ccc(-c4nc(-c5ccc6ccccc6c5)nc5c4sc4ccccc45)cc3)cc21 ; c1ccc(-c2ccccc2-c2nc(-c3ccc(-c4ccc5c(c4)C4(c6ccccc6Oc6ccccc64)c4ccccc4-5)cc3)c3sc4ccccc4c3n2)cc1 ; c1ccc(-c2nc(-c3cccc(-c4ccc5c(c4)C4(c6ccccc6Oc6ccccc64)c4ccccc4-5)c3)c3sc4ccccc4c3n2)cc1 ; c1ccc(-c2ccc(-c3nc(-c4cccc(-c5ccc6c(c5)C5(c7ccccc7Oc7ccccc75)c5ccccc5-6)c4)c4sc5ccccc5c4n3)cc2)cc1]

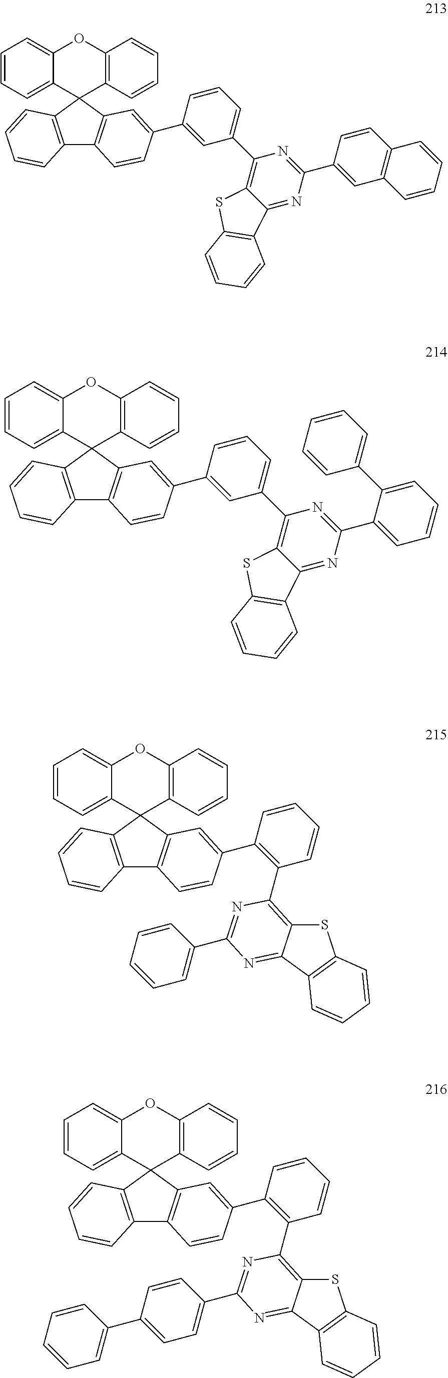 [SMILES: c1cc(-c2ccc3c(c2)C2(c4ccccc4Oc4ccccc42)c2ccccc2-3)cc(-c2nc(-c3ccc4ccccc4c3)nc3c2sc2ccccc23)c1 ; c1ccc(-c2ccccc2-c2nc(-c3cccc(-c4ccc5c(c4)C4(c6ccccc6Oc6ccccc64)c4ccccc4-5)c3)c3sc4ccccc4c3n2)cc1 ; c1ccc(-c2nc(-c3ccccc3-c3ccc4c(c3)C3(c5ccccc5Oc5ccccc53)c3ccccc3-4)c3sc4ccccc4c3n2)cc1 ; c1ccc(-c2ccc(-c3nc(-c4ccccc4-c4ccc5c(c4)C4(c6ccccc6Oc6ccccc64)c4ccccc4-5)c4sc5ccccc5c4n3)cc2)cc1]

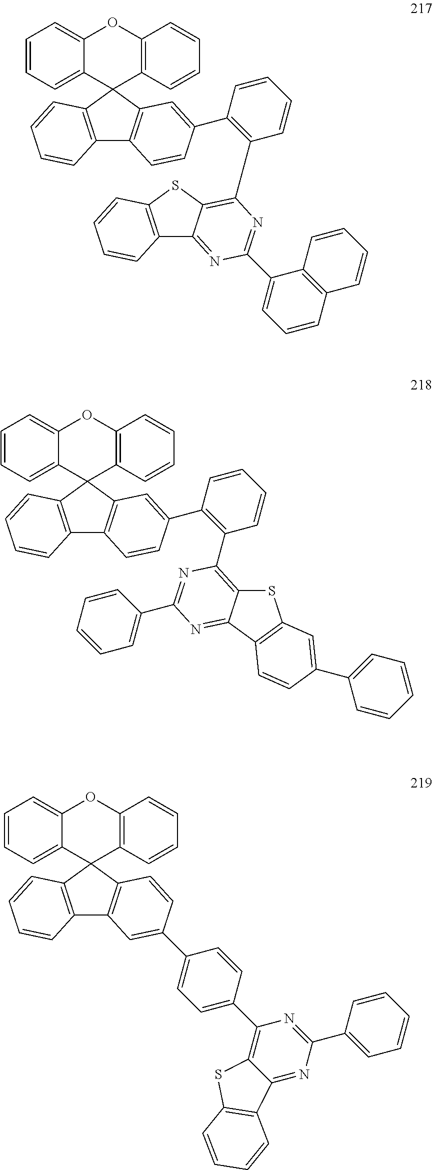 [SMILES: c1ccc2c(c1)Oc1ccccc1C21c2ccccc2-c2ccc(-c3ccccc3-c3nc(-c4cccc5ccccc45)nc4c3sc3ccccc34)cc21 ; c1ccc(-c2ccc3c(c2)sc2c(-c4ccccc4-c4ccc5c(c4)C4(c6ccccc6Oc6ccccc64)c4ccccc4-5)nc(-c4ccccc4)nc23)cc1 ; c1ccc(-c2nc(-c3ccc(-c4ccc5c(c4)-c4ccccc4C54c5ccccc5Oc5ccccc54)cc3)c3sc4ccccc4c3n2)cc1]

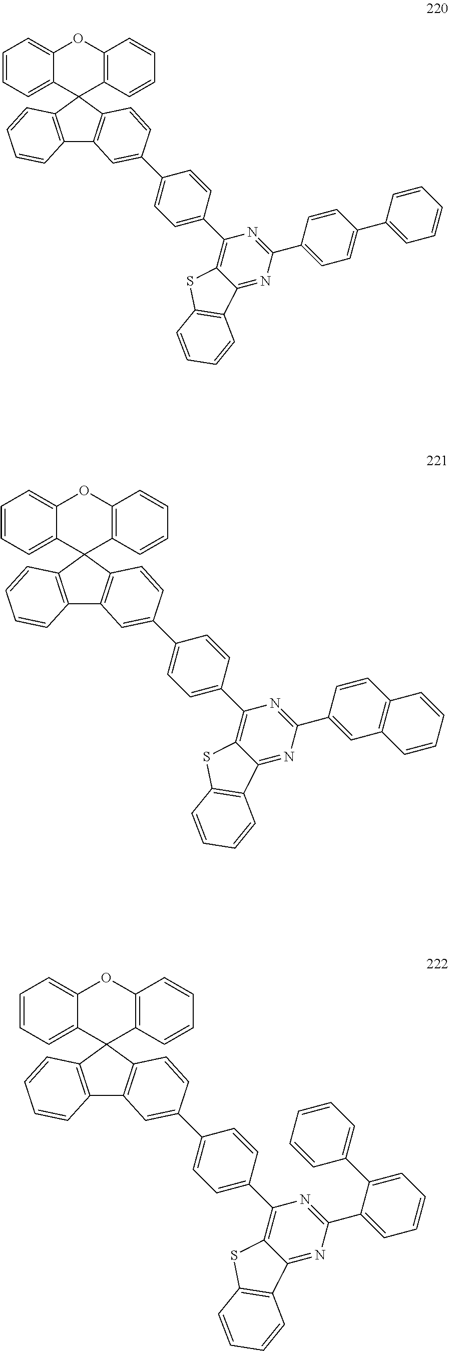 [SMILES: c1ccc(-c2ccc(-c3nc(-c4ccc(-c5ccc6c(c5)-c5ccccc5C65c6ccccc6Oc6ccccc65)cc4)c4sc5ccccc5c4n3)cc2)cc1 ; c1ccc2c(c1)Oc1ccccc1C21c2ccccc2-c2cc(-c3ccc(-c4nc(-c5ccc6ccccc6c5)nc5c4sc4ccccc45)cc3)ccc21 ; c1ccc(-c2ccccc2-c2nc(-c3ccc(-c4ccc5c(c4)-c4ccccc4C54c5ccccc5Oc5ccccc54)cc3)c3sc4ccccc4c3n2)cc1]

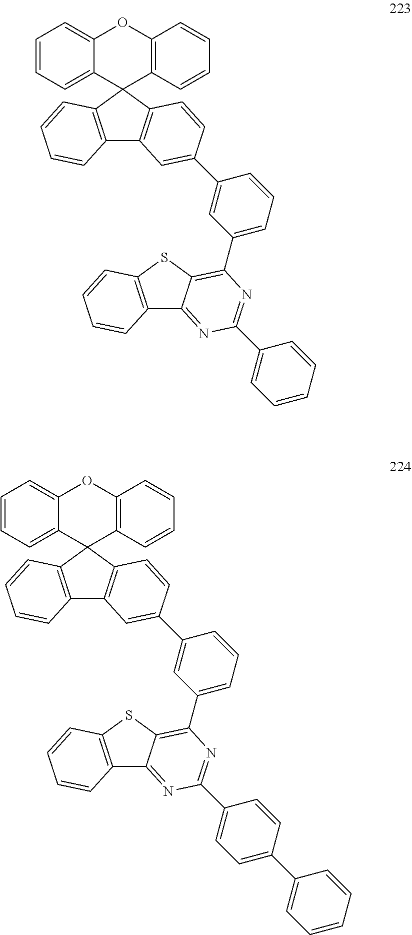 [SMILES: c1ccc(-c2nc(-c3cccc(-c4ccc5c(c4)-c4ccccc4C54c5ccccc5Oc5ccccc54)c3)c3sc4ccccc4c3n2)cc1 ; c1ccc(-c2ccc(-c3nc(-c4cccc(-c5ccc6c(c5)-c5ccccc5C65c6ccccc6Oc6ccccc65)c4)c4sc5ccccc5c4n3)cc2)cc1]

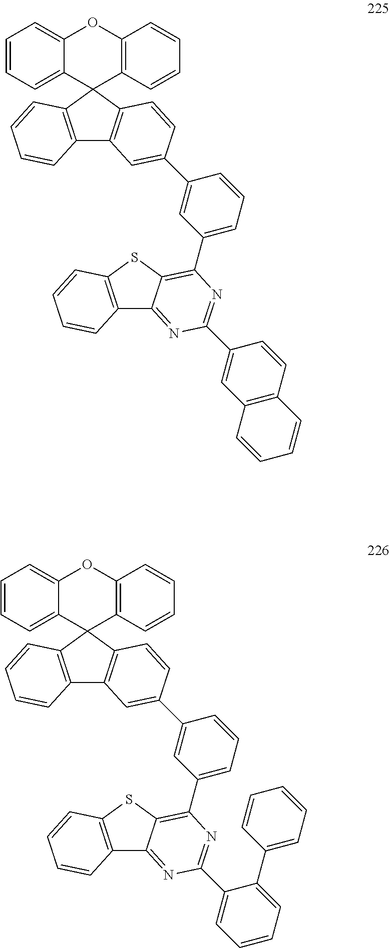 [SMILES: c1cc(-c2ccc3c(c2)-c2ccccc2C32c3ccccc3Oc3ccccc32)cc(-c2nc(-c3ccc4ccccc4c3)nc3c2sc2ccccc23)c1 ; c1ccc(-c2ccccc2-c2nc(-c3cccc(-c4ccc5c(c4)-c4ccccc4C54c5ccccc5Oc5ccccc54)c3)c3sc4ccccc4c3n2)cc1]

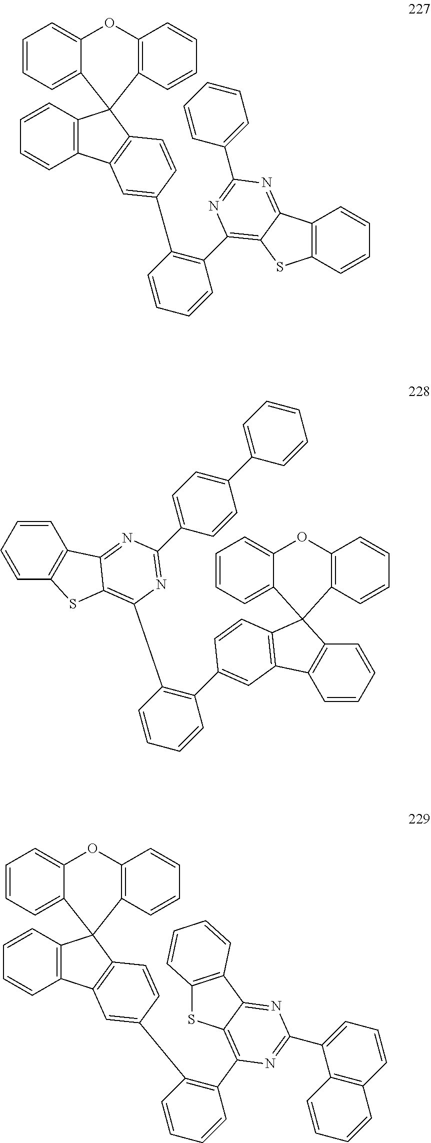 [SMILES: c1ccc(-c2nc(-c3ccccc3-c3ccc4c(c3)-c3ccccc3C43c4ccccc4Oc4ccccc43)c3sc4ccccc4c3n2)cc1 ; c1ccc(-c2ccc(-c3nc(-c4ccccc4-c4ccc5c(c4)-c4ccccc4C54c5ccccc5Oc5ccccc54)c4sc5ccccc5c4n3)cc2)cc1 ; c1ccc2c(c1)Oc1ccccc1C21c2ccccc2-c2cc(-c3ccccc3-c3nc(-c4cccc5ccccc45)nc4c3sc3ccccc34)ccc21]

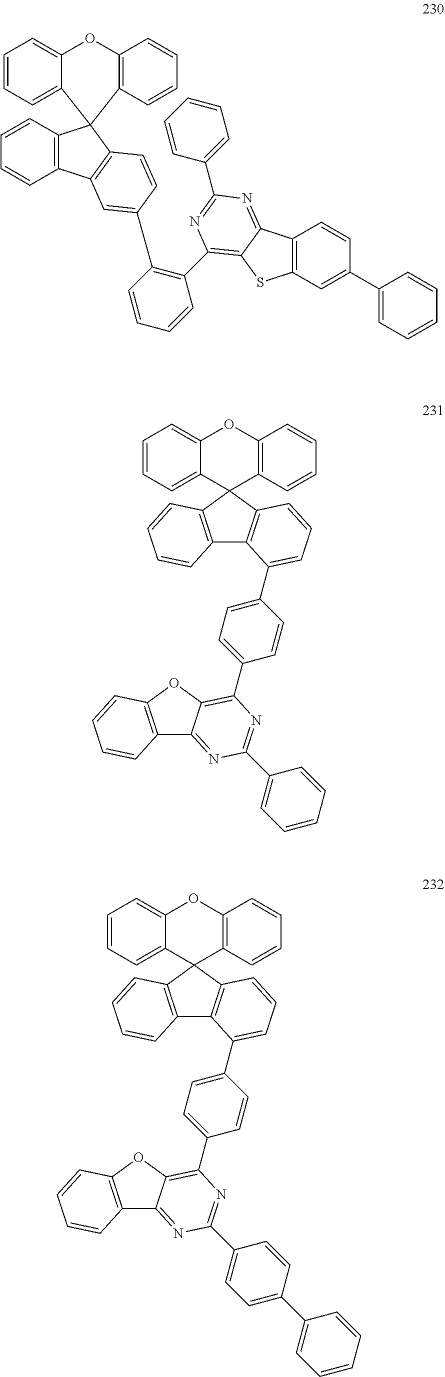 [SMILES: c1ccc(-c2ccc3c(c2)sc2c(-c4ccccc4-c4ccc5c(c4)-c4ccccc4C54c5ccccc5Oc5ccccc54)nc(-c4ccccc4)nc23)cc1 ; c1ccc(-c2nc(-c3ccc(-c4cccc5c4-c4ccccc4C54c5ccccc5Oc5ccccc54)cc3)c3oc4ccccc4c3n2)cc1 ; c1ccc(-c2ccc(-c3nc(-c4ccc(-c5cccc6c5-c5ccccc5C65c6ccccc6Oc6ccccc65)cc4)c4oc5ccccc5c4n3)cc2)cc1]

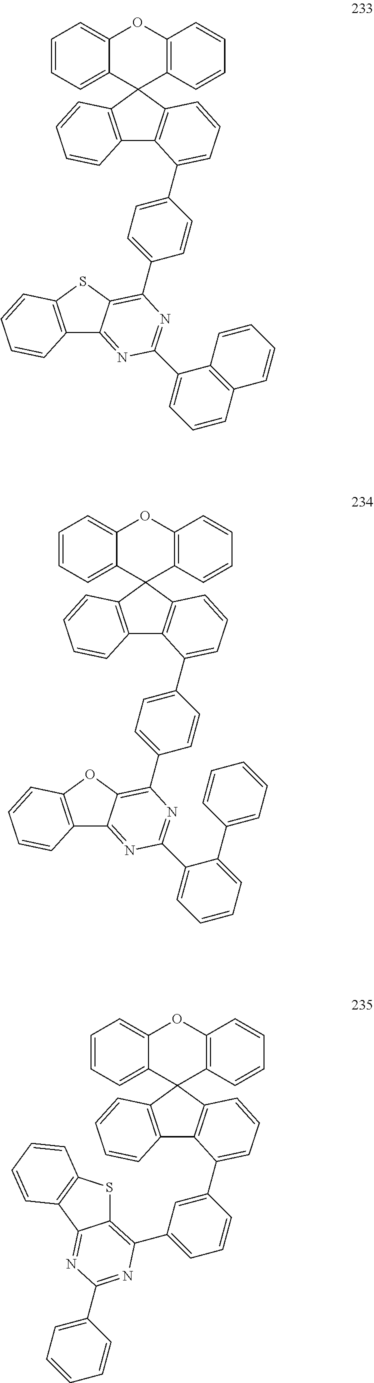 [SMILES: c1ccc2c(c1)Oc1ccccc1C21c2ccccc2-c2c(-c3ccc(-c4nc(-c5cccc6ccccc56)nc5c4sc4ccccc45)cc3)cccc21 ; c1ccc(-c2ccccc2-c2nc(-c3ccc(-c4cccc5c4-c4ccccc4C54c5ccccc5Oc5ccccc54)cc3)c3oc4ccccc4c3n2)cc1 ; c1ccc(-c2nc(-c3cccc(-c4cccc5c4-c4ccccc4C54c5ccccc5Oc5ccccc54)c3)c3sc4ccccc4c3n2)cc1]

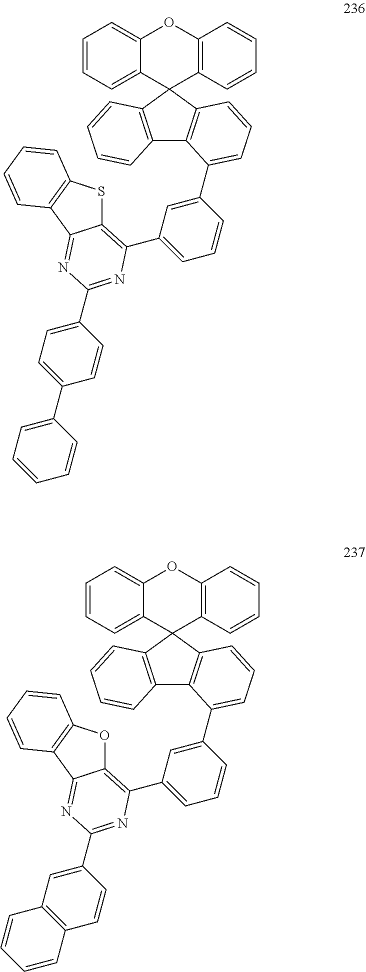 [SMILES: c1ccc(-c2ccc(-c3nc(-c4cccc(-c5cccc6c5-c5ccccc5C65c6ccccc6Oc6ccccc65)c4)c4sc5ccccc5c4n3)cc2)cc1 ; c1cc(-c2cccc3c2-c2ccccc2C32c3ccccc3Oc3ccccc32)cc(-c2nc(-c3ccc4ccccc4c3)nc3c2oc2ccccc23)c1]

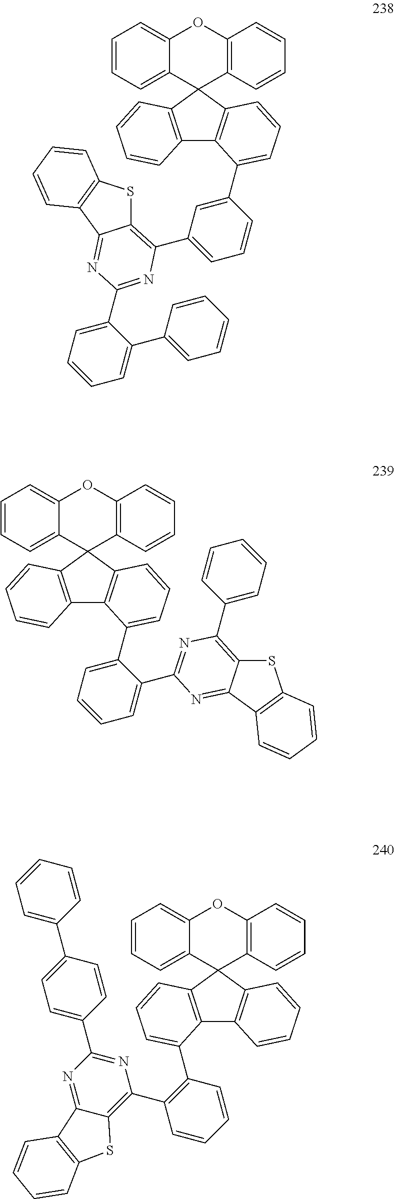 [SMILES: c1ccc(-c2ccccc2-c2nc(-c3cccc(-c4cccc5c4-c4ccccc4C54c5ccccc5Oc5ccccc54)c3)c3sc4ccccc4c3n2)cc1 ; c1ccc(-c2nc(-c3ccccc3-c3cccc4c3-c3ccccc3C43c4ccccc4Oc4ccccc43)nc3c2sc2ccccc23)cc1 ; c1ccc(-c2ccc(-c3nc(-c4ccccc4-c4cccc5c4-c4ccccc4C54c5ccccc5Oc5ccccc54)c4sc5ccccc5c4n3)cc2)cc1]

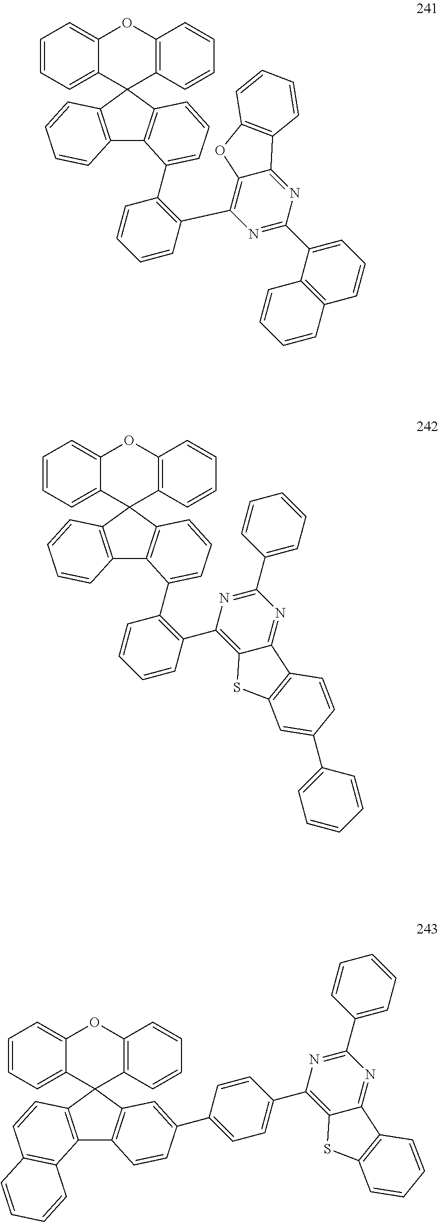 [SMILES: c1ccc2c(c1)Oc1ccccc1C21c2ccccc2-c2c(-c3ccccc3-c3nc(-c4cccc5ccccc45)nc4c3oc3ccccc34)cccc21 ; c1ccc(-c2ccc3c(c2)sc2c(-c4ccccc4-c4cccc5c4-c4ccccc4C54c5ccccc5Oc5ccccc54)nc(-c4ccccc4)nc23)cc1 ; c1ccc(-c2nc(-c3ccc(-c4ccc5c(c4)C4(c6ccccc6Oc6ccccc64)c4ccc6ccccc6c4-5)cc3)c3sc4ccccc4c3n2)cc1]

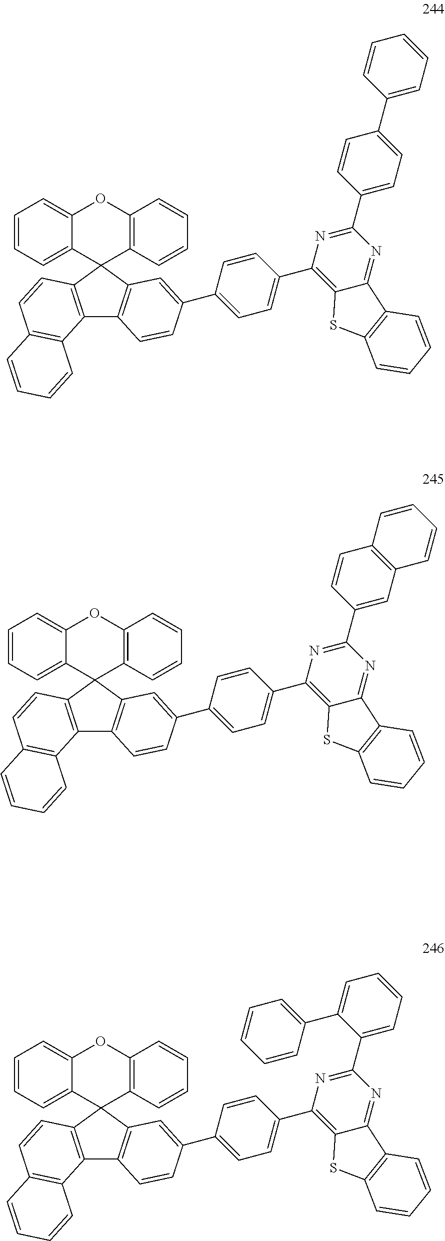 [SMILES: c1ccc(-c2ccc(-c3nc(-c4ccc(-c5ccc6c(c5)C5(c7ccccc7Oc7ccccc75)c5ccc7ccccc7c5-6)cc4)c4sc5ccccc5c4n3)cc2)cc1 ; c1ccc2c(c1)Oc1ccccc1C21c2cc(-c3ccc(-c4nc(-c5ccc6ccccc6c5)nc5c4sc4ccccc45)cc3)ccc2-c2c1ccc1ccccc21 ; c1ccc(-c2ccccc2-c2nc(-c3ccc(-c4ccc5c(c4)C4(c6ccccc6Oc6ccccc64)c4ccc6ccccc6c4-5)cc3)c3sc4ccccc4c3n2)cc1]

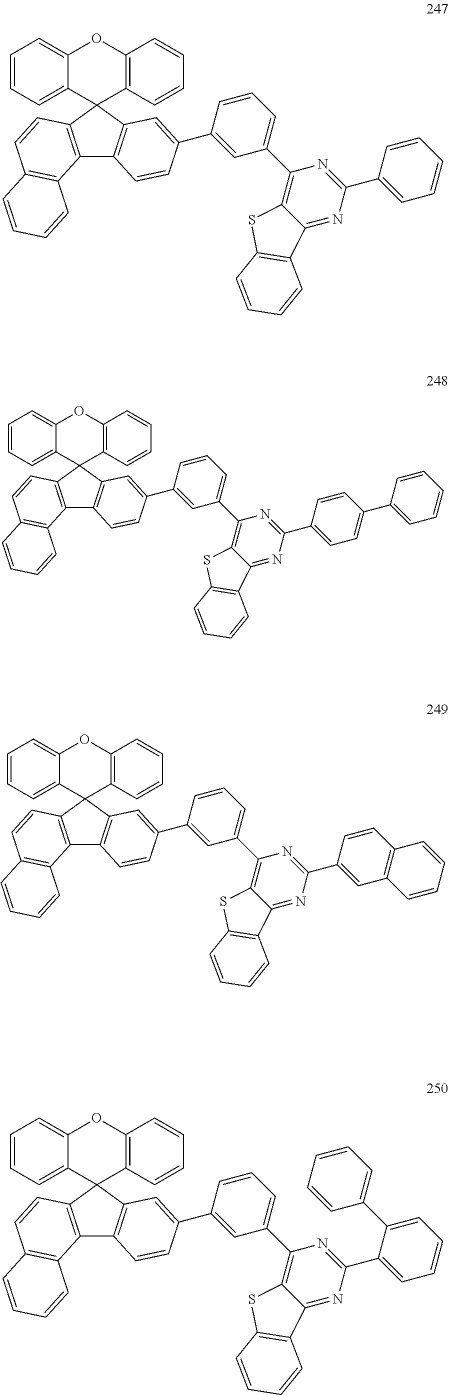 [SMILES: c1ccc(-c2nc(-c3cccc(-c4ccc5c(c4)C4(c6ccccc6Oc6ccccc64)c4ccc6ccccc6c4-5)c3)c3sc4ccccc4c3n2)cc1 ; c1ccc(-c2ccc(-c3nc(-c4cccc(-c5ccc6c(c5)C5(c7ccccc7Oc7ccccc75)c5ccc7ccccc7c5-6)c4)c4sc5ccccc5c4n3)cc2)cc1 ; c1cc(-c2ccc3c(c2)C2(c4ccccc4Oc4ccccc42)c2ccc4ccccc4c2-3)cc(-c2nc(-c3ccc4ccccc4c3)nc3c2sc2ccccc23)c1 ; c1ccc(-c2ccccc2-c2nc(-c3cccc(-c4ccc5c(c4)C4(c6ccccc6Oc6ccccc64)c4ccc6ccccc6c4-5)c3)c3sc4ccccc4c3n2)cc1]

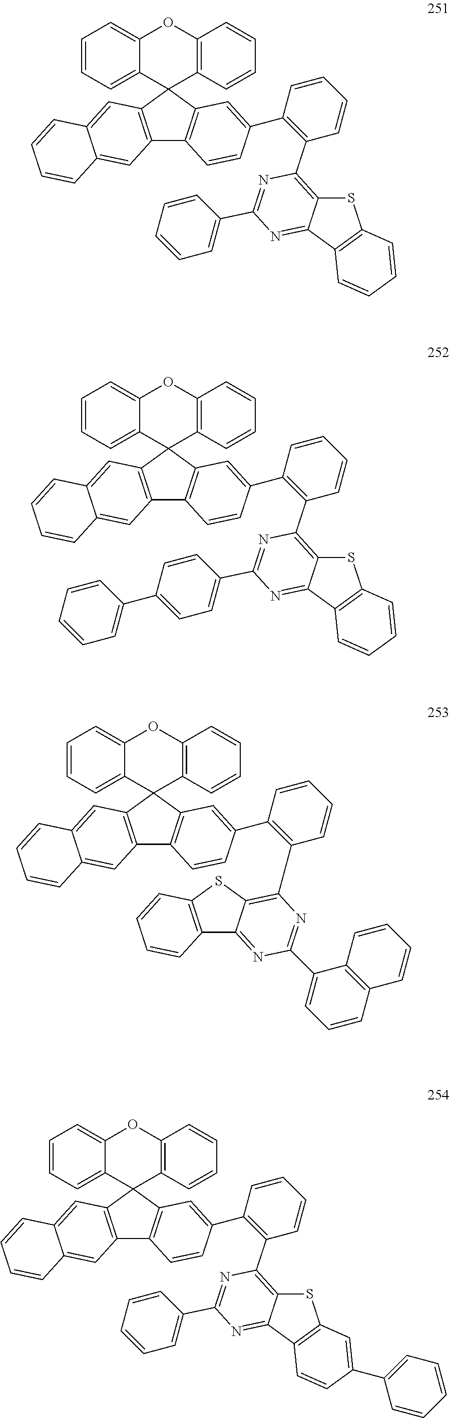 [SMILES: c1ccc(-c2nc(-c3ccccc3-c3ccc4c(c3)C3(c5ccccc5Oc5ccccc53)c3cc5ccccc5cc3-4)c3sc4ccccc4c3n2)cc1 ; c1ccc(-c2ccc(-c3nc(-c4ccccc4-c4ccc5c(c4)C4(c6ccccc6Oc6ccccc64)c4cc6ccccc6cc4-5)c4sc5ccccc5c4n3)cc2)cc1 ; c1ccc2c(c1)Oc1ccccc1C21c2cc(-c3ccccc3-c3nc(-c4cccc5ccccc45)nc4c3sc3ccccc34)ccc2-c2cc3ccccc3cc21 ; c1ccc(-c2ccc3c(c2)sc2c(-c4ccccc4-c4ccc5c(c4)C4(c6ccccc6Oc6ccccc64)c4cc6ccccc6cc4-5)nc(-c4ccccc4)nc23)cc1]

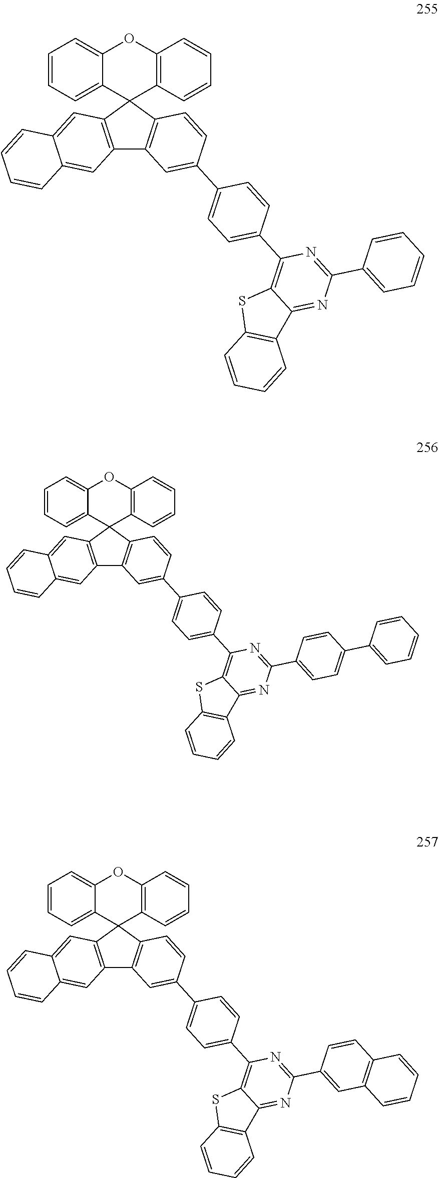 [SMILES: c1ccc(-c2nc(-c3ccc(-c4ccc5c(c4)-c4cc6ccccc6cc4C54c5ccccc5Oc5ccccc54)cc3)c3sc4ccccc4c3n2)cc1 ; c1ccc(-c2ccc(-c3nc(-c4ccc(-c5ccc6c(c5)-c5cc7ccccc7cc5C65c6ccccc6Oc6ccccc65)cc4)c4sc5ccccc5c4n3)cc2)cc1 ; c1ccc2c(c1)Oc1ccccc1C21c2ccc(-c3ccc(-c4nc(-c5ccc6ccccc6c5)nc5c4sc4ccccc45)cc3)cc2-c2cc3ccccc3cc21]

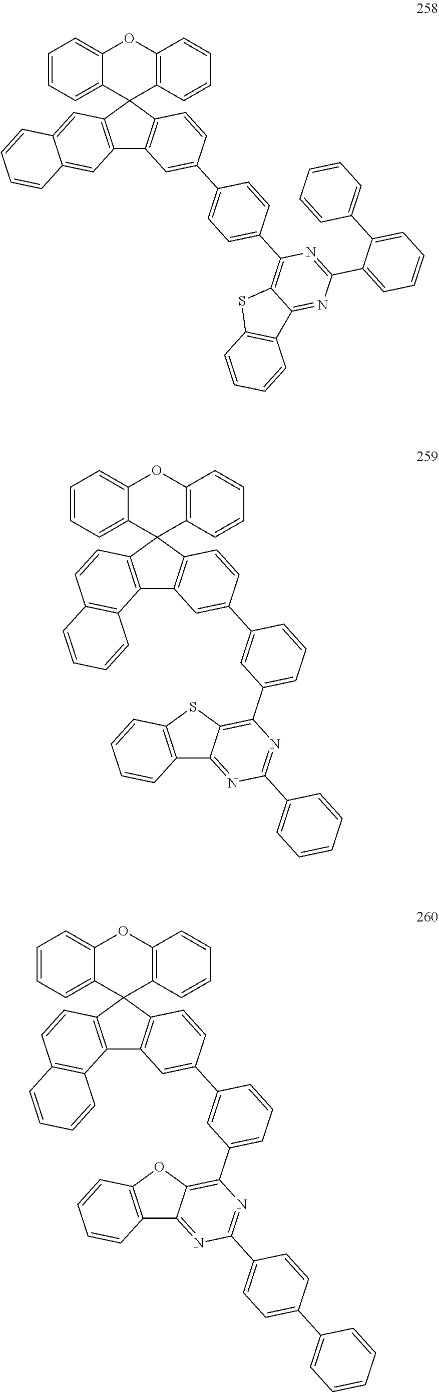 [SMILES: c1ccc(-c2ccccc2-c2nc(-c3ccc(-c4ccc5c(c4)-c4cc6ccccc6cc4C54c5ccccc5Oc5ccccc54)cc3)c3sc4ccccc4c3n2)cc1 ; c1ccc(-c2nc(-c3cccc(-c4ccc5c(c4)-c4c(ccc6ccccc46)C54c5ccccc5Oc5ccccc54)c3)c3sc4ccccc4c3n2)cc1 ; c1ccc(-c2ccc(-c3nc(-c4cccc(-c5ccc6c(c5)-c5c(ccc7ccccc57)C65c6ccccc6Oc6ccccc65)c4)c4oc5ccccc5c4n3)cc2)cc1]

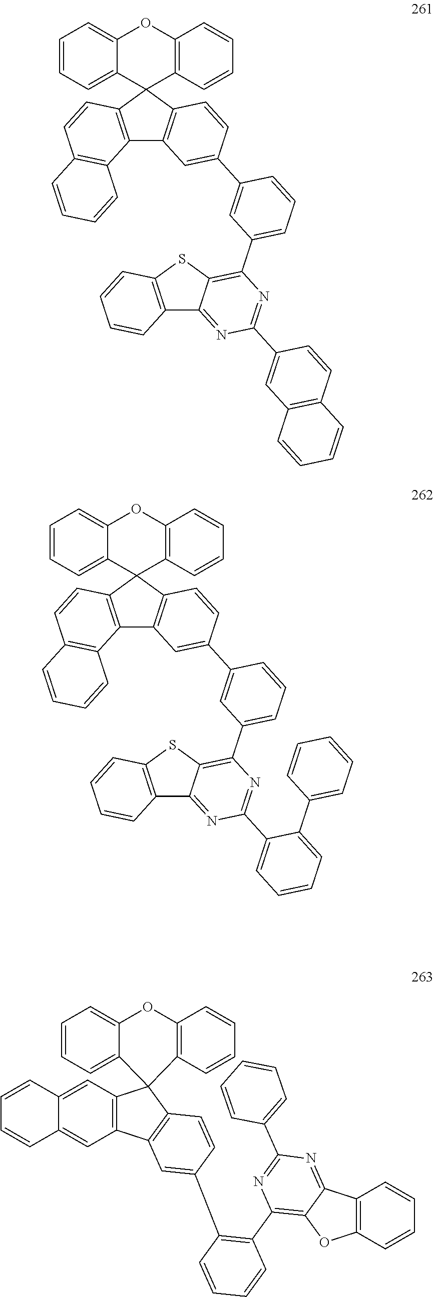 [SMILES: c1cc(-c2ccc3c(c2)-c2c(ccc4ccccc24)C32c3ccccc3Oc3ccccc32)cc(-c2nc(-c3ccc4ccccc4c3)nc3c2sc2ccccc23)c1 ; c1ccc(-c2ccccc2-c2nc(-c3cccc(-c4ccc5c(c4)-c4c(ccc6ccccc46)C54c5ccccc5Oc5ccccc54)c3)c3sc4ccccc4c3n2)cc1 ; c1ccc(-c2nc(-c3ccccc3-c3ccc4c(c3)-c3cc5ccccc5cc3C43c4ccccc4Oc4ccccc43)c3oc4ccccc4c3n2)cc1]

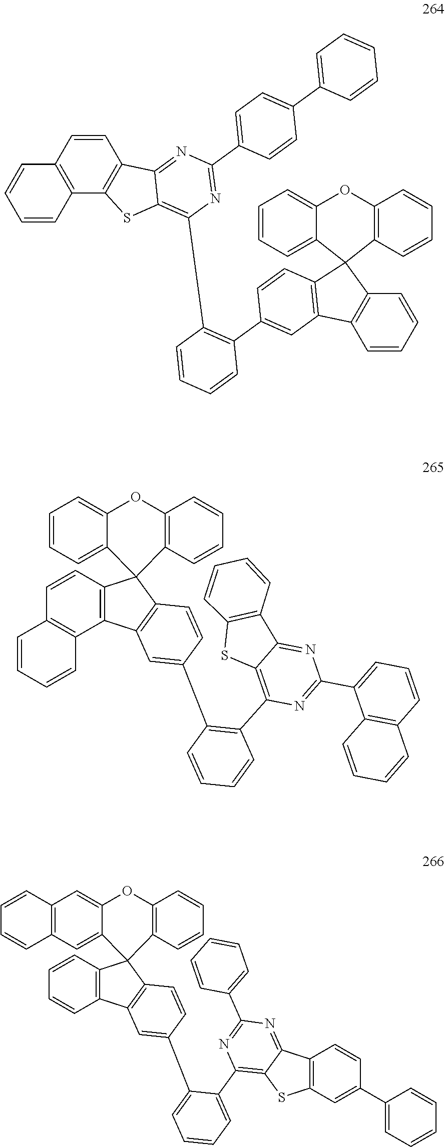 [SMILES: c1ccc(-c2ccc(-c3nc(-c4ccccc4-c4ccc5c(c4)-c4ccccc4C54c5ccccc5Oc5ccccc54)c4sc5c6ccccc6ccc5c4n3)cc2)cc1 ; c1ccc2c(c1)Oc1ccccc1C21c2ccc(-c3ccccc3-c3nc(-c4cccc5ccccc45)nc4c3sc3ccccc34)cc2-c2c1ccc1ccccc21 ; c1ccc(-c2ccc3c(c2)sc2c(-c4ccccc4-c4ccc5c(c4)-c4ccccc4C54c5ccccc5Oc5cc6ccccc6cc54)nc(-c4ccccc4)nc23)cc1]

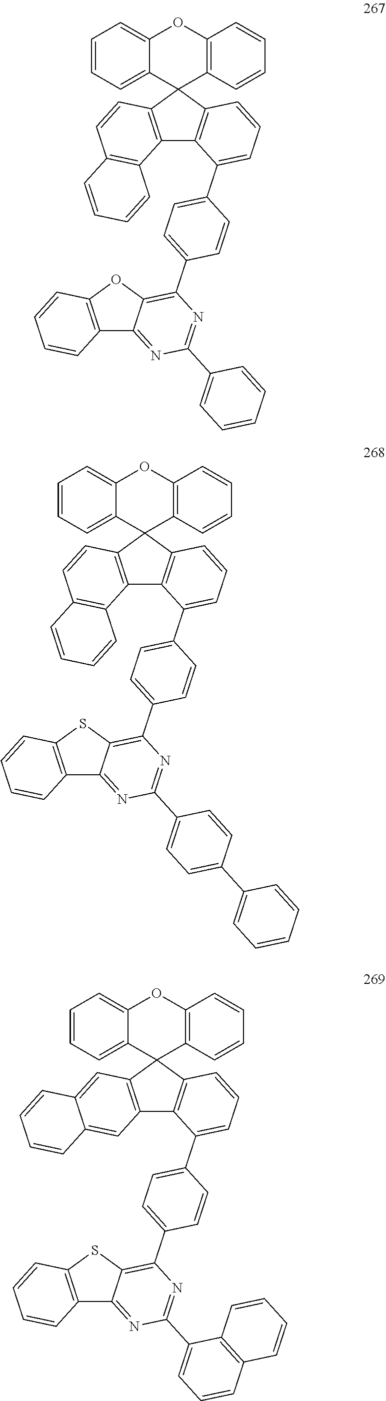

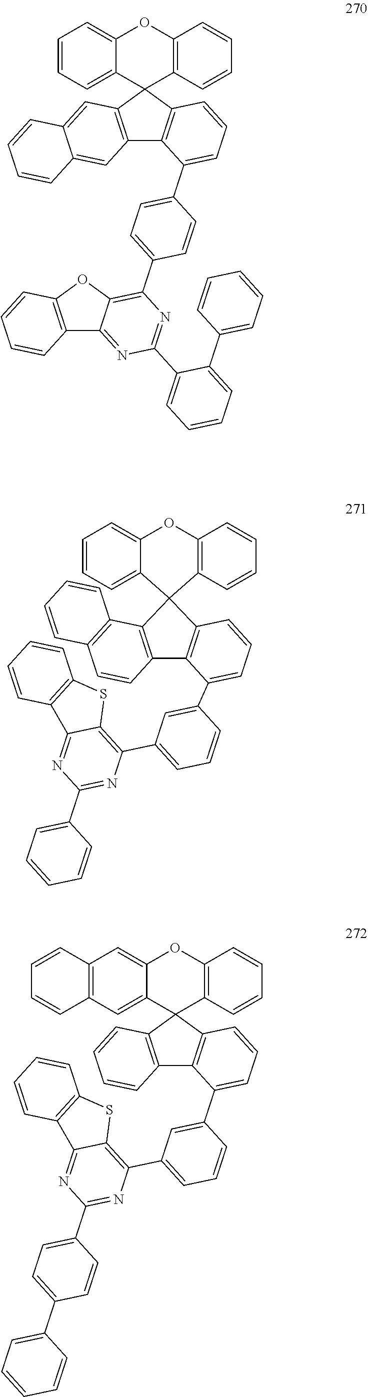 [SMILES: c1ccc(-c2ccccc2-c2nc(-c3ccc(-c4cccc5c4-c4cc6ccccc6cc4C54c5ccccc5Oc5ccccc54)cc3)c3oc4ccccc4c3n2)cc1 ; c1ccc(-c2nc(-c3cccc(-c4cccc5c4-c4ccc6ccccc6c4C54c5ccccc5Oc5ccccc54)c3)c3sc4ccccc4c3n2)cc1 ; c1ccc(-c2ccc(-c3nc(-c4cccc(-c5cccc6c5-c5ccccc5C65c6ccccc6Oc6cc7ccccc7cc65)c4)c4sc5ccccc5c4n3)cc2)cc1]

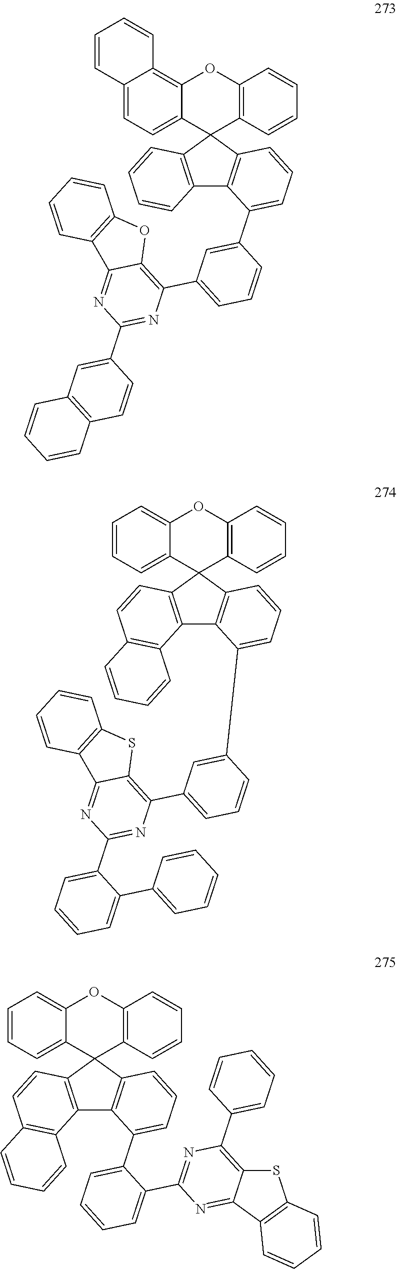 [SMILES: c1cc(-c2cccc3c2-c2ccccc2C32c3ccccc3Oc3c2ccc2ccccc32)cc(-c2nc(-c3ccc4ccccc4c3)nc3c2oc2ccccc23)c1 ; c1ccc(-c2ccccc2-c2nc(-c3cccc(-c4cccc5c4-c4c(ccc6ccccc46)C54c5ccccc5Oc5ccccc54)c3)c3sc4ccccc4c3n2)cc1 ; c1ccc(-c2nc(-c3ccccc3-c3cccc4c3-c3c(ccc5ccccc35)C43c4ccccc4Oc4ccccc43)nc3c2sc2ccccc23)cc1]

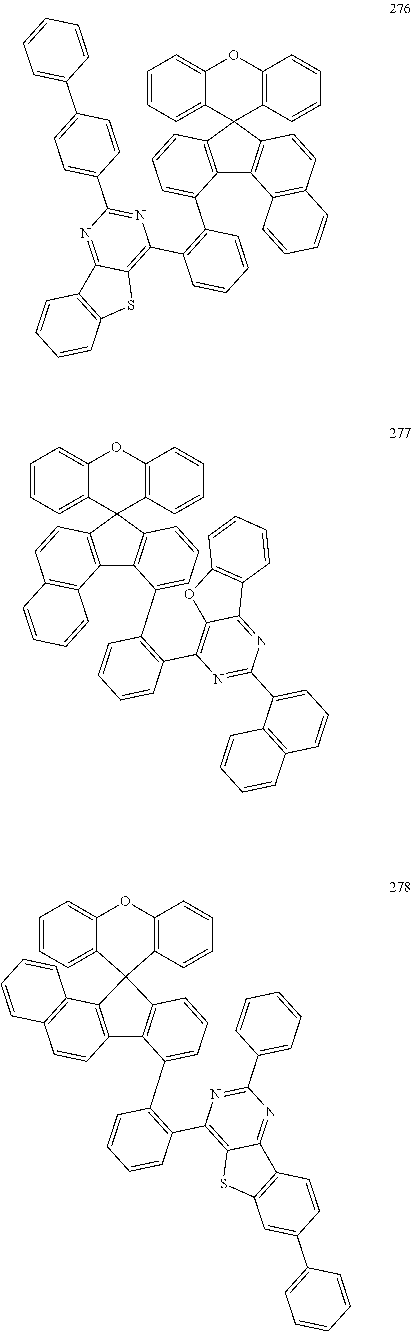 [SMILES: c1ccc(-c2ccc(-c3nc(-c4ccccc4-c4cccc5c4-c4c(ccc6ccccc46)C54c5ccccc5Oc5ccccc54)c4sc5ccccc5c4n3)cc2)cc1 ; c1ccc2c(c1)Oc1ccccc1C21c2cccc(-c3ccccc3-c3nc(-c4cccc5ccccc45)nc4c3oc3ccccc34)c2-c2c1ccc1ccccc21 ; c1ccc(-c2ccc3c(c2)sc2c(-c4ccccc4-c4cccc5c4-c4ccc6ccccc6c4C54c5ccccc5Oc5ccccc54)nc(-c4ccccc4)nc23)cc1]

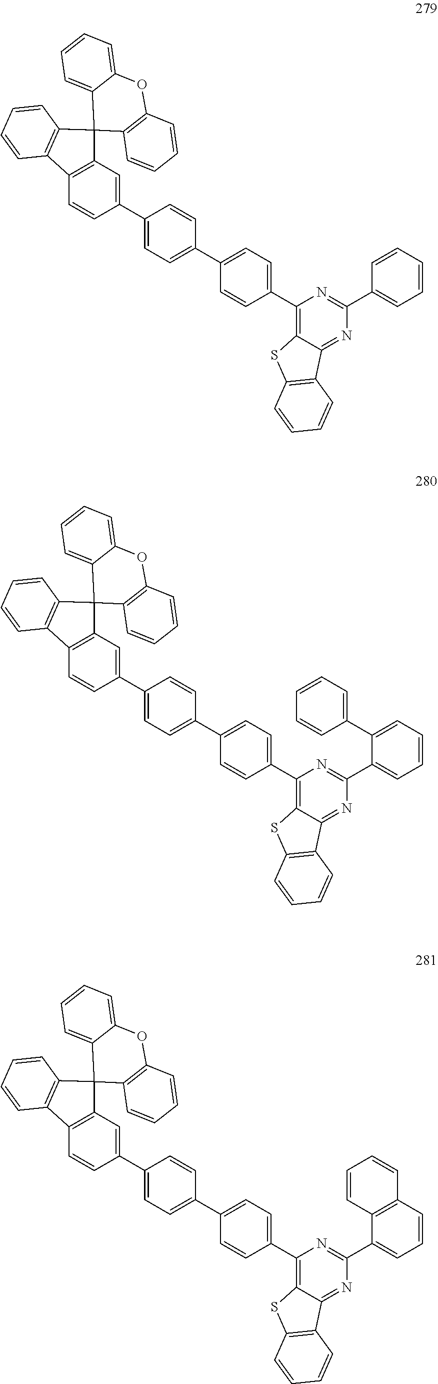 [SMILES: c1ccc(-c2nc(-c3ccc(-c4ccc(-c5ccc6c(c5)C5(c7ccccc7Oc7ccccc75)c5ccccc5-6)cc4)cc3)c3sc4ccccc4c3n2)cc1 ; c1ccc(-c2ccccc2-c2nc(-c3ccc(-c4ccc(-c5ccc6c(c5)C5(c7ccccc7Oc7ccccc75)c5ccccc5-6)cc4)cc3)c3sc4ccccc4c3n2)cc1 ; c1ccc2c(c1)Oc1ccccc1C21c2ccccc2-c2ccc(-c3ccc(-c4ccc(-c5nc(-c6cccc7ccccc67)nc6c5sc5ccccc56)cc4)cc3)cc21]

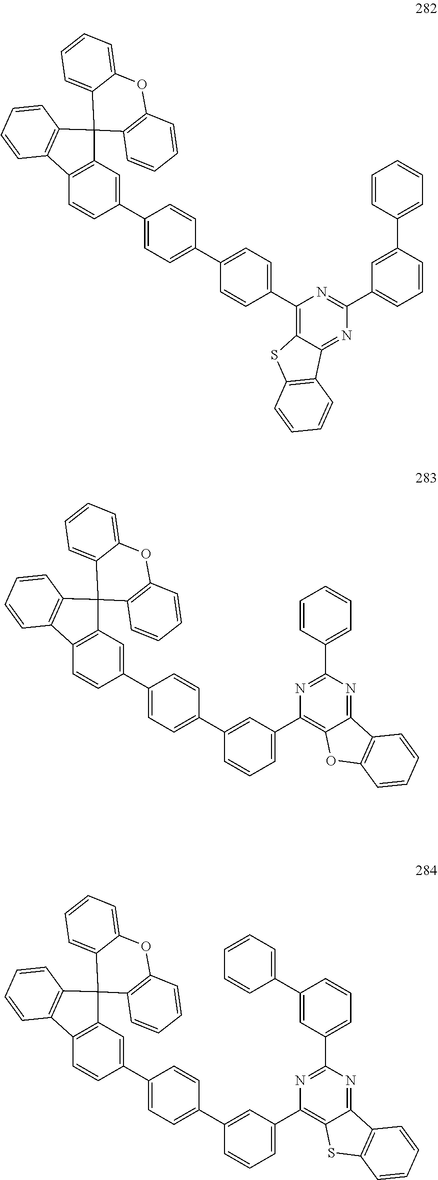 [SMILES: c1ccc(-c2cccc(-c3nc(-c4ccc(-c5ccc(-c6ccc7c(c6)C6(c8ccccc8Oc8ccccc86)c6ccccc6-7)cc5)cc4)c4sc5ccccc5c4n3)c2)cc1 ; c1ccc(-c2nc(-c3cccc(-c4ccc(-c5ccc6c(c5)C5(c7ccccc7Oc7ccccc75)c5ccccc5-6)cc4)c3)c3oc4ccccc4c3n2)cc1 ; c1ccc(-c2cccc(-c3nc(-c4cccc(-c5ccc(-c6ccc7c(c6)C6(c8ccccc8Oc8ccccc86)c6ccccc6-7)cc5)c4)c4sc5ccccc5c4n3)c2)cc1]

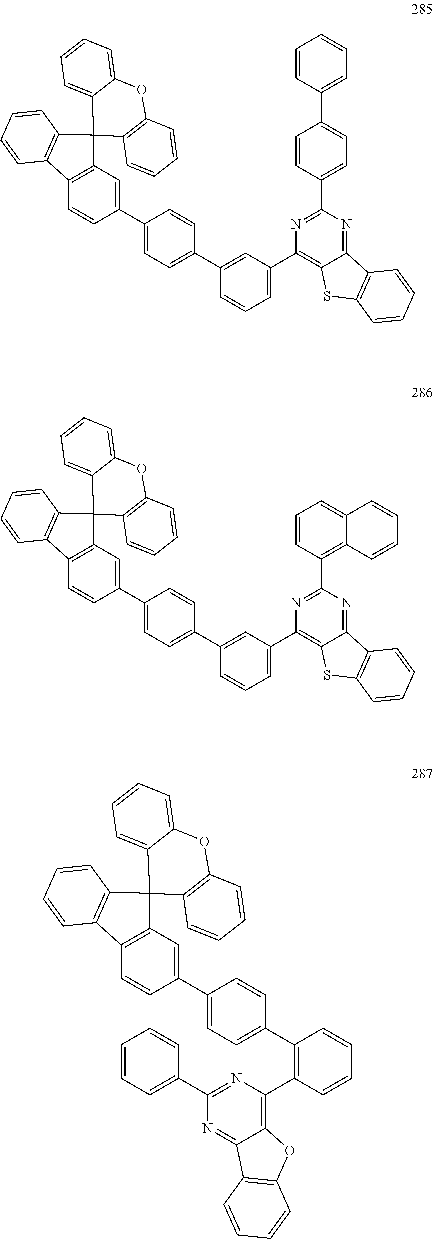 [SMILES: c1ccc(-c2ccc(-c3nc(-c4cccc(-c5ccc(-c6ccc7c(c6)C6(c8ccccc8Oc8ccccc86)c6ccccc6-7)cc5)c4)c4sc5ccccc5c4n3)cc2)cc1 ; c1cc(-c2ccc(-c3ccc4c(c3)C3(c5ccccc5Oc5ccccc53)c3ccccc3-4)cc2)cc(-c2nc(-c3cccc4ccccc34)nc3c2sc2ccccc23)c1 ; c1ccc(-c2nc(-c3ccccc3-c3ccc(-c4ccc5c(c4)C4(c6ccccc6Oc6ccccc64)c4ccccc4-5)cc3)c3oc4ccccc4c3n2)cc1]

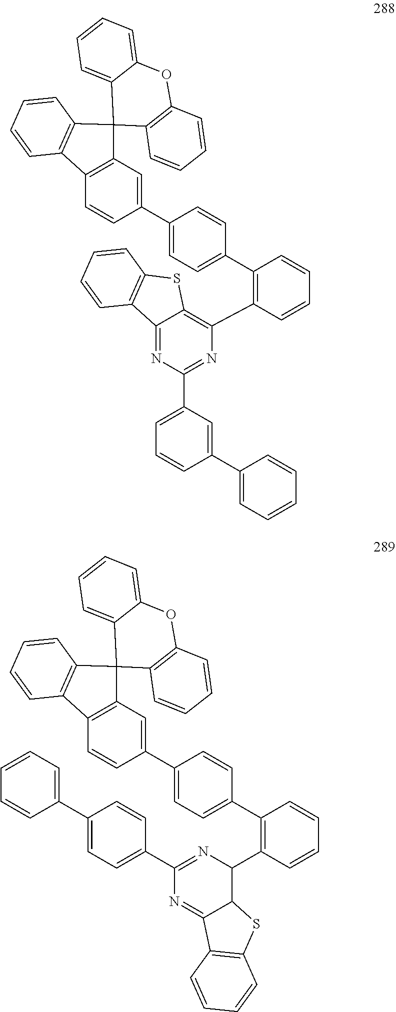 [SMILES: c1ccc(-c2cccc(-c3nc(-c4ccccc4-c4ccc(-c5ccc6c(c5)C5(c7ccccc7Oc7ccccc75)c5ccccc5-6)cc4)c4sc5ccccc5c4n3)c2)cc1 ; c1ccc(-c2ccc(C3=NC(c4ccccc4-c4ccc(-c5ccc6c(c5)C5(c7ccccc7Oc7ccccc75)c5ccccc5-6)cc4)C4Sc5ccccc5C4=N3)cc2)cc1]

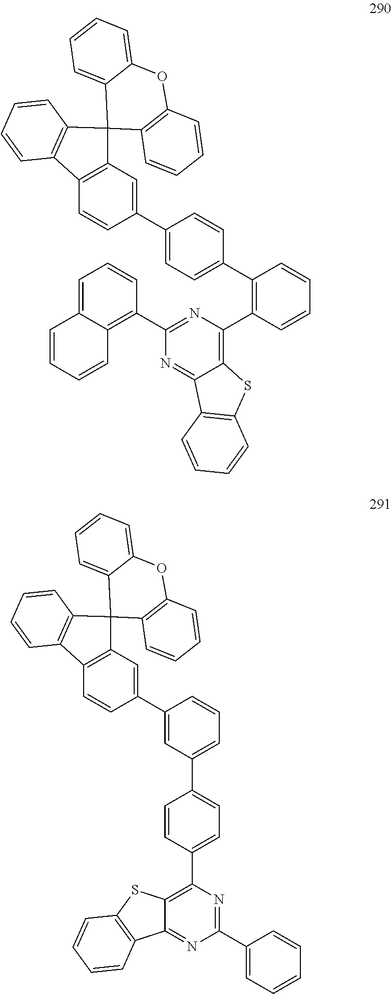 [SMILES: c1ccc2c(c1)Oc1ccccc1C21c2ccccc2-c2ccc(-c3ccc(-c4ccccc4-c4nc(-c5cccc6ccccc56)nc5c4sc4ccccc45)cc3)cc21 ; c1ccc(-c2nc(-c3ccc(-c4cccc(-c5ccc6c(c5)C5(c7ccccc7Oc7ccccc75)c5ccccc5-6)c4)cc3)c3sc4ccccc4c3n2)cc1]

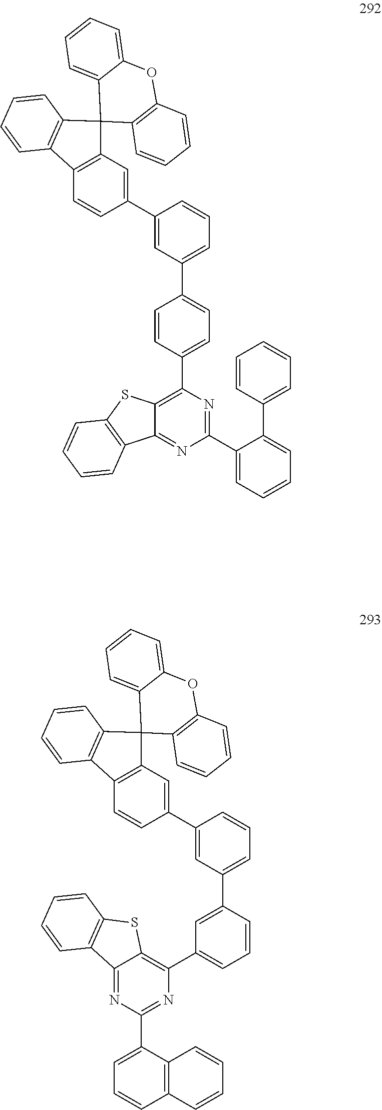 [SMILES: c1ccc(-c2ccccc2-c2nc(-c3ccc(-c4cccc(-c5ccc6c(c5)C5(c7ccccc7Oc7ccccc75)c5ccccc5-6)c4)cc3)c3sc4ccccc4c3n2)cc1 ; c1cc(-c2cccc(-c3nc(-c4cccc5ccccc45)nc4c3sc3ccccc34)c2)cc(-c2ccc3c(c2)C2(c4ccccc4Oc4ccccc42)c2ccccc2-3)c1]

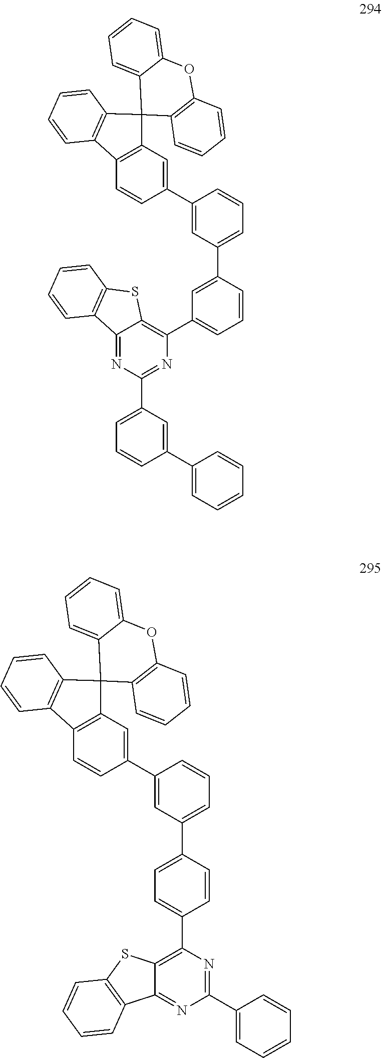 [SMILES: c1ccc(-c2cccc(-c3nc(-c4cccc(-c5cccc(-c6ccc7c(c6)C6(c8ccccc8Oc8ccccc86)c6ccccc6-7)c5)c4)c4sc5ccccc5c4n3)c2)cc1 ; c1ccc(-c2nc(-c3ccc(-c4cccc(-c5ccc6c(c5)C5(c7ccccc7Oc7ccccc75)c5ccccc5-6)c4)cc3)c3sc4ccccc4c3n2)cc1]

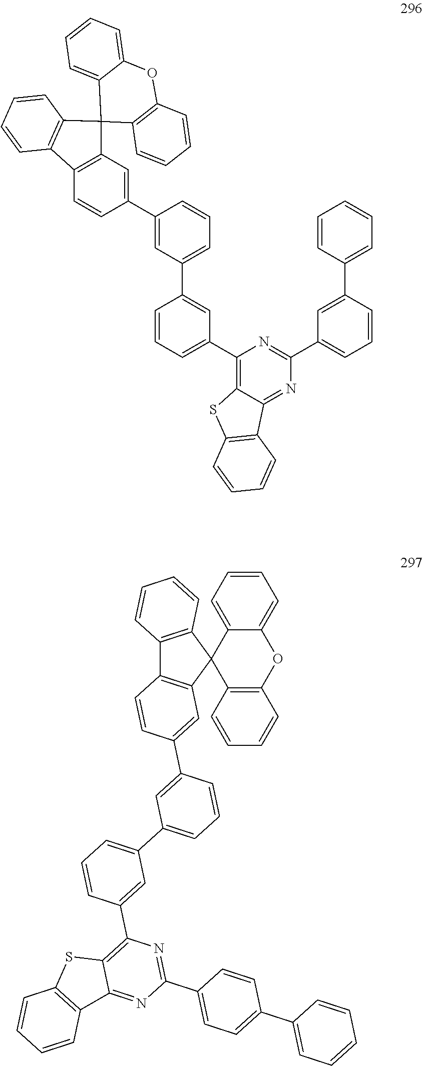 [SMILES: c1ccc(-c2cccc(-c3nc(-c4cccc(-c5cccc(-c6ccc7c(c6)C6(c8ccccc8Oc8ccccc86)c6ccccc6-7)c5)c4)c4sc5ccccc5c4n3)c2)cc1 ; c1ccc(-c2ccc(-c3nc(-c4cccc(-c5cccc(-c6ccc7c(c6)C6(c8ccccc8Oc8ccccc86)c6ccccc6-7)c5)c4)c4sc5ccccc5c4n3)cc2)cc1]

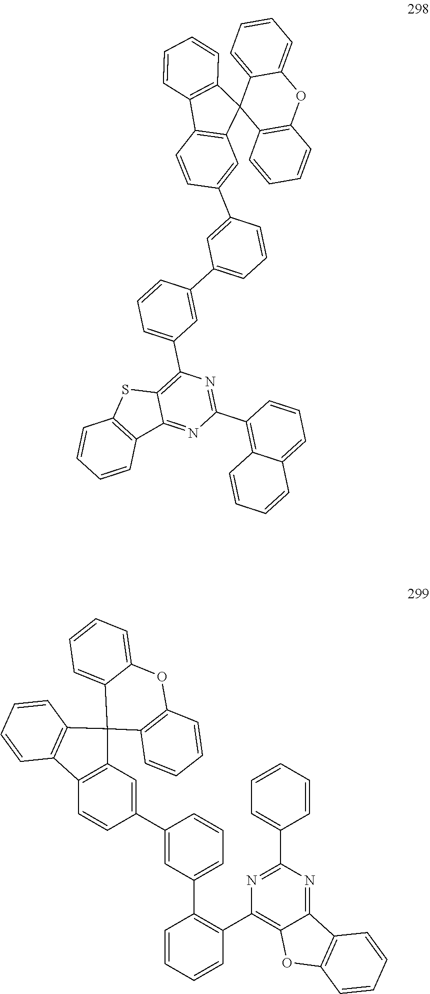 [SMILES: c1cc(-c2cccc(-c3nc(-c4cccc5ccccc45)nc4c3sc3ccccc34)c2)cc(-c2ccc3c(c2)C2(c4ccccc4Oc4ccccc42)c2ccccc2-3)c1 ; c1ccc(-c2nc(-c3ccccc3-c3cccc(-c4ccc5c(c4)C4(c6ccccc6Oc6ccccc64)c4ccccc4-5)c3)c3oc4ccccc4c3n2)cc1]

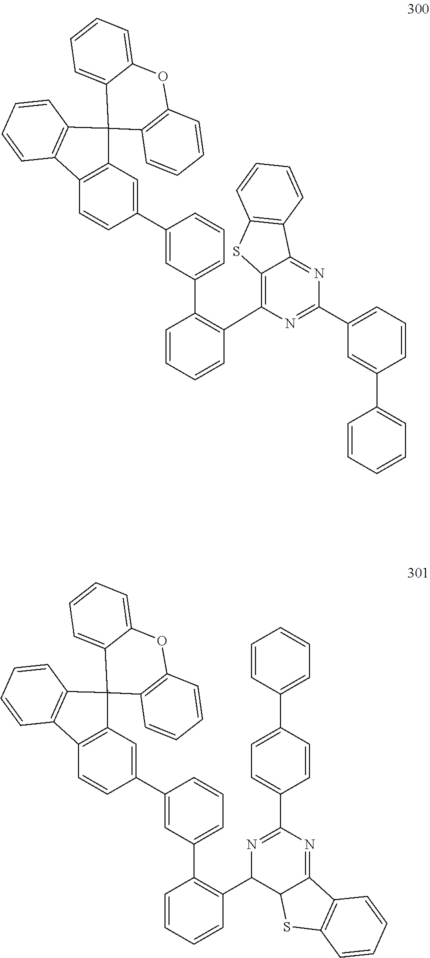 [SMILES: c1ccc(-c2cccc(-c3nc(-c4ccccc4-c4cccc(-c5ccc6c(c5)C5(c7ccccc7Oc7ccccc75)c5ccccc5-6)c4)c4sc5ccccc5c4n3)c2)cc1 ; c1ccc(-c2ccc(C3=NC(c4ccccc4-c4cccc(-c5ccc6c(c5)C5(c7ccccc7Oc7ccccc75)c5ccccc5-6)c4)C4Sc5ccccc5C4=N3)cc2)cc1]

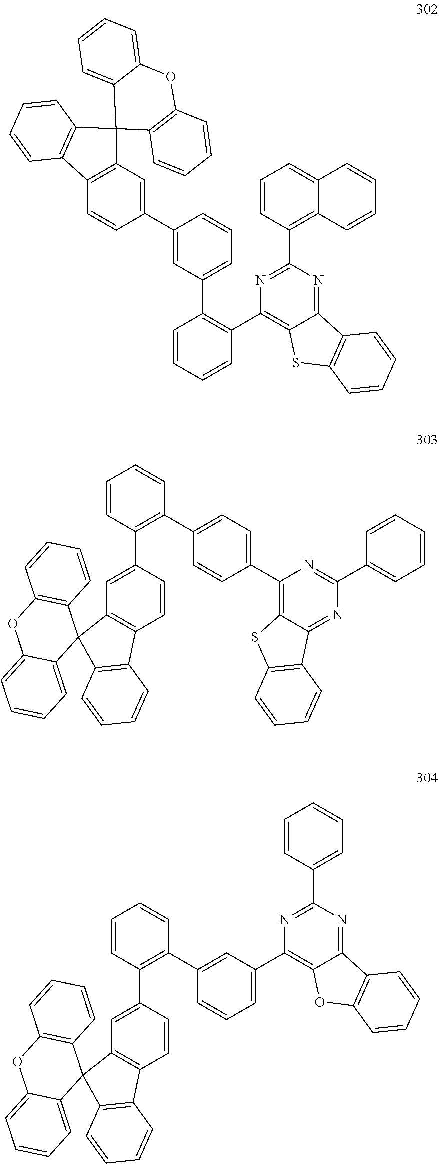 [SMILES: c1cc(-c2ccc3c(c2)C2(c4ccccc4Oc4ccccc42)c2ccccc2-3)cc(-c2ccccc2-c2nc(-c3cccc4ccccc34)nc3c2sc2ccccc23)c1 ; c1ccc(-c2nc(-c3ccc(-c4ccccc4-c4ccc5c(c4)C4(c6ccccc6Oc6ccccc64)c4ccccc4-5)cc3)c3sc4ccccc4c3n2)cc1 ; c1ccc(-c2nc(-c3cccc(-c4ccccc4-c4ccc5c(c4)C4(c6ccccc6Oc6ccccc64)c4ccccc4-5)c3)c3oc4ccccc4c3n2)cc1]

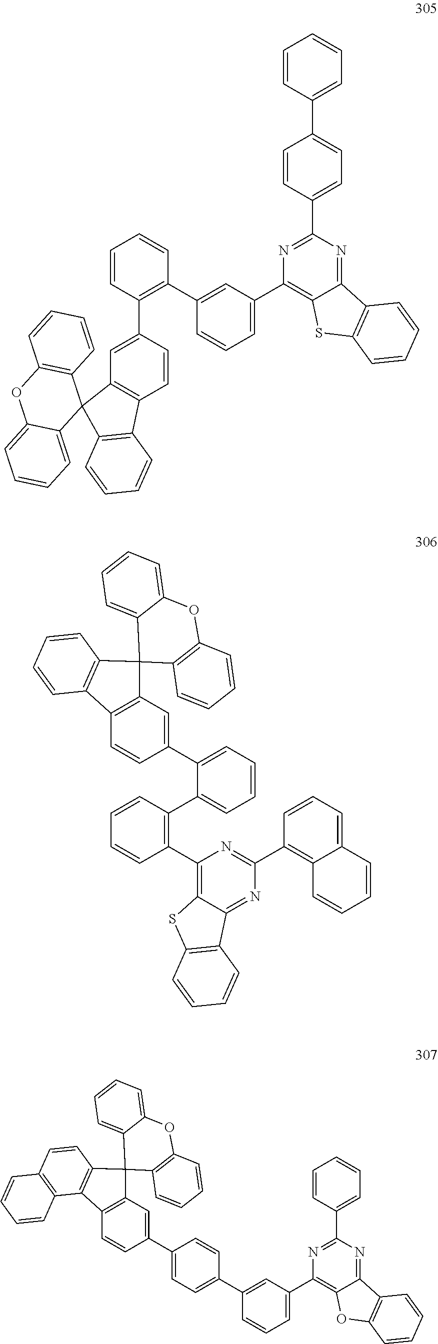 [SMILES: c1ccc(-c2ccc(-c3nc(-c4cccc(-c5ccccc5-c5ccc6c(c5)C5(c7ccccc7Oc7ccccc75)c5ccccc5-6)c4)c4sc5ccccc5c4n3)cc2)cc1 ; c1ccc2c(c1)Oc1ccccc1C21c2ccccc2-c2ccc(-c3ccccc3-c3ccccc3-c3nc(-c4cccc5ccccc45)nc4c3sc3ccccc34)cc21 ; c1ccc(-c2nc(-c3cccc(-c4ccc(-c5ccc6c(c5)C5(c7ccccc7Oc7ccccc75)c5ccc7ccccc7c5-6)cc4)c3)c3oc4ccccc4c3n2)cc1]

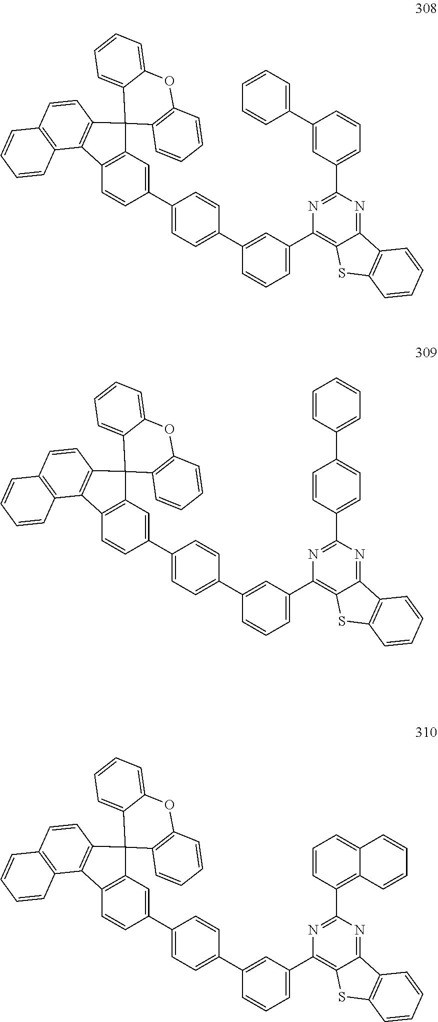 [SMILES: c1ccc(-c2cccc(-c3nc(-c4cccc(-c5ccc(-c6ccc7c(c6)C6(c8ccccc8Oc8ccccc86)c6ccc8ccccc8c6-7)cc5)c4)c4sc5ccccc5c4n3)c2)cc1 ; c1ccc(-c2ccc(-c3nc(-c4cccc(-c5ccc(-c6ccc7c(c6)C6(c8ccccc8Oc8ccccc86)c6ccc8ccccc8c6-7)cc5)c4)c4sc5ccccc5c4n3)cc2)cc1 ; c1cc(-c2ccc(-c3ccc4c(c3)C3(c5ccccc5Oc5ccccc53)c3ccc5ccccc5c3-4)cc2)cc(-c2nc(-c3cccc4ccccc34)nc3c2sc2ccccc23)c1]

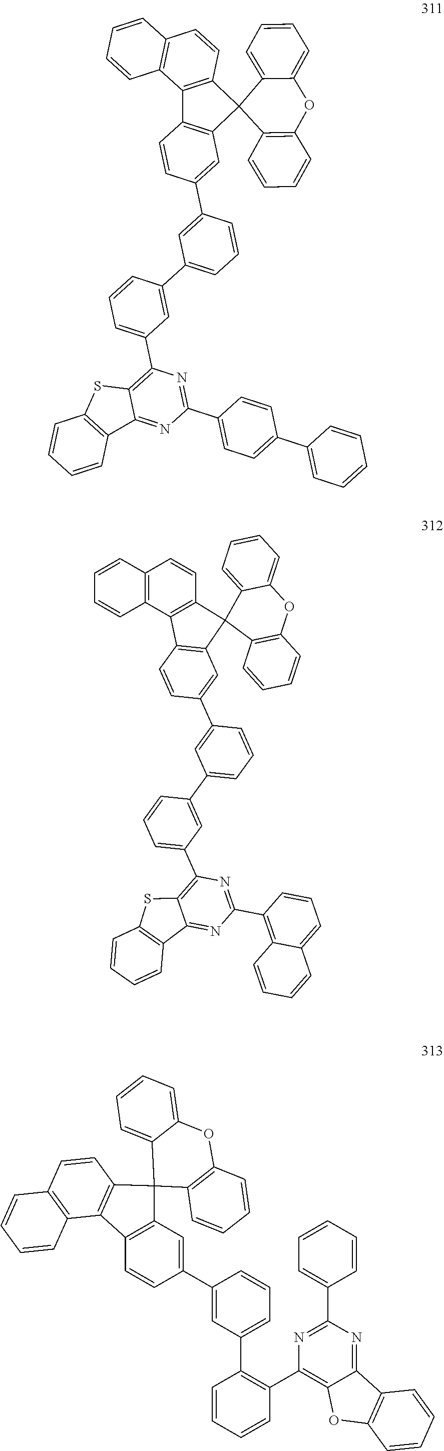 [SMILES: c1ccc(-c2ccc(-c3nc(-c4cccc(-c5cccc(-c6ccc7c(c6)C6(c8ccccc8Oc8ccccc86)c6ccc8ccccc8c6-7)c5)c4)c4sc5ccccc5c4n3)cc2)cc1 ; c1cc(-c2cccc(-c3nc(-c4cccc5ccccc45)nc4c3sc3ccccc34)c2)cc(-c2ccc3c(c2)C2(c4ccccc4Oc4ccccc42)c2ccc4ccccc4c2-3)c1 ; c1ccc(-c2nc(-c3ccccc3-c3cccc(-c4ccc5c(c4)C4(c6ccccc6Oc6ccccc64)c4ccc6ccccc6c4-5)c3)c3oc4ccccc4c3n2)cc1]

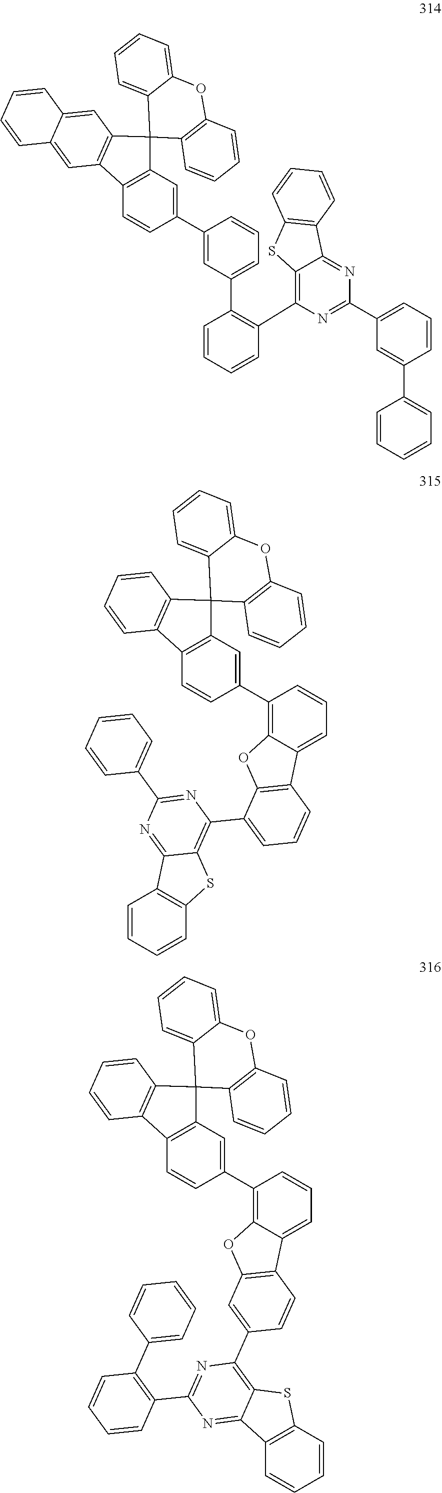

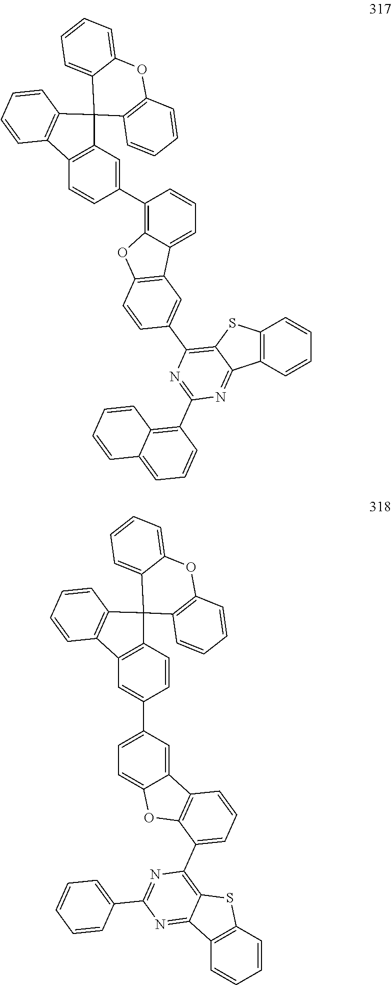 [SMILES: c1ccc2c(c1)Oc1ccccc1C21c2ccccc2-c2ccc(-c3cccc4c3oc3ccc(-c5nc(-c6cccc7ccccc67)nc6c5sc5ccccc56)cc34)cc21 ; c1ccc(-c2nc(-c3cccc4c3oc3ccc(-c5ccc6c(c5)-c5ccccc5C65c6ccccc6Oc6ccccc65)cc34)c3sc4ccccc4c3n2)cc1]

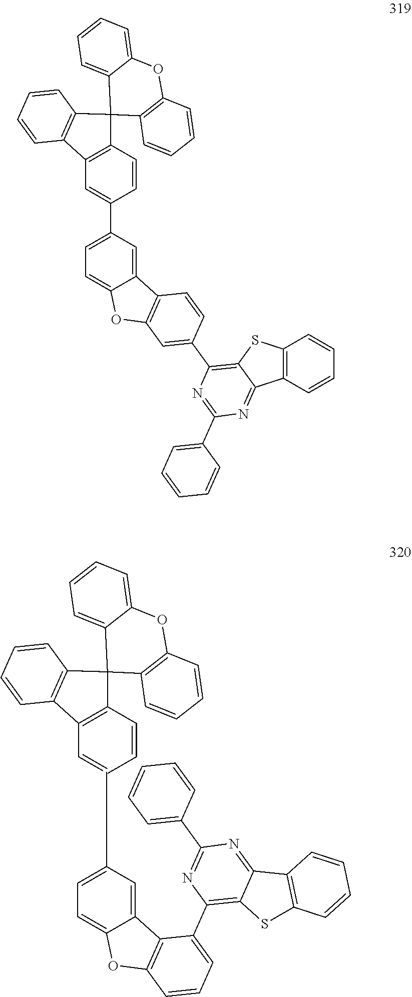

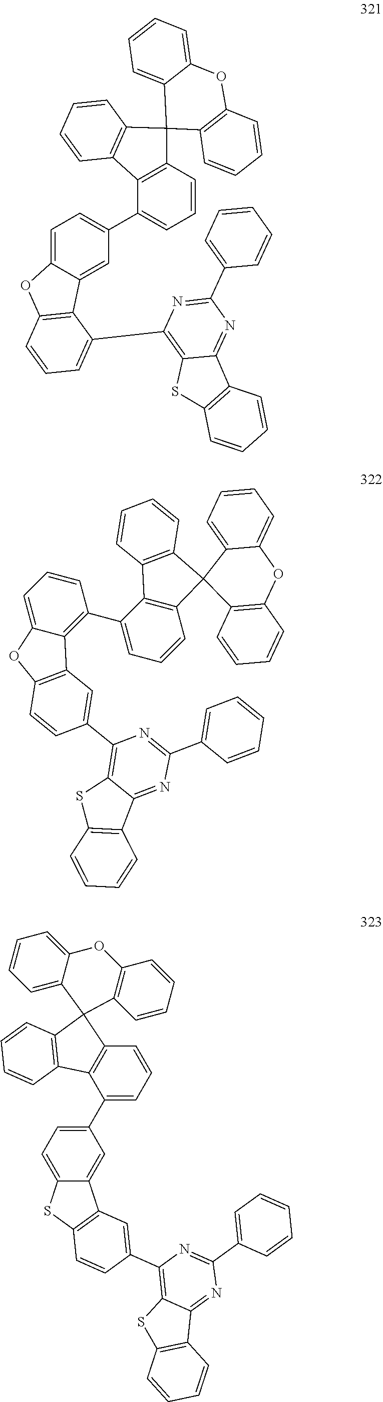

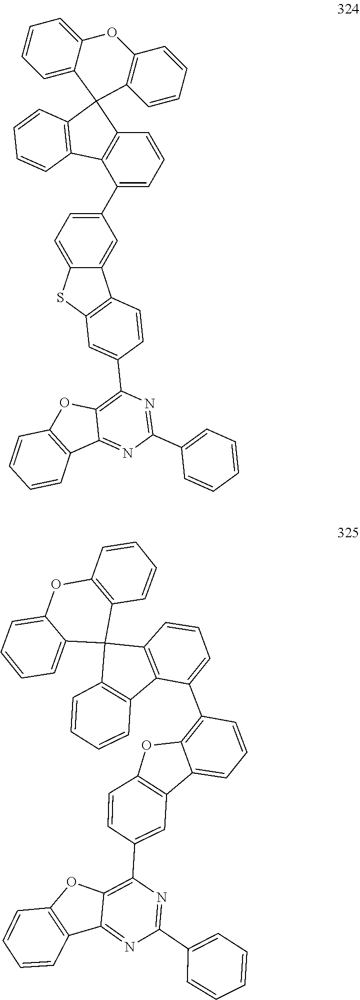 [SMILES: c1ccc(-c2nc(-c3ccc4c(c3)sc3ccc(-c5cccc6c5-c5ccccc5C65c6ccccc6Oc6ccccc65)cc34)c3oc4ccccc4c3n2)cc1 ; c1ccc(-c2nc(-c3ccc4oc5c(-c6cccc7c6-c6ccccc6C76c7ccccc7Oc7ccccc76)cccc5c4c3)c3oc4ccccc4c3n2)cc1]

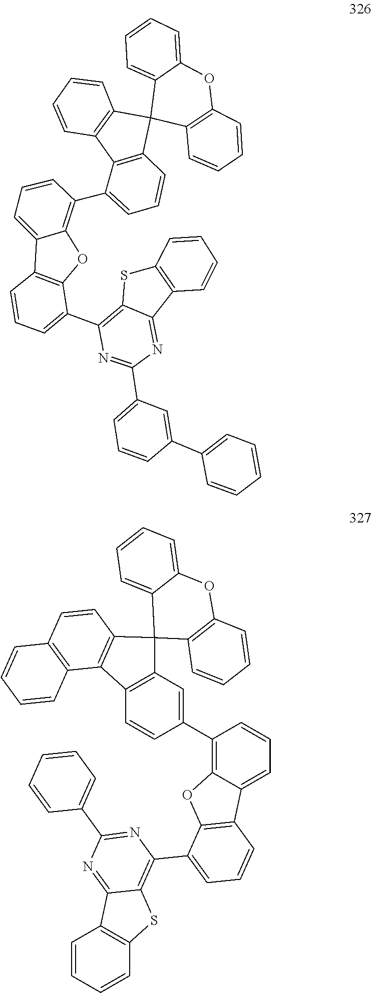 [SMILES: c1ccc(-c2cccc(-c3nc(-c4cccc5c4oc4c(-c6cccc7c6-c6ccccc6C76c7ccccc7Oc7ccccc76)cccc45)c4sc5ccccc5c4n3)c2)cc1 ; c1ccc(-c2nc(-c3cccc4c3oc3c(-c5ccc6c(c5)C5(c7ccccc7Oc7ccccc75)c5ccc7ccccc7c5-6)cccc34)c3sc4ccccc4c3n2)cc1]

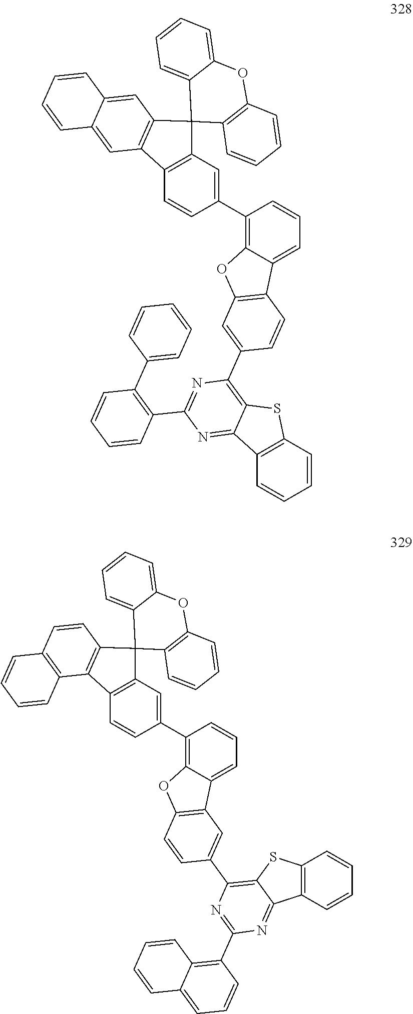 [SMILES: c1ccc(-c2ccccc2-c2nc(-c3ccc4c(c3)oc3c(-c5ccc6c(c5)C5(c7ccccc7Oc7ccccc75)c5cc7ccccc7cc5-6)cccc34)c3sc4ccccc4c3n2)cc1 ; c1ccc2c(c1)Oc1ccccc1C21c2cc(-c3cccc4c3oc3ccc(-c5nc(-c6cccc7ccccc67)nc6c5sc5ccccc56)cc34)ccc2-c2c1ccc1ccccc21]

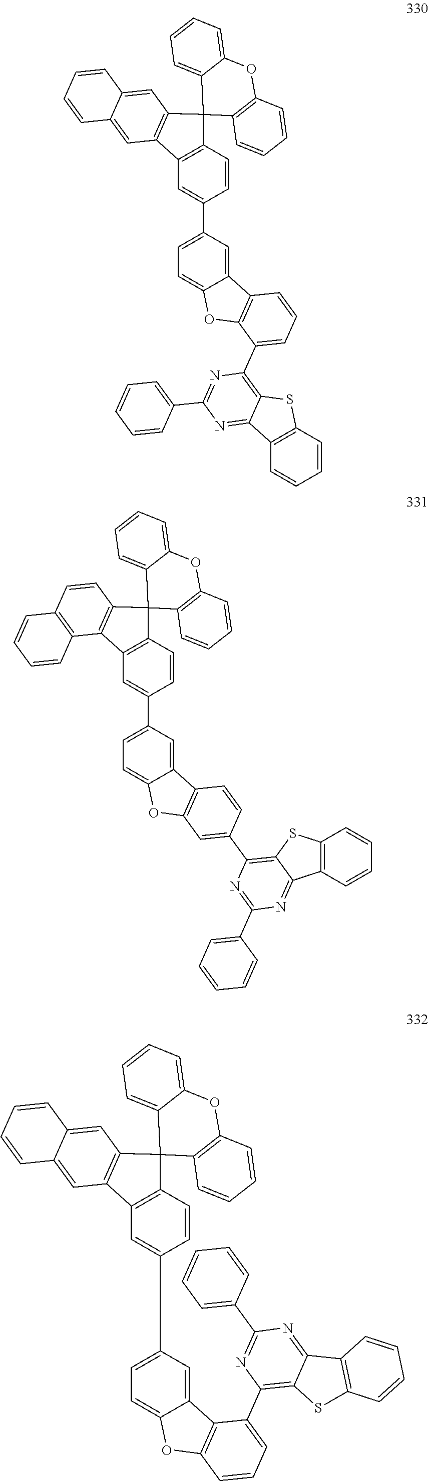 [SMILES: c1ccc(-c2nc(-c3cccc4c3oc3ccc(-c5ccc6c(c5)-c5cc7ccccc7cc5C65c6ccccc6Oc6ccccc65)cc34)c3sc4ccccc4c3n2)cc1 ; c1ccc(-c2nc(-c3ccc4c(c3)oc3ccc(-c5ccc6c(c5)-c5c(ccc7ccccc57)C65c6ccccc6Oc6ccccc65)cc34)c3sc4ccccc4c3n2)cc1 ; c1ccc(-c2nc(-c3cccc4oc5ccc(-c6ccc7c(c6)-c6cc8ccccc8cc6C76c7ccccc7Oc7ccccc76)cc5c34)c3sc4ccccc4c3n2)cc1]

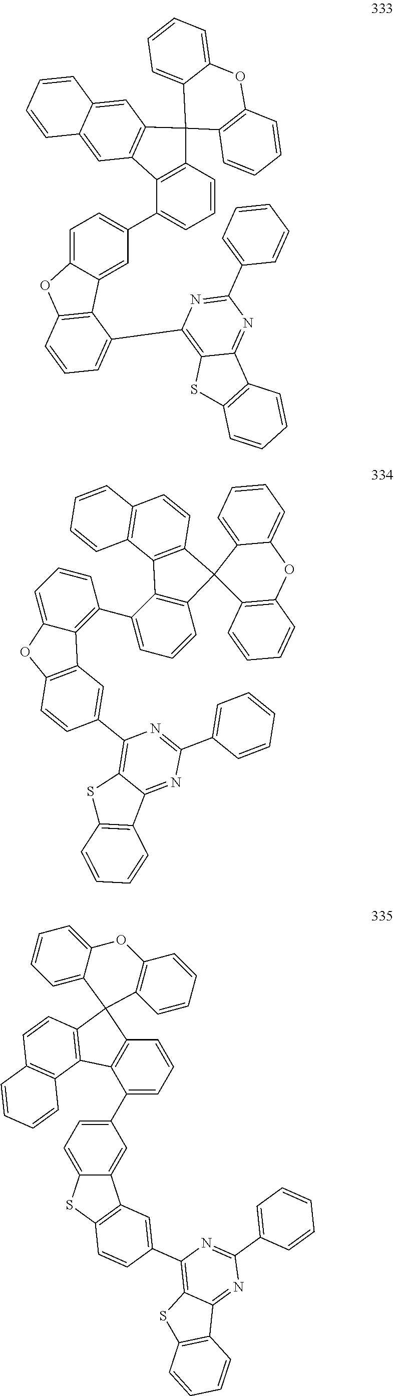

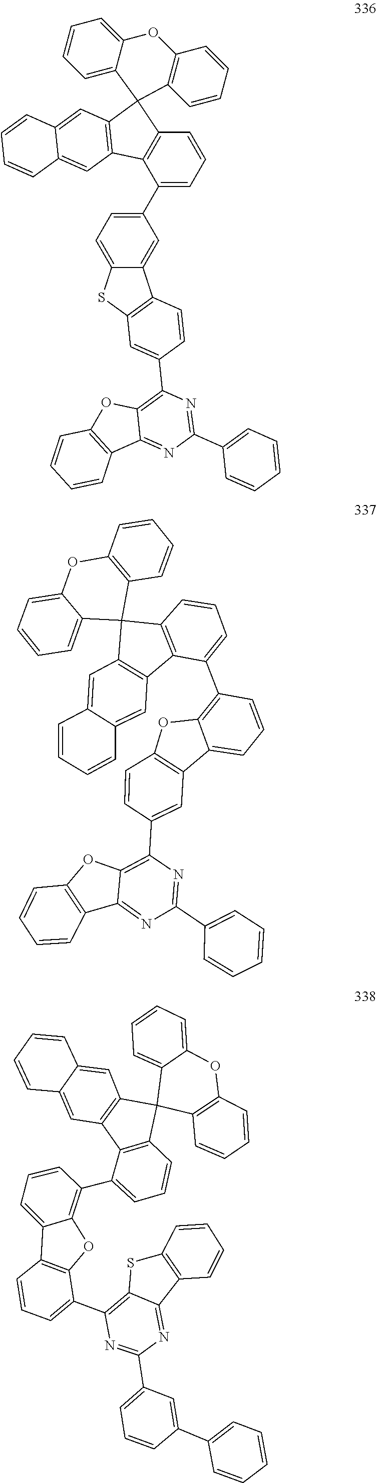 [SMILES: c1ccc(-c2nc(-c3ccc4c(c3)sc3ccc(-c5cccc6c5-c5cc7ccccc7cc5C65c6ccccc6Oc6ccccc65)cc34)c3oc4ccccc4c3n2)cc1 ; c1ccc(-c2nc(-c3ccc4oc5c(-c6cccc7c6-c6cc8ccccc8cc6C76c7ccccc7Oc7ccccc76)cccc5c4c3)c3oc4ccccc4c3n2)cc1 ; c1ccc(-c2cccc(-c3nc(-c4cccc5c4oc4c(-c6cccc7c6-c6cc8ccccc8cc6C76c7ccccc7Oc7ccccc76)cccc45)c4sc5ccccc5c4n3)c2)cc1]

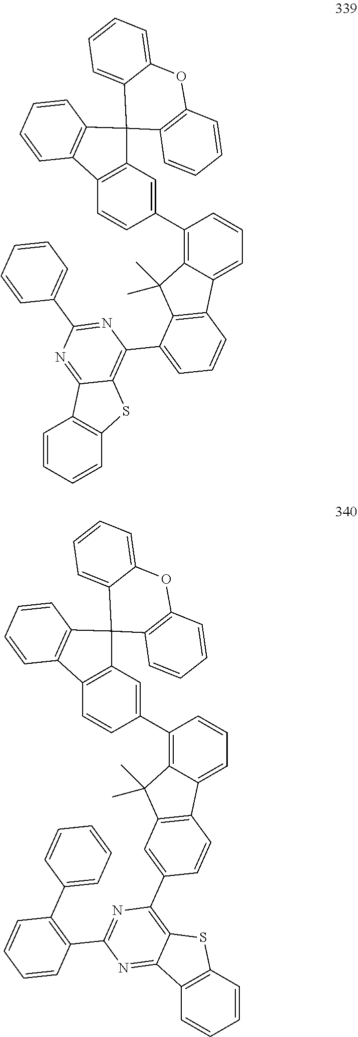 [SMILES: CC1(C)c2c(-c3ccc4c(c3)C3(c5ccccc5Oc5ccccc53)c3ccccc3-4)cccc2-c2cccc(-c3nc(-c4ccccc4)nc4c3sc3ccccc34)c21 ; CC1(C)c2cc(-c3nc(-c4ccccc4-c4ccccc4)nc4c3sc3ccccc34)ccc2-c2cccc(-c3ccc4c(c3)C3(c5ccccc5Oc5ccccc53)c3ccccc3-4)c21]

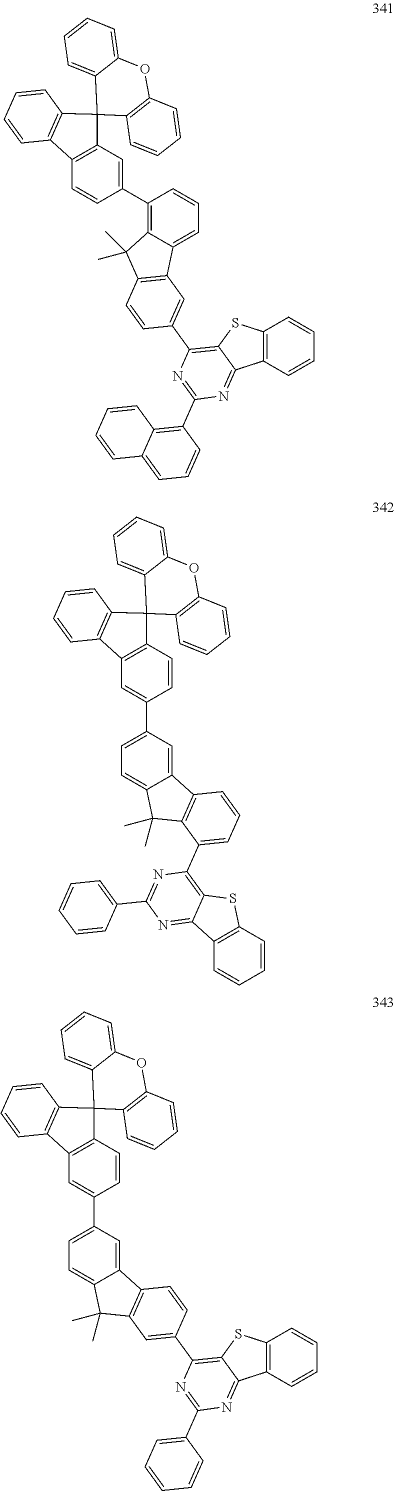 [SMILES: CC1(C)c2ccc(-c3nc(-c4cccc5ccccc45)nc4c3sc3ccccc34)cc2-c2cccc(-c3ccc4c(c3)C3(c5ccccc5Oc5ccccc53)c3ccccc3-4)c21 ; CC1(C)c2ccc(-c3ccc4c(c3)-c3ccccc3C43c4ccccc4Oc4ccccc43)cc2-c2cccc(-c3nc(-c4ccccc4)nc4c3sc3ccccc34)c21 ; CC1(C)c2ccc(-c3ccc4c(c3)-c3ccccc3C43c4ccccc4Oc4ccccc43)cc2-c2ccc(-c3nc(-c4ccccc4)nc4c3sc3ccccc34)cc21]

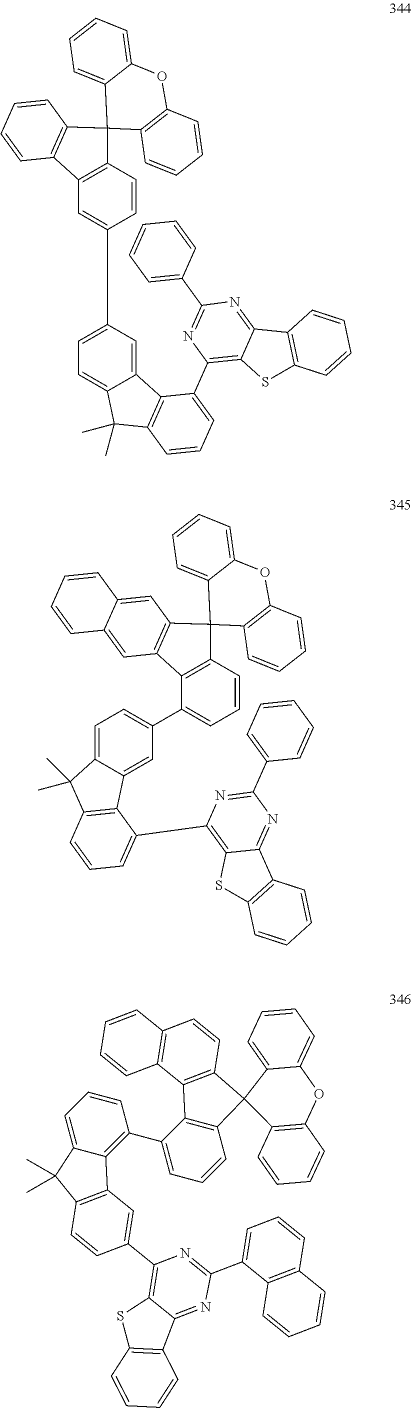 [SMILES: CC1(C)c2ccc(-c3ccc4c(c3)-c3ccccc3C43c4ccccc4Oc4ccccc43)cc2-c2c(-c3nc(-c4ccccc4)nc4c3sc3ccccc34)cccc21 ; CC1(C)c2ccc(-c3cccc4c3-c3cc5ccccc5cc3C43c4ccccc4Oc4ccccc43)cc2-c2c(-c3nc(-c4ccccc4)nc4c3sc3ccccc34)cccc21 ; CC1(C)c2ccc(-c3nc(-c4cccc5ccccc45)nc4c3sc3ccccc34)cc2-c2c(-c3cccc4c3-c3c(ccc5ccccc35)C43c4ccccc4Oc4ccccc43)cccc21]

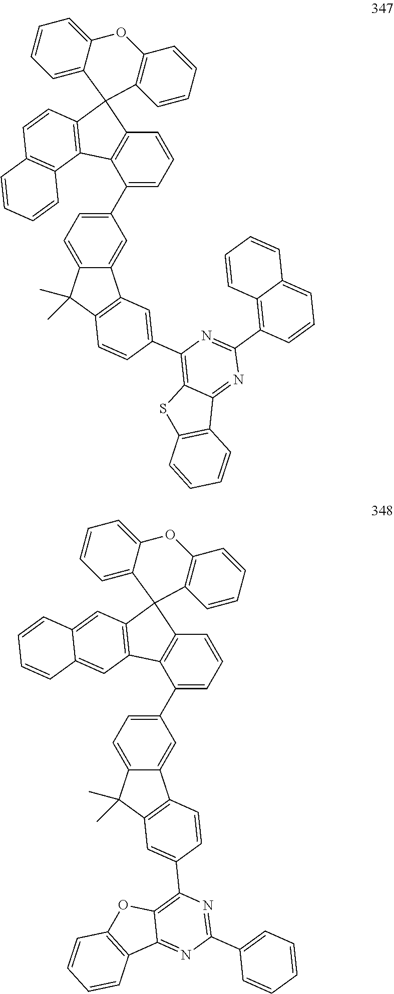 [SMILES: CC1(C)c2ccc(-c3cccc4c3-c3c(ccc5ccccc35)C43c4ccccc4Oc4ccccc43)cc2-c2cc(-c3nc(-c4cccc5ccccc45)nc4c3sc3ccccc34)ccc21 ; CC1(C)c2ccc(-c3cccc4c3-c3cc5ccccc5cc3C43c4ccccc4Oc4ccccc43)cc2-c2ccc(-c3nc(-c4ccccc4)nc4c3oc3ccccc34)cc21]

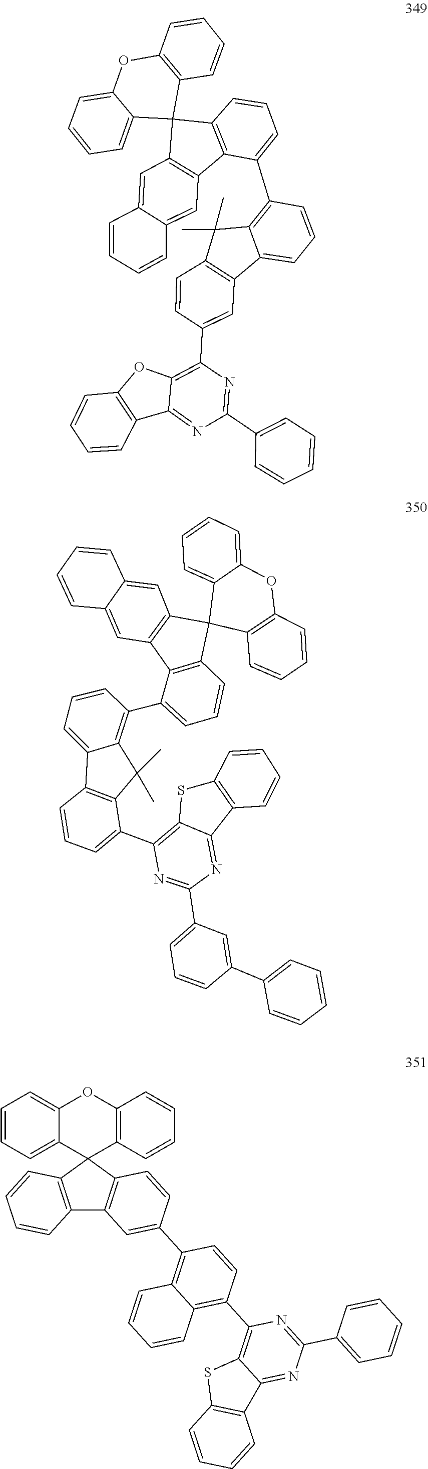 [SMILES: CC1(C)c2ccc(-c3nc(-c4ccccc4)nc4c3oc3ccccc34)cc2-c2cccc(-c3cccc4c3-c3cc5ccccc5cc3C43c4ccccc4Oc4ccccc43)c21 ; CC1(C)c2c(-c3cccc4c3-c3cc5ccccc5cc3C43c4ccccc4Oc4ccccc43)cccc2-c2cccc(-c3nc(-c4cccc(-c5ccccc5)c4)nc4c3sc3ccccc34)c21 ; c1ccc(-c2nc(-c3ccc(-c4ccc5c(c4)-c4ccccc4C54c5ccccc5Oc5ccccc54)c4ccccc34)c3sc4ccccc4c3n2)cc1]

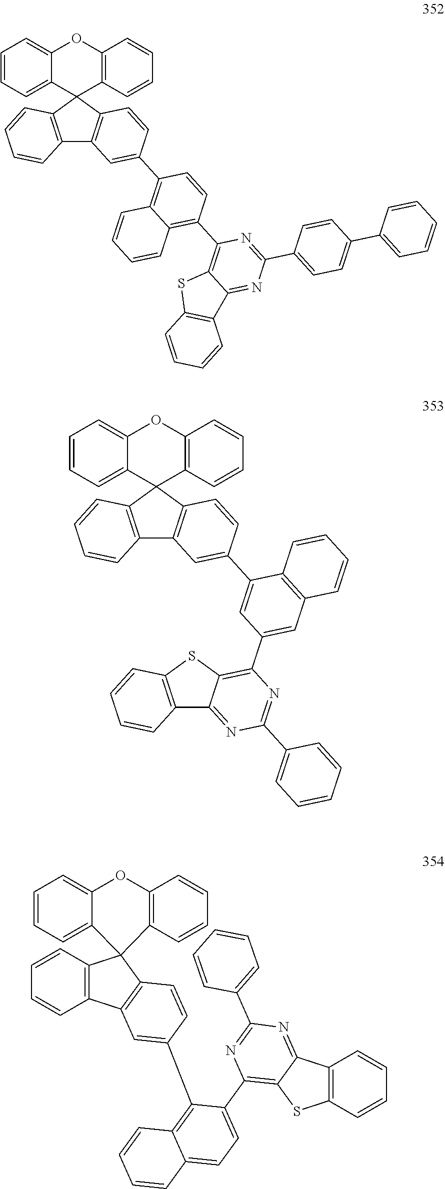 [SMILES: c1ccc(-c2ccc(-c3nc(-c4ccc(-c5ccc6c(c5)-c5ccccc5C65c6ccccc6Oc6ccccc65)c5ccccc45)c4sc5ccccc5c4n3)cc2)cc1 ; c1ccc(-c2nc(-c3cc(-c4ccc5c(c4)-c4ccccc4C54c5ccccc5Oc5ccccc54)c4ccccc4c3)c3sc4ccccc4c3n2)cc1 ; c1ccc(-c2nc(-c3ccc4ccccc4c3-c3ccc4c(c3)-c3ccccc3C43c4ccccc4Oc4ccccc43)c3sc4ccccc4c3n2)cc1]

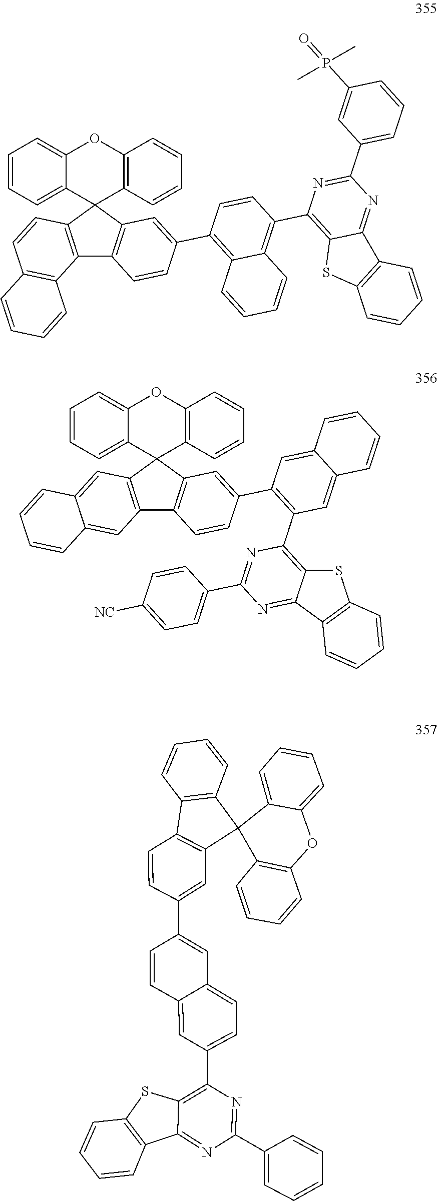 [SMILES: CP(C)(=O)c1cccc(-c2nc(-c3ccc(-c4ccc5c(c4)C4(c6ccccc6Oc6ccccc64)c4ccc6ccccc6c4-5)c4ccccc34)c3sc4ccccc4c3n2)c1 ; N#Cc1ccc(-c2nc(-c3cc4ccccc4cc3-c3ccc4c(c3)C3(c5ccccc5Oc5ccccc53)c3cc5ccccc5cc3-4)c3sc4ccccc4c3n2)cc1 ; c1ccc(-c2nc(-c3ccc4cc(-c5ccc6c(c5)C5(c7ccccc7Oc7ccccc75)c5ccccc5-6)ccc4c3)c3sc4ccccc4c3n2)cc1]

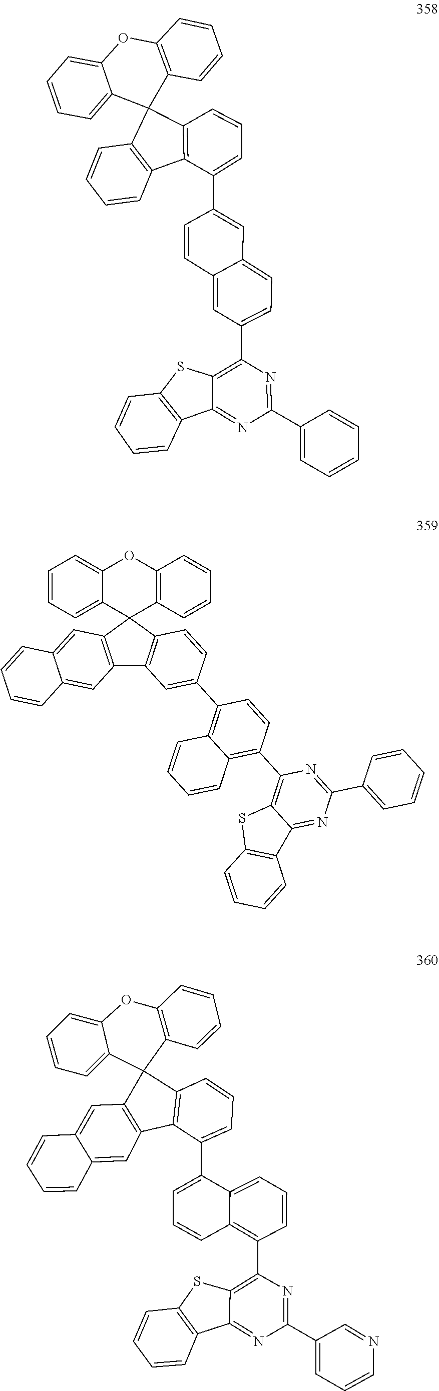 [SMILES: c1ccc(-c2nc(-c3ccc4cc(-c5cccc6c5-c5ccccc5C65c6ccccc6Oc6ccccc65)ccc4c3)c3sc4ccccc4c3n2)cc1 ; c1ccc(-c2nc(-c3ccc(-c4ccc5c(c4)-c4cc6ccccc6cc4C54c5ccccc5Oc5ccccc54)c4ccccc34)c3sc4ccccc4c3n2)cc1 ; c1cncc(-c2nc(-c3cccc4c(-c5cccc6c5-c5cc7ccccc7cc5C65c6ccccc6Oc6ccccc65)cccc34)c3sc4ccccc4c3n2)c1]

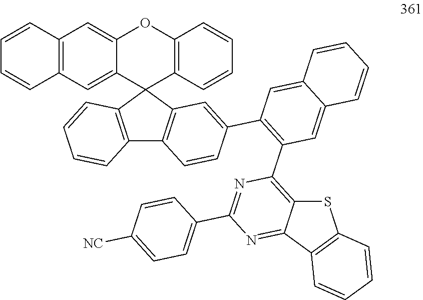 [SMILES: N#Cc1ccc(-c2nc(-c3cc4ccccc4cc3-c3ccc4c(c3)C3(c5ccccc5Oc5cc6ccccc6cc53)c3ccccc3-4)c3sc4ccccc4c3n2)cc1]

As used herein, “alkyl” refers to a monovalent substituent derived from a linear or branched chain saturated hydrocarbon having 1 to 40 carbon atoms. Examples of such alkyl may include, but are not limited to, methyl, ethyl, propyl, isobutyl, sec-butyl, pentyl, iso-amyl, hexyl or the like.

As used herein, “alkenyl” refers to a monovalent substituent derived from a linear or branched chain unsaturated hydrocarbon having 2 to 40 carbon atoms, having at least one carbon-carbon double bond. Examples of such alkenyl may include, but are not limited to, vinyl, allyl, isopropenyl, 2-butenyl or the like.

As used herein, “alkynyl” refers to a monovalent substituent derived from a linear or branched chain unsaturated hydrocarbon having 2 to 40 carbon atoms, having at least one carbon-carbon triple bond. Examples of such alkynyl may include, but are not limited to, ethynyl, 2-propynyl or the like.

As used herein, “aryl” refers to a monovalent substituent derived from a C₆ to C₄₀ aromatic hydrocarbon which is in a structure with a single ring or two or more rings combined with each other. In addition, a form in which two or more rings are pendant (e.g., simply attached) to or fused with each other may also be included. Examples of such aryl may include, but are not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 60 nuclear atoms. In such an embodiment, one or more carbons in the ring, preferably one to three carbons, are substituted with a heteroatom such as N, O, S or Se. In addition, a form in which two or more rings are pendant to or fused with each other may be included and a form fused with an aryl group may be included. Examples of such heteroaryl may include, but are not limited to, a 6-membered monocyclic ring including, for example, pyridyl, pyrazinyl, pyrimidinyl, pyridazinyl and triazinyl; a polycyclic ring including, for example, phenoxathienyl, indolinzinyl, indolyl purinyl, quinolyl, benzothiazole, and carbazolyl; 2-furanyl; N-imidazolyl; 2-isoxazolyl; 2-pyridinyl; 2-pyrimidinyl or the like.

As used herein, “aryloxy” is a monovalent substituent represented by RO—, where R refers to aryl having 5 to 40 carbon atoms. Examples of such aryloxy may include, but are not limited to, phenyloxy, naphthyloxy, diphenyloxy or the like.

As used herein, “alkyloxy” refers to a monovalent substituent represented by R′O—, where R′ refers to alkyl having 1 to 40 carbon atoms. Such alkyloxy may include a linear, branched or cyclic structure. Examples of such alkyloxy may include, but are not limited to, methoxy, ethoxy, n-propoxy, 1-propoxy, t-butoxy, n-butoxy, pentoxy or the like.

As used herein, “arylamine” refers to amine substituted with a C₆ to C₄₀ aryl.

As used herein, “cycloalkyl” refers to a monovalent substituent derived from a monocyclic or polycyclic non-aromatic hydrocarbon having 3 to 40 carbon atoms. Examples of such cycloalkyl may include, but are not limited to, cyclopropyl, cyclopentyl, cyclohexyl, norbornyl, adamantine or the like.

As used herein, “heterocycloalkyl” refers to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but are not limited to, morpholine, piperazine or the like.

As used herein, “alkylsilyl” refers to silyl in which substitution with alkyl having 1 to 40 carbon atoms has been made, and “arylsilyl” refers to silyl in which substitution with aryl having 5 to 40 carbon atoms has been made.

As used herein, the term “condensed ring (e.g., fused ring)” refers to a condensed aliphatic ring, a condensed aromatic ring, a condensed heteroaliphatic ring, a condensed heteroaromatic ring, or a combination thereof.

### <Electron Transport Layer Material>

The present invention provides an electron transport layer including the compound represented by Chemical Formula 1.

The electron transport layer (ETL) serves to move electrons injected from a cathode to an adjacent layer, specifically a light emitting layer.

The compound represented by Chemical Formula 1 may be used alone as an electron transport layer (ETL) material, or may be used in combination with an electron transport layer material known in the art. It may preferably be used alone.

The electron transport layer material that may be used in combination with the compound of Chemical Formula 1 includes an electron transport material commonly known in the art. Non-limiting examples of applicable electron transport materials may include oxazole-based compounds, isoxazole-based compounds, triazole-based compounds, isothiazole-based compounds, oxadiazole-based compounds, thiadiazole-based compounds, perylene-based compounds, aluminum complexes (e.g., tris(8-quinolinolato)-aluminium (Alq₃), BAlq, SAlq, Almq₃), gallium complexes (e.g., Gaq′2OPiv, Gaq′2OAc, 2(Gaq′2)), etc. These may be used alone or two or more types may be used in combination.

In the present invention, when the compound of Chemical Formula 1 and the material for the electron transport layer are used in combination, a mixing ratio thereof is not particularly limited, and may be appropriately adjusted within a range known in the art.

### <Electron Transport Auxiliary Layer Material>

In addition, the present invention provides an electron transport auxiliary layer including the compound represented by Chemical Formula 1.

The electron transport auxiliary layer is disposed between the light emitting layer and the electron transport layer and serves to substantially prevent diffusion of excitons or holes generated in the light emitting layer into the electron transport layer.

The compound represented by Chemical Formula 1 may be used alone as an electron transport auxiliary layer material, or may be combined with an electron transport layer material known in the art. It may preferably be used alone.

The electron transport auxiliary layer material that may be used in combination with the compound of Chemical Formula 1 includes an electron transport material commonly known in the art. For example, the electron transport auxiliary layer may include an oxadiazole derivative, a triazole derivative, a phenanthroline derivative (e.g., BCP), a heterocyclic derivative containing nitrogen, and the like.

In the present invention, when the compound of Chemical Formula 1 and the material for the electron transport auxiliary layer are used in combination, a mixing ratio thereof is not particularly limited, and may be appropriately adjusted within a range known in the art.

### <Organic Electroluminescent Device>

The present invention provides an organic electroluminescent device (“organic EL device”) including the compound represented by Chemical Formula 1.

More specifically, the organic EL device according to the present invention includes an anode (e.g., a positive electrode), a cathode (e.g., a negative electrode), and one or more organic layers disposed between the anode and the cathode, and at least one of the one or more organic layers includes the compound represented by Chemical Formula 1. In such an embodiment, the compound may be used alone or as a combination of two or more kinds thereof.

The one or more organic layers may be any one or more of a light emitting layer, a light emitting auxiliary layer, a hole injection layer, a hole transport layer, an electron injection layer, an electron transport layer, and an electron transport auxiliary layer, and at least one of the organic layers may include the compound represented by Chemical Formula 1. Specifically, the organic layer including the compound represented by Chemical Formula 1 may preferably be a light emitting layer, an electron transport layer, and an electron transport auxiliary layer

The light emitting layer of the organic EL device according to the present invention may include a host material, and in such a case, may include the compound of Chemical Formula 1 as the host material. In addition, the light emitting layer of the organic EL device of the present invention may include another compound other than the compound represented by Chemical Formula 1 as a host.

When the compound represented by Chemical Formula 1 is included as a material for the light emitting layer of the organic EL device, preferably a phosphorescent host material of blue, green, and red colors, a binding force between holes and electrons in the light emitting layer increases, so the efficiency (luminous efficiency and power efficiency), service life, luminance and driving voltage of the organic EL device may be improved. Specifically, the compound represented by Chemical Formula 1 may preferably be included in the organic EL device as a blue and/or green phosphorescent host, fluorescent host, or dopant material. In particular, the compound represented by Formula 1 of the present invention is preferably an electron transport layer material because it has excellent electron injection and transport abilities.

The structure of the organic EL device of the present invention is not particularly limited, but a non-limiting example thereof may be a structure in which a substrate, an anode, a hole injection layer, a hole transport layer, a light emitting auxiliary layer, a light emitting layer, an electron transport layer and a cathode are sequentially stacked. In such a case, at least one of the the light emitting auxiliary layer, the light emitting layer, the electron transport layer and the electron injection layer may include the compound represented by Chemical Formula 1. In an embodiment, an electron injection layer may be further stacked on the electron transport layer.

In addition, the structure of the organic EL device of the present invention may have a structure in which an insulating layer or an adhesive layer is inserted at an interface between the electrode and the organic layer.

The organic EL device of the present invention may be prepared using materials and methods known in the art, except that one or more layers of the aforementioned organic layers include the compound represented by Chemical Formula 1.

The organic layer may be formed by a vacuum deposition method or a solution coating method. Examples of the solution coating method may include, but are not limited to, spin coating, dip coating, doctor blading, inkjet printing, thermal transfer or the like.

The substrate used in preparation of the organic EL device of the present invention is not particularly limited, and non-limiting examples thereof may include silicon wafers, quartz, glass plates, metal plates, plastic films, sheets or the like.

In addition, examples of an anode material may include, but are not limited to, a metal such as vanadium, chromium, copper, zinc, and gold or an alloy thereof; metal oxides such as zinc oxide, indium oxide, indium tin oxide (ITO), or indium zinc oxide (IZO); combination of oxide with metal such as ZnO:Al or SnO₂:Sb; conductive polymers such as polythiophene, poly(3-methylthiophene), poly [3,4-(ethylene-1,2-dioxy) thiophene] (PEDT), polypyrrole or polyaniline; and carbon black or the like.

In addition, examples of a cathode material may include, but are not limited to, a metal such as magnesium, calcium, sodium, potassium, titanium, indium, yttrium, lithium, gadolinium, aluminum, silver, tin, or lead or an alloy thereof; a multi-layered material such as LiF/Al or LiO₂/Al or the like.

In addition, materials for the hole injection layer, the light emitting layer, the electron injection layer, and the electron transport layer are not particularly limited and conventional materials known in the art may be used without limitation.

Hereinafter, the present invention will be described in detail with reference to the following examples. However, the following examples are merely to illustrate the invention, and the present invention is not limited to the following embodiments.

### PREPARATION EXAMPLE 1

### Synthesis of Core 1

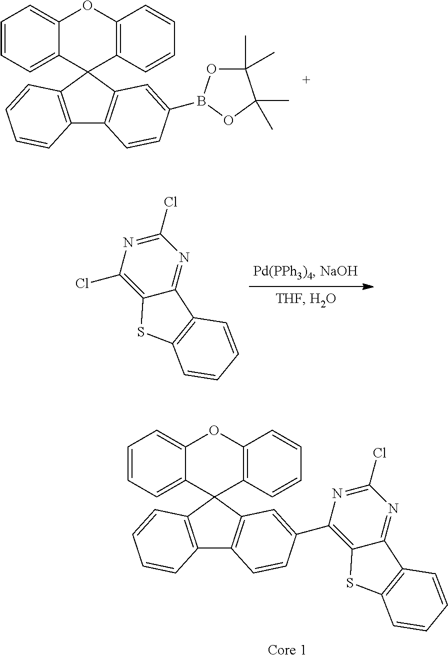 [SMILES: CC1(C)OB(c2ccc3c(c2)C2(c4ccccc4Oc4ccccc42)c2ccccc2-3)OC1(C)C ; Clc1nc(Cl)c2sc3ccccc3c2n1 ; Clc1nc(-c2ccc3c(c2)C2(c4ccccc4Oc4ccccc42)c2ccccc2-3)c2sc3ccccc3c2n1]

4,4,5,5-tetramethyl-2-(spiro[fluorene-9,9′-xanthen]-2-yl)-1,3,2-dioxaborolane (92 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentrating, the resultant product was recrystallized with toluene to obtain Core 1 (83 g, yield 75%).

[LCMS]: 550

### PREPARATION EXAMPLE 2

### Synthesis of Core 2

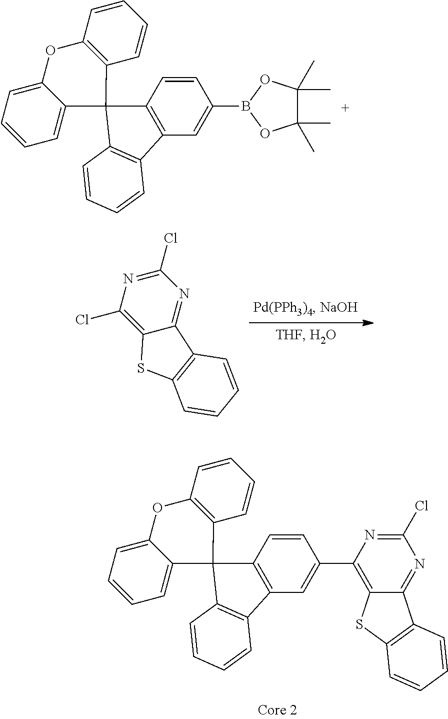

4,4,5,5-tetramethyl-3-(spiro[fluorene-9,9′-xanthen]-2-yl)-1,3,2-dioxaborolane (92 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 2 (96 g, yield 86%).

[LCMS]: 550

### PREPARATION EXAMPLE 3

### Synthesis of Core 3

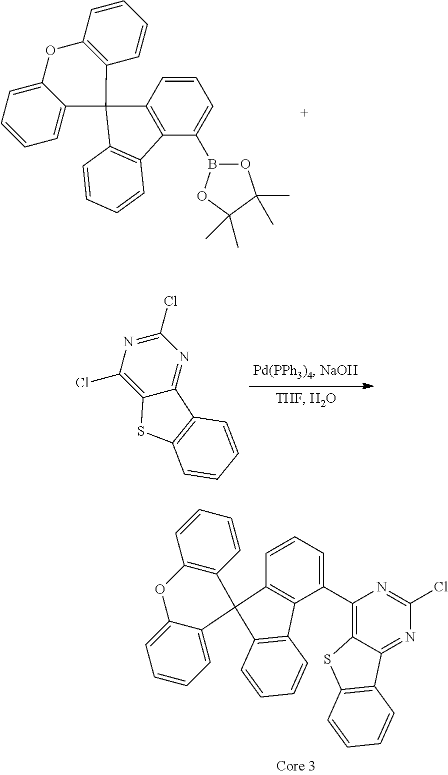

4,4,5,5-tetramethyl-4-(spiro[fluorene-9,9′-xanthen]-2-yl)-1,3,2-dioxaborolane (92 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 3 (86 g, yield 77%).

[LCMS]: 550

### PREPARATION EXAMPLE 4

### Synthesis of Core 4

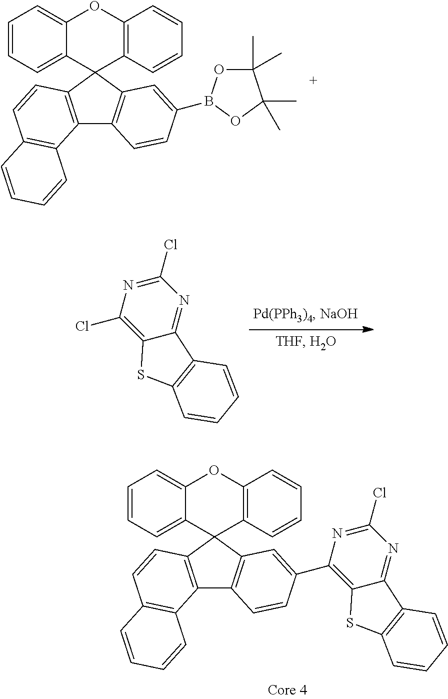 [SMILES: CC1(C)OB(c2ccc3c(c2)C2(c4ccccc4Oc4ccccc42)c2ccc4ccccc4c2-3)OC1(C)C ; Clc1nc(Cl)c2sc3ccccc3c2n1 ; Clc1nc(-c2ccc3c(c2)C2(c4ccccc4Oc4ccccc42)c2ccc4ccccc4c2-3)c2sc3ccccc3c2n1]

4,4,5,5-tetramethyl-2-(spiro[benzo[c]fluorene-7,9′-xanthen]-9-yl)-1,3,2-dioxaborolane (102 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 4 (79 g, yield 66%).

[LCMS]: 600

### PREPARATION EXAMPLE 5

### Synthesis of Core 5

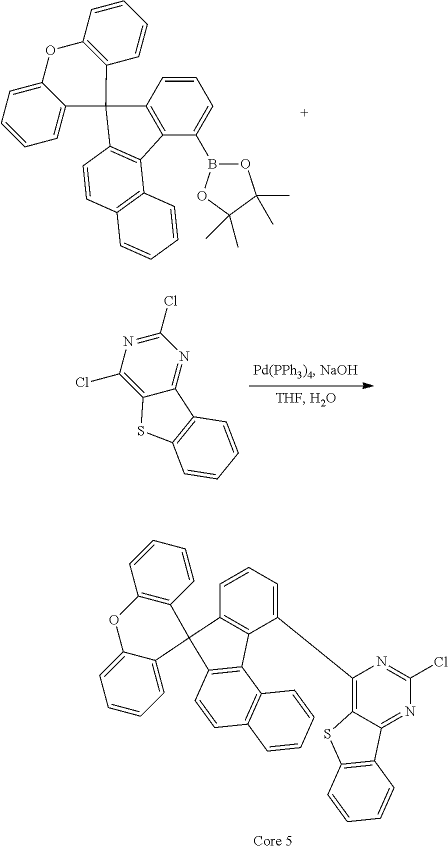

4,4,5,5-tetramethyl-4-(spiro[benzo[c]fluorene-7,9′-xanthen]-9-yl)-1,3,2-dioxaborolane (102 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 5 (94 g, yield 78%).

[LCMS]: 600

### PREPARATION EXAMPLE 6

### Synthesis of Core 6

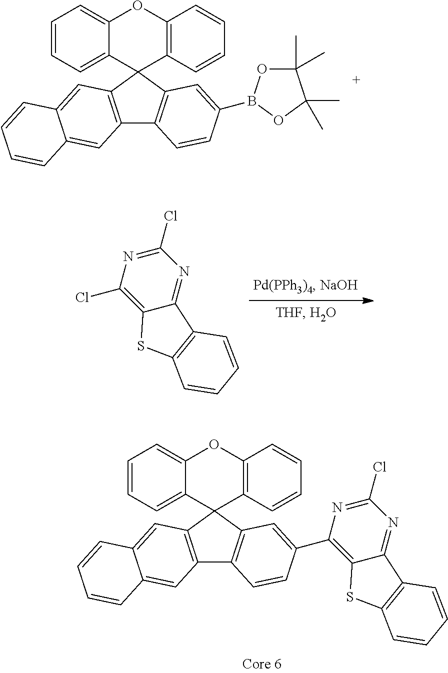 [SMILES: CC1(C)OB(c2ccc3c(c2)C2(c4ccccc4Oc4ccccc42)c2cc4ccccc4cc2-3)OC1(C)C ; Clc1nc(Cl)c2sc3ccccc3c2n1 ; Clc1nc(-c2ccc3c(c2)C2(c4ccccc4Oc4ccccc42)c2cc4ccccc4cc2-3)c2sc3ccccc3c2n1]

4,4,5,5-tetramethyl-2-(spiro[benzo[b]fluorene-11,9′-xanthen]-2-yl)-1,3,2-dioxaborolane (102 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 6 (78 g, yield 65%).

[LCMS]: 600

### PREPARATION EXAMPLE 7

### Synthesis of Core 7

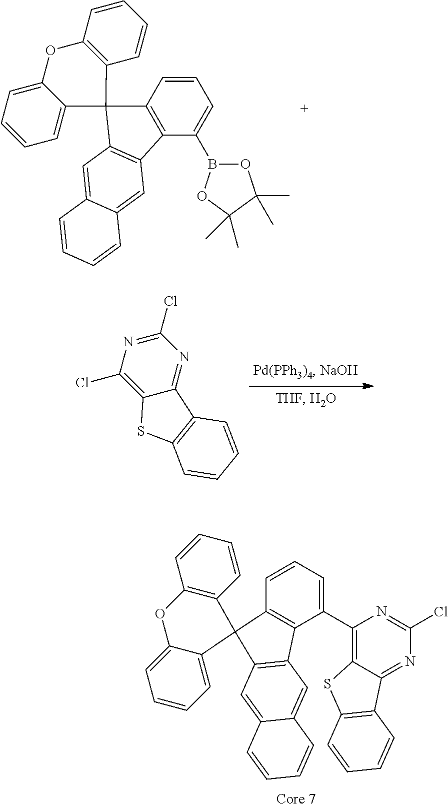 [SMILES: CC1(C)OB(c2cccc3c2-c2cc4ccccc4cc2C32c3ccccc3Oc3ccccc32)OC1(C)C ; Clc1nc(Cl)c2sc3ccccc3c2n1 ; Clc1nc(-c2cccc3c2-c2cc4ccccc4cc2C32c3ccccc3Oc3ccccc32)c2sc3ccccc3c2n1]

4,4,5,5-tetramethyl-4-(spiro[benzo[b]fluorene-11,9′-xanthen]-2-yl)-1,3,2-dioxaborolane (102 g, 200 mmol), 2,4-dichlorobenzo[4,5]thieno[3,2-d]pyrimidine (51 g, 200 mmol), Pd(PPh₃)₄ (9.2 g, 8 mmol), and NaOH (24 g, 600 mmol) were added to 1,000 ml of THF and 300 ml of H₂O and stirred at 75° C. for 8 hours. After completion of the reaction, a produced solid was filtered. Then, the solid was dissolved with toluene, filtered with silica, and then concentrated. After concentration, the resultant product was recrystallized with toluene to obtain Core 7 (96 g, yield 80%).

[LCMS]: 600

### SYNTHESIS EXAMPLE 1

### Synthesis of Compound 2

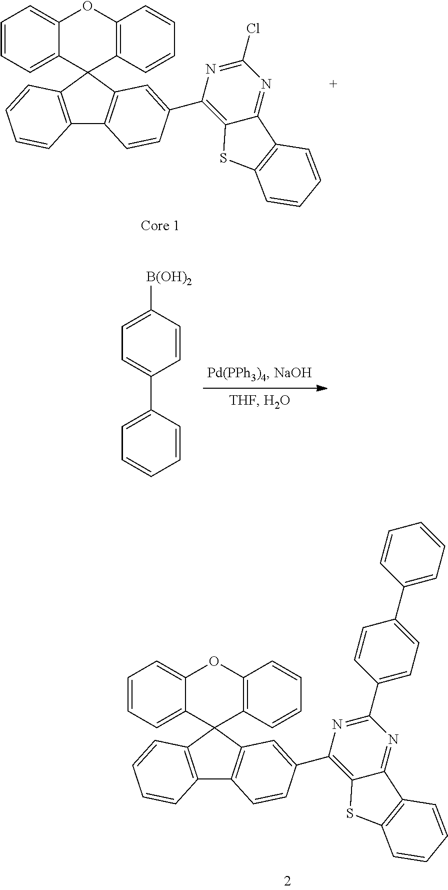 [SMILES: Clc1nc(-c2ccc3c(c2)C2(c4ccccc4Oc4ccccc42)c2ccccc2-3)c2sc3ccccc3c2n1 ; OB(O)c1ccc(-c2ccccc2)cc1 ; c1ccc(-c2ccc(-c3nc(-c4ccc5c(c4)C4(c6ccccc6Oc6ccccc64)c4ccccc4-5)c4sc5ccccc5c4n3)cc2)cc1]

Core 1 (5.5 g, 10.0 mmol), [1,1′-Biphenyl]-4-ylboronic acid (2.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 2 (4.2 g, yield 63%).

[LCMS]: 668

### SYNTHESIS EXAMPLE 2

### Synthesis of Compound 7

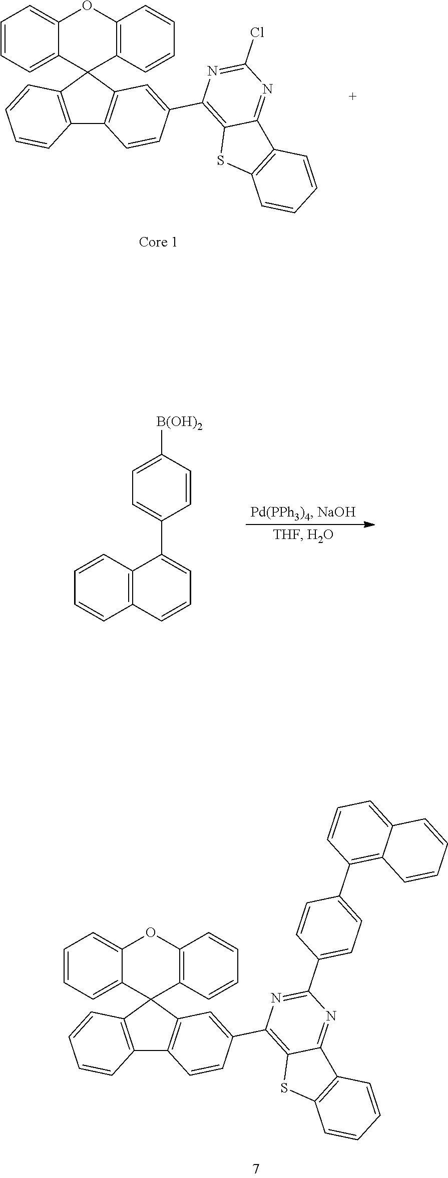

Core 1 (5.5 g, 10.0 mmol), (4-(Naphthalen-1-yl)phenyl)boronic acid (2.5 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 7 (4.7 g, yield 65%).

[LCMS]: 718

### SYNTHESIS EXAMPLE 3

### Synthesis of Compound 30

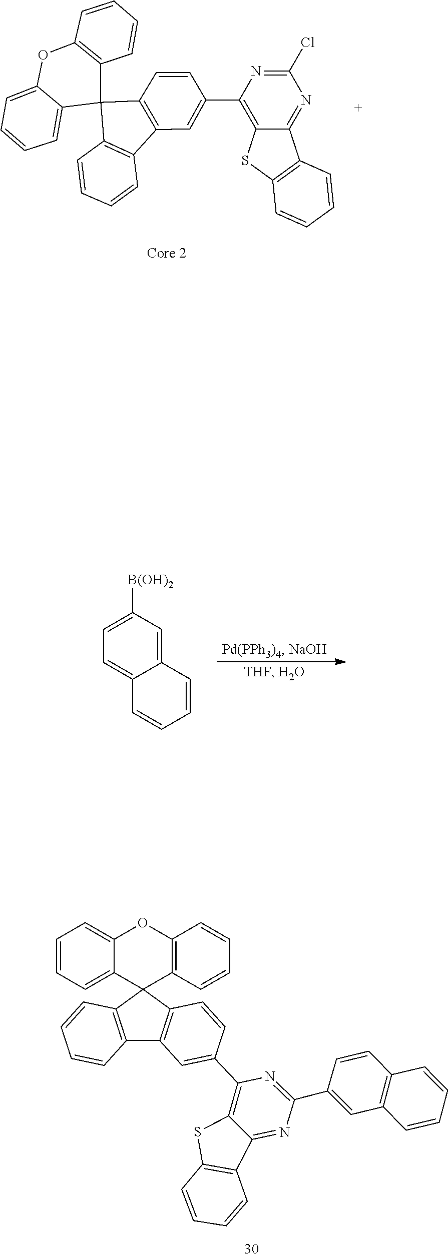

Core 2 (5.5 g, 10.0 mmol), naphthalen-2-ylboronic acid (1.7 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 30 (3.8 g, yield 60%).

[LCMS]: 642

### SYNTHESIS EXAMPLE 4

### Synthesis of Compound 50

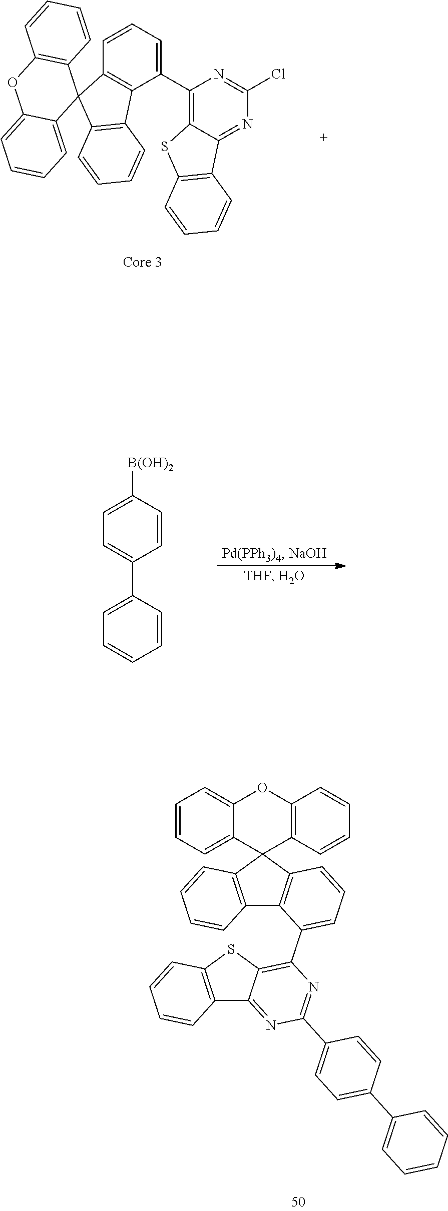

Core 3 (5.5 g, 10.0 mmol), [1,1′-Biphenyl]-4-ylboronic acid (2.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 50 (4.7 g, yield 70%).

[LCMS]: 668

### SYNTHESIS EXAMPLE 5

### Synthesis of Compound 61

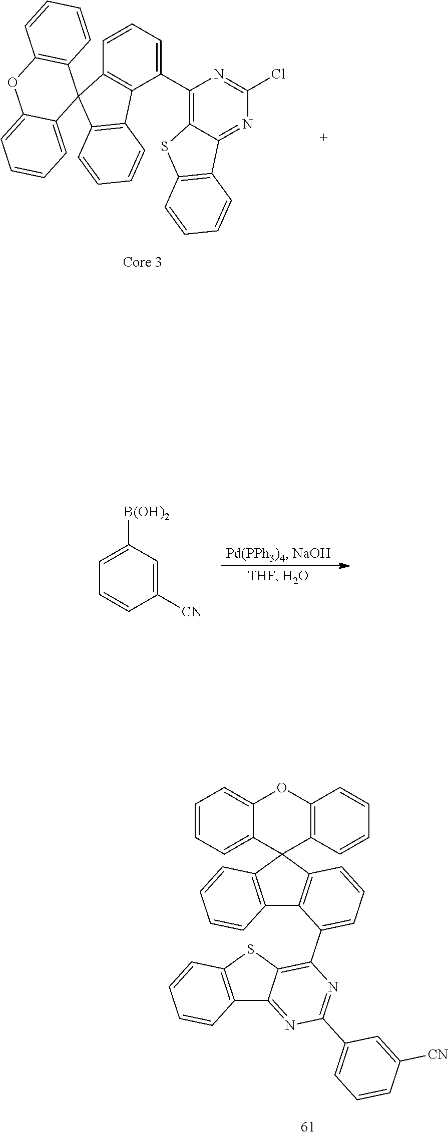

Core 3 (5.5 g, 10.0 mmol), (3-Cyanophenyl)boronic acid (1.5 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 61 (3.4 g, yield 55%).

[LCMS]: 617

### SYNTHESIS EXAMPLE 6

### Synthesis of Compound 98

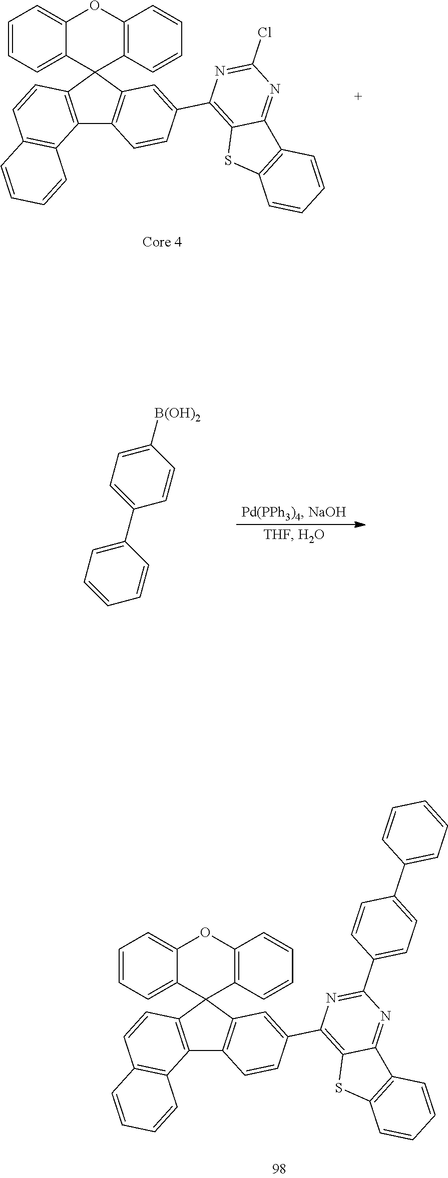

Core 4 (6.0 g, 10.0 mmol), [1,1′-Biphenyl]-4-ylboronic acid (2.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 98 (3.9 g, yield 54%).

[LCMS]: 718

### SYNTHESIS EXAMPLE 7

### Synthesis of Compound 125

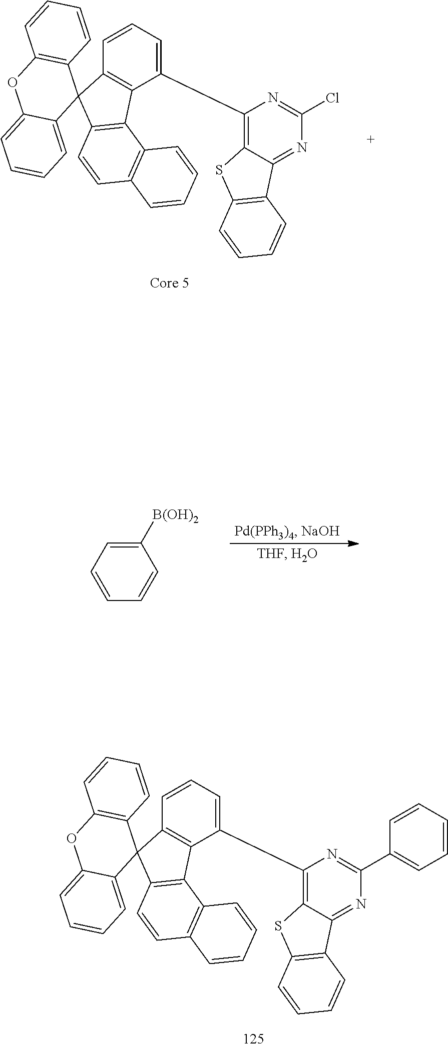

Core 5 (6.0 g, 10.0 mmol), Phenylboronic acid (1.2 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 125 (5.0 g, yield 78%).

[LCMS]: 642

### SYNTHESIS EXAMPLE 8

### Synthesis of Compound 138

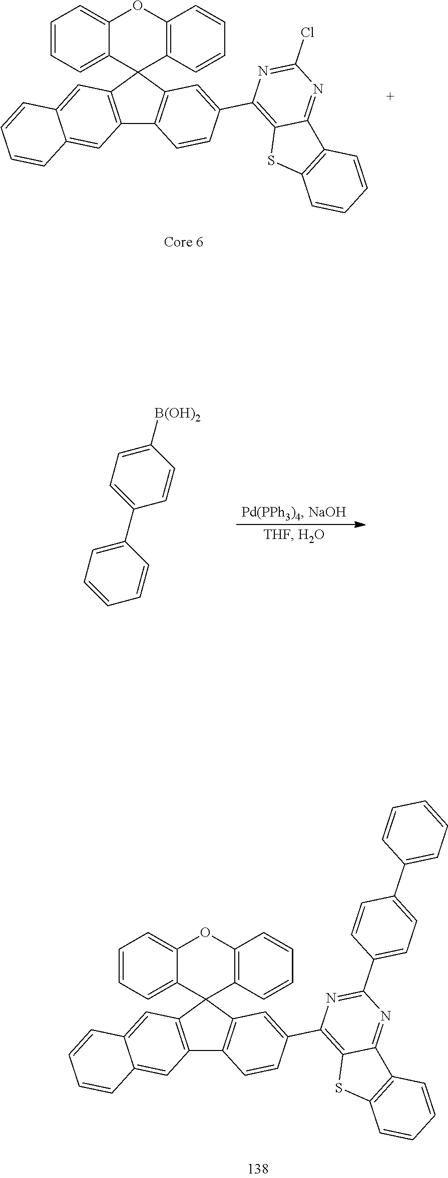

Core 6 (6.0 g, 10.0 mmol), [1,1′-Biphenyl]-4-ylboronic acid (2.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 138 (4.8 g, yield 67%).

[LCMS]: 718

### SYNTHESIS EXAMPLE 9

### Synthesis of Compound 161

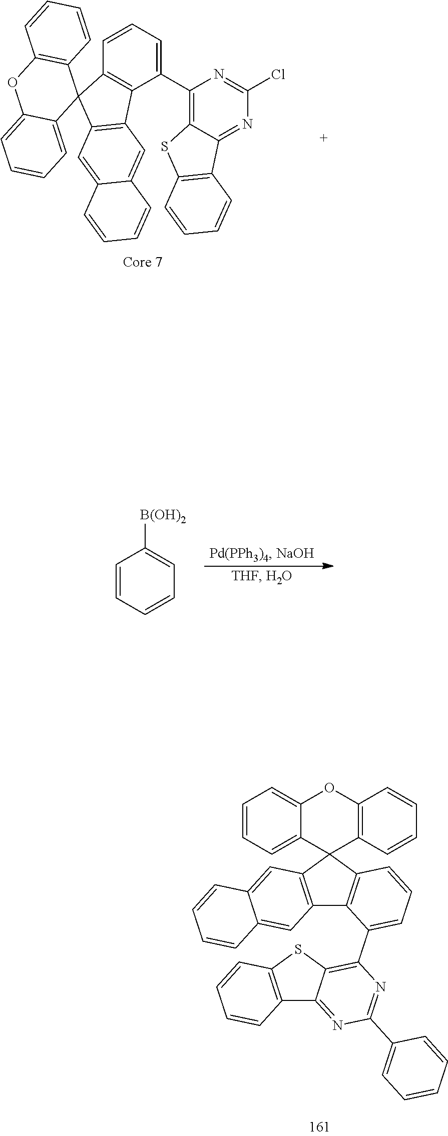

Core 7 (6.0 g, 10.0 mmol), Phenylboronic acid (1.2 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 161 (3.3 g, yield 51%).

[LCMS]: 642

### SYNTHESIS EXAMPLE 10

### Synthesis of Compound 211

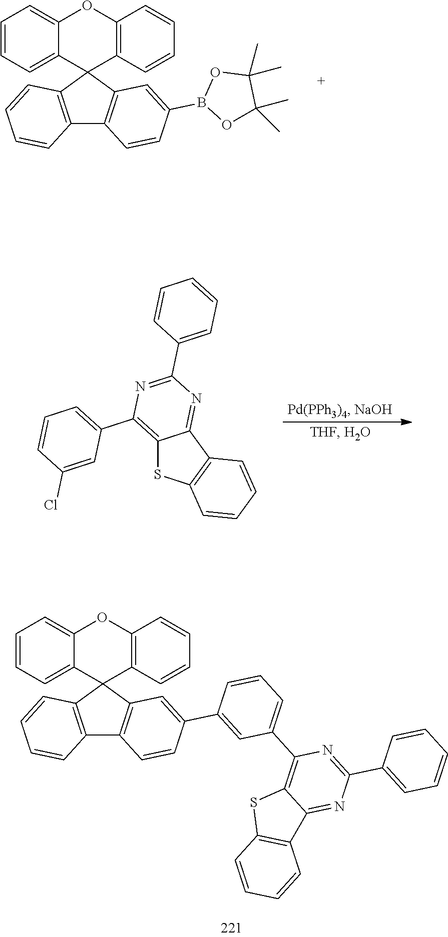

4,4,5,5-Tetramethyl-2-(spiro[fluorene-9,9′-xanthen]-2-yl)-1,3,2-dioxaborolane (4.6 g, 10.0 mmol), 4-(3-Chlorophenyl)-2-phenylbenzo [4,5]thieno[3,2-d]pyrimidine (3.7 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 211 (4.9 g, yield 74%).

[LCMS]: 668

### SYNTHESIS EXAMPLE 11

### Synthesis of Compound 231

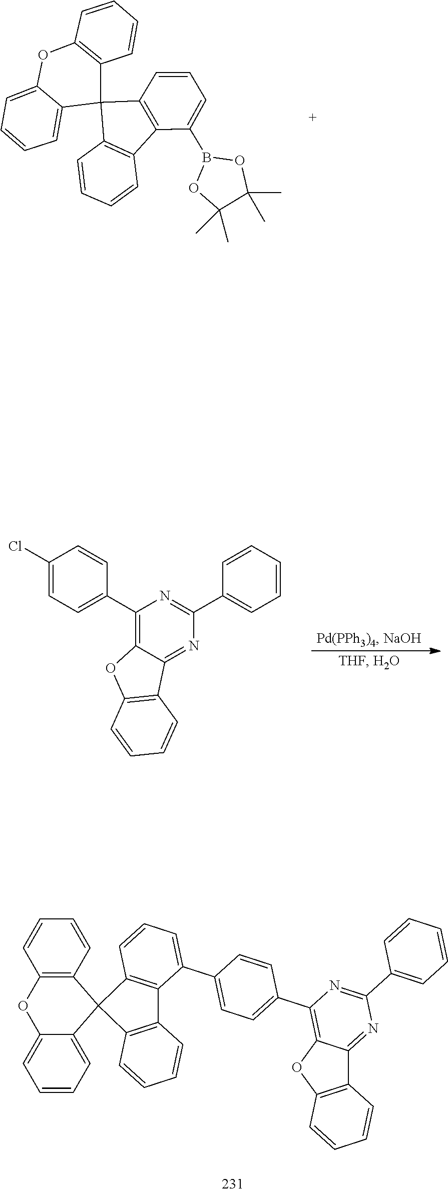

4,4,5,5-Tetramethyl-4-(spiro[fluorene-9,9′-xanthen]-2-yl)-1,3,2-dioxaborolane (4.6 g, 10.0 mmol), 4-(4-Chlorophenyl)-2-phenylbenzofuro[3,2-d]pyrimidine (3.6 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 231 (5.2 g, yield 80%).

[LCMS]: 652

### SYNTHESIS EXAMPLE 12

### Synthesis of Compound 286

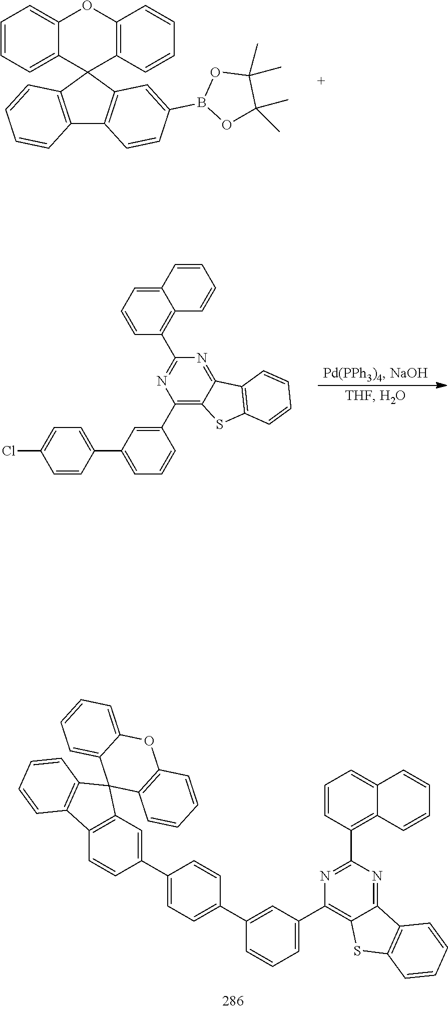

4,4,5,5-Tetramethyl-2-(spiro[fluorene-9,9′-xanthen]-2-yl)-1,3,2-dioxaborolane (4.6 g, 10.0 mmol), 4-(4′-Chloro-[1,1′-biphenyl]-3-yl)-2-(naphthalen-1-yl)benzo[4,5]thieno [3,2-d]pyrimidine (5.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 286 (5.1 g, yield 65%).

[LCMS]: 794

### SYNTHESIS EXAMPLE 13

### Synthesis of Compound 295

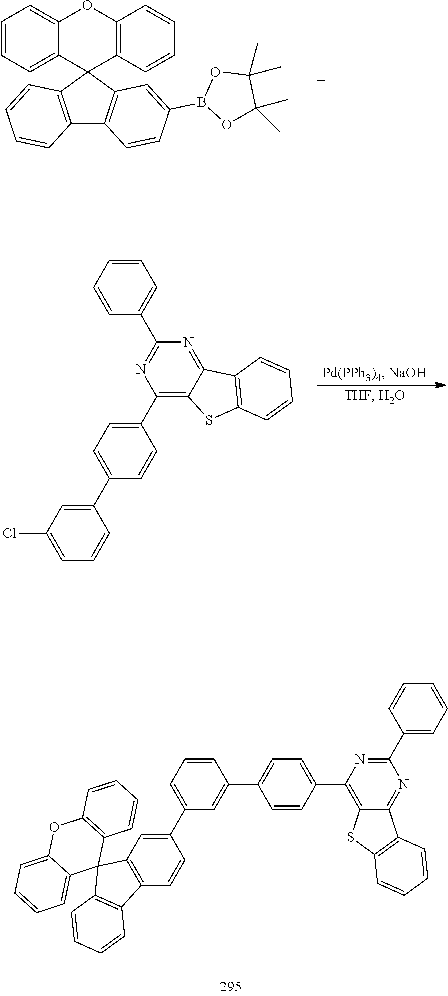

4,4,5,5-Tetramethyl-2-(spiro[fluorene-9,9′-xanthen]-2-yl)-1,3,2-dioxaborolane (4.6 g, 10.0 mmol), 4-(3′-Chloro-[1,1′-biphenyl]-4-yl)-2 -phenylbenzo[4,5]thieno[3,2-d]pyrimidine (4.5 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 295 (5.5 g, yield 74%).

[LCMS]: 744

### SYNTHESIS EXAMPLE 14

### Synthesis of Compound 311

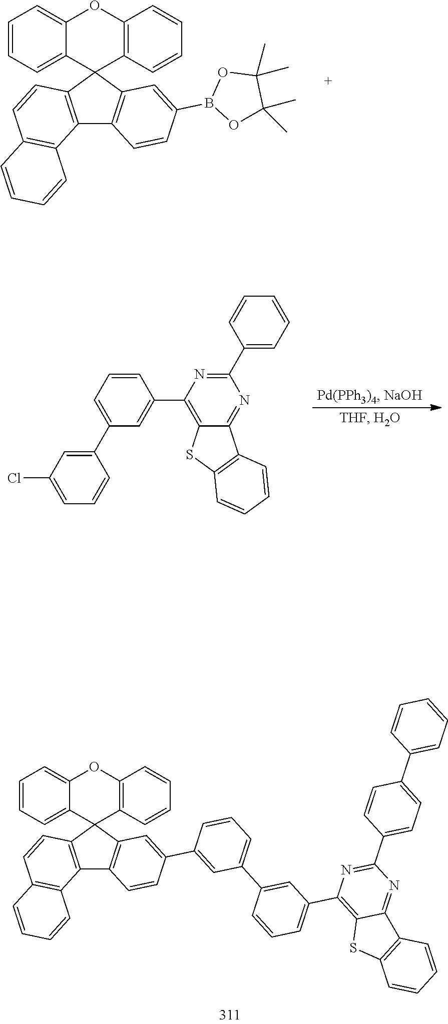

4,4,5,5-Tetramethyl-2-(spiro[fluorene-9,9′-xanthen]-2-yl)-1,3,2-dioxaborolane (5.1 g, 10.0 mmol), 4-(3′-chloro-[1,1′-biphenyl]-3-yl)-2-phenylbenzo[4,5]thieno[3,2-d]pyrimidine (4.5 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 311 (7.6 g, yield 87%).

[LCMS]: 870

### SYNTHESIS EXAMPLE 15

### Synthesis of Compound 312

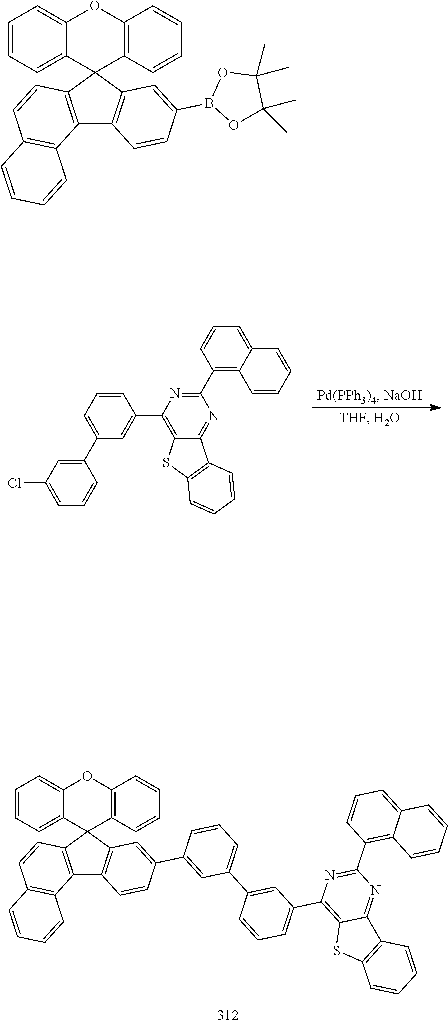

4,4,5,5-Tetramethyl-2-(spiro[fluorene-9,9′-xanthen]-2-yl)-1,3,2-dioxaborolane (5.1 g, 10.0 mmol), 4-(3′-Chloro-[1,1′-biphenyl]-3-yl)-2-(naphthalen-1-yl)benzo[4,5]thieno[3,2-d]pyrimidine (5.0 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, Compound 312 (5.9 g, yield 70%).

[LCMS]: 844

### SYNTHESIS EXAMPLE 16

### Synthesis of Compound 321

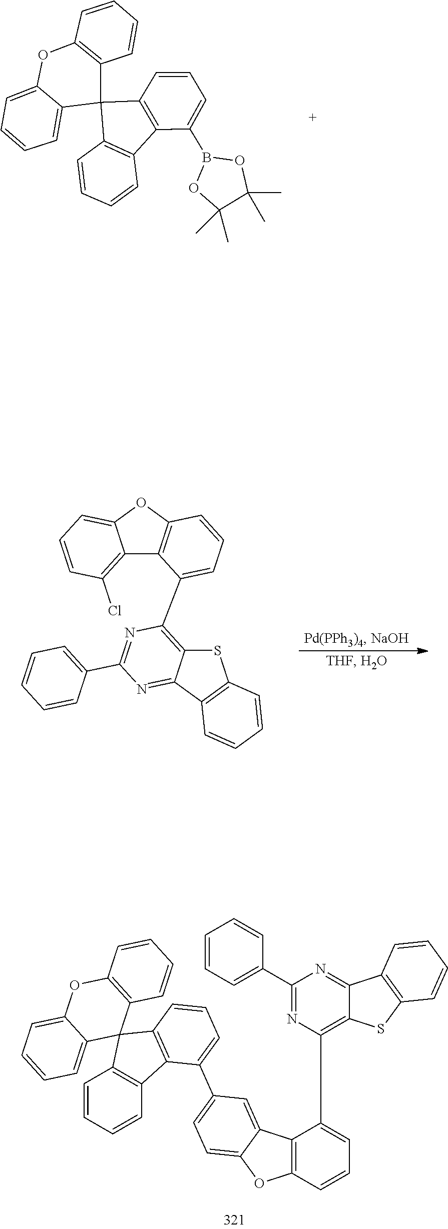

4,4,5,5 -Tetramethyl-2 -(spiro[fluorene-9,9′-xanthen]-4-yl)-1,3,2-dioxaborolane (4.6 g, 10.0 mmol), 4-(9-Chlorodibenzo[b,d]furan-1-yl)-2-phenylbenzo[4,5]thieno[3,2-d]pyrimidine (4.6 g, 10.0 mmol), Pd(PPh₃)₄ (0.46 g, 0.4 mmol), and NaOH (1.2 g, 30.0 mmol) were added to 50 ml of THF and 25 ml of H₂O and stirred at 80° C. for 8 hours. After completion of the reaction, 20 ml of water was added and stirred. A produced solid was filtered. After filtering, the solid was dissolved in toluene, followed by silica filtering, and then recrystallized with toluene to obtain the target compound, 321 (6.6 g, yield 87%).

[LCMS]: 758

### EXAMPLE 1

### Manufacturing of Blue Organic EL Device

The compound synthesized in Synthesis Example 1 was subjected to high-purity sublimation purification in a conventionally known method, and then a blue organic EL device was manufactured according to the following procedure.

First, a glass substrate coated with indium tin oxide (ITO) to a thickness of 1500 Å was washed with distilled water ultrasonically. After washing with distilled water was completed, the glass substrate was ultrasonically cleaned with a solvent, such as isopropyl alcohol, acetone and methanol, dried, transferred to a UV OZONE cleaner (Power sonic 405, Hwasin Tech) cleaned for 5 minutes using UV, and then transferred to a vacuum evaporator.

On the ITO transparent electrode prepared as above, DS-205 (Doosan Electronics Co., Ltd., 80 nm)/NPB (15 nm)/ADN+5% DS-405 (Doosan Electronics Co., Ltd.) (30 nm)/Compound 2/LiF (1 nm)/Al (200 nm) were stacked in order to prepare an organic EL device. In such a case, the structures of the NPB and AND used are as follows.

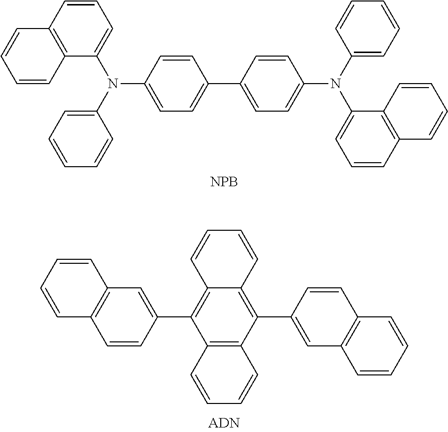

### EXAMPLES 2 to 16

### Manufacturing of Blue Organic EL Devices

Blue organic EL devices were manufactured in the same manner as in Example 1, except that compounds listed in Table 1 were respectively used as an electron transport layer material instead of Compound 2 used as a light emitting layer material in Example 1.

### COMPARATIVE EXAMPLES 1 to 3

### Manufacturing of Blue Organic EL Devices

Blue organic EL devices were manufactured in the same manner as in Example 1, except that the following compounds Alq₃, T-1, and T-2 were used as the electron transport layer material instead of Compound 2.

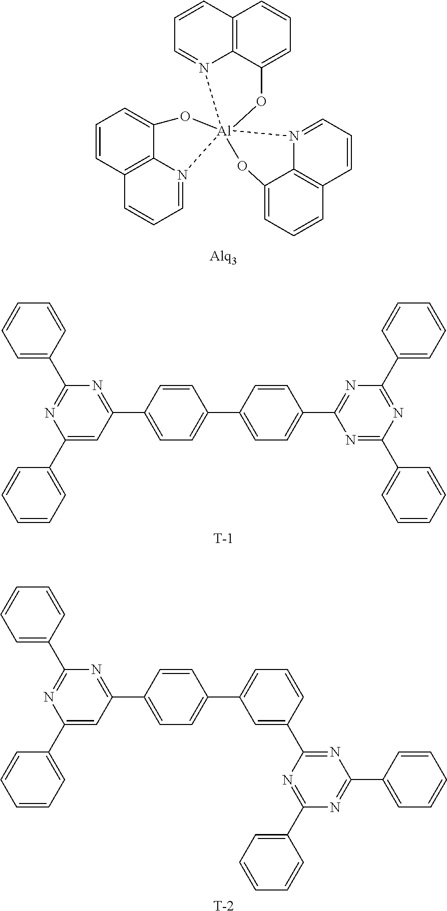

### EVALUATION EXAMPLE 1

For each of the blue organic EL devices manufactured in Examples 1 to 16 and Comparative Examples 1 to 3, driving voltage, current efficiency and service life at a current density of 10 mA/cm² were measured, and the results are shown in Table 1 below.

| | | Driving | Emission | Current |
|---|---|---|---|---|
| | Electron transport | voltage | peak | efficiency |
| Sample | layer material | (V) | (nm) | (cd/A) |
| Ex. 1 | Compound 2 | 3.2 | 454 | 10.0 |
| Ex. 2 | Compound 7 | 3.3 | 456 | 10.1 |
| Ex. 3 | Compound 30 | 3.2 | 457 | 9.9 |
| Ex. 4 | Compound 50 | 3.5 | 452 | 8.7 |
| Ex. 5 | Compound 61 | 3.1 | 455 | 10.3 |
| Ex. 6 | Compound 98 | 3.6 | 452 | 10.1 |
| Ex. 7 | Compound 125 | 3.7 | 453 | 9.9 |
| Ex. 8 | Compound 138 | 3.2 | 454 | 8.8 |
| Ex. 9 | Compound 161 | 3.8 | 455 | 8.7 |
| Ex. 10 | Compound 211 | 3.9 | 456 | 10.3 |
| Ex. 11 | Compound 231 | 4.0 | 456 | 10.1 |
| Ex. 12 | Compound 286 | 3.6 | 456 | 9.9 |
| Ex. 13 | Compound 295 | 3.7 | 456 | 9.9 |
| Ex. 14 | Compound 311 | 3.8 | 456 | 8.8 |
| Ex. 15 | Compound 312 | 3.5 | 456 | 8.7 |
| Ex. 16 | Compound 321 | 3.6 | 456 | 8.2 |
| Comp. Ex. 1 | Alq₃ | 5.4 | 458 | 5.5 |
| Comp. Ex. 2 | T-1 | 4.5 | 459 | 5.9 |
| Comp. Ex. 3 | T-2 | 4.4 | 458 | 6.0 |

As shown in Table 1, it was appreciated that the blue organic EL devices (Examples 1 to 16) using the compound according to the present invention for the electron transport layer exhibited excellent performance in terms of the driving voltage, the emission peak and the current efficiency, as compared to the blue organic EL device (Comparative Example 1) using conventional Alq₃ for the electron transport layer.

### EXAMPLE 17

### Manufacturing of Blue Organic EL Device

A blue organic EL device was manufactured in the same manner as in Example 1, except that on the ITO transparent electrode prepared in Example 1, DS-205 (Doosan Electronics Co., Ltd.) (80 nm)/NPB (15 nm)/ADN +5% DS-405 (Doosan Electronics Co., Ltd.) (30 nm)/Compound 2 (5 nm)/ Alq₃ (25 nm)/LiF (1 nm)/Al (200 nm) were stacked in order.

### EXAMPLES 18 to 32

### Manufacturing of Blue Organic EL Devices

Blue organic EL devices were manufactured in the same manner as in Example 18, except that compounds listed in Table 2 were respectively used as an electron transport auxiliary layer material instead of Compound 2 used as an electron transport auxiliary layer material in Example 18.

### COMPARATIVE EXAMPLES 4 to 6

### Manufacturing of Blue Organic EL Devices

Blue organic EL devices were manufactured in the same manner as in Example 1, except that compounds Alq₃, T-1, and T-2 were used instead of Compound 2 as the electron transport auxiliary layer material.

### EVALUATION EXAMPLE 2

For each of the blue organic EL devices manufactured in Examples 17 to 32 and Comparative Examples 4 to 6, driving voltage, current efficiency and service life at a current density of 10 mA/cm² were measured, and the results are shown in Table 2 below.

TABLE 2

| | | Driving | Emission | Current |
|---|---|---|---|---|
| | Electron transport | voltage | peak | efficiency |
| Sample | auxiliary layer material | (V) | (nm) | (cd/A) |
| Ex. 17 | Compound 2 | 3.8 | 454 | 8.5 |
| Ex. 18 | Compound 7 | 3.7 | 456 | 8.9 |
| Ex. 19 | Compound 30 | 3.9 | 457 | 8.7 |
| Ex. 20 | Compound 50 | 3.5 | 452 | 9.3 |
| Ex. 21 | Compound 61 | 3.4 | 455 | 9.5 |
| Ex. 22 | Compound 98 | 3.6 | 452 | 9.2 |
| Ex. 23 | Compound 125 | 3.7 | 453 | 9.5 |
| Ex. 24 | Compound 138 | 3.2 | 454 | 8.8 |
| Ex. 25 | Compound 161 | 3.8 | 455 | 8.7 |
| Ex. 26 | Compound 211 | 3.2 | 454 | 10.0 |
| Ex. 27 | Compound 231 | 3.2 | 454 | 10.0 |
| Ex. 28 | Compound 286 | 3.3 | 456 | 10.1 |
| Ex. 29 | Compound 295 | 3.2 | 457 | 9.9 |
| Ex. 30 | Compound 311 | 3.5 | 452 | 8.7 |
| Ex. 31 | Compound 312 | 3.1 | 455 | 10.3 |
| Ex. 32 | Compound 321 | 3.6 | 452 | 10.1 |
| Comp. Ex. 4 | — | 4.8 | 458 | 6.0 |
| Comp. Ex. 5 | T-1 | 4.7 | 457 | 6.1 |
| Comp. Ex. 6 | T-2 | 4.6 | 456 | 6.2 |

As shown in Table 2, it was appreciated that the blue organic EL devices (Examples 17 to 32) using the compound according to the present invention for the electron transport auxiliary layer exhibited excellent performance in terms of the driving voltage, the emission peak and the current efficiency, as compared to the blue organic EL device (Comparative Example 4) not including the electron transport auxiliary layer.

1. A compound represented by the following Chemical Formula 1: 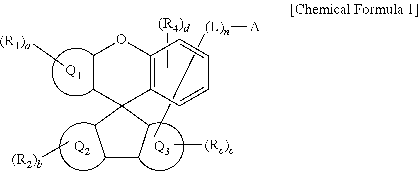
wherein in Chemical Formula 1,
Q₁ to Q₃ are the same as or different from each other, each independently being a C₆ to C₁₀ aromatic ring;
L is a single bond or is selected from: a C₆ to C₁₈ arylene group and a heteroarylene group having 5 to 18 nuclear atoms;
R₁ to R₄ are the same as or different from each other, each independently being selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms,
A is a substituent represented by the following Chemical Formula a, 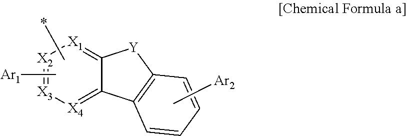
wherein in the above Chemical Formula a,
* means a site bonded to Chemical Formula 1,
Y is S or O;
X₁ to X₄ are the same as or different from each other, each independently being CH or N, provided that at least two of X₁ to X₄ are N,
Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, a C₆ to C₆₀ arylamine group, a C₅ to C₆₀ arylheteroarylamine group, and a heteroarylamine group having 5 to 60 nuclear atoms,
each of a to d and n is an integer in a range from 0 to 3, and
the arylene group and the heteroarylene group of L and the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphanyl group, the monoarylphosphinyl group, the diarylphosphinyl group, the arylamine group, the arylheteroarylamine group, and the heteroarylamine group of R₁ to R₄ and Ar₁ and Ar₂ are each independently substitutable with one or more kinds of substituents selected from: deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, a C₆ to C₆₀ arylamine group, a C₅ to C₆₀ arylheteroarylamine group, and a heteroarylamine group having 5 to 60 nuclear atoms, and when the substituents are plural in number, the substituents are the same as or different from each other.

2. The compound of claim 1, wherein the compound represented by Chemical Formula 1 is represented by any one of Chemical Formulas 2 to 5: 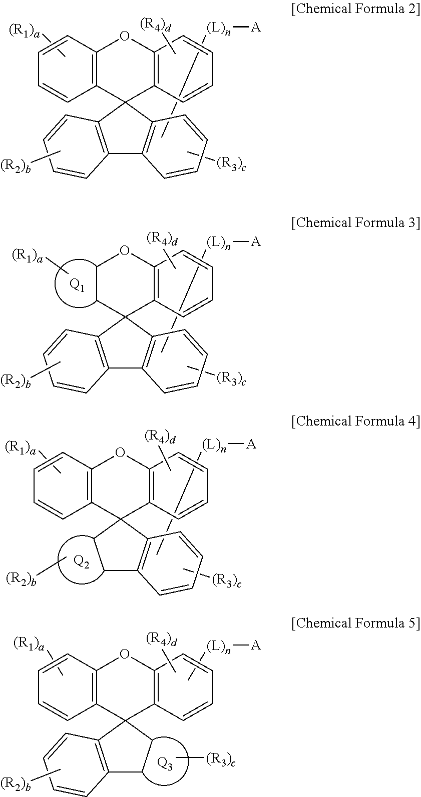
wherein in Chemical Formulas 2 to 5,
each of Q₁ to Q₃ is a C₁₀ aromatic ring, and
L, R₁ to R₄, A, a to d, and n are as defined in claim 1, respectively.

3. The compound of claim 1, wherein the compound represented by Chemical Formula 1 is represented by any one of Chemical Formulas 6 to 12: 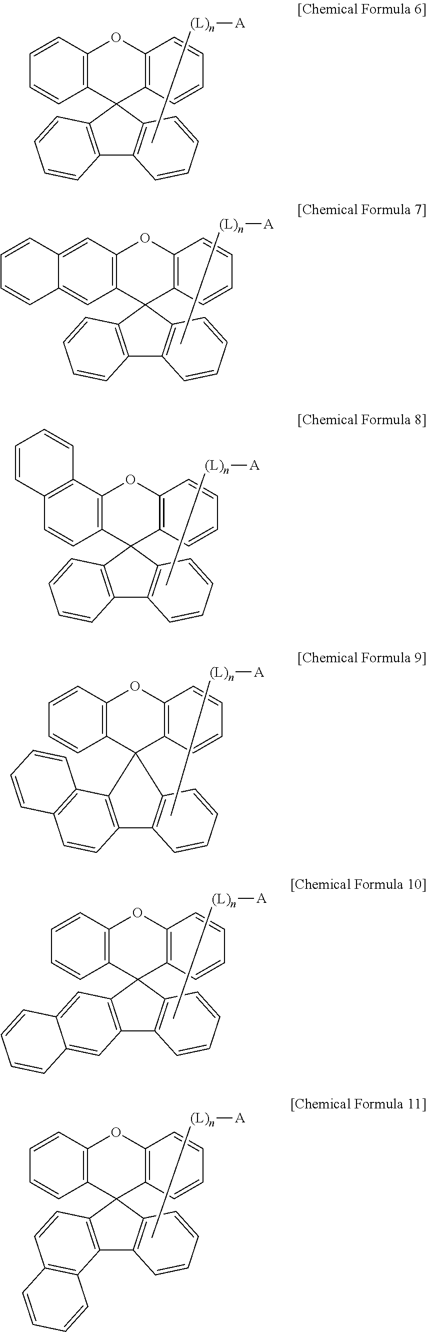 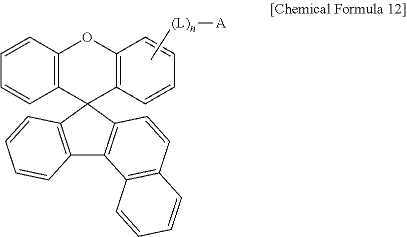
wherein in Chemical Formulas 6 to 12,
L, A and n are as defined in claim 1, respectively.

4. The compound of claim 1, wherein the compound represented by Chemical Formula 1 is represented by any one of Chemical Formulas 13 to 18: 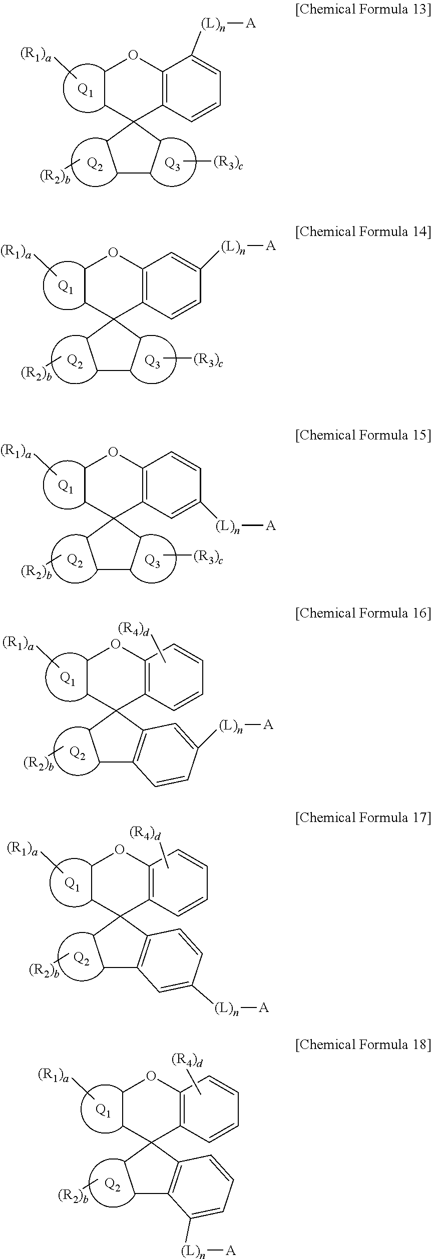
wherein in Chemical Formulas 13 to 18,
Q₁ to Q₃, L, R₁ to R₄, A, a to d, and n are as defined in claim 1, respectively.

5. The compound of claim 1, wherein L is a linker of a single bond or selected from the following structural formulas: 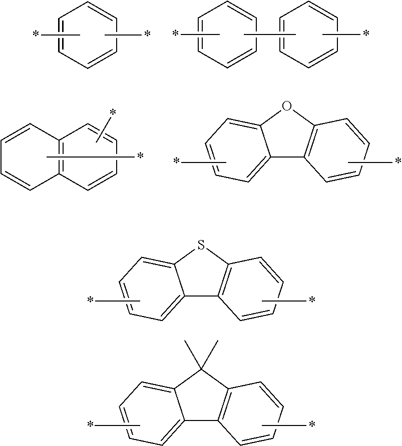
wherein in the above structural formulas,
* means a site where a bond with the Chemical Formula 1 is made.

6. The compound of claim 1, wherein two of X₁ to X₄ are N.

7. The compound of claim 1, wherein A is represented by the following Chemical Formula a-1: 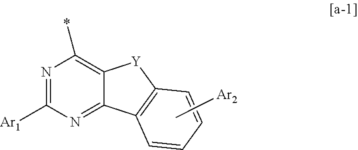
wherein in the above Chemical Formula a-1,
* means a site where a bond with the Chemical Formula 1 is made.
Y, Ar₁ and Ar₂ are as defined in claim 1, respectively.

8. The compound of claim 1, wherein Ar₁ is selected from: a C₆ to C₆₀ aryl group and a heteroaryl group having 5 to 60 nuclear atoms, and the aryl group and the heteroaryl group of Ar₁ are substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₁ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and when the substituents are plural in number, the substituents are the same as or different from each other.

9. The compound of claim 1, wherein the compound represented by Chemical Formula 1 is selected from compounds represented by the following Chemical Formulas:

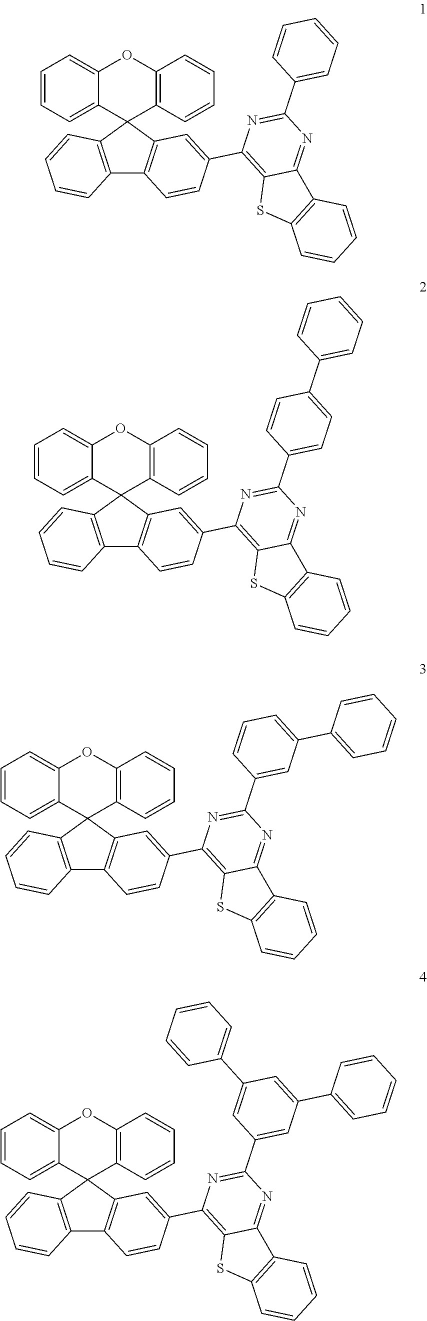

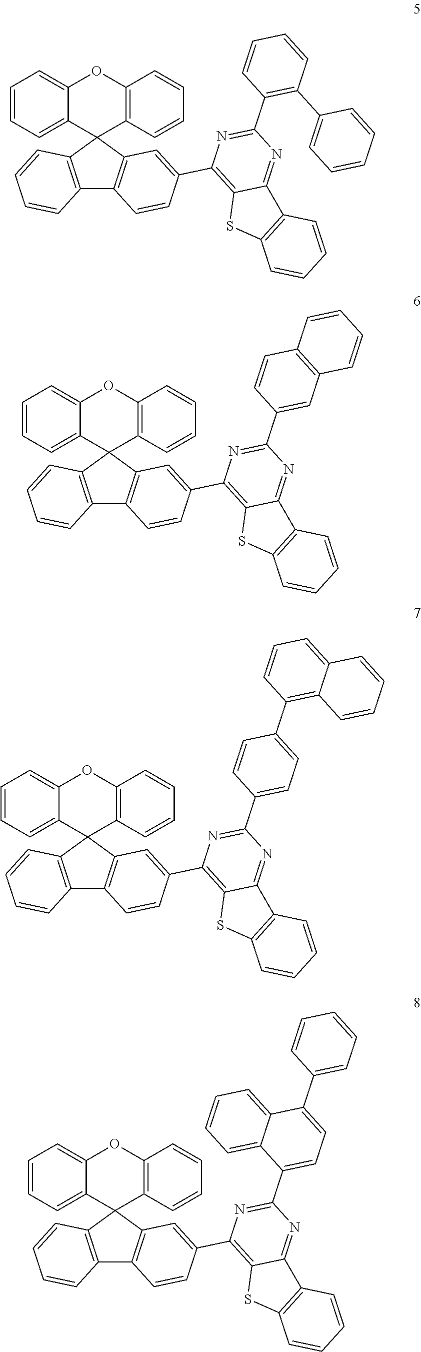

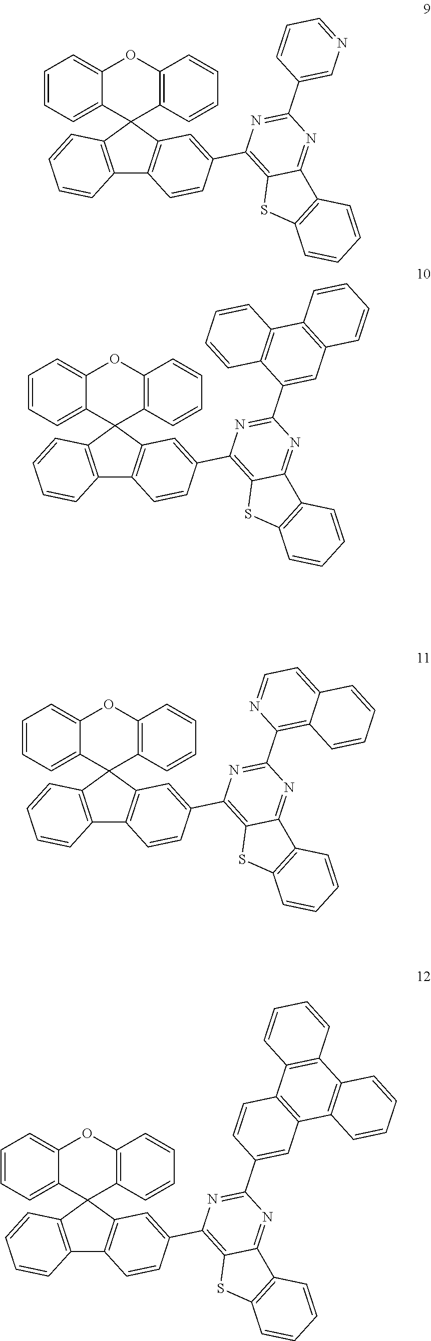

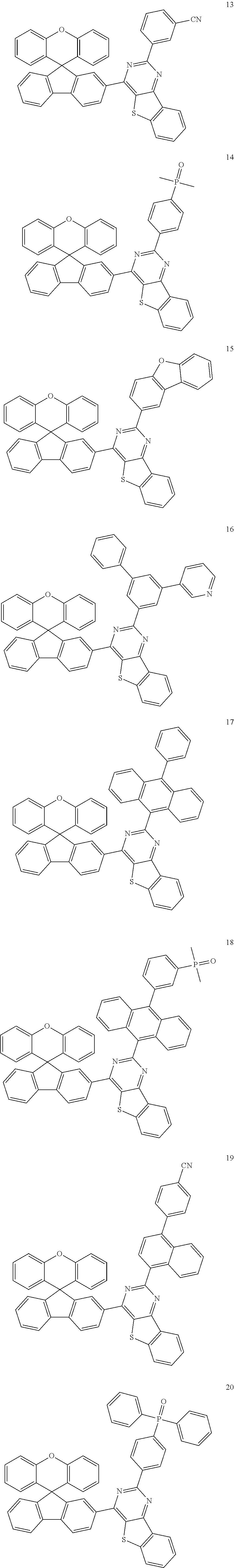

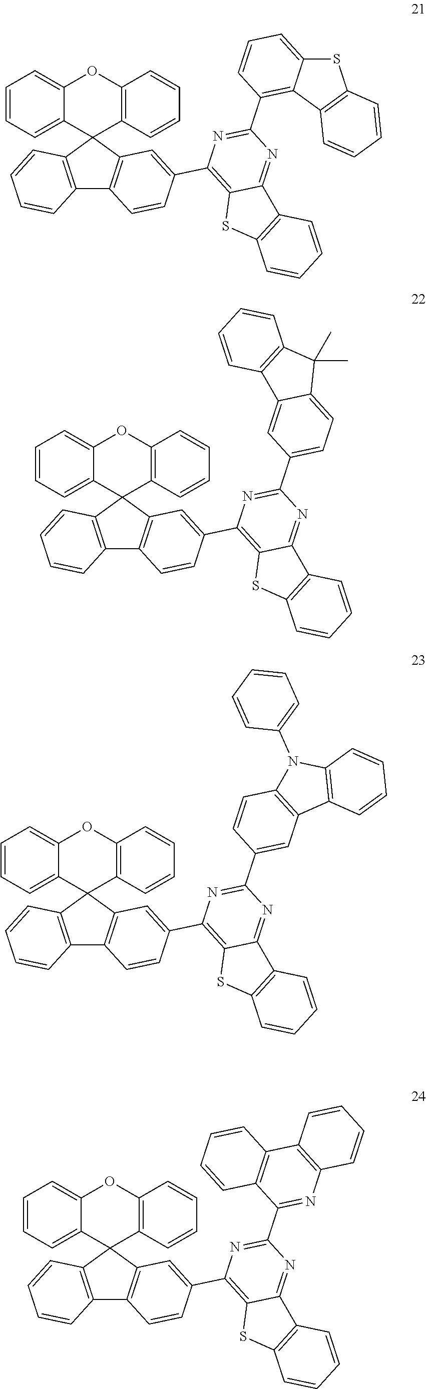

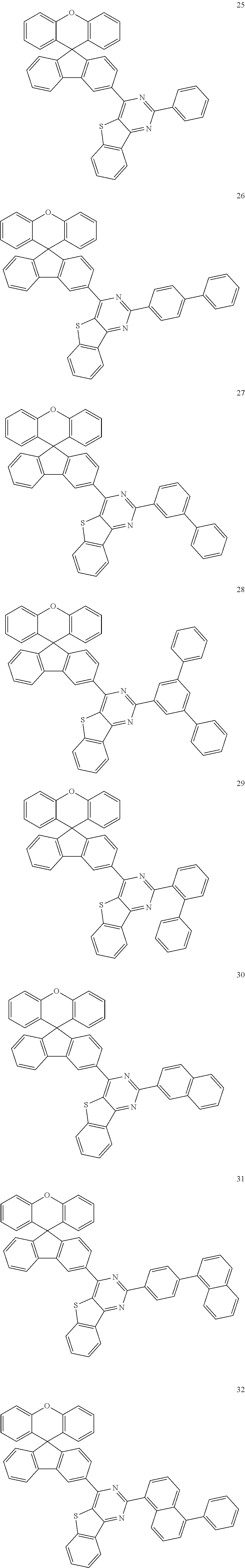

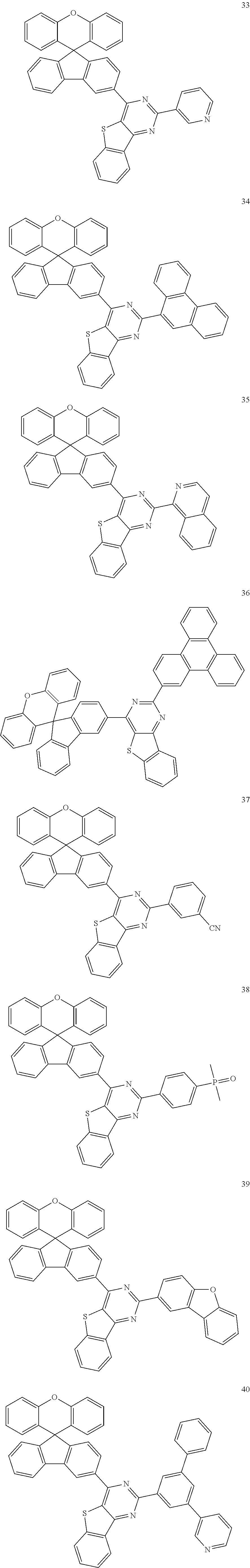

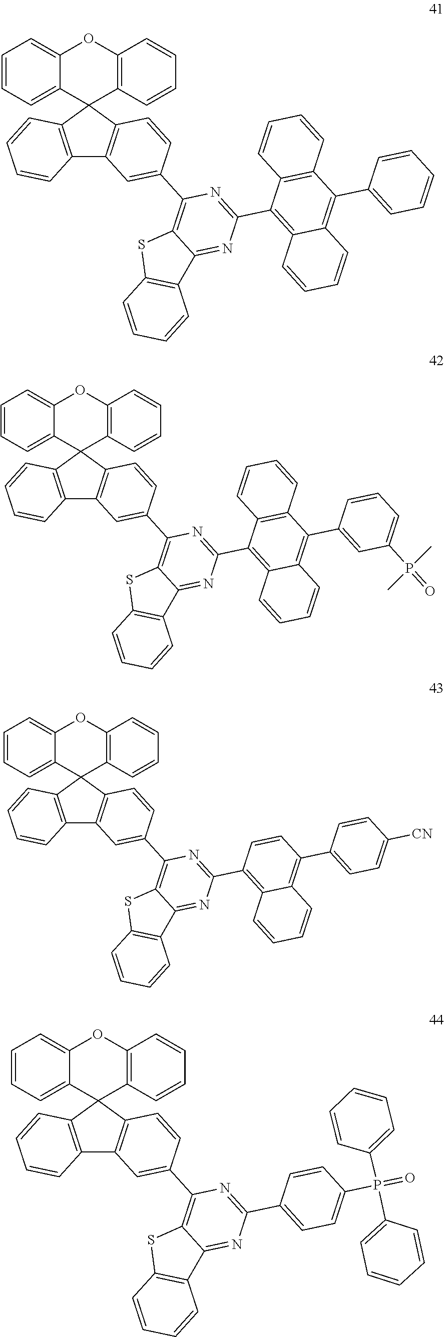 [SMILES: c1ccc(-c2c3ccccc3c(-c3nc(-c4ccc5c(c4)-c4ccccc4C54c5ccccc5Oc5ccccc54)c4sc5ccccc5c4n3)c3ccccc23)cc1 ; CP(C)(=O)c1cccc(-c2c3ccccc3c(-c3nc(-c4ccc5c(c4)-c4ccccc4C54c5ccccc5Oc5ccccc54)c4sc5ccccc5c4n3)c3ccccc23)c1 ; N#Cc1ccc(-c2ccc(-c3nc(-c4ccc5c(c4)-c4ccccc4C54c5ccccc5Oc5ccccc54)c4sc5ccccc5c4n3)c3ccccc23)cc1 ; O=P(c1ccccc1)(c1ccccc1)c1ccc(-c2nc(-c3ccc4c(c3)-c3ccccc3C43c4ccccc4Oc4ccccc43)c3sc4ccccc4c3n2)cc1]

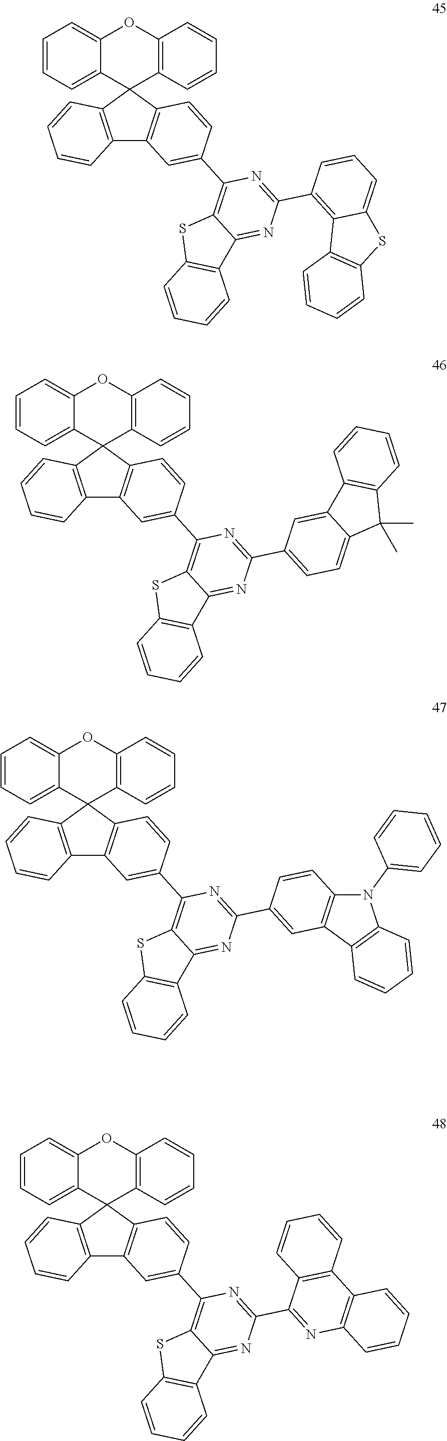 [SMILES: c1ccc2c(c1)Oc1ccccc1C21c2ccccc2-c2cc(-c3nc(-c4cccc5sc6ccccc6c45)nc4c3sc3ccccc34)ccc21 ; CC1(C)c2ccccc2-c2cc(-c3nc(-c4ccc5c(c4)-c4ccccc4C54c5ccccc5Oc5ccccc54)c4sc5ccccc5c4n3)ccc21 ; c1ccc(-n2c3ccccc3c3cc(-c4nc(-c5ccc6c(c5)-c5ccccc5C65c6ccccc6Oc6ccccc65)c5sc6ccccc6c5n4)ccc32)cc1 ; c1ccc2c(c1)Oc1ccccc1C21c2ccccc2-c2cc(-c3nc(-c4nc5ccccc5c5ccccc45)nc4c3sc3ccccc34)ccc21]

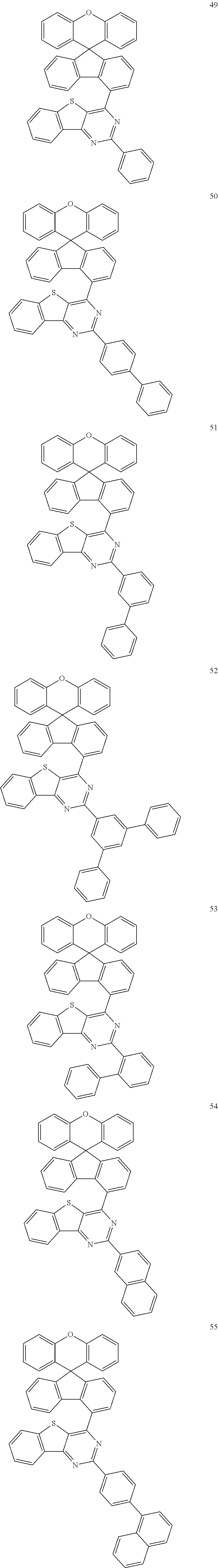

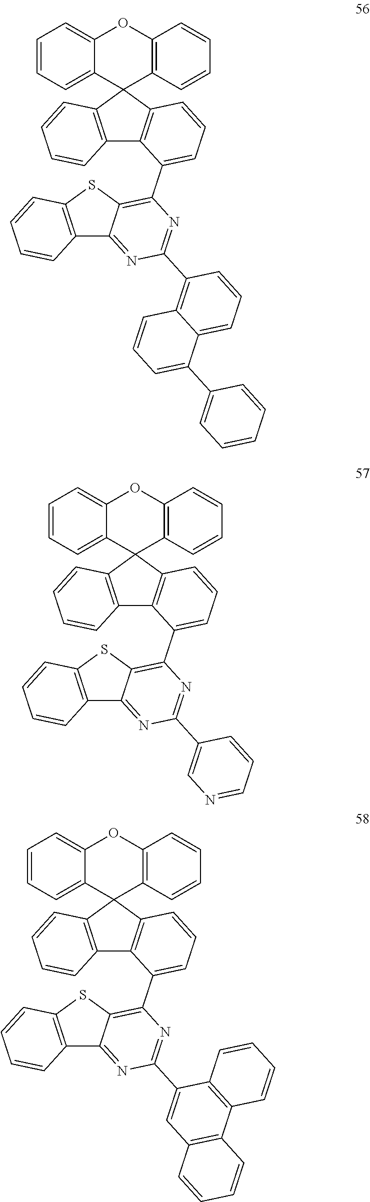 [SMILES: c1ccc(-c2cccc3c(-c4nc(-c5cccc6c5-c5ccccc5C65c6ccccc6Oc6ccccc65)c5sc6ccccc6c5n4)cccc23)cc1 ; c1cncc(-c2nc(-c3cccc4c3-c3ccccc3C43c4ccccc4Oc4ccccc43)c3sc4ccccc4c3n2)c1 ; c1ccc2c(c1)Oc1ccccc1C21c2ccccc2-c2c(-c3nc(-c4cc5ccccc5c5ccccc45)nc4c3sc3ccccc34)cccc21]

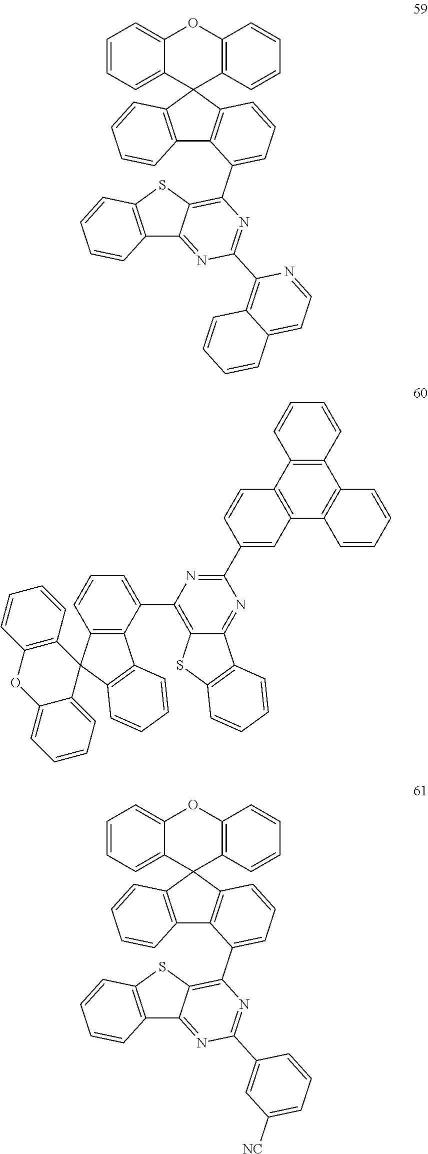 [SMILES: c1ccc2c(c1)Oc1ccccc1C21c2ccccc2-c2c(-c3nc(-c4nccc5ccccc45)nc4c3sc3ccccc34)cccc21 ; c1ccc2c(c1)Oc1ccccc1C21c2ccccc2-c2c(-c3nc(-c4ccc5c6ccccc6c6ccccc6c5c4)nc4c3sc3ccccc34)cccc21 ; N#Cc1cccc(-c2nc(-c3cccc4c3-c3ccccc3C43c4ccccc4Oc4ccccc43)c3sc4ccccc4c3n2)c1]

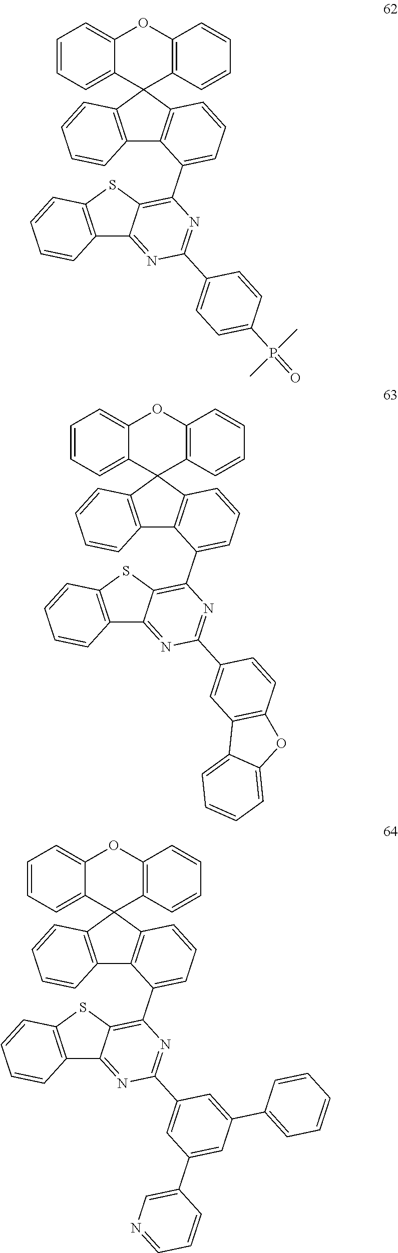 [SMILES: CP(C)(=O)c1ccc(-c2nc(-c3cccc4c3-c3ccccc3C43c4ccccc4Oc4ccccc43)c3sc4ccccc4c3n2)cc1 ; c1ccc2c(c1)Oc1ccccc1C21c2ccccc2-c2c(-c3nc(-c4ccc5oc6ccccc6c5c4)nc4c3sc3ccccc34)cccc21 ; c1ccc(-c2cc(-c3cccnc3)cc(-c3nc(-c4cccc5c4-c4ccccc4C54c5ccccc5Oc5ccccc54)c4sc5ccccc5c4n3)c2)cc1]

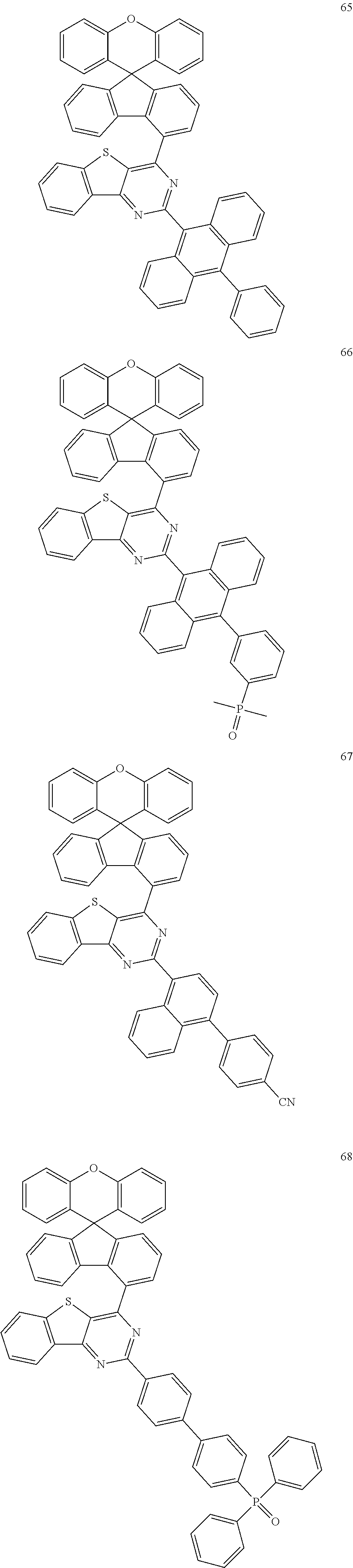 [SMILES: c1ccc(-c2c3ccccc3c(-c3nc(-c4cccc5c4-c4ccccc4C54c5ccccc5Oc5ccccc54)c4sc5ccccc5c4n3)c3ccccc23)cc1 ; CP(C)(=O)c1cccc(-c2c3ccccc3c(-c3nc(-c4cccc5c4-c4ccccc4C54c5ccccc5Oc5ccccc54)c4sc5ccccc5c4n3)c3ccccc23)c1 ; N#Cc1ccc(-c2ccc(-c3nc(-c4cccc5c4-c4ccccc4C54c5ccccc5Oc5ccccc54)c4sc5ccccc5c4n3)c3ccccc23)cc1 ; O=P(c1ccccc1)(c1ccccc1)c1ccc(-c2ccc(-c3nc(-c4cccc5c4-c4ccccc4C54c5ccccc5Oc5ccccc54)c4sc5ccccc5c4n3)cc2)cc1]

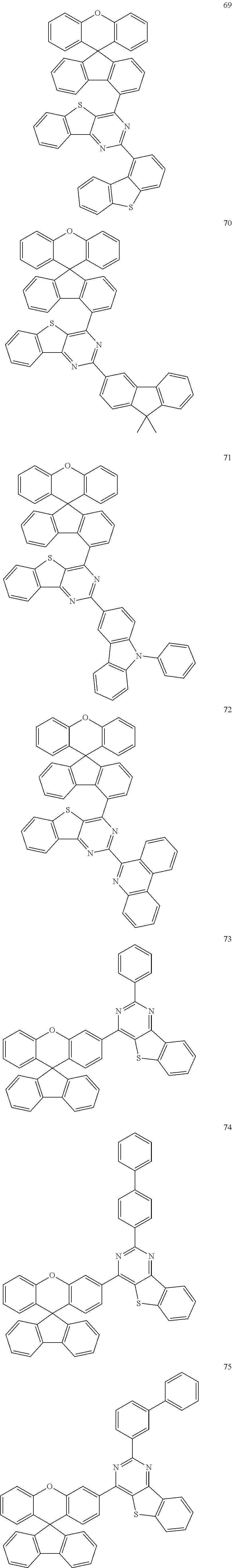

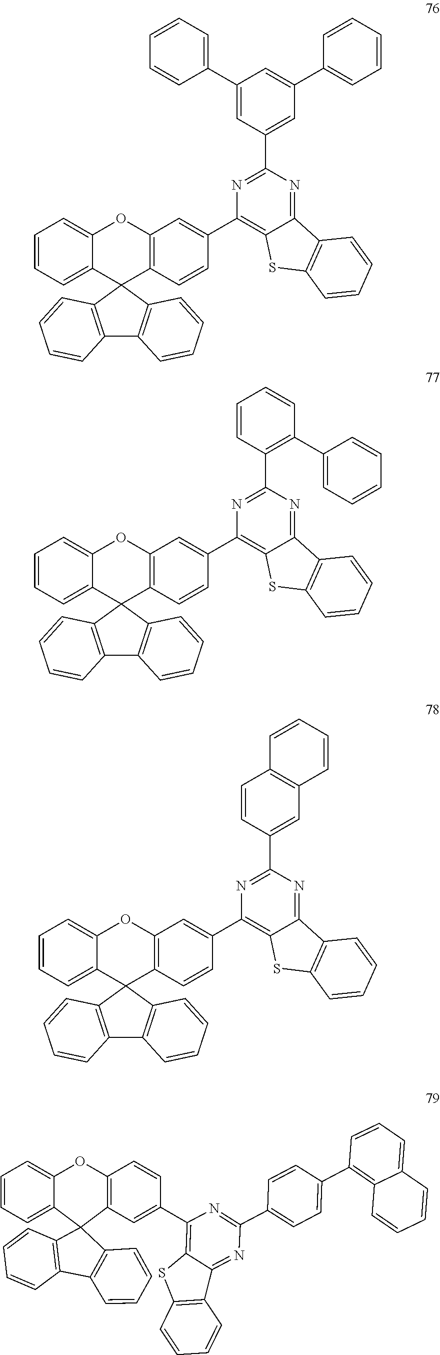 [SMILES: c1ccc(-c2cc(-c3ccccc3)cc(-c3nc(-c4ccc5c(c4)Oc4ccccc4C54c5ccccc5-c5ccccc54)c4sc5ccccc5c4n3)c2)cc1 ; c1ccc(-c2ccccc2-c2nc(-c3ccc4c(c3)Oc3ccccc3C43c4ccccc4-c4ccccc43)c3sc4ccccc4c3n2)cc1 ; c1ccc2c(c1)Oc1cc(-c3nc(-c4ccc5ccccc5c4)nc4c3sc3ccccc34)ccc1C21c2ccccc2-c2ccccc21 ; c1ccc2c(c1)Oc1ccc(-c3nc(-c4ccc(-c5cccc6ccccc56)cc4)nc4c3sc3ccccc34)cc1C21c2ccccc2-c2ccccc21]

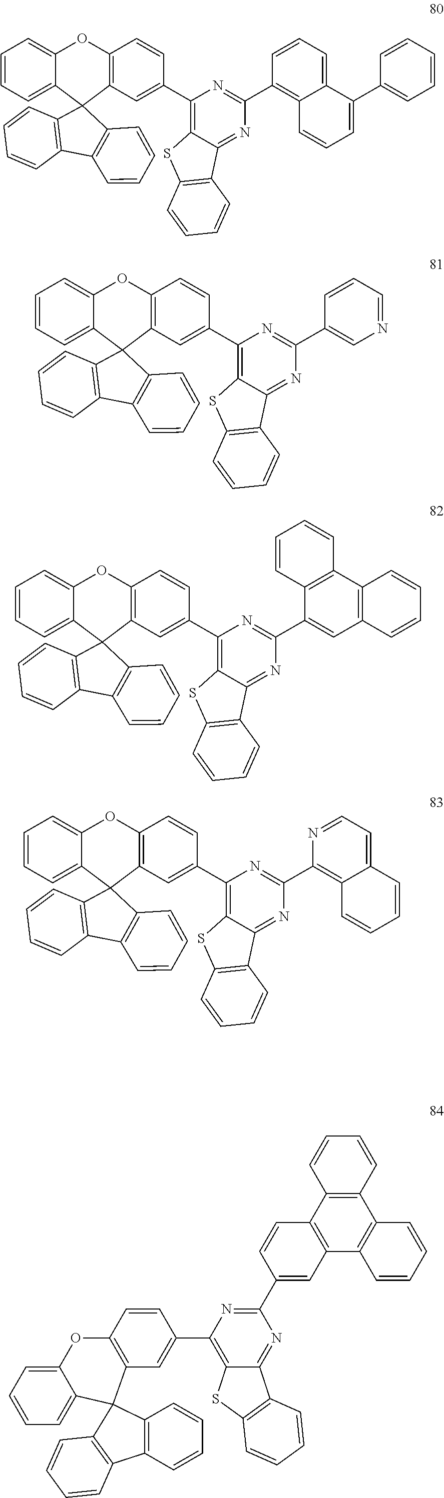

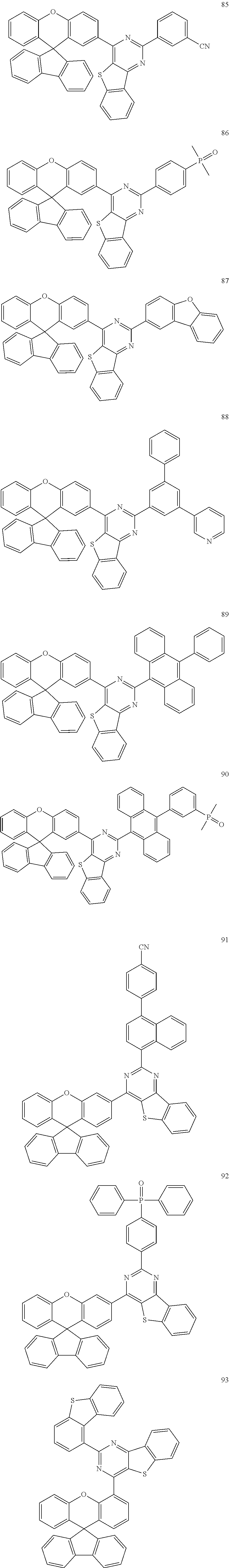

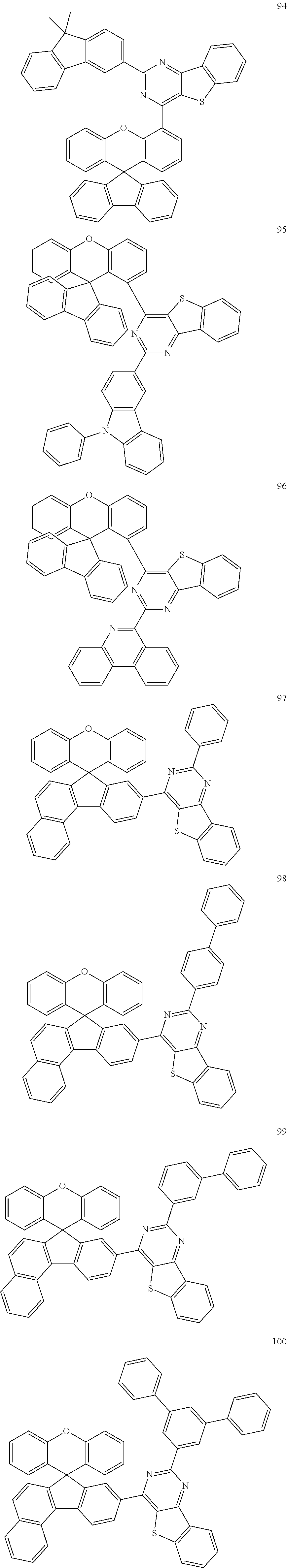

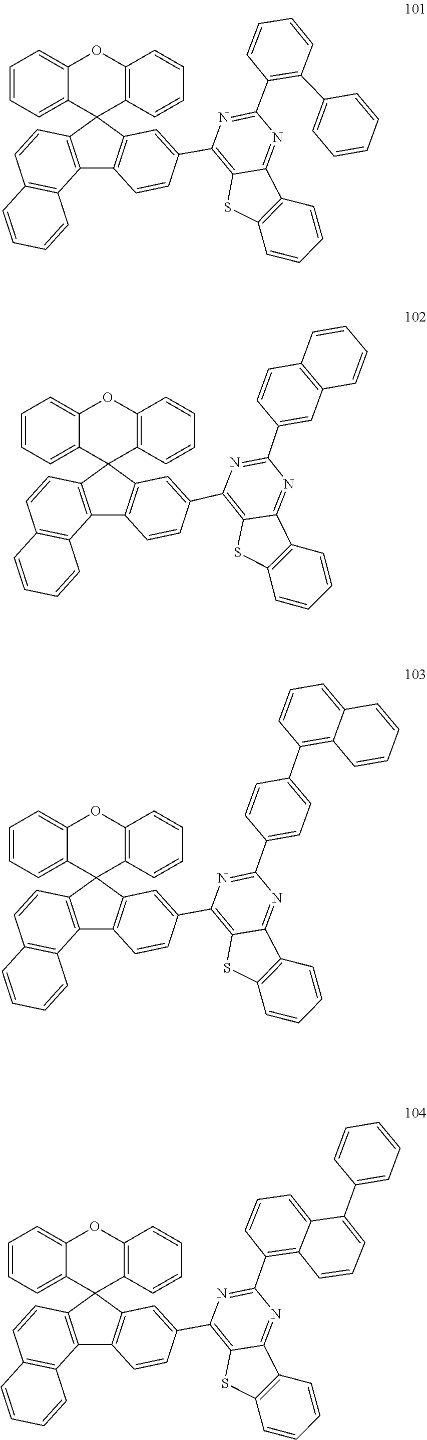 [SMILES: c1ccc(-c2ccccc2-c2nc(-c3ccc4c(c3)C3(c5ccccc5Oc5ccccc53)c3ccc5ccccc5c3-4)c3sc4ccccc4c3n2)cc1 ; c1ccc2c(c1)Oc1ccccc1C21c2cc(-c3nc(-c4ccc5ccccc5c4)nc4c3sc3ccccc34)ccc2-c2c1ccc1ccccc21 ; c1ccc2c(c1)Oc1ccccc1C21c2cc(-c3nc(-c4ccc(-c5cccc6ccccc56)cc4)nc4c3sc3ccccc34)ccc2-c2c1ccc1ccccc21 ; c1ccc(-c2cccc3c(-c4nc(-c5ccc6c(c5)C5(c7ccccc7Oc7ccccc75)c5ccc7ccccc7c5-6)c5sc6ccccc6c5n4)cccc23)cc1]

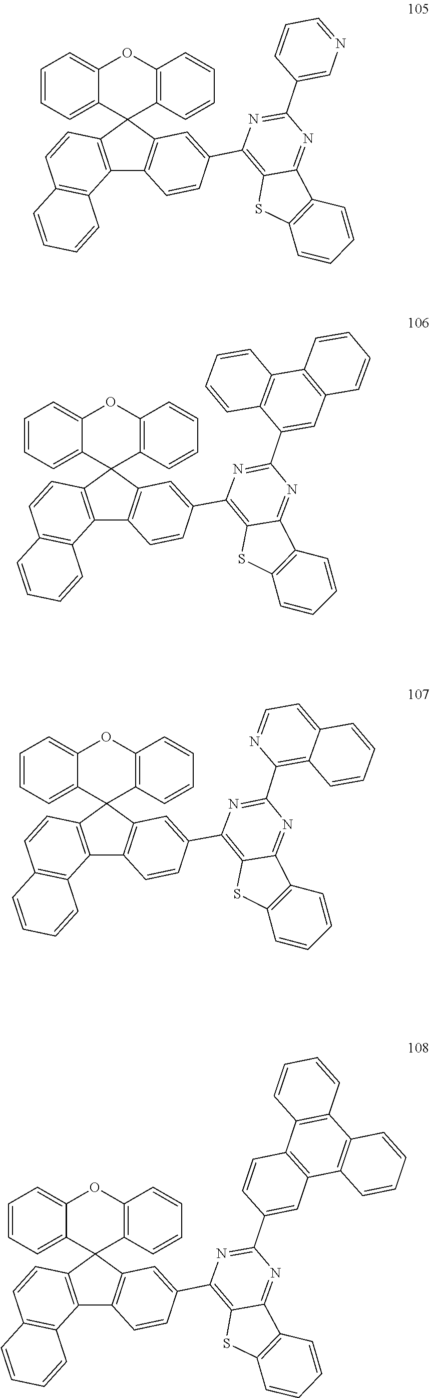 [SMILES: c1cncc(-c2nc(-c3ccc4c(c3)C3(c5ccccc5Oc5ccccc53)c3ccc5ccccc5c3-4)c3sc4ccccc4c3n2)c1 ; c1ccc2c(c1)Oc1ccccc1C21c2cc(-c3nc(-c4cc5ccccc5c5ccccc45)nc4c3sc3ccccc34)ccc2-c2c1ccc1ccccc21 ; c1ccc2c(c1)Oc1ccccc1C21c2cc(-c3nc(-c4nccc5ccccc45)nc4c3sc3ccccc34)ccc2-c2c1ccc1ccccc21 ; c1ccc2c(c1)Oc1ccccc1C21c2cc(-c3nc(-c4ccc5c6ccccc6c6ccccc6c5c4)nc4c3sc3ccccc34)ccc2-c2c1ccc1ccccc21]

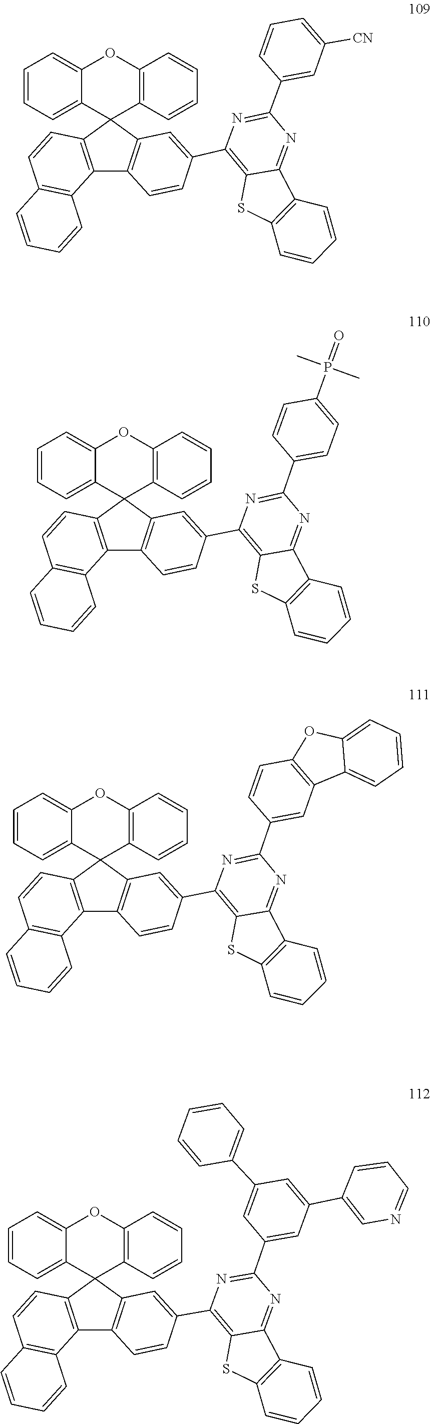 [SMILES: N#Cc1cccc(-c2nc(-c3ccc4c(c3)C3(c5ccccc5Oc5ccccc53)c3ccc5ccccc5c3-4)c3sc4ccccc4c3n2)c1 ; CP(C)(=O)c1ccc(-c2nc(-c3ccc4c(c3)C3(c5ccccc5Oc5ccccc53)c3ccc5ccccc5c3-4)c3sc4ccccc4c3n2)cc1 ; c1ccc2c(c1)Oc1ccccc1C21c2cc(-c3nc(-c4ccc5oc6ccccc6c5c4)nc4c3sc3ccccc34)ccc2-c2c1ccc1ccccc21 ; c1ccc(-c2cc(-c3cccnc3)cc(-c3nc(-c4ccc5c(c4)C4(c6ccccc6Oc6ccccc64)c4ccc6ccccc6c4-5)c4sc5ccccc5c4n3)c2)cc1]

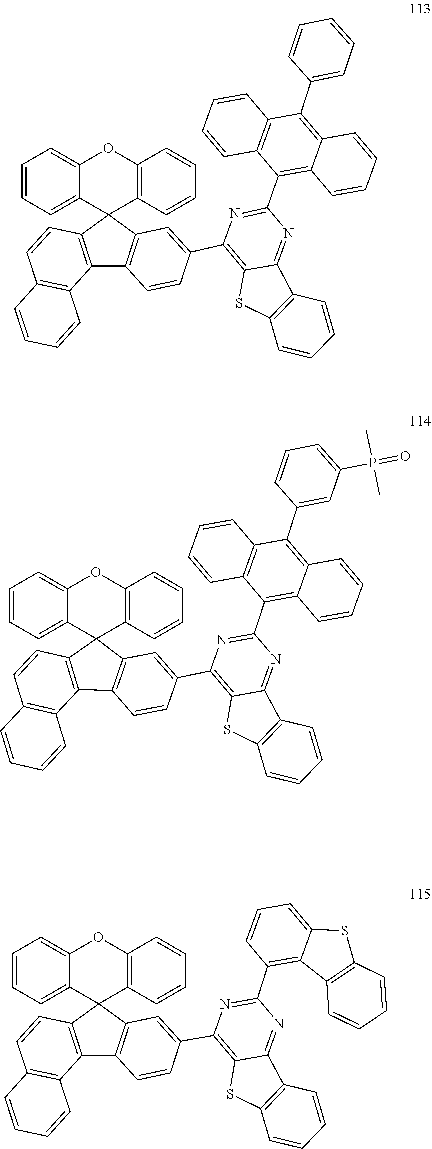 [SMILES: c1ccc(-c2c3ccccc3c(-c3nc(-c4ccc5c(c4)C4(c6ccccc6Oc6ccccc64)c4ccc6ccccc6c4-5)c4sc5ccccc5c4n3)c3ccccc23)cc1 ; CP(C)(=O)c1cccc(-c2c3ccccc3c(-c3nc(-c4ccc5c(c4)C4(c6ccccc6Oc6ccccc64)c4ccc6ccccc6c4-5)c4sc5ccccc5c4n3)c3ccccc23)c1 ; c1ccc2c(c1)Oc1ccccc1C21c2cc(-c3nc(-c4cccc5sc6ccccc6c45)nc4c3sc3ccccc34)ccc2-c2c1ccc1ccccc21]

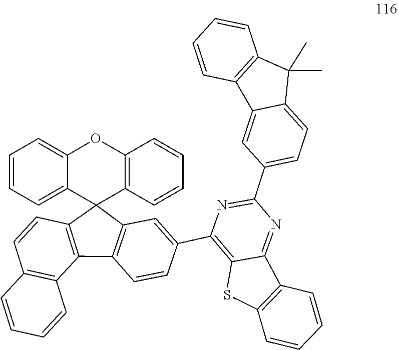 [SMILES: CC1(C)c2ccccc2-c2cc(-c3nc(-c4ccc5c(c4)C4(c6ccccc6Oc6ccccc64)c4ccc6ccccc6c4-5)c4sc5ccccc5c4n3)ccc21]

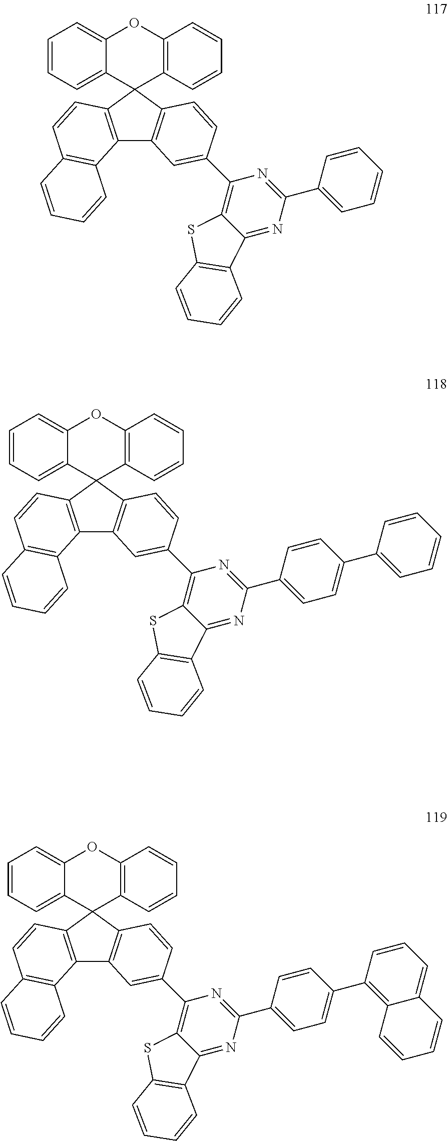 [SMILES: c1ccc(-c2nc(-c3ccc4c(c3)-c3c(ccc5ccccc35)C43c4ccccc4Oc4ccccc43)c3sc4ccccc4c3n2)cc1 ; c1ccc(-c2ccc(-c3nc(-c4ccc5c(c4)-c4c(ccc6ccccc46)C54c5ccccc5Oc5ccccc54)c4sc5ccccc5c4n3)cc2)cc1 ; c1ccc2c(c1)Oc1ccccc1C21c2ccc(-c3nc(-c4ccc(-c5cccc6ccccc56)cc4)nc4c3sc3ccccc34)cc2-c2c1ccc1ccccc21]

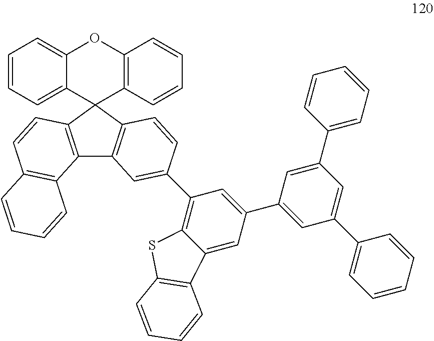 [SMILES: c1ccc(-c2cc(-c3ccccc3)cc(-c3cc(-c4ccc5c(c4)-c4c(ccc6ccccc46)C54c5ccccc5Oc5ccccc54)c4sc5ccccc5c4c3)c2)cc1]

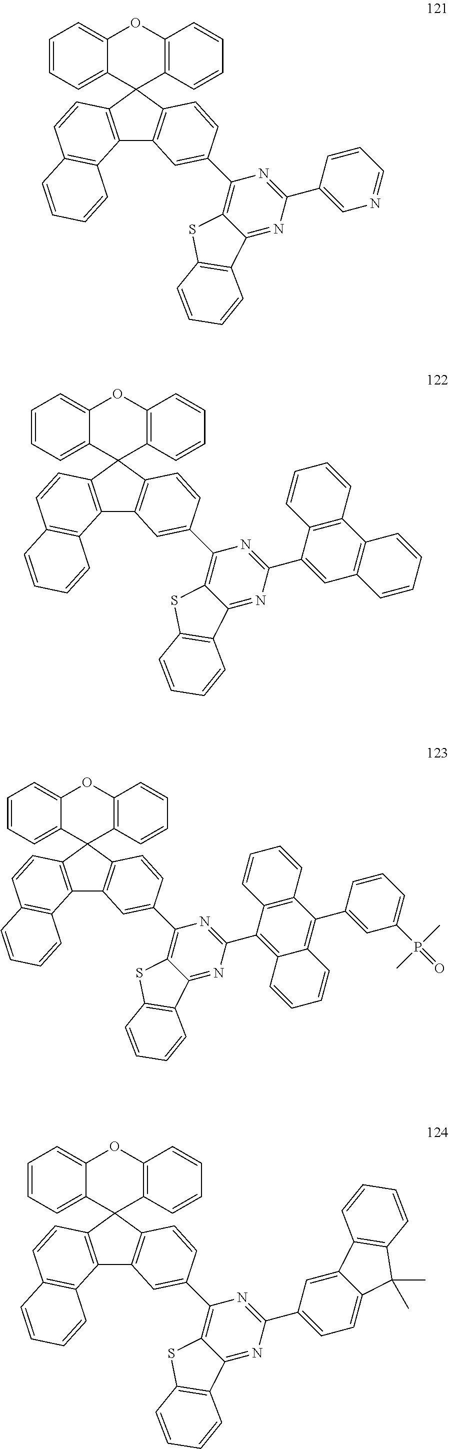 [SMILES: c1cncc(-c2nc(-c3ccc4c(c3)-c3c(ccc5ccccc35)C43c4ccccc4Oc4ccccc43)c3sc4ccccc4c3n2)c1 ; c1ccc2c(c1)Oc1ccccc1C21c2ccc(-c3nc(-c4cc5ccccc5c5ccccc45)nc4c3sc3ccccc34)cc2-c2c1ccc1ccccc21 ; CP(C)(=O)c1cccc(-c2c3ccccc3c(-c3nc(-c4ccc5c(c4)-c4c(ccc6ccccc46)C54c5ccccc5Oc5ccccc54)c4sc5ccccc5c4n3)c3ccccc23)c1 ; CC1(C)c2ccccc2-c2cc(-c3nc(-c4ccc5c(c4)-c4c(ccc6ccccc46)C54c5ccccc5Oc5ccccc54)c4sc5ccccc5c4n3)ccc21]

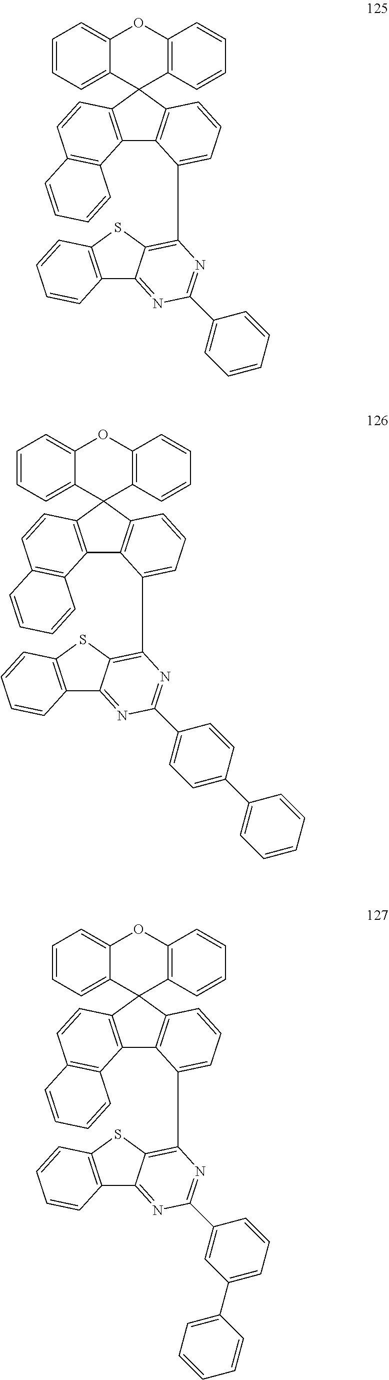 [SMILES: c1ccc(-c2nc(-c3cccc4c3-c3c(ccc5ccccc35)C43c4ccccc4Oc4ccccc43)c3sc4ccccc4c3n2)cc1 ; c1ccc(-c2ccc(-c3nc(-c4cccc5c4-c4c(ccc6ccccc46)C54c5ccccc5Oc5ccccc54)c4sc5ccccc5c4n3)cc2)cc1 ; c1ccc(-c2cccc(-c3nc(-c4cccc5c4-c4c(ccc6ccccc46)C54c5ccccc5Oc5ccccc54)c4sc5ccccc5c4n3)c2)cc1]

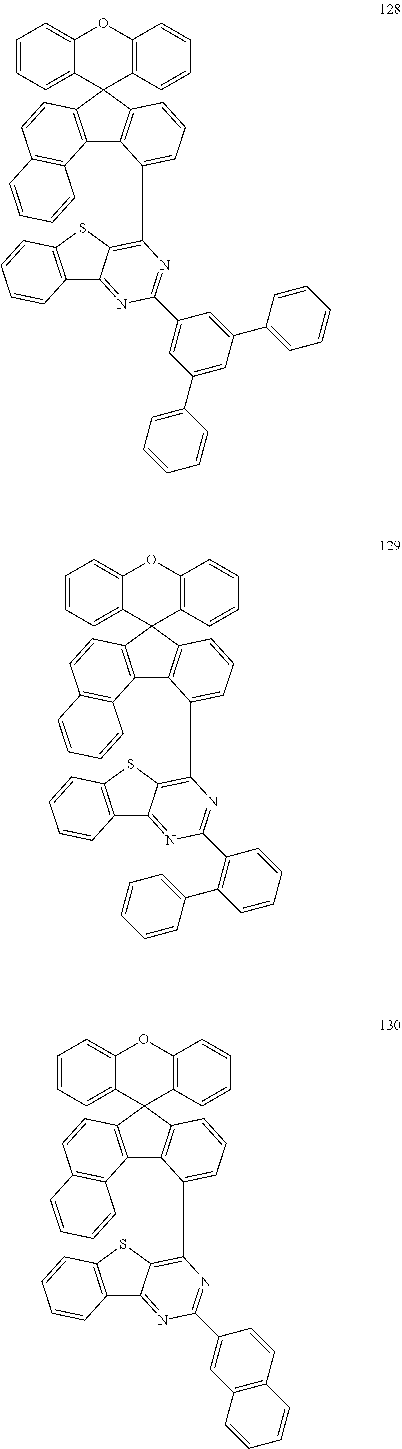 [SMILES: c1ccc(-c2cc(-c3ccccc3)cc(-c3nc(-c4cccc5c4-c4c(ccc6ccccc46)C54c5ccccc5Oc5ccccc54)c4sc5ccccc5c4n3)c2)cc1 ; c1ccc(-c2ccccc2-c2nc(-c3cccc4c3-c3c(ccc5ccccc35)C43c4ccccc4Oc4ccccc43)c3sc4ccccc4c3n2)cc1 ; c1ccc2c(c1)Oc1ccccc1C21c2cccc(-c3nc(-c4ccc5ccccc5c4)nc4c3sc3ccccc34)c2-c2c1ccc1ccccc21]

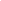

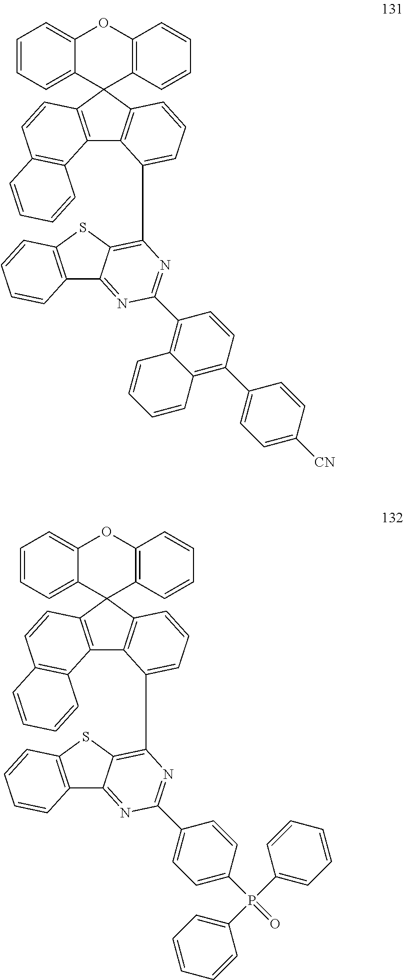 [SMILES: N#Cc1ccc(-c2ccc(-c3nc(-c4cccc5c4-c4c(ccc6ccccc46)C54c5ccccc5Oc5ccccc54)c4sc5ccccc5c4n3)c3ccccc23)cc1 ; O=P(c1ccccc1)(c1ccccc1)c1ccc(-c2nc(-c3cccc4c3-c3c(ccc5ccccc35)C43c4ccccc4Oc4ccccc43)c3sc4ccccc4c3n2)cc1]

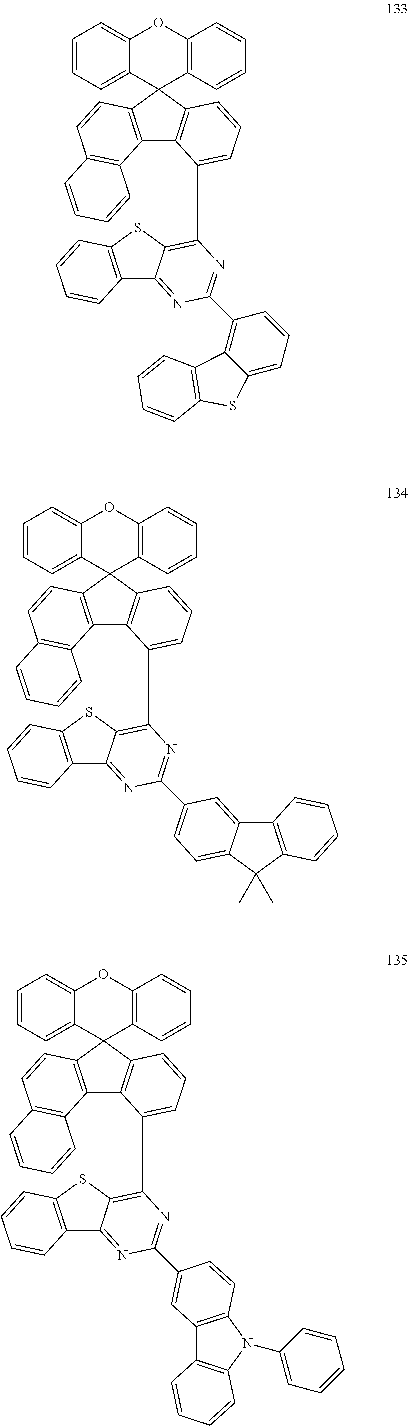 [SMILES: c1ccc2c(c1)Oc1ccccc1C21c2cccc(-c3nc(-c4cccc5sc6ccccc6c45)nc4c3sc3ccccc34)c2-c2c1ccc1ccccc21 ; CC1(C)c2ccccc2-c2cc(-c3nc(-c4cccc5c4-c4c(ccc6ccccc46)C54c5ccccc5Oc5ccccc54)c4sc5ccccc5c4n3)ccc21 ; c1ccc(-n2c3ccccc3c3cc(-c4nc(-c5cccc6c5-c5c(ccc7ccccc57)C65c6ccccc6Oc6ccccc65)c5sc6ccccc6c5n4)ccc32)cc1]

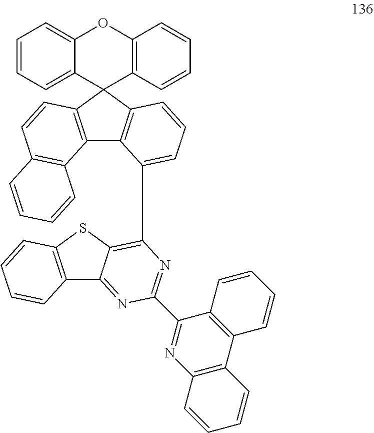 [SMILES: c1ccc2c(c1)Oc1ccccc1C21c2cccc(-c3nc(-c4nc5ccccc5c5ccccc45)nc4c3sc3ccccc34)c2-c2c1ccc1ccccc21]

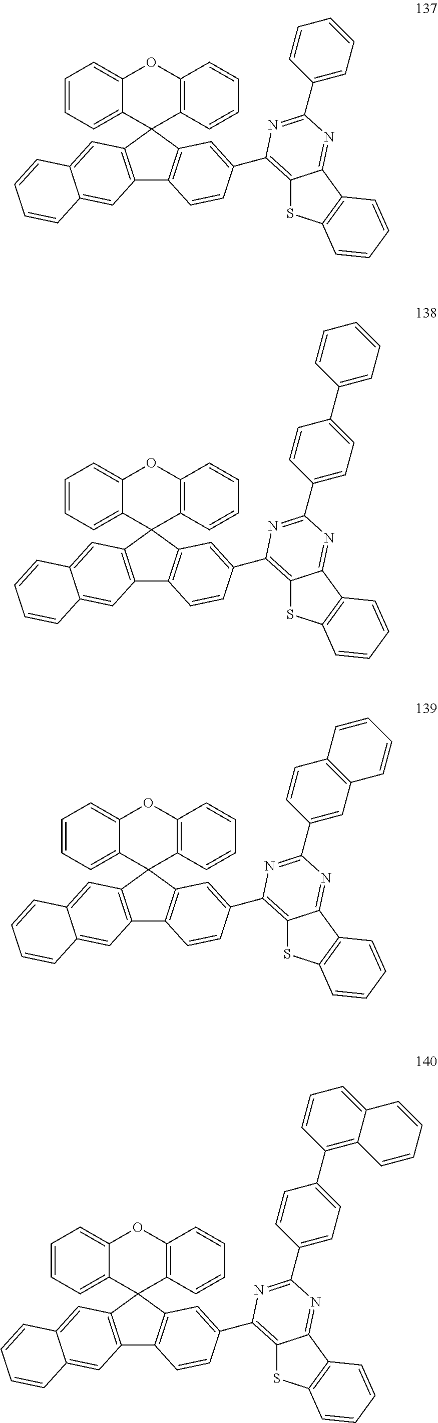 [SMILES: c1ccc(-c2nc(-c3ccc4c(c3)C3(c5ccccc5Oc5ccccc53)c3cc5ccccc5cc3-4)c3sc4ccccc4c3n2)cc1 ; c1ccc(-c2ccc(-c3nc(-c4ccc5c(c4)C4(c6ccccc6Oc6ccccc64)c4cc6ccccc6cc4-5)c4sc5ccccc5c4n3)cc2)cc1 ; c1ccc2c(c1)Oc1ccccc1C21c2cc(-c3nc(-c4ccc5ccccc5c4)nc4c3sc3ccccc34)ccc2-c2cc3ccccc3cc21 ; c1ccc2c(c1)Oc1ccccc1C21c2cc(-c3nc(-c4ccc(-c5cccc6ccccc56)cc4)nc4c3sc3ccccc34)ccc2-c2cc3ccccc3cc21]

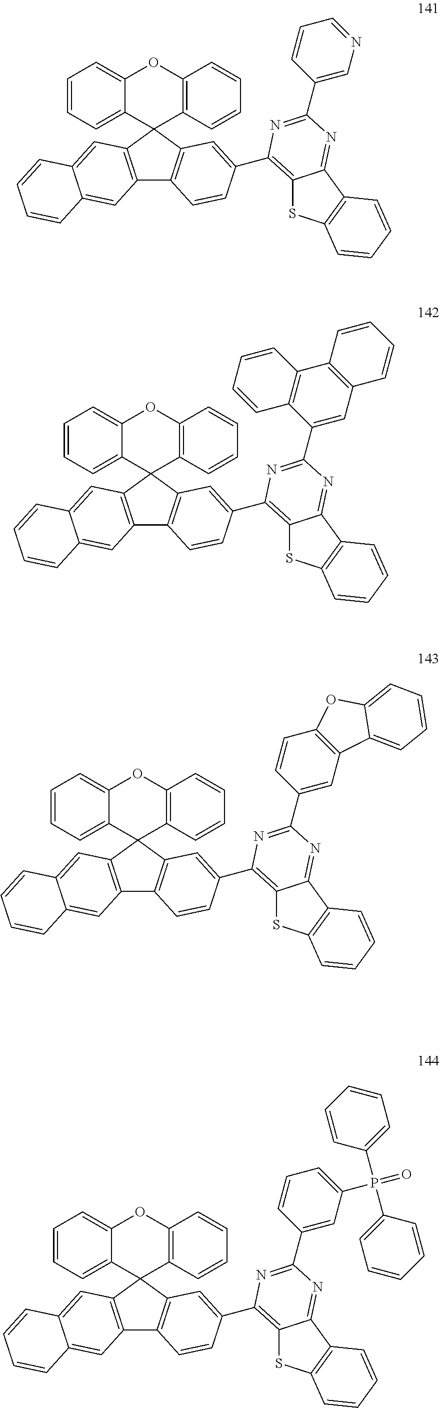 [SMILES: c1cncc(-c2nc(-c3ccc4c(c3)C3(c5ccccc5Oc5ccccc53)c3cc5ccccc5cc3-4)c3sc4ccccc4c3n2)c1 ; c1ccc2c(c1)Oc1ccccc1C21c2cc(-c3nc(-c4cc5ccccc5c5ccccc45)nc4c3sc3ccccc34)ccc2-c2cc3ccccc3cc21 ; c1ccc2c(c1)Oc1ccccc1C21c2cc(-c3nc(-c4ccc5oc6ccccc6c5c4)nc4c3sc3ccccc34)ccc2-c2cc3ccccc3cc21 ; O=P(c1ccccc1)(c1ccccc1)c1cccc(-c2nc(-c3ccc4c(c3)C3(c5ccccc5Oc5ccccc53)c3cc5ccccc5cc3-4)c3sc4ccccc4c3n2)c1]

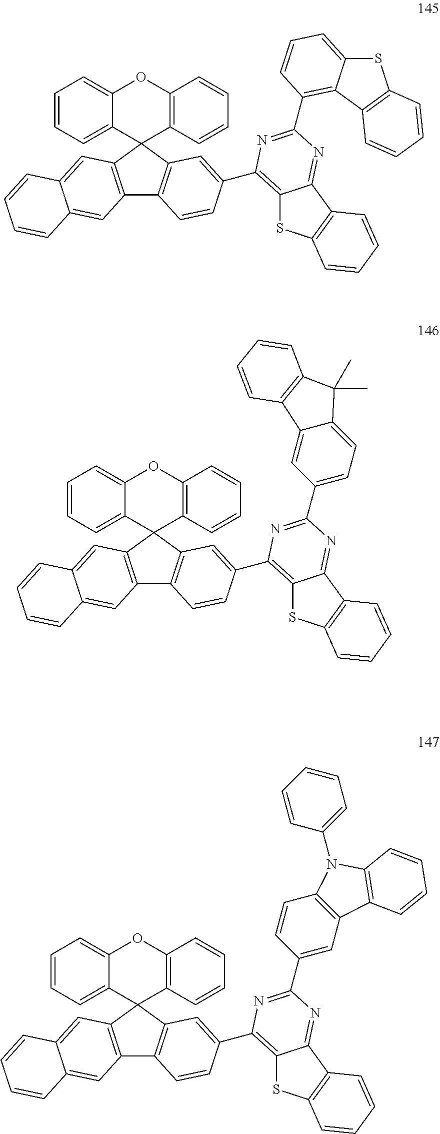 [SMILES: c1ccc2c(c1)Oc1ccccc1C21c2cc(-c3nc(-c4cccc5sc6ccccc6c45)nc4c3sc3ccccc34)ccc2-c2cc3ccccc3cc21 ; CC1(C)c2ccccc2-c2cc(-c3nc(-c4ccc5c(c4)C4(c6ccccc6Oc6ccccc64)c4cc6ccccc6cc4-5)c4sc5ccccc5c4n3)ccc21 ; c1ccc(-n2c3ccccc3c3cc(-c4nc(-c5ccc6c(c5)C5(c7ccccc7Oc7ccccc75)c5cc7ccccc7cc5-6)c5sc6ccccc6c5n4)ccc32)cc1]

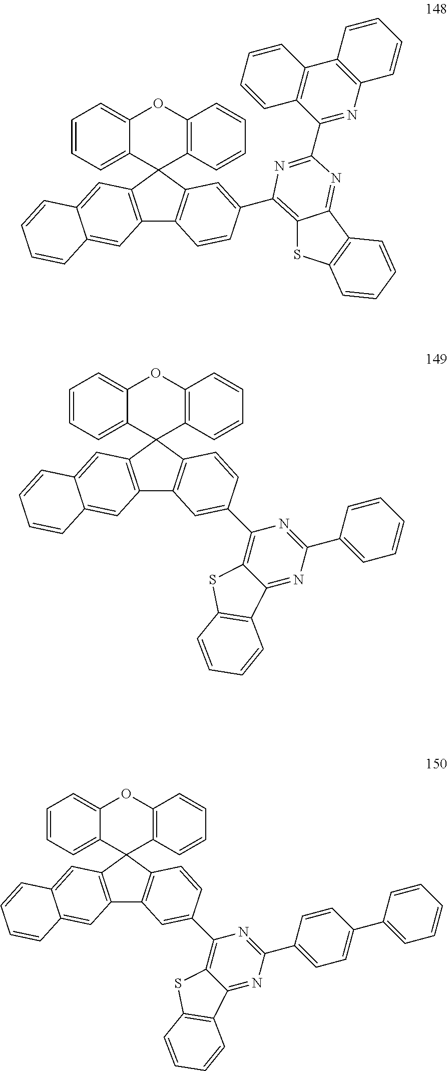 [SMILES: c1ccc2c(c1)Oc1ccccc1C21c2cc(-c3nc(-c4nc5ccccc5c5ccccc45)nc4c3sc3ccccc34)ccc2-c2cc3ccccc3cc21 ; c1ccc(-c2nc(-c3ccc4c(c3)-c3cc5ccccc5cc3C43c4ccccc4Oc4ccccc43)c3sc4ccccc4c3n2)cc1 ; c1ccc(-c2ccc(-c3nc(-c4ccc5c(c4)-c4cc6ccccc6cc4C54c5ccccc5Oc5ccccc54)c4sc5ccccc5c4n3)cc2)cc1]

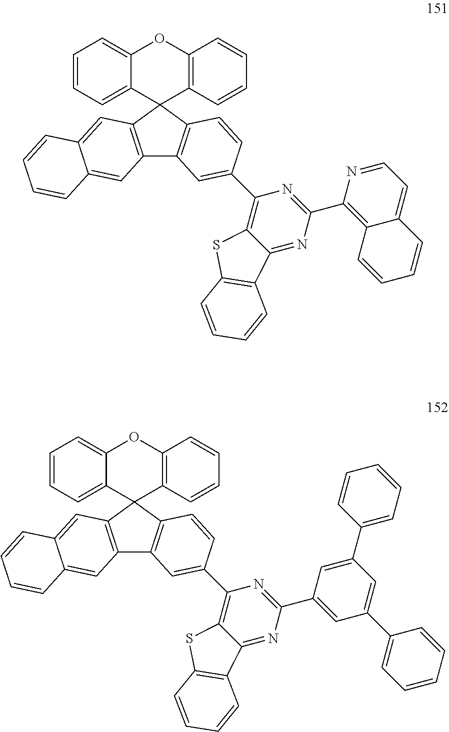 [SMILES: c1ccc2c(c1)Oc1ccccc1C21c2ccc(-c3nc(-c4nccc5ccccc45)nc4c3sc3ccccc34)cc2-c2cc3ccccc3cc21 ; c1ccc(-c2cc(-c3ccccc3)cc(-c3nc(-c4ccc5c(c4)-c4cc6ccccc6cc4C54c5ccccc5Oc5ccccc54)c4sc5ccccc5c4n3)c2)cc1]

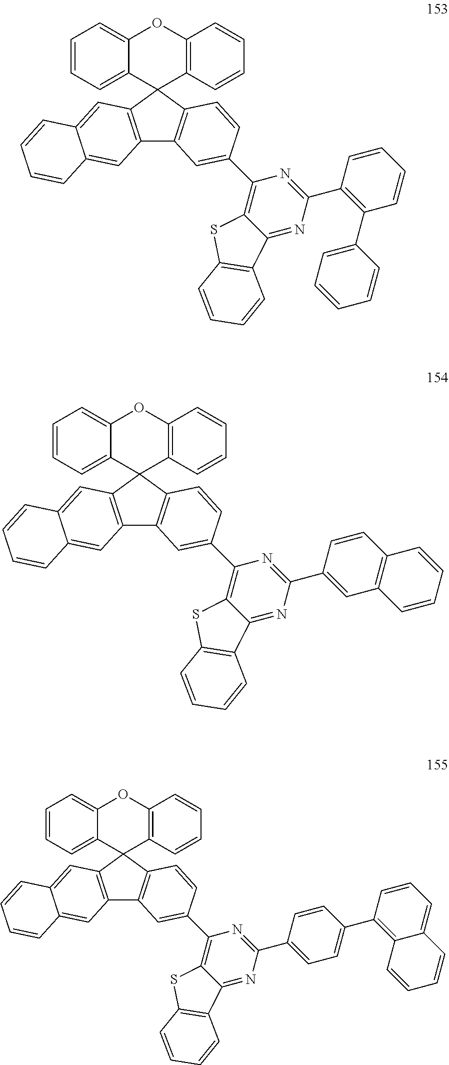 [SMILES: c1ccc(-c2ccccc2-c2nc(-c3ccc4c(c3)-c3cc5ccccc5cc3C43c4ccccc4Oc4ccccc43)c3sc4ccccc4c3n2)cc1 ; c1ccc2c(c1)Oc1ccccc1C21c2ccc(-c3nc(-c4ccc5ccccc5c4)nc4c3sc3ccccc34)cc2-c2cc3ccccc3cc21 ; c1ccc2c(c1)Oc1ccccc1C21c2ccc(-c3nc(-c4ccc(-c5cccc6ccccc56)cc4)nc4c3sc3ccccc34)cc2-c2cc3ccccc3cc21]

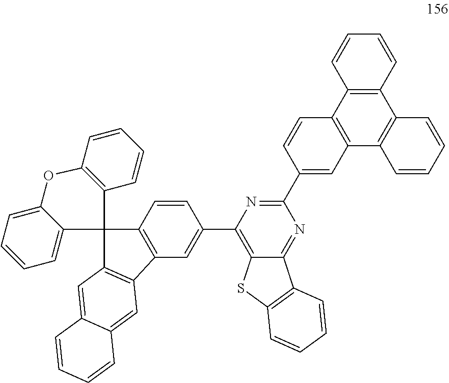 [SMILES: c1ccc2c(c1)Oc1ccccc1C21c2ccc(-c3nc(-c4ccc5c6ccccc6c6ccccc6c5c4)nc4c3sc3ccccc34)cc2-c2cc3ccccc3cc21]

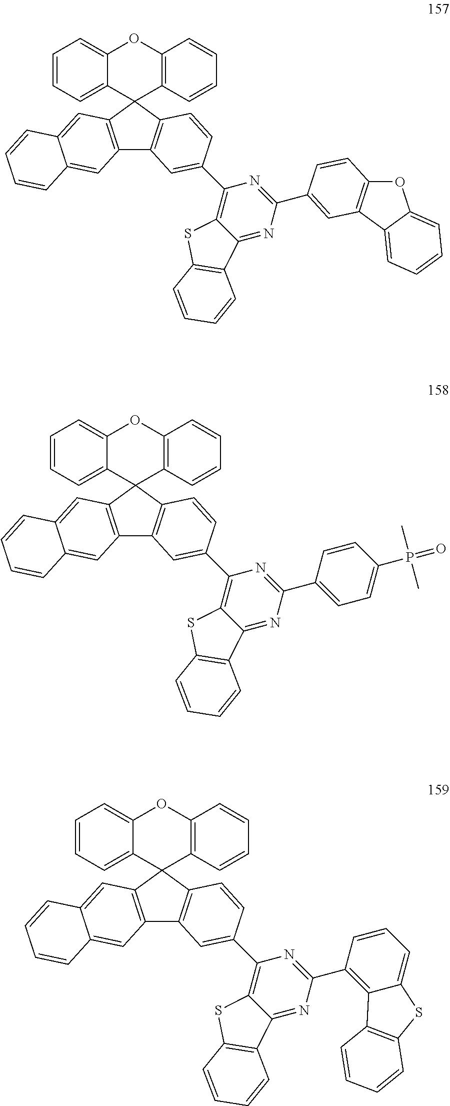 [SMILES: c1ccc2c(c1)Oc1ccccc1C21c2ccc(-c3nc(-c4ccc5oc6ccccc6c5c4)nc4c3sc3ccccc34)cc2-c2cc3ccccc3cc21 ; CP(C)(=O)c1ccc(-c2nc(-c3ccc4c(c3)-c3cc5ccccc5cc3C43c4ccccc4Oc4ccccc43)c3sc4ccccc4c3n2)cc1 ; c1ccc2c(c1)Oc1ccccc1C21c2ccc(-c3nc(-c4cccc5sc6ccccc6c45)nc4c3sc3ccccc34)cc2-c2cc3ccccc3cc21]

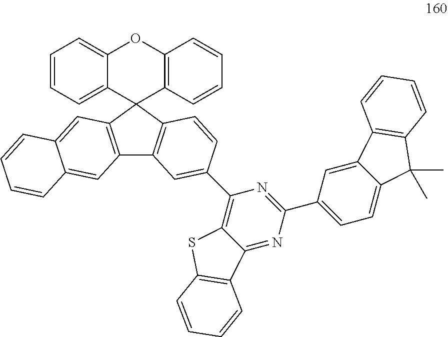 [SMILES: CC1(C)c2ccccc2-c2cc(-c3nc(-c4ccc5c(c4)-c4cc6ccccc6cc4C54c5ccccc5Oc5ccccc54)c4sc5ccccc5c4n3)ccc21]

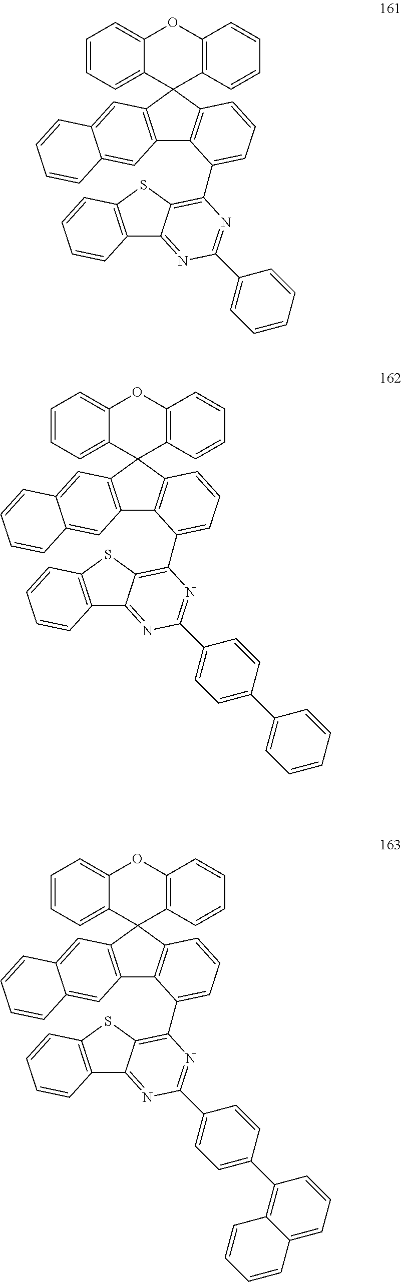 [SMILES: c1ccc(-c2nc(-c3cccc4c3-c3cc5ccccc5cc3C43c4ccccc4Oc4ccccc43)c3sc4ccccc4c3n2)cc1 ; c1ccc(-c2ccc(-c3nc(-c4cccc5c4-c4cc6ccccc6cc4C54c5ccccc5Oc5ccccc54)c4sc5ccccc5c4n3)cc2)cc1 ; c1ccc2c(c1)Oc1ccccc1C21c2cc3ccccc3cc2-c2c(-c3nc(-c4ccc(-c5cccc6ccccc56)cc4)nc4c3sc3ccccc34)cccc21]

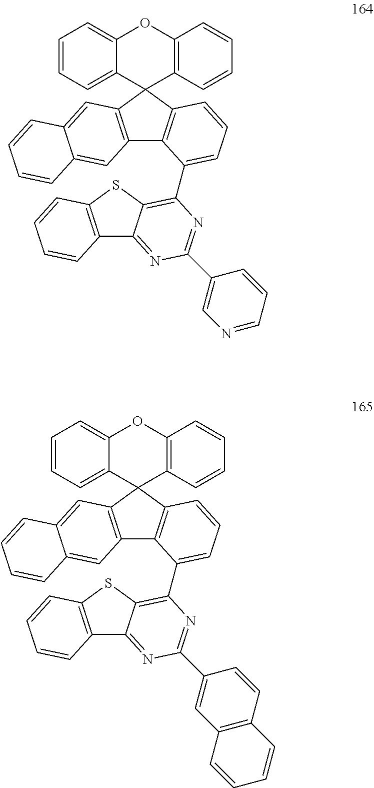 [SMILES: c1cncc(-c2nc(-c3cccc4c3-c3cc5ccccc5cc3C43c4ccccc4Oc4ccccc43)c3sc4ccccc4c3n2)c1 ; c1ccc2c(c1)Oc1ccccc1C21c2cc3ccccc3cc2-c2c(-c3nc(-c4ccc5ccccc5c4)nc4c3sc3ccccc34)cccc21]

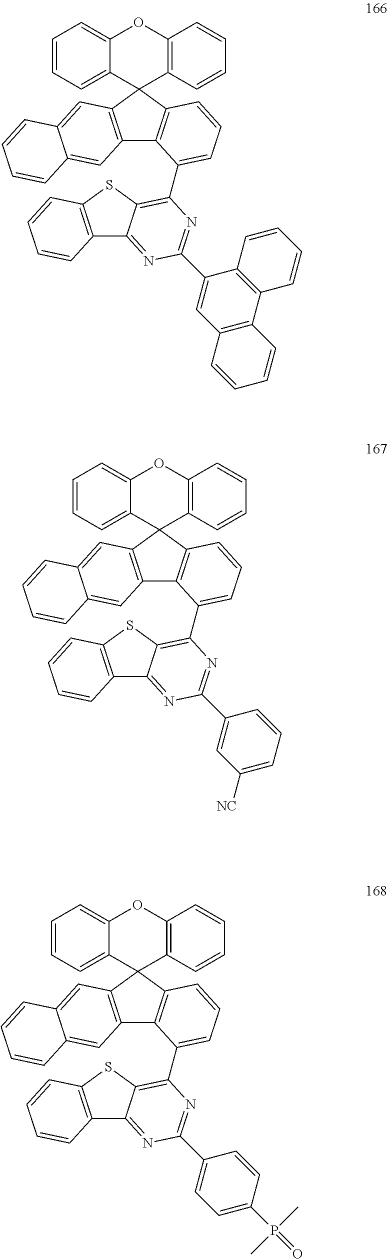 [SMILES: c1ccc2c(c1)Oc1ccccc1C21c2cc3ccccc3cc2-c2c(-c3nc(-c4cc5ccccc5c5ccccc45)nc4c3sc3ccccc34)cccc21 ; N#Cc1cccc(-c2nc(-c3cccc4c3-c3cc5ccccc5cc3C43c4ccccc4Oc4ccccc43)c3sc4ccccc4c3n2)c1 ; CP(C)(=O)c1ccc(-c2nc(-c3cccc4c3-c3cc5ccccc5cc3C43c4ccccc4Oc4ccccc43)c3sc4ccccc4c3n2)cc1]

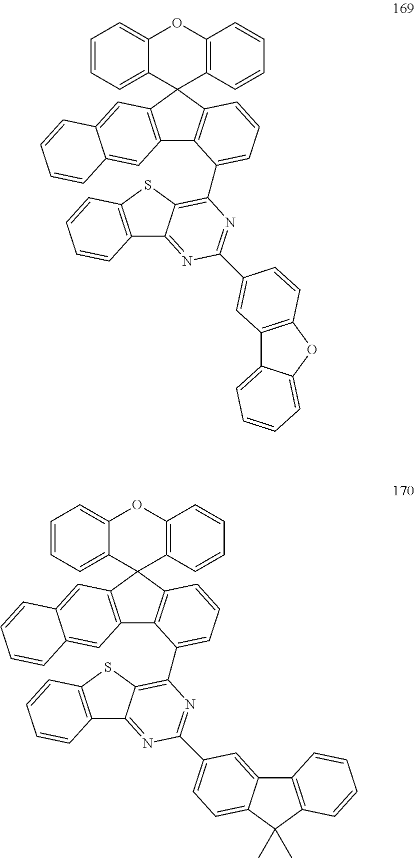 [SMILES: c1ccc2c(c1)Oc1ccccc1C21c2cc3ccccc3cc2-c2c(-c3nc(-c4ccc5oc6ccccc6c5c4)nc4c3sc3ccccc34)cccc21 ; CC1(C)c2ccccc2-c2cc(-c3nc(-c4cccc5c4-c4cc6ccccc6cc4C54c5ccccc5Oc5ccccc54)c4sc5ccccc5c4n3)ccc21]

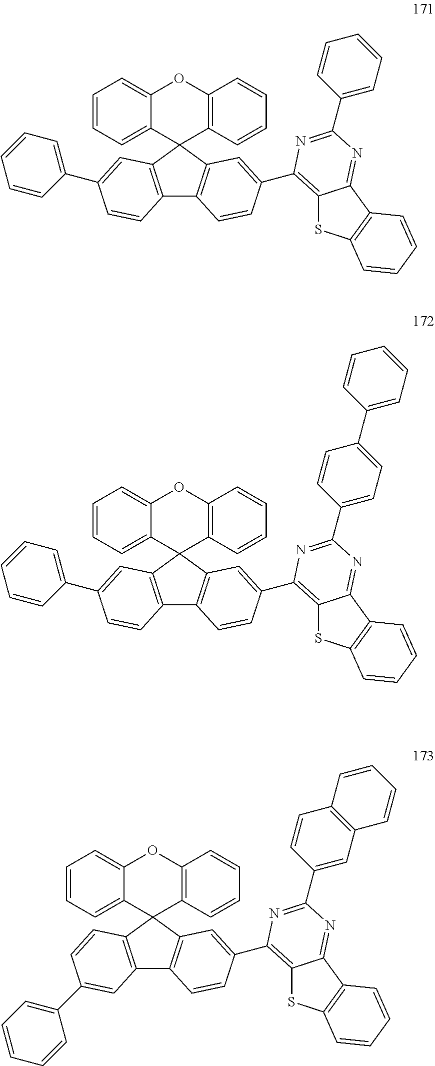 [SMILES: c1ccc(-c2ccc3c(c2)C2(c4ccccc4Oc4ccccc42)c2cc(-c4nc(-c5ccccc5)nc5c4sc4ccccc45)ccc2-3)cc1 ; c1ccc(-c2ccc(-c3nc(-c4ccc5c(c4)C4(c6ccccc6Oc6ccccc64)c4cc(-c6ccccc6)ccc4-5)c4sc5ccccc5c4n3)cc2)cc1 ; c1ccc(-c2ccc3c(c2)-c2ccc(-c4nc(-c5ccc6ccccc6c5)nc5c4sc4ccccc45)cc2C32c3ccccc3Oc3ccccc32)cc1]

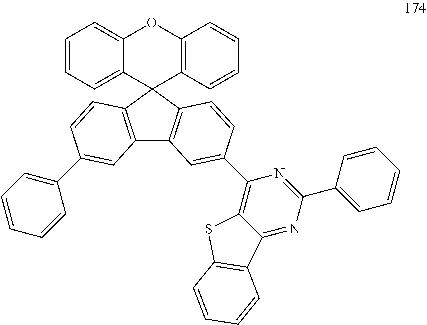 [SMILES: c1ccc(-c2ccc3c(c2)-c2cc(-c4nc(-c5ccccc5)nc5c4sc4ccccc45)ccc2C32c3ccccc3Oc3ccccc32)cc1]

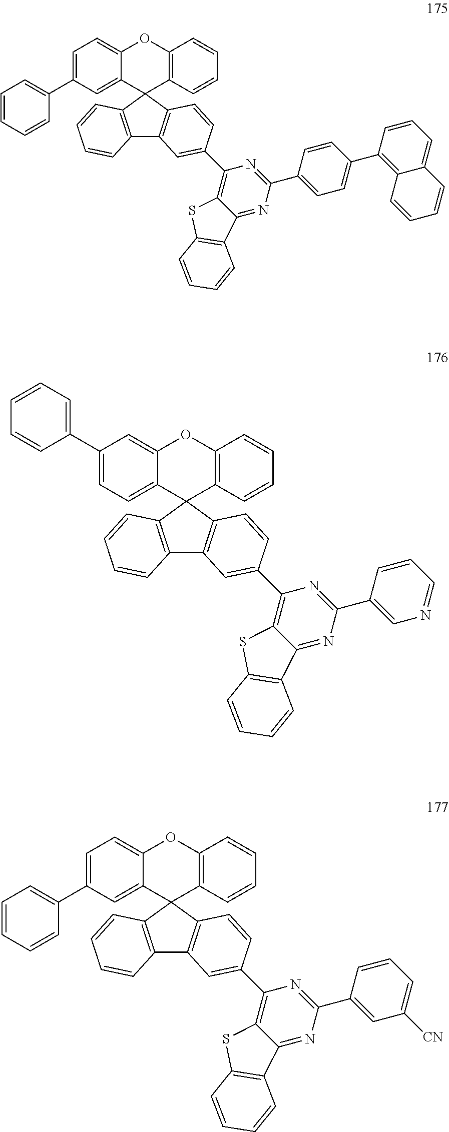 [SMILES: c1ccc(-c2ccc3c(c2)C2(c4ccccc4O3)c3ccccc3-c3cc(-c4nc(-c5ccc(-c6cccc7ccccc67)cc5)nc5c4sc4ccccc45)ccc32)cc1 ; c1ccc(-c2ccc3c(c2)Oc2ccccc2C32c3ccccc3-c3cc(-c4nc(-c5cccnc5)nc5c4sc4ccccc45)ccc32)cc1 ; N#Cc1cccc(-c2nc(-c3ccc4c(c3)-c3ccccc3C43c4ccccc4Oc4ccc(-c5ccccc5)cc43)c3sc4ccccc4c3n2)c1]

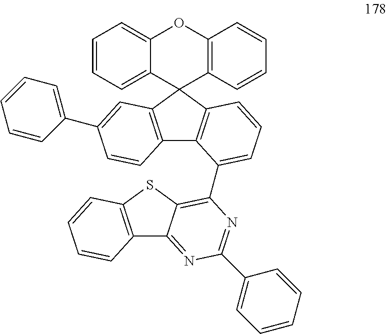 [SMILES: c1ccc(-c2ccc3c(c2)C2(c4ccccc4Oc4ccccc42)c2cccc(-c4nc(-c5ccccc5)nc5c4sc4ccccc45)c2-3)cc1]

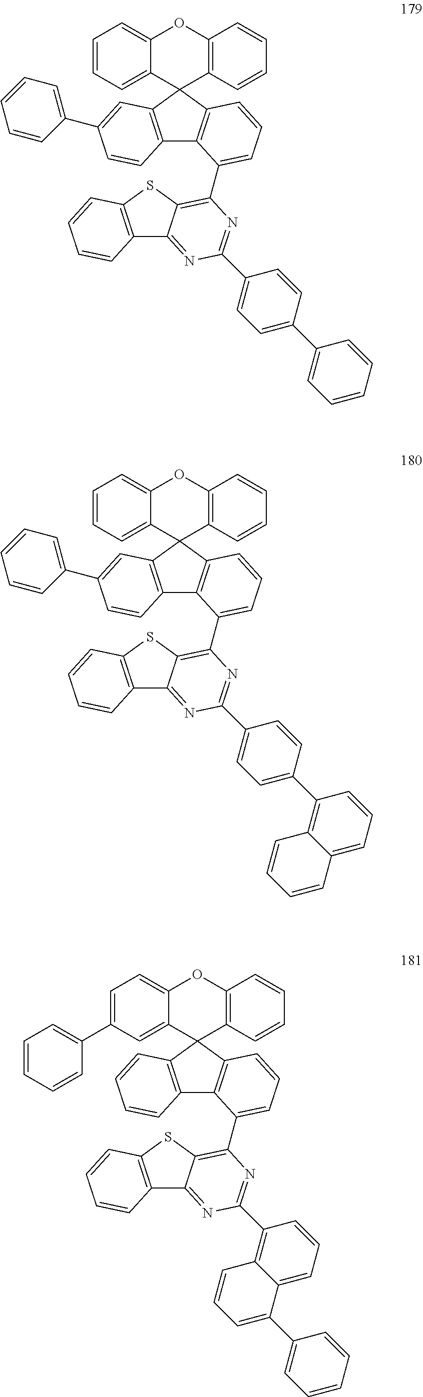 [SMILES: c1ccc(-c2ccc(-c3nc(-c4cccc5c4-c4ccc(-c6ccccc6)cc4C54c5ccccc5Oc5ccccc54)c4sc5ccccc5c4n3)cc2)cc1 ; c1ccc(-c2ccc3c(c2)C2(c4ccccc4Oc4ccccc42)c2cccc(-c4nc(-c5ccc(-c6cccc7ccccc67)cc5)nc5c4sc4ccccc45)c2-3)cc1 ; c1ccc(-c2ccc3c(c2)C2(c4ccccc4O3)c3ccccc3-c3c(-c4nc(-c5cccc6c(-c7ccccc7)cccc56)nc5c4sc4ccccc45)cccc32)cc1]

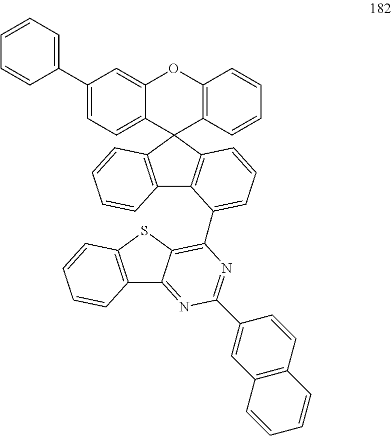 [SMILES: c1ccc(-c2ccc3c(c2)Oc2ccccc2C32c3ccccc3-c3c(-c4nc(-c5ccc6ccccc6c5)nc5c4sc4ccccc45)cccc32)cc1]

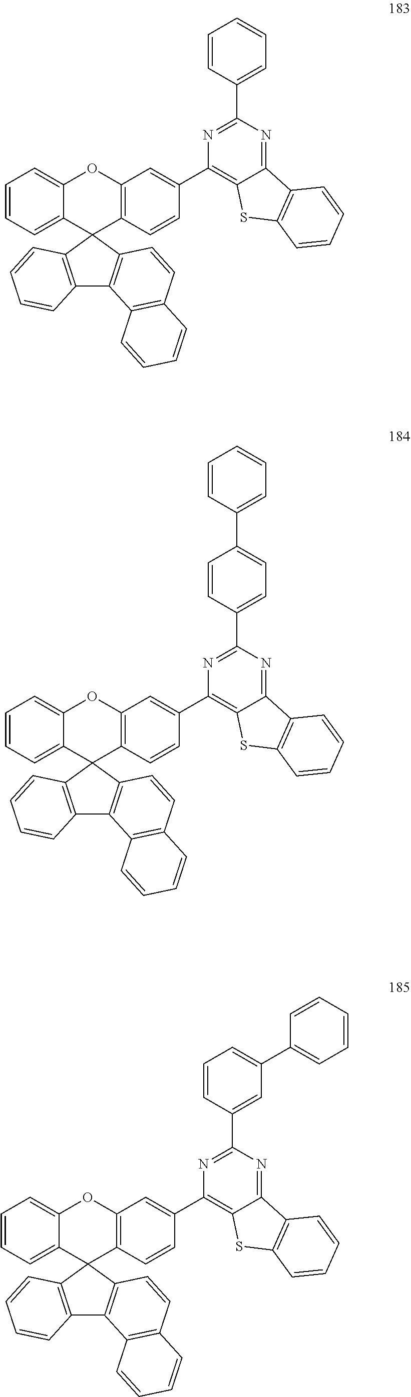 [SMILES: c1ccc(-c2nc(-c3ccc4c(c3)Oc3ccccc3C43c4ccccc4-c4c3ccc3ccccc43)c3sc4ccccc4c3n2)cc1 ; c1ccc(-c2ccc(-c3nc(-c4ccc5c(c4)Oc4ccccc4C54c5ccccc5-c5c4ccc4ccccc54)c4sc5ccccc5c4n3)cc2)cc1 ; c1ccc(-c2cccc(-c3nc(-c4ccc5c(c4)Oc4ccccc4C54c5ccccc5-c5c4ccc4ccccc54)c4sc5ccccc5c4n3)c2)cc1]

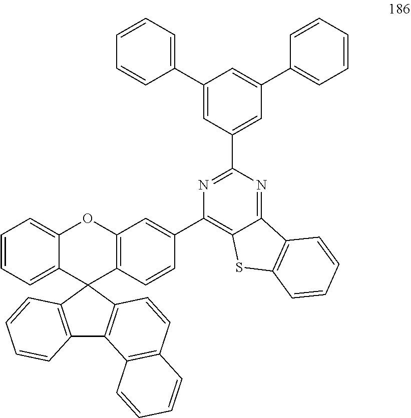 [SMILES: c1ccc(-c2cc(-c3ccccc3)cc(-c3nc(-c4ccc5c(c4)Oc4ccccc4C54c5ccccc5-c5c4ccc4ccccc54)c4sc5ccccc5c4n3)c2)cc1]

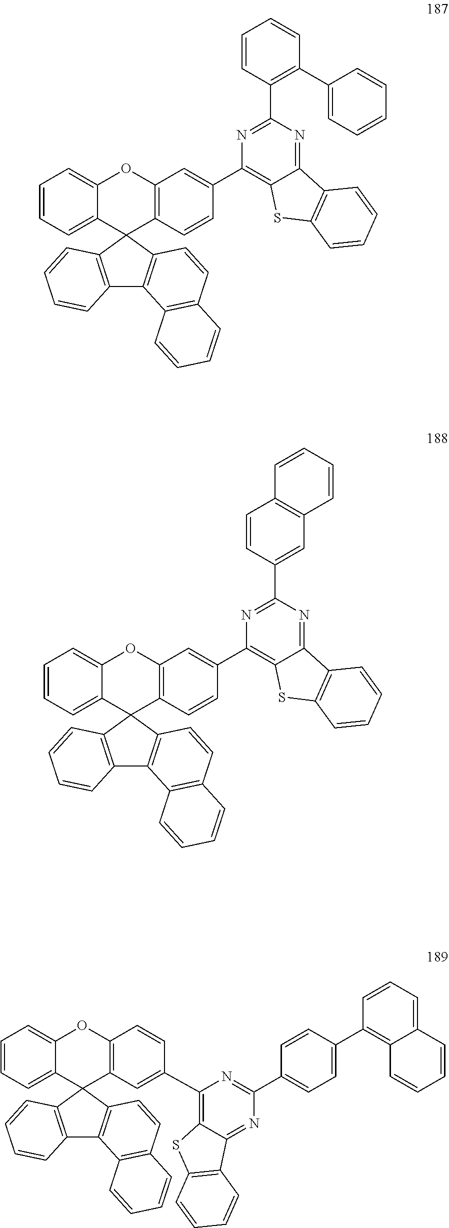 [SMILES: c1ccc(-c2ccccc2-c2nc(-c3ccc4c(c3)Oc3ccccc3C43c4ccccc4-c4c3ccc3ccccc43)c3sc4ccccc4c3n2)cc1 ; c1ccc2c(c1)Oc1cc(-c3nc(-c4ccc5ccccc5c4)nc4c3sc3ccccc34)ccc1C21c2ccccc2-c2c1ccc1ccccc21 ; c1ccc2c(c1)Oc1ccc(-c3nc(-c4ccc(-c5cccc6ccccc56)cc4)nc4c3sc3ccccc34)cc1C21c2ccccc2-c2c1ccc1ccccc21]

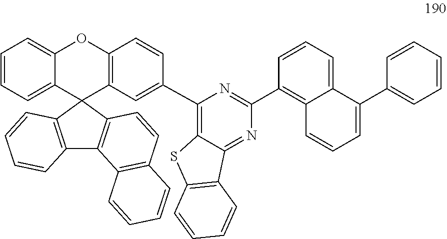 [SMILES: c1ccc(-c2cccc3c(-c4nc(-c5ccc6c(c5)C5(c7ccccc7O6)c6ccccc6-c6c5ccc5ccccc65)c5sc6ccccc6c5n4)cccc23)cc1]

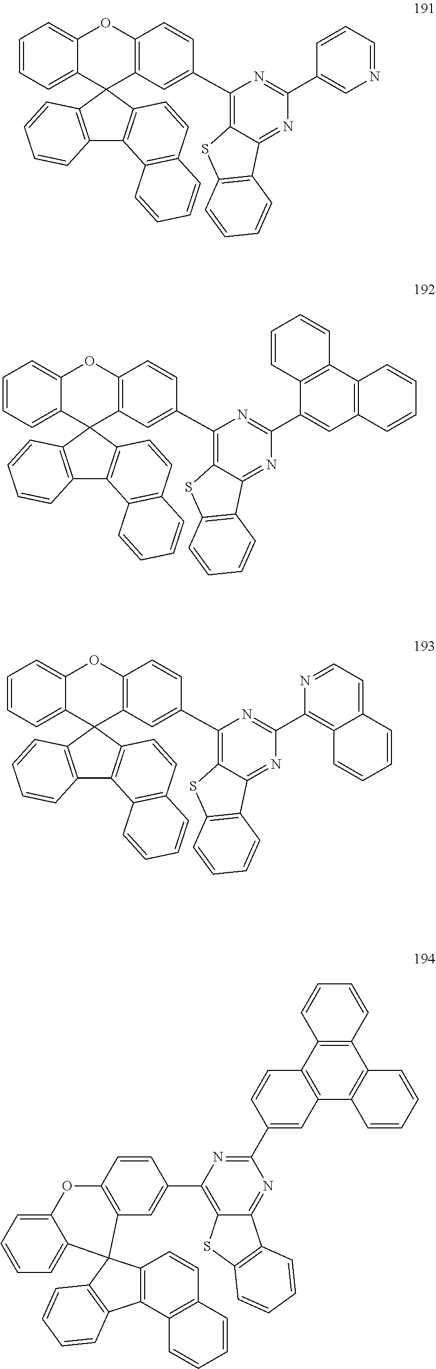 [SMILES: c1cncc(-c2nc(-c3ccc4c(c3)C3(c5ccccc5O4)c4ccccc4-c4c3ccc3ccccc43)c3sc4ccccc4c3n2)c1 ; c1ccc2c(c1)Oc1ccc(-c3nc(-c4cc5ccccc5c5ccccc45)nc4c3sc3ccccc34)cc1C21c2ccccc2-c2c1ccc1ccccc21 ; c1ccc2c(c1)Oc1ccc(-c3nc(-c4nccc5ccccc45)nc4c3sc3ccccc34)cc1C21c2ccccc2-c2c1ccc1ccccc21 ; c1ccc2c(c1)Oc1ccc(-c3nc(-c4ccc5c6ccccc6c6ccccc6c5c4)nc4c3sc3ccccc34)cc1C21c2ccccc2-c2c1ccc1ccccc21]

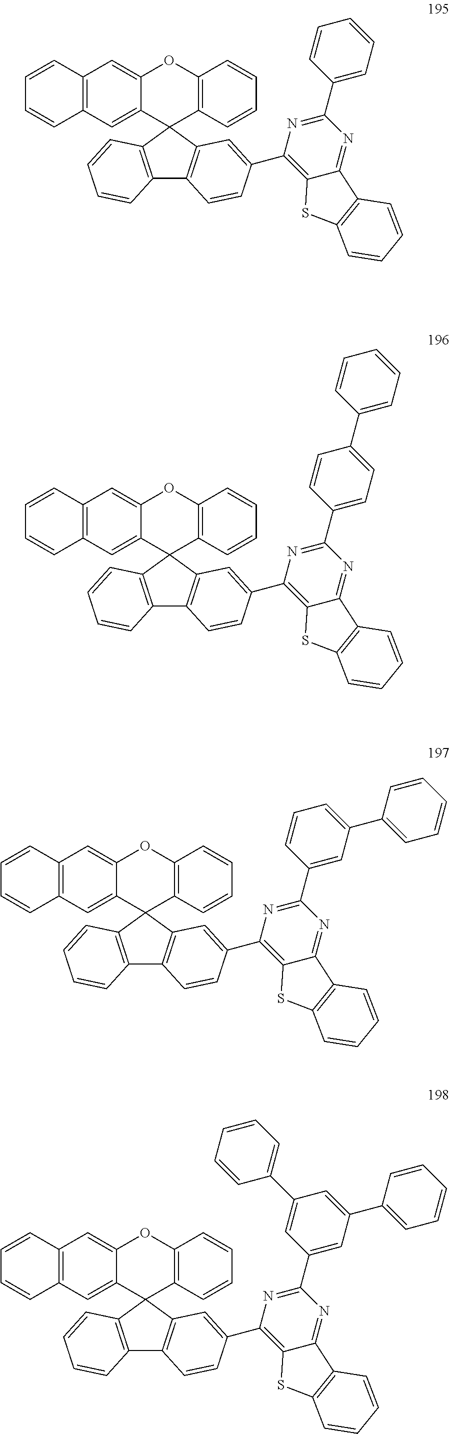 [SMILES: c1ccc(-c2nc(-c3ccc4c(c3)C3(c5ccccc5Oc5cc6ccccc6cc53)c3ccccc3-4)c3sc4ccccc4c3n2)cc1 ; c1ccc(-c2ccc(-c3nc(-c4ccc5c(c4)C4(c6ccccc6Oc6cc7ccccc7cc64)c4ccccc4-5)c4sc5ccccc5c4n3)cc2)cc1 ; c1ccc(-c2cccc(-c3nc(-c4ccc5c(c4)C4(c6ccccc6Oc6cc7ccccc7cc64)c4ccccc4-5)c4sc5ccccc5c4n3)c2)cc1 ; c1ccc(-c2cc(-c3ccccc3)cc(-c3nc(-c4ccc5c(c4)C4(c6ccccc6Oc6cc7ccccc7cc64)c4ccccc4-5)c4sc5ccccc5c4n3)c2)cc1]

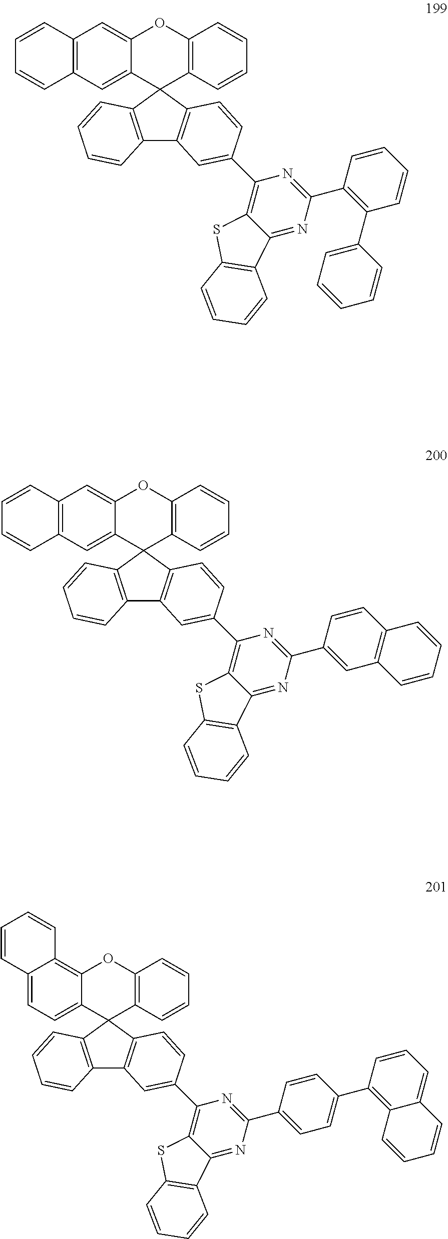 [SMILES: c1ccc(-c2ccccc2-c2nc(-c3ccc4c(c3)-c3ccccc3C43c4ccccc4Oc4cc5ccccc5cc43)c3sc4ccccc4c3n2)cc1 ; c1ccc2c(c1)Oc1cc3ccccc3cc1C21c2ccccc2-c2cc(-c3nc(-c4ccc5ccccc5c4)nc4c3sc3ccccc34)ccc21 ; c1ccc2c(c1)Oc1c(ccc3ccccc13)C21c2ccccc2-c2cc(-c3nc(-c4ccc(-c5cccc6ccccc56)cc4)nc4c3sc3ccccc34)ccc21]

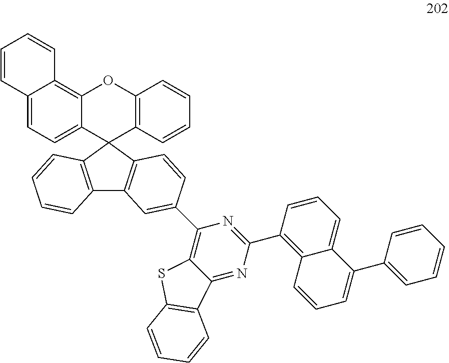 [SMILES: c1ccc(-c2cccc3c(-c4nc(-c5ccc6c(c5)-c5ccccc5C65c6ccccc6Oc6c5ccc5ccccc65)c5sc6ccccc6c5n4)cccc23)cc1]

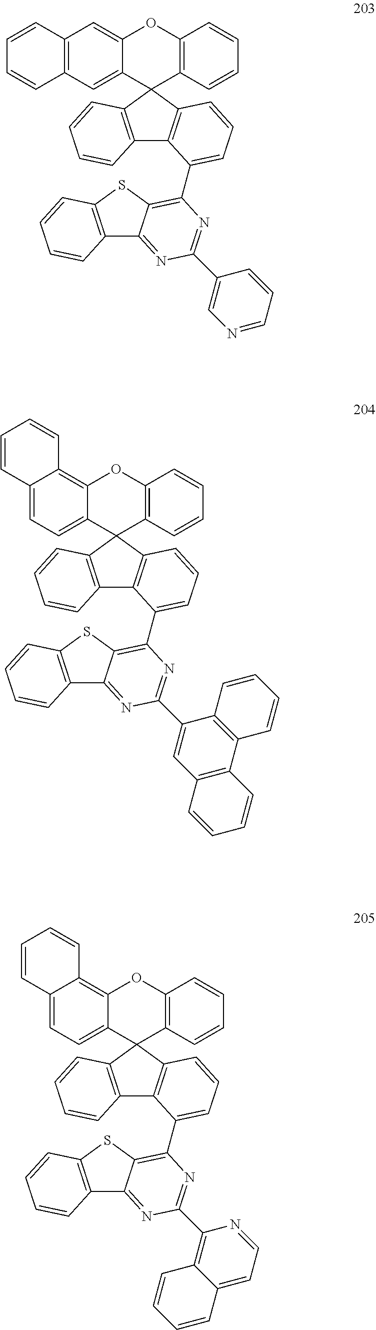 [SMILES: c1cncc(-c2nc(-c3cccc4c3-c3ccccc3C43c4ccccc4Oc4cc5ccccc5cc43)c3sc4ccccc4c3n2)c1 ; c1ccc2c(c1)Oc1c(ccc3ccccc13)C21c2ccccc2-c2c(-c3nc(-c4cc5ccccc5c5ccccc45)nc4c3sc3ccccc34)cccc21 ; c1ccc2c(c1)Oc1c(ccc3ccccc13)C21c2ccccc2-c2c(-c3nc(-c4nccc5ccccc45)nc4c3sc3ccccc34)cccc21]

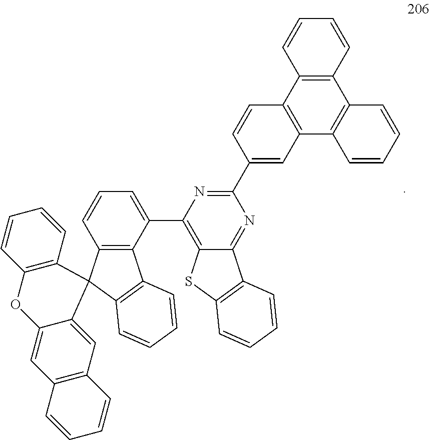 [SMILES: c1ccc2c(c1)Oc1cc3ccccc3cc1C21c2ccccc2-c2c(-c3nc(-c4ccc5c6ccccc6c6ccccc6c5c4)nc4c3sc3ccccc34)cccc21]

10. The compound of claim 1, wherein the compound represented by Chemical Formula 1 is selected from compounds represented by the following Chemical Formulas:

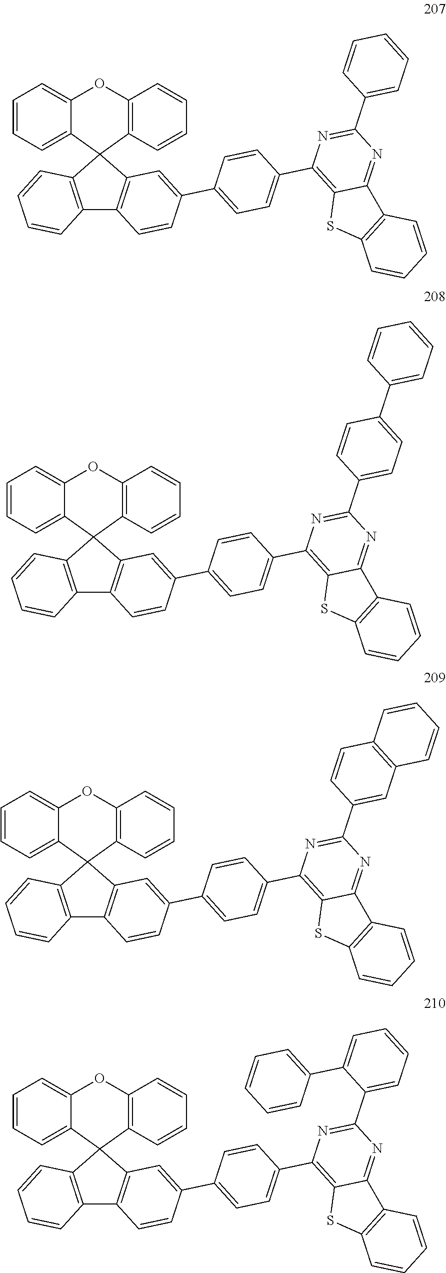

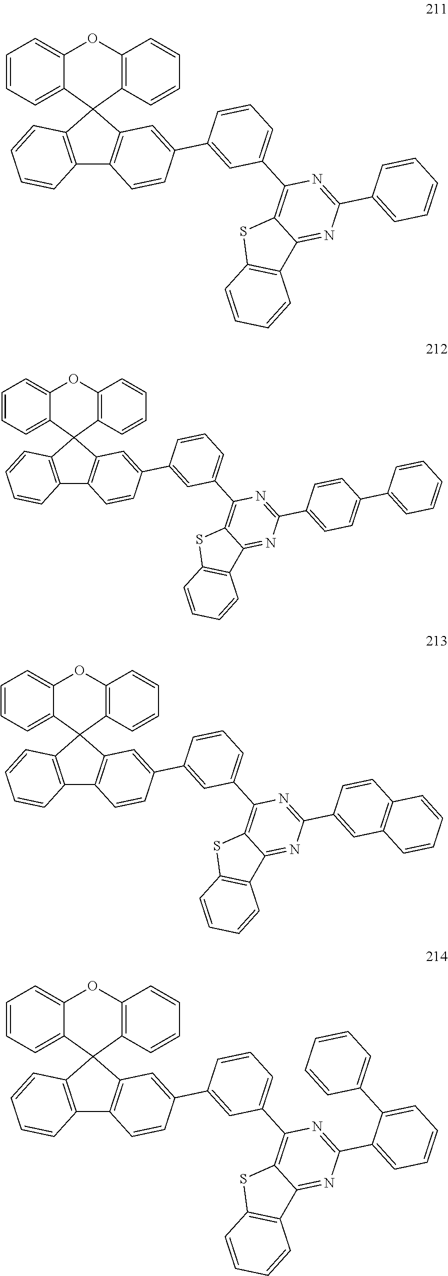 [SMILES: c1ccc(-c2nc(-c3cccc(-c4ccc5c(c4)C4(c6ccccc6Oc6ccccc64)c4ccccc4-5)c3)c3sc4ccccc4c3n2)cc1 ; c1ccc(-c2ccc(-c3nc(-c4cccc(-c5ccc6c(c5)C5(c7ccccc7Oc7ccccc75)c5ccccc5-6)c4)c4sc5ccccc5c4n3)cc2)cc1 ; c1cc(-c2ccc3c(c2)C2(c4ccccc4Oc4ccccc42)c2ccccc2-3)cc(-c2nc(-c3ccc4ccccc4c3)nc3c2sc2ccccc23)c1 ; c1ccc(-c2ccccc2-c2nc(-c3cccc(-c4ccc5c(c4)C4(c6ccccc6Oc6ccccc64)c4ccccc4-5)c3)c3sc4ccccc4c3n2)cc1]

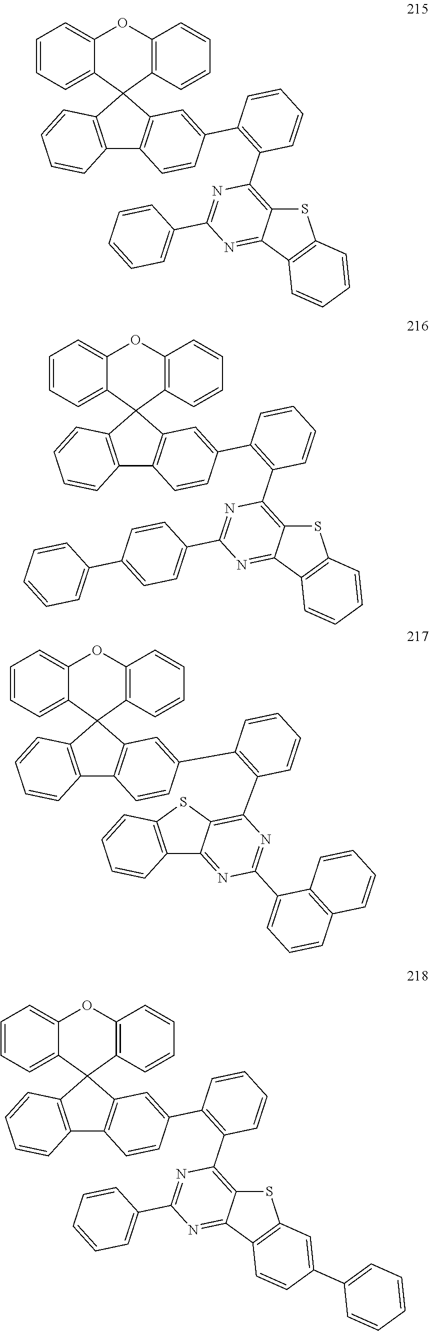

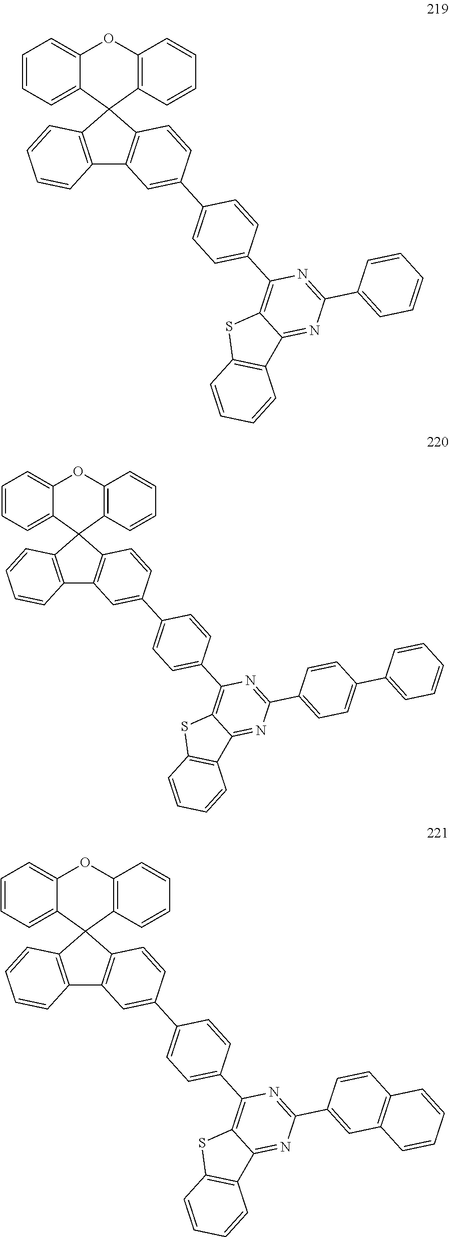 [SMILES: c1ccc(-c2nc(-c3ccc(-c4ccc5c(c4)-c4ccccc4C54c5ccccc5Oc5ccccc54)cc3)c3sc4ccccc4c3n2)cc1 ; c1ccc(-c2ccc(-c3nc(-c4ccc(-c5ccc6c(c5)-c5ccccc5C65c6ccccc6Oc6ccccc65)cc4)c4sc5ccccc5c4n3)cc2)cc1 ; c1ccc2c(c1)Oc1ccccc1C21c2ccccc2-c2cc(-c3ccc(-c4nc(-c5ccc6ccccc6c5)nc5c4sc4ccccc45)cc3)ccc21]

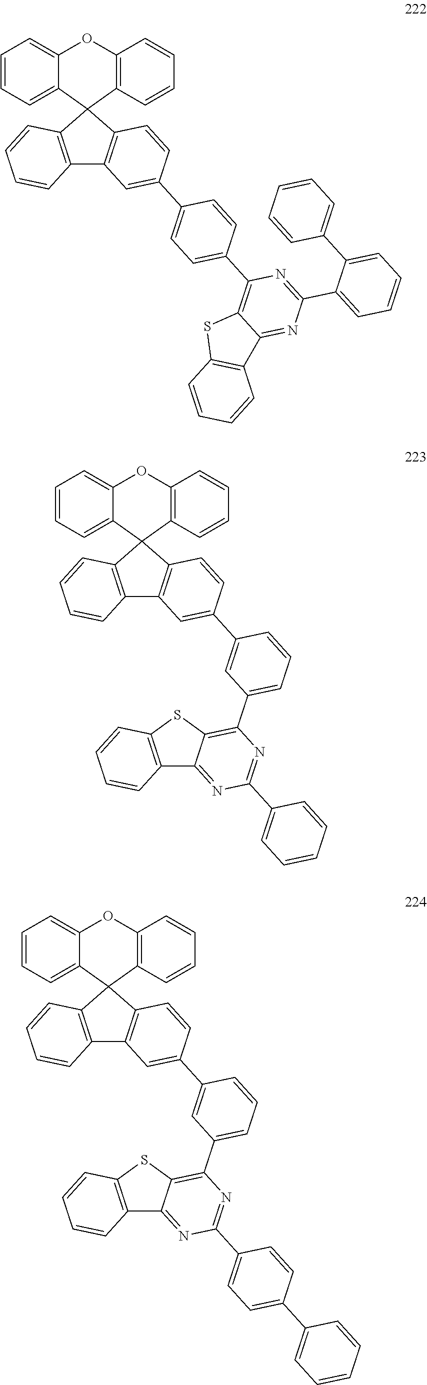 [SMILES: c1ccc(-c2ccccc2-c2nc(-c3ccc(-c4ccc5c(c4)-c4ccccc4C54c5ccccc5Oc5ccccc54)cc3)c3sc4ccccc4c3n2)cc1 ; c1ccc(-c2nc(-c3cccc(-c4ccc5c(c4)-c4ccccc4C54c5ccccc5Oc5ccccc54)c3)c3sc4ccccc4c3n2)cc1 ; c1ccc(-c2ccc(-c3nc(-c4cccc(-c5ccc6c(c5)-c5ccccc5C65c6ccccc6Oc6ccccc65)c4)c4sc5ccccc5c4n3)cc2)cc1]

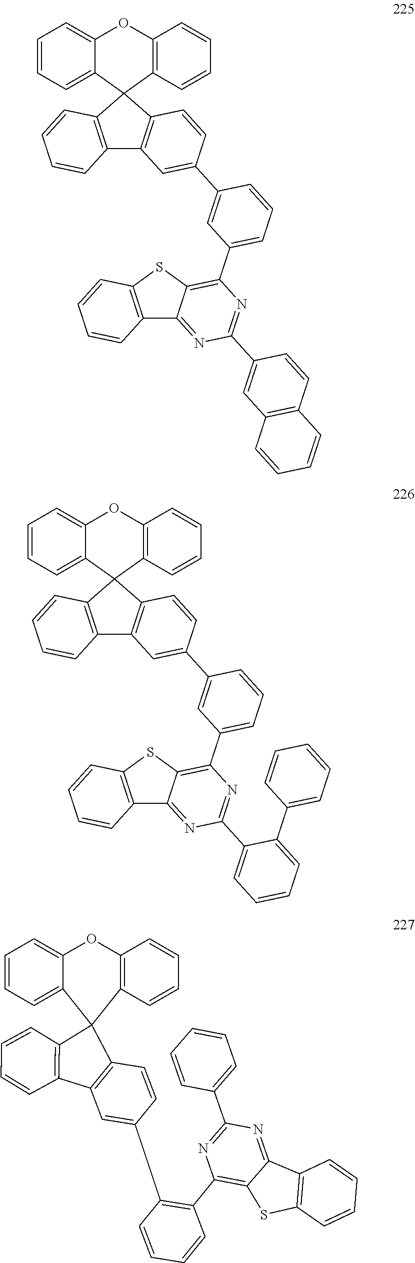 [SMILES: c1cc(-c2ccc3c(c2)-c2ccccc2C32c3ccccc3Oc3ccccc32)cc(-c2nc(-c3ccc4ccccc4c3)nc3c2sc2ccccc23)c1 ; c1ccc(-c2ccccc2-c2nc(-c3cccc(-c4ccc5c(c4)-c4ccccc4C54c5ccccc5Oc5ccccc54)c3)c3sc4ccccc4c3n2)cc1 ; c1ccc(-c2nc(-c3ccccc3-c3ccc4c(c3)-c3ccccc3C43c4ccccc4Oc4ccccc43)c3sc4ccccc4c3n2)cc1]

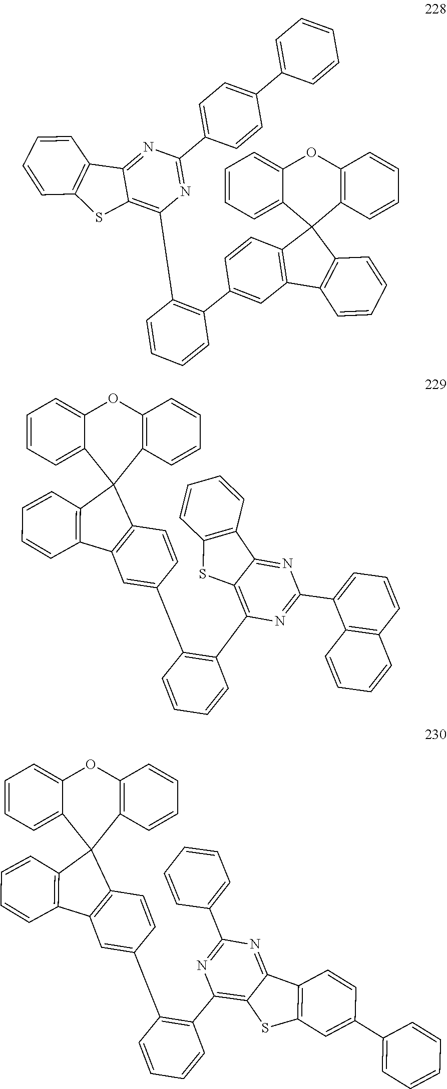

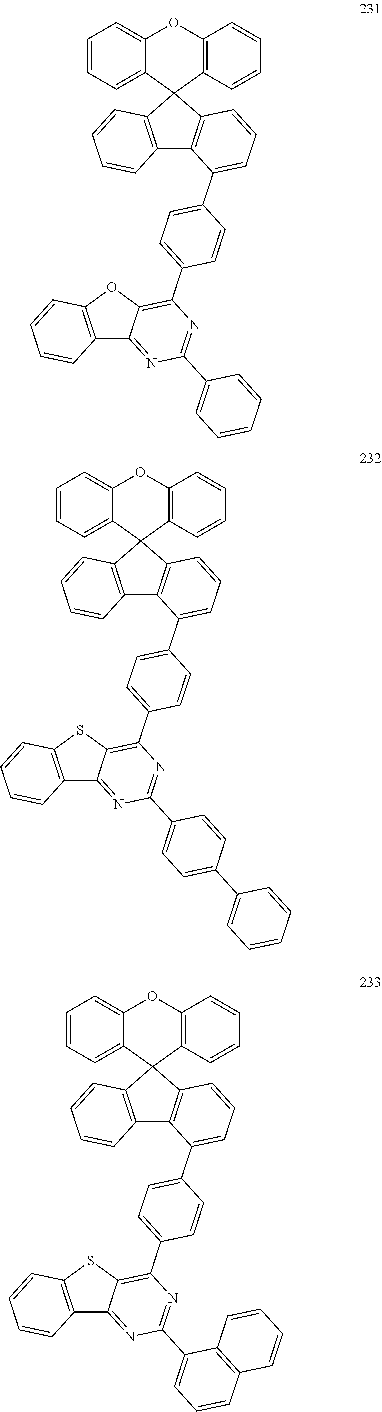

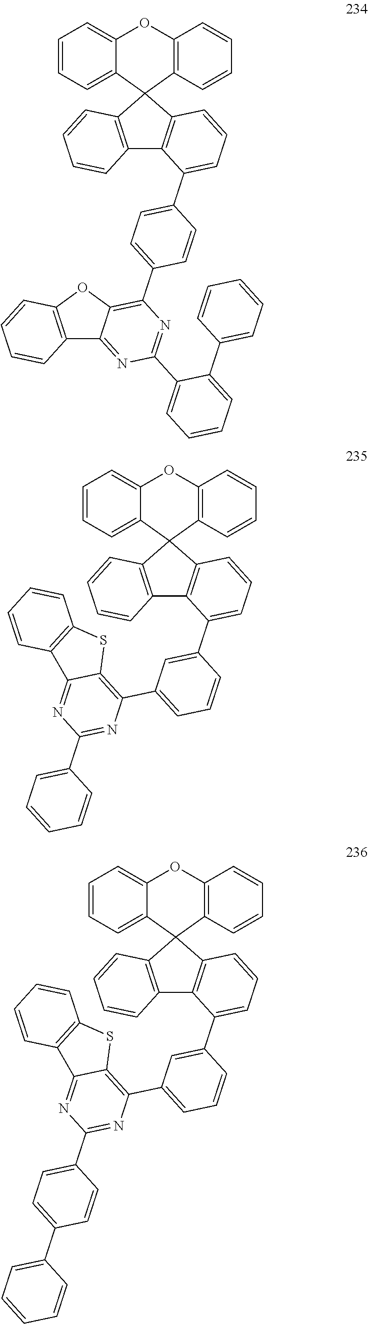

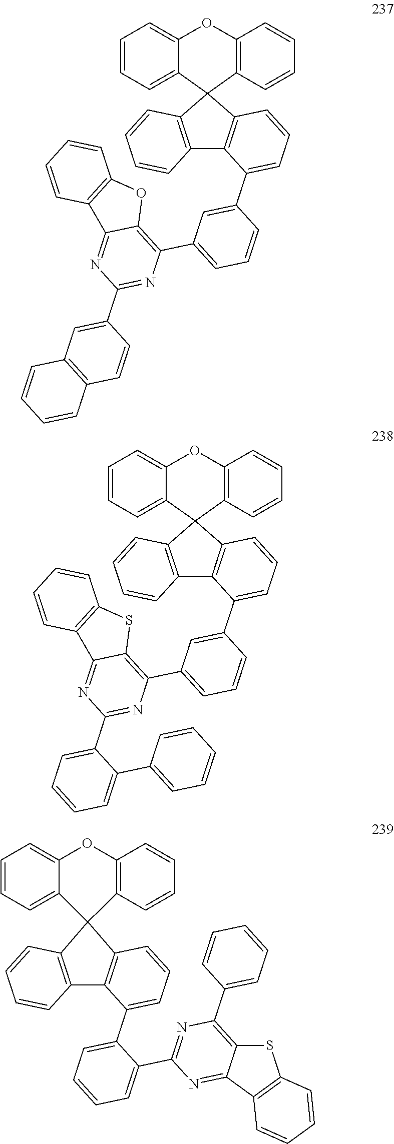

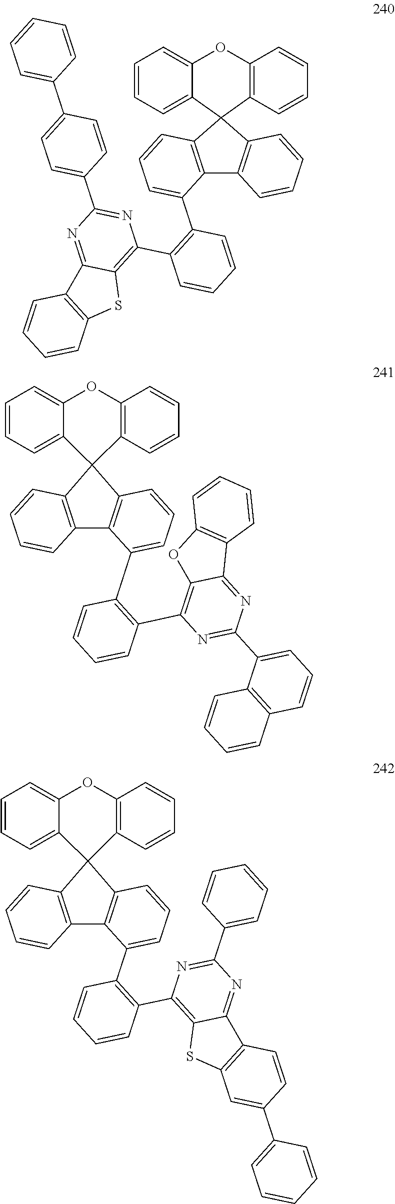

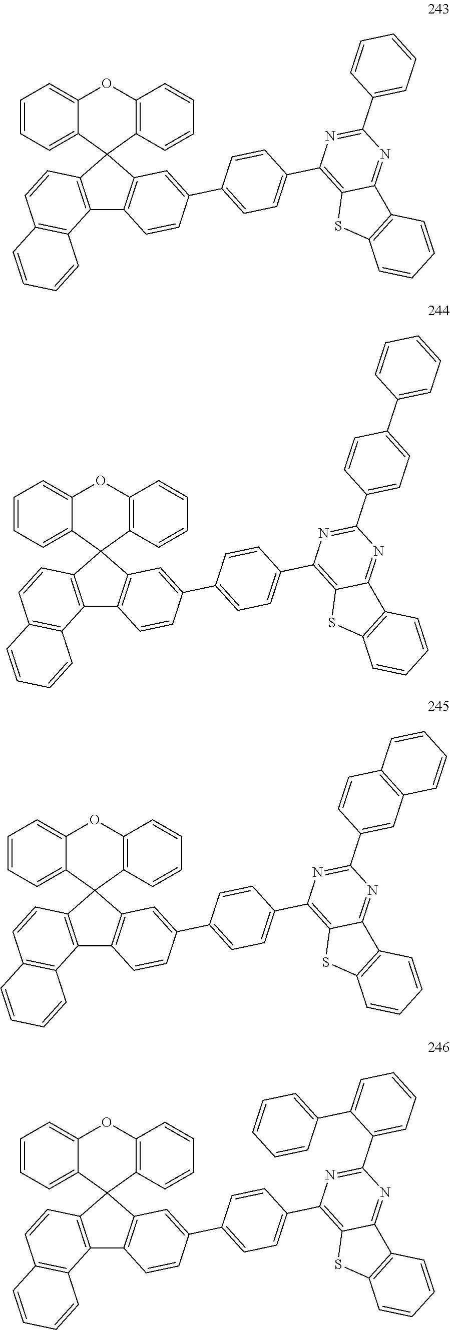 [SMILES: c1ccc(-c2nc(-c3ccc(-c4ccc5c(c4)C4(c6ccccc6Oc6ccccc64)c4ccc6ccccc6c4-5)cc3)c3sc4ccccc4c3n2)cc1 ; c1ccc(-c2ccc(-c3nc(-c4ccc(-c5ccc6c(c5)C5(c7ccccc7Oc7ccccc75)c5ccc7ccccc7c5-6)cc4)c4sc5ccccc5c4n3)cc2)cc1 ; c1ccc2c(c1)Oc1ccccc1C21c2cc(-c3ccc(-c4nc(-c5ccc6ccccc6c5)nc5c4sc4ccccc45)cc3)ccc2-c2c1ccc1ccccc21 ; c1ccc(-c2ccccc2-c2nc(-c3ccc(-c4ccc5c(c4)C4(c6ccccc6Oc6ccccc64)c4ccc6ccccc6c4-5)cc3)c3sc4ccccc4c3n2)cc1]

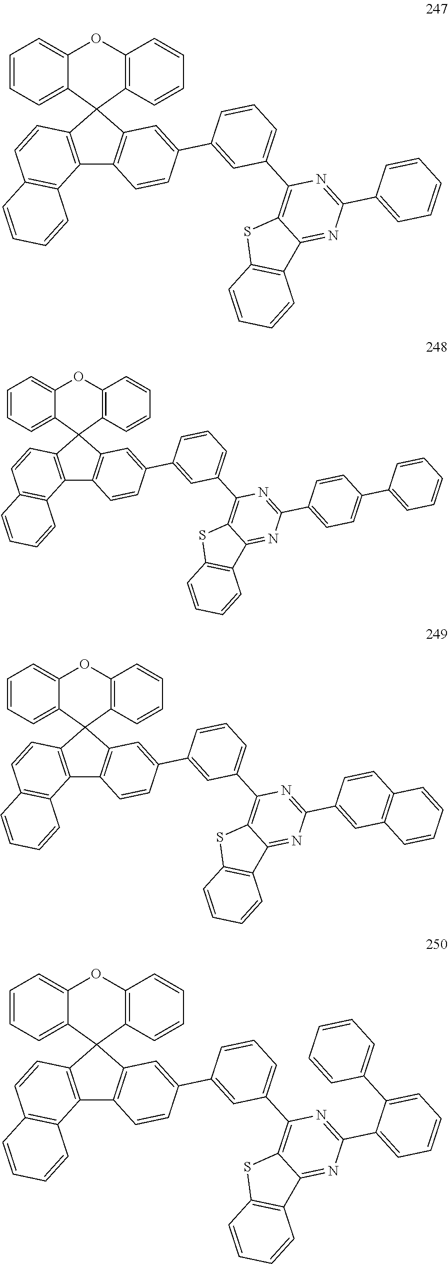 [SMILES: c1ccc(-c2nc(-c3cccc(-c4ccc5c(c4)C4(c6ccccc6Oc6ccccc64)c4ccc6ccccc6c4-5)c3)c3sc4ccccc4c3n2)cc1 ; c1ccc(-c2ccc(-c3nc(-c4cccc(-c5ccc6c(c5)C5(c7ccccc7Oc7ccccc75)c5ccc7ccccc7c5-6)c4)c4sc5ccccc5c4n3)cc2)cc1 ; c1cc(-c2ccc3c(c2)C2(c4ccccc4Oc4ccccc42)c2ccc4ccccc4c2-3)cc(-c2nc(-c3ccc4ccccc4c3)nc3c2sc2ccccc23)c1 ; c1ccc(-c2ccccc2-c2nc(-c3cccc(-c4ccc5c(c4)C4(c6ccccc6Oc6ccccc64)c4ccc6ccccc6c4-5)c3)c3sc4ccccc4c3n2)cc1]

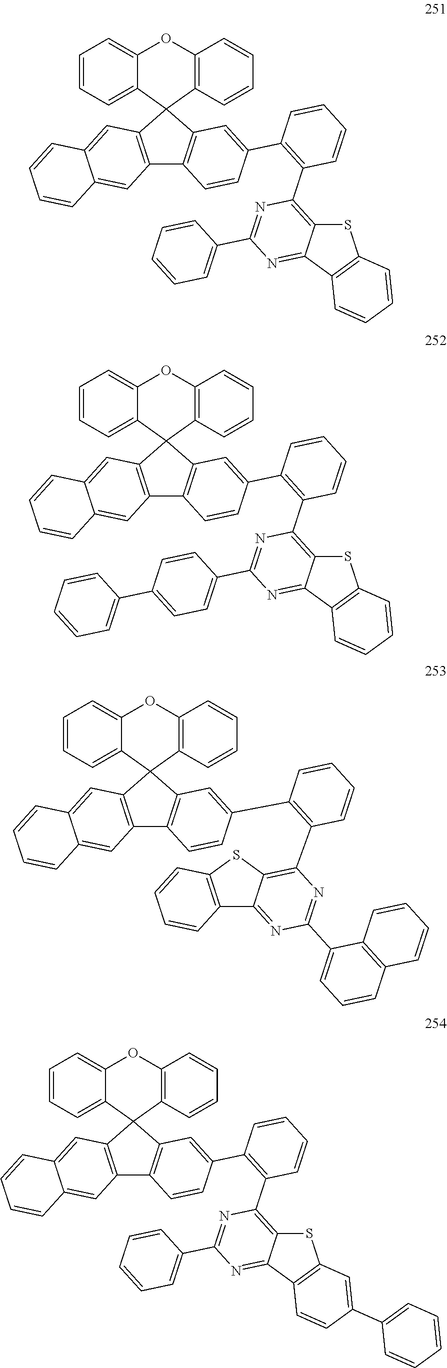 [SMILES: c1ccc(-c2nc(-c3ccccc3-c3ccc4c(c3)C3(c5ccccc5Oc5ccccc53)c3cc5ccccc5cc3-4)c3sc4ccccc4c3n2)cc1 ; c1ccc(-c2ccc(-c3nc(-c4ccccc4-c4ccc5c(c4)C4(c6ccccc6Oc6ccccc64)c4cc6ccccc6cc4-5)c4sc5ccccc5c4n3)cc2)cc1 ; c1ccc2c(c1)Oc1ccccc1C21c2cc(-c3ccccc3-c3nc(-c4cccc5ccccc45)nc4c3sc3ccccc34)ccc2-c2cc3ccccc3cc21 ; c1ccc(-c2ccc3c(c2)sc2c(-c4ccccc4-c4ccc5c(c4)C4(c6ccccc6Oc6ccccc64)c4cc6ccccc6cc4-5)nc(-c4ccccc4)nc23)cc1]

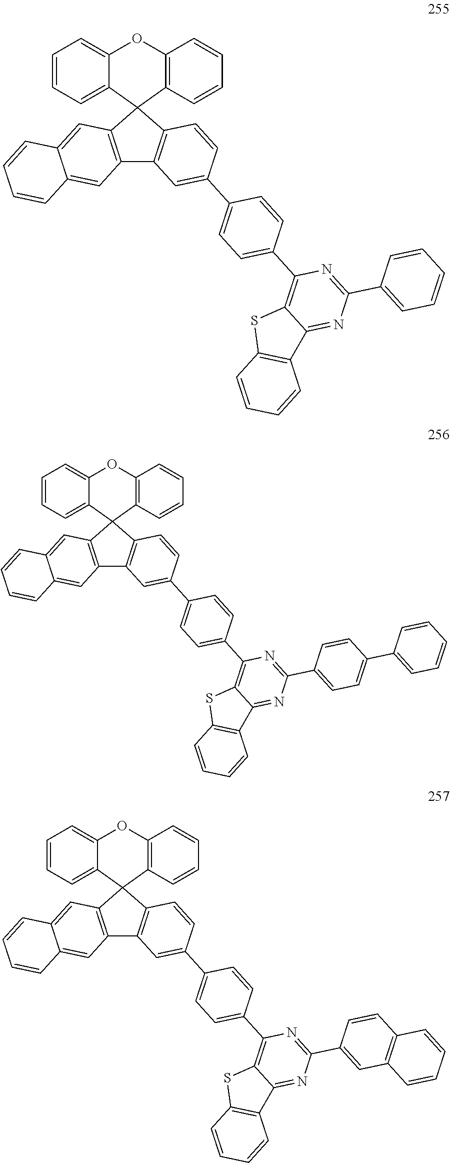 [SMILES: c1ccc(-c2nc(-c3ccc(-c4ccc5c(c4)-c4cc6ccccc6cc4C54c5ccccc5Oc5ccccc54)cc3)c3sc4ccccc4c3n2)cc1 ; c1ccc(-c2ccc(-c3nc(-c4ccc(-c5ccc6c(c5)-c5cc7ccccc7cc5C65c6ccccc6Oc6ccccc65)cc4)c4sc5ccccc5c4n3)cc2)cc1 ; c1ccc2c(c1)Oc1ccccc1C21c2ccc(-c3ccc(-c4nc(-c5ccc6ccccc6c5)nc5c4sc4ccccc45)cc3)cc2-c2cc3ccccc3cc21]

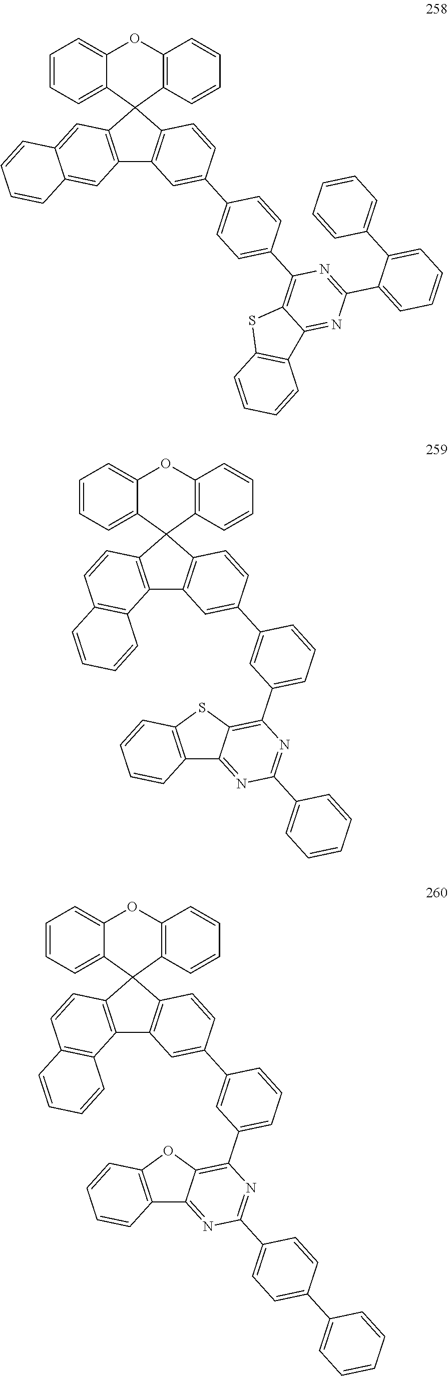 [SMILES: c1ccc(-c2ccccc2-c2nc(-c3ccc(-c4ccc5c(c4)-c4cc6ccccc6cc4C54c5ccccc5Oc5ccccc54)cc3)c3sc4ccccc4c3n2)cc1 ; c1ccc(-c2nc(-c3cccc(-c4ccc5c(c4)-c4c(ccc6ccccc46)C54c5ccccc5Oc5ccccc54)c3)c3sc4ccccc4c3n2)cc1 ; c1ccc(-c2ccc(-c3nc(-c4cccc(-c5ccc6c(c5)-c5c(ccc7ccccc57)C65c6ccccc6Oc6ccccc65)c4)c4oc5ccccc5c4n3)cc2)cc1]

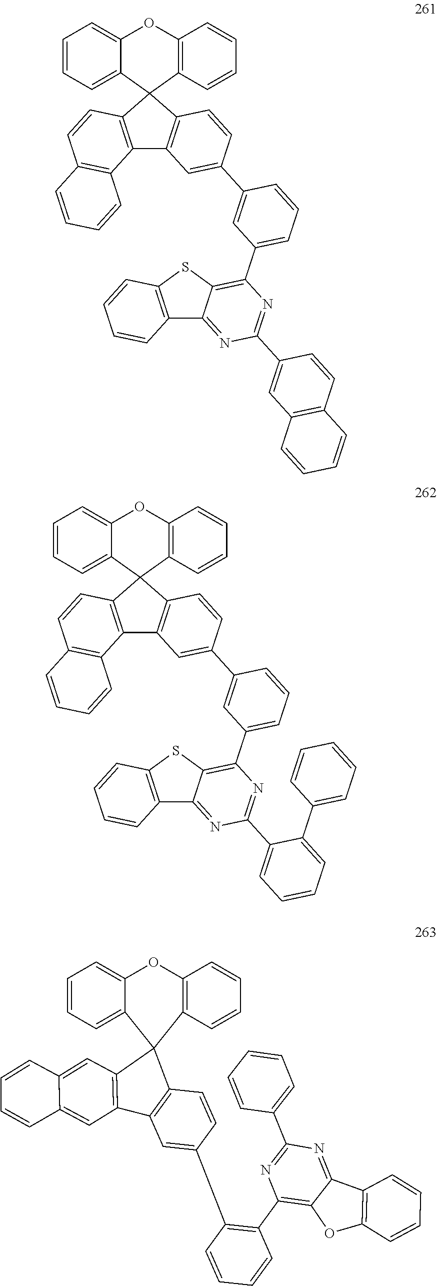 [SMILES: c1cc(-c2ccc3c(c2)-c2c(ccc4ccccc24)C32c3ccccc3Oc3ccccc32)cc(-c2nc(-c3ccc4ccccc4c3)nc3c2sc2ccccc23)c1 ; c1ccc(-c2ccccc2-c2nc(-c3cccc(-c4ccc5c(c4)-c4c(ccc6ccccc46)C54c5ccccc5Oc5ccccc54)c3)c3sc4ccccc4c3n2)cc1 ; c1ccc(-c2nc(-c3ccccc3-c3ccc4c(c3)-c3cc5ccccc5cc3C43c4ccccc4Oc4ccccc43)c3oc4ccccc4c3n2)cc1]

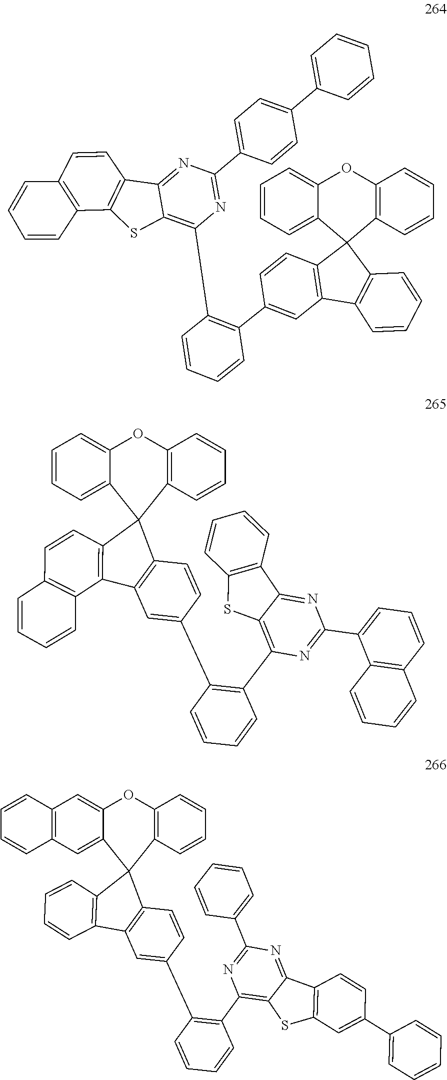 [SMILES: c1ccc(-c2ccc(-c3nc(-c4ccccc4-c4ccc5c(c4)-c4ccccc4C54c5ccccc5Oc5ccccc54)c4sc5c6ccccc6ccc5c4n3)cc2)cc1 ; c1ccc2c(c1)Oc1ccccc1C21c2ccc(-c3ccccc3-c3nc(-c4cccc5ccccc45)nc4c3sc3ccccc34)cc2-c2c1ccc1ccccc21 ; c1ccc(-c2ccc3c(c2)sc2c(-c4ccccc4-c4ccc5c(c4)-c4ccccc4C54c5ccccc5Oc5cc6ccccc6cc54)nc(-c4ccccc4)nc23)cc1]

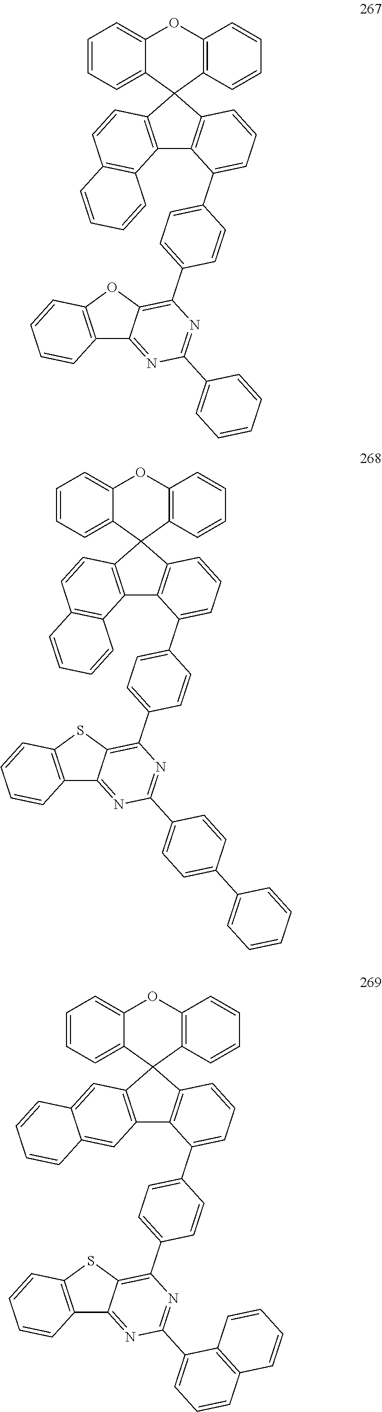

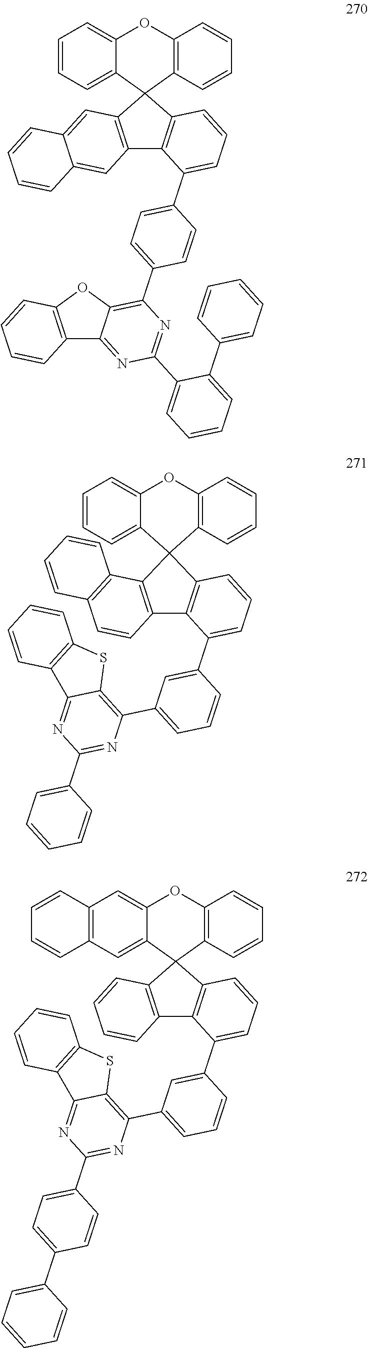

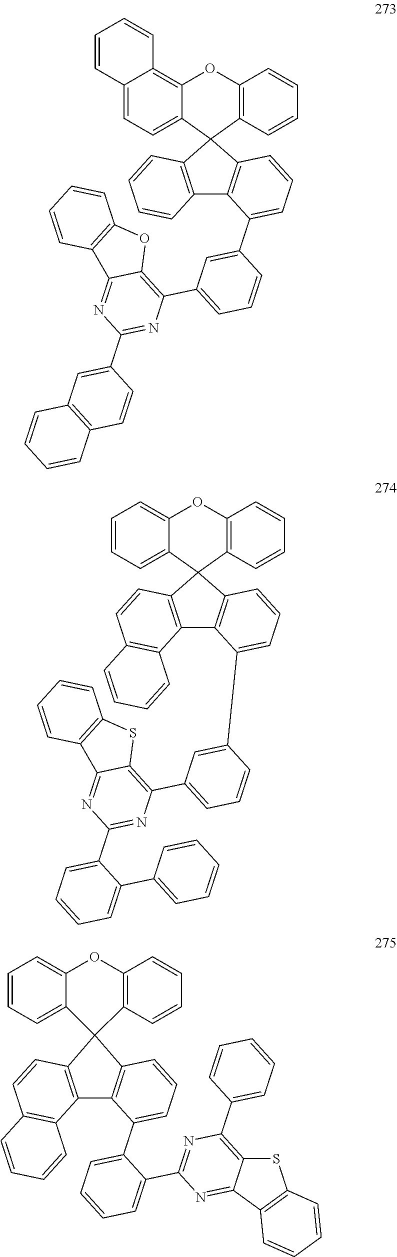

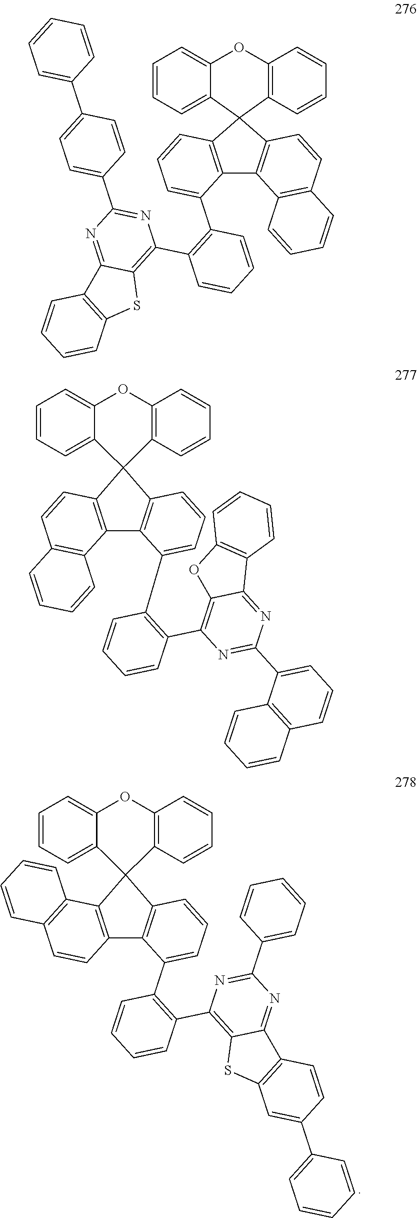

11. The compound of claim 1, wherein the compound represented by Chemical Formula 1 is selected from compounds represented by the following Chemical Formulas:

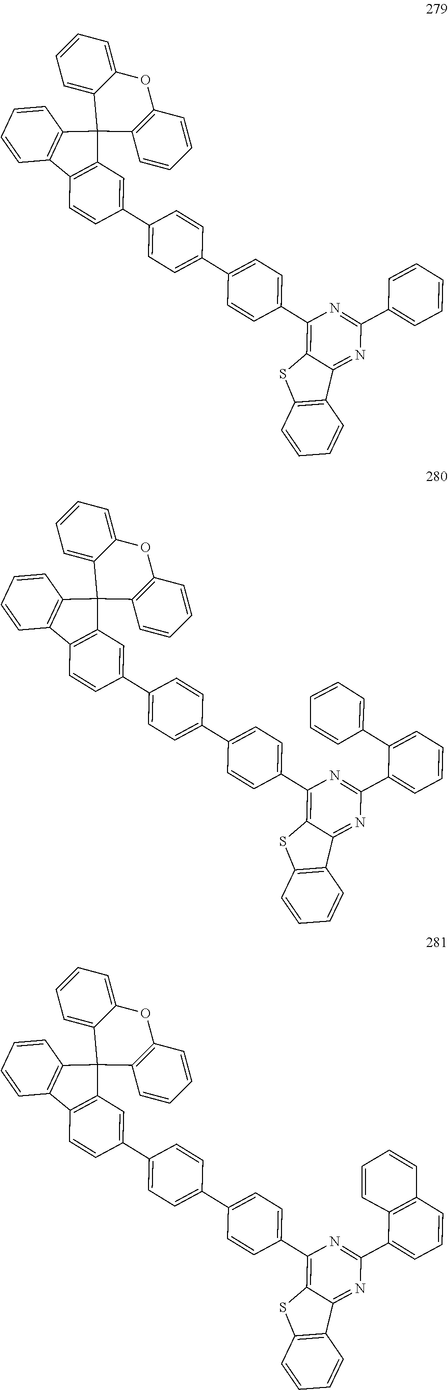 [SMILES: c1ccc(-c2nc(-c3ccc(-c4ccc(-c5ccc6c(c5)C5(c7ccccc7Oc7ccccc75)c5ccccc5-6)cc4)cc3)c3sc4ccccc4c3n2)cc1 ; c1ccc(-c2ccccc2-c2nc(-c3ccc(-c4ccc(-c5ccc6c(c5)C5(c7ccccc7Oc7ccccc75)c5ccccc5-6)cc4)cc3)c3sc4ccccc4c3n2)cc1 ; c1ccc2c(c1)Oc1ccccc1C21c2ccccc2-c2ccc(-c3ccc(-c4ccc(-c5nc(-c6cccc7ccccc67)nc6c5sc5ccccc56)cc4)cc3)cc21]

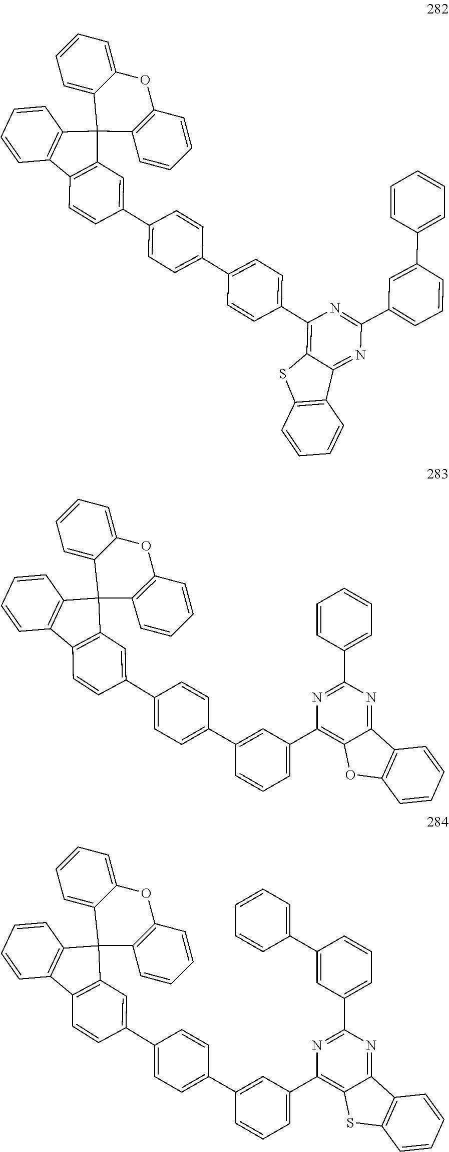 [SMILES: c1ccc(-c2cccc(-c3nc(-c4ccc(-c5ccc(-c6ccc7c(c6)C6(c8ccccc8Oc8ccccc86)c6ccccc6-7)cc5)cc4)c4sc5ccccc5c4n3)c2)cc1 ; c1ccc(-c2nc(-c3cccc(-c4ccc(-c5ccc6c(c5)C5(c7ccccc7Oc7ccccc75)c5ccccc5-6)cc4)c3)c3oc4ccccc4c3n2)cc1 ; c1ccc(-c2cccc(-c3nc(-c4cccc(-c5ccc(-c6ccc7c(c6)C6(c8ccccc8Oc8ccccc86)c6ccccc6-7)cc5)c4)c4sc5ccccc5c4n3)c2)cc1]

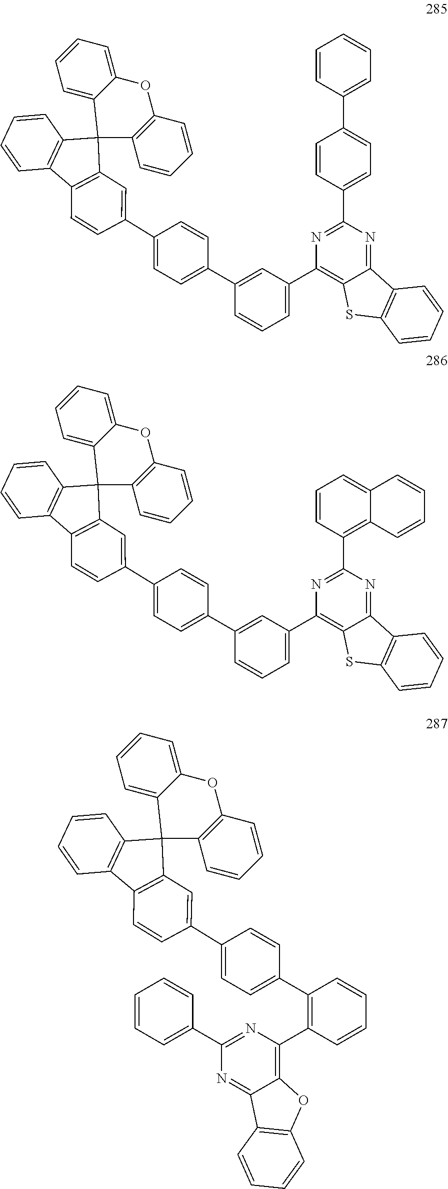 [SMILES: c1ccc(-c2ccc(-c3nc(-c4cccc(-c5ccc(-c6ccc7c(c6)C6(c8ccccc8Oc8ccccc86)c6ccccc6-7)cc5)c4)c4sc5ccccc5c4n3)cc2)cc1 ; c1cc(-c2ccc(-c3ccc4c(c3)C3(c5ccccc5Oc5ccccc53)c3ccccc3-4)cc2)cc(-c2nc(-c3cccc4ccccc34)nc3c2sc2ccccc23)c1 ; c1ccc(-c2nc(-c3ccccc3-c3ccc(-c4ccc5c(c4)C4(c6ccccc6Oc6ccccc64)c4ccccc4-5)cc3)c3oc4ccccc4c3n2)cc1]

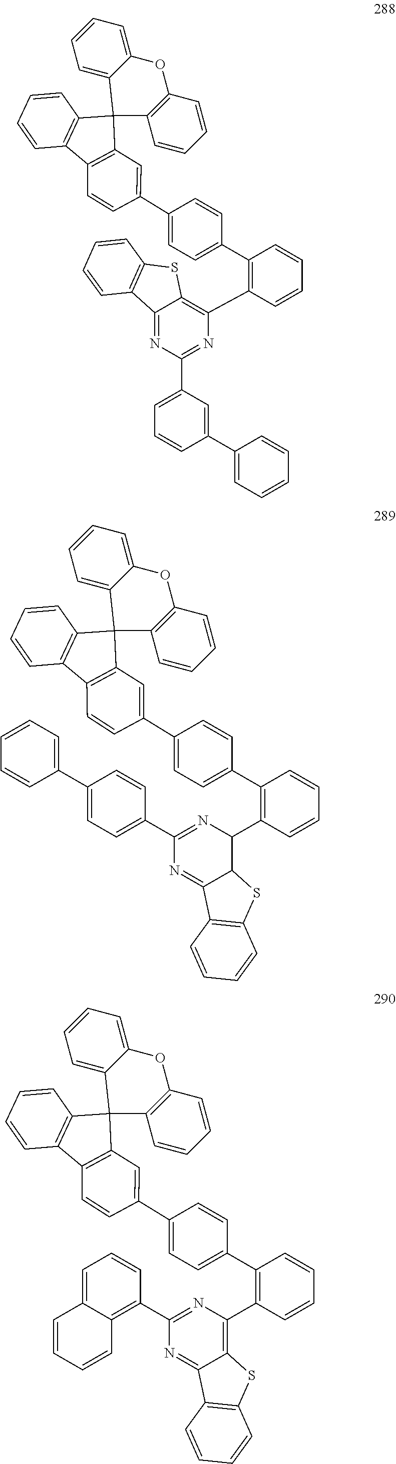 [SMILES: c1ccc(-c2cccc(-c3nc(-c4ccccc4-c4ccc(-c5ccc6c(c5)C5(c7ccccc7Oc7ccccc75)c5ccccc5-6)cc4)c4sc5ccccc5c4n3)c2)cc1 ; c1ccc(-c2ccc(C3=NC(c4ccccc4-c4ccc(-c5ccc6c(c5)C5(c7ccccc7Oc7ccccc75)c5ccccc5-6)cc4)C4Sc5ccccc5C4=N3)cc2)cc1 ; c1ccc2c(c1)Oc1ccccc1C21c2ccccc2-c2ccc(-c3ccc(-c4ccccc4-c4nc(-c5cccc6ccccc56)nc5c4sc4ccccc45)cc3)cc21]

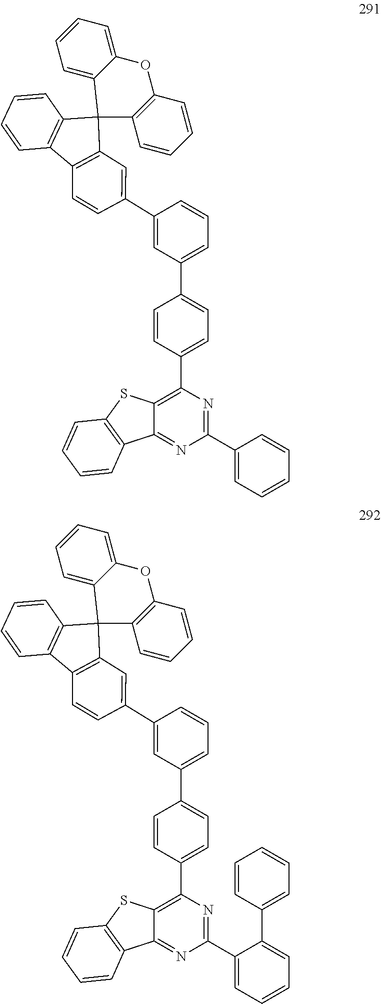 [SMILES: c1ccc(-c2nc(-c3ccc(-c4cccc(-c5ccc6c(c5)C5(c7ccccc7Oc7ccccc75)c5ccccc5-6)c4)cc3)c3sc4ccccc4c3n2)cc1 ; c1ccc(-c2ccccc2-c2nc(-c3ccc(-c4cccc(-c5ccc6c(c5)C5(c7ccccc7Oc7ccccc75)c5ccccc5-6)c4)cc3)c3sc4ccccc4c3n2)cc1]

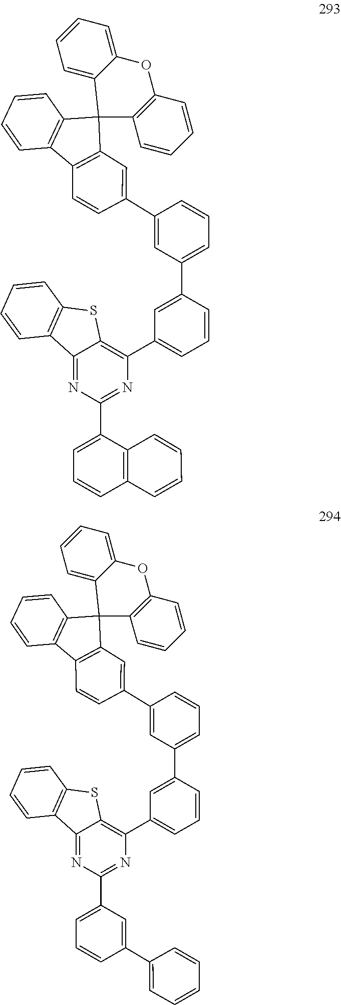

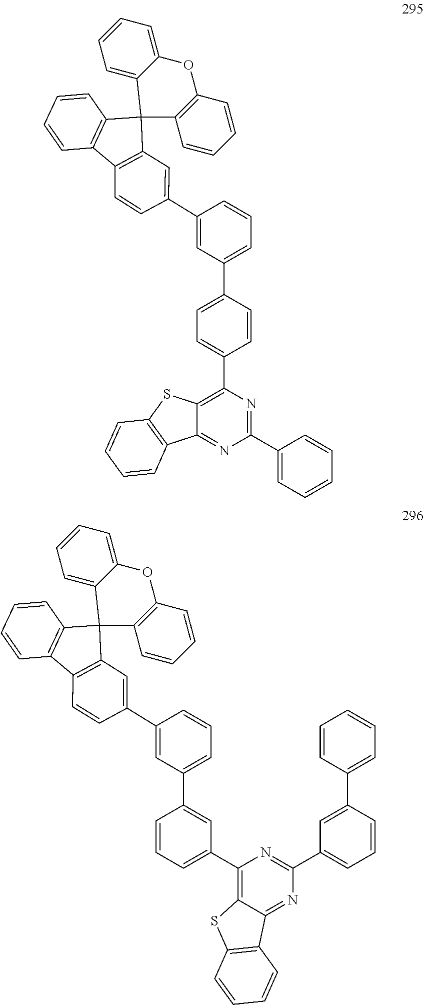 [SMILES: c1ccc(-c2nc(-c3ccc(-c4cccc(-c5ccc6c(c5)C5(c7ccccc7Oc7ccccc75)c5ccccc5-6)c4)cc3)c3sc4ccccc4c3n2)cc1 ; c1ccc(-c2cccc(-c3nc(-c4cccc(-c5cccc(-c6ccc7c(c6)C6(c8ccccc8Oc8ccccc86)c6ccccc6-7)c5)c4)c4sc5ccccc5c4n3)c2)cc1]

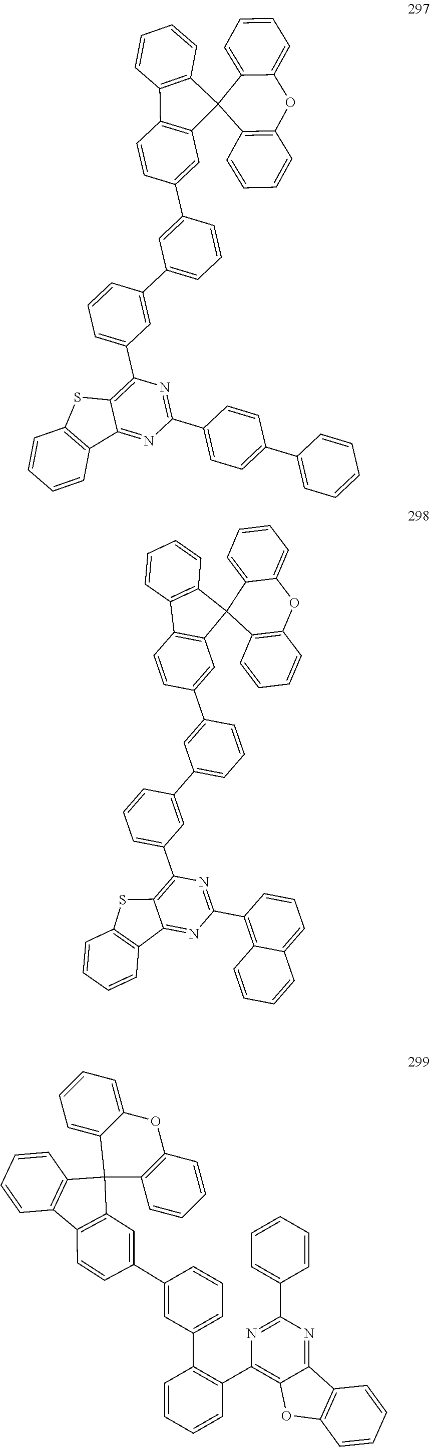 [SMILES: c1ccc(-c2ccc(-c3nc(-c4cccc(-c5cccc(-c6ccc7c(c6)C6(c8ccccc8Oc8ccccc86)c6ccccc6-7)c5)c4)c4sc5ccccc5c4n3)cc2)cc1 ; c1cc(-c2cccc(-c3nc(-c4cccc5ccccc45)nc4c3sc3ccccc34)c2)cc(-c2ccc3c(c2)C2(c4ccccc4Oc4ccccc42)c2ccccc2-3)c1 ; c1ccc(-c2nc(-c3ccccc3-c3cccc(-c4ccc5c(c4)C4(c6ccccc6Oc6ccccc64)c4ccccc4-5)c3)c3oc4ccccc4c3n2)cc1]

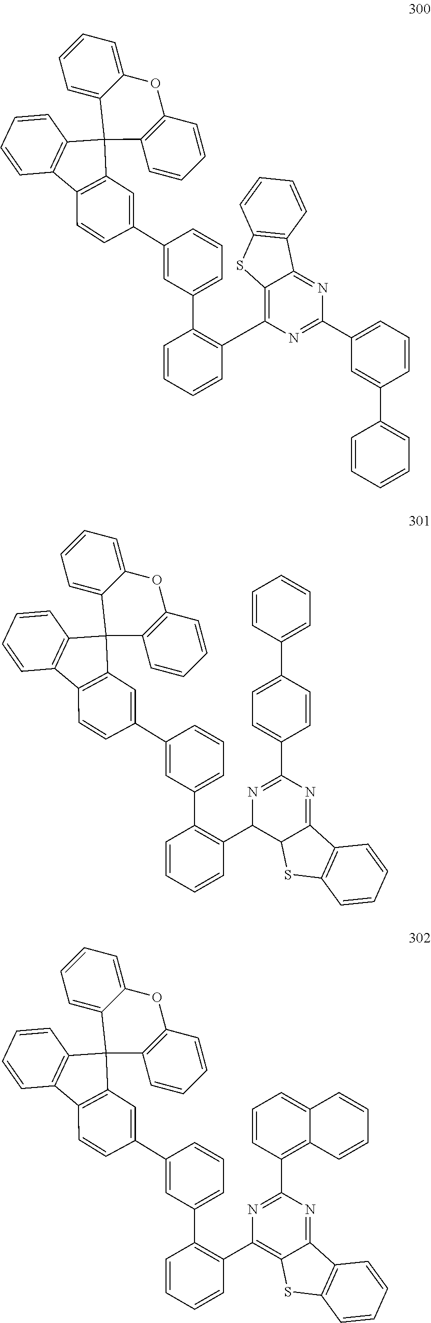 [SMILES: c1ccc(-c2cccc(-c3nc(-c4ccccc4-c4cccc(-c5ccc6c(c5)C5(c7ccccc7Oc7ccccc75)c5ccccc5-6)c4)c4sc5ccccc5c4n3)c2)cc1 ; c1ccc(-c2ccc(C3=NC(c4ccccc4-c4cccc(-c5ccc6c(c5)C5(c7ccccc7Oc7ccccc75)c5ccccc5-6)c4)C4Sc5ccccc5C4=N3)cc2)cc1 ; c1cc(-c2ccc3c(c2)C2(c4ccccc4Oc4ccccc42)c2ccccc2-3)cc(-c2ccccc2-c2nc(-c3cccc4ccccc34)nc3c2sc2ccccc23)c1]

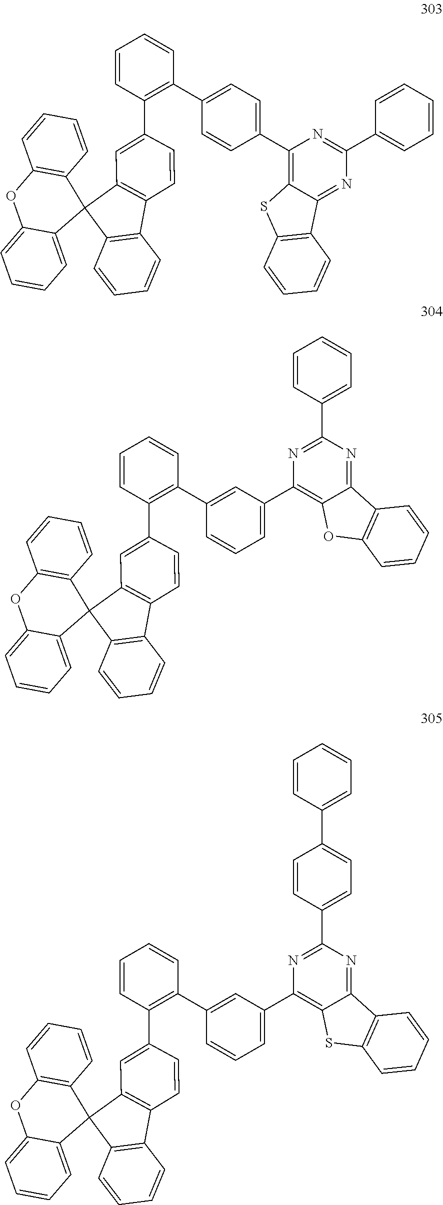

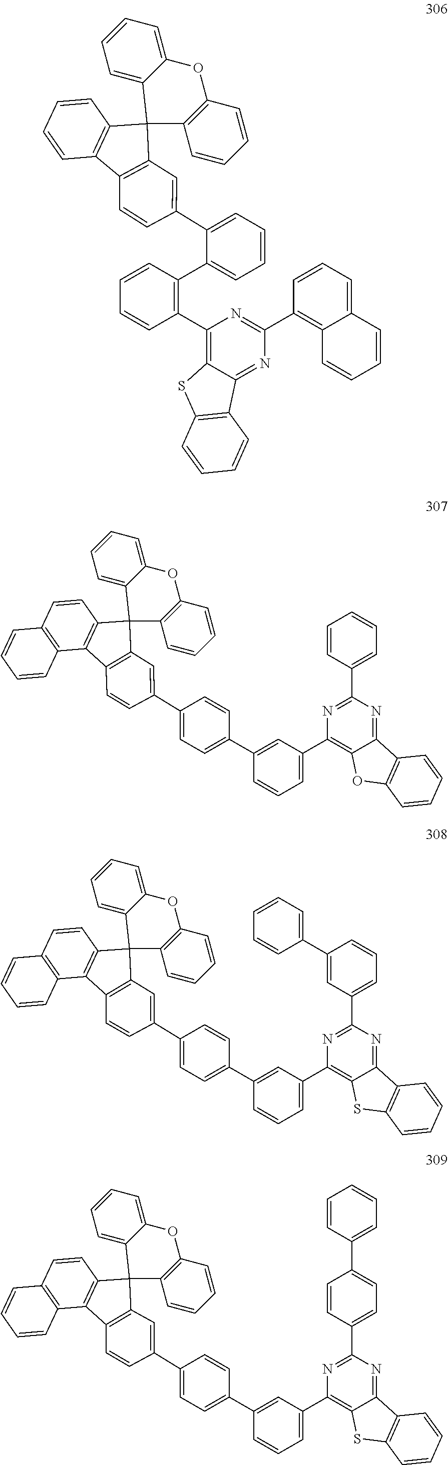 [SMILES: c1ccc2c(c1)Oc1ccccc1C21c2ccccc2-c2ccc(-c3ccccc3-c3ccccc3-c3nc(-c4cccc5ccccc45)nc4c3sc3ccccc34)cc21 ; c1ccc(-c2nc(-c3cccc(-c4ccc(-c5ccc6c(c5)C5(c7ccccc7Oc7ccccc75)c5ccc7ccccc7c5-6)cc4)c3)c3oc4ccccc4c3n2)cc1 ; c1ccc(-c2cccc(-c3nc(-c4cccc(-c5ccc(-c6ccc7c(c6)C6(c8ccccc8Oc8ccccc86)c6ccc8ccccc8c6-7)cc5)c4)c4sc5ccccc5c4n3)c2)cc1 ; c1ccc(-c2ccc(-c3nc(-c4cccc(-c5ccc(-c6ccc7c(c6)C6(c8ccccc8Oc8ccccc86)c6ccc8ccccc8c6-7)cc5)c4)c4sc5ccccc5c4n3)cc2)cc1]

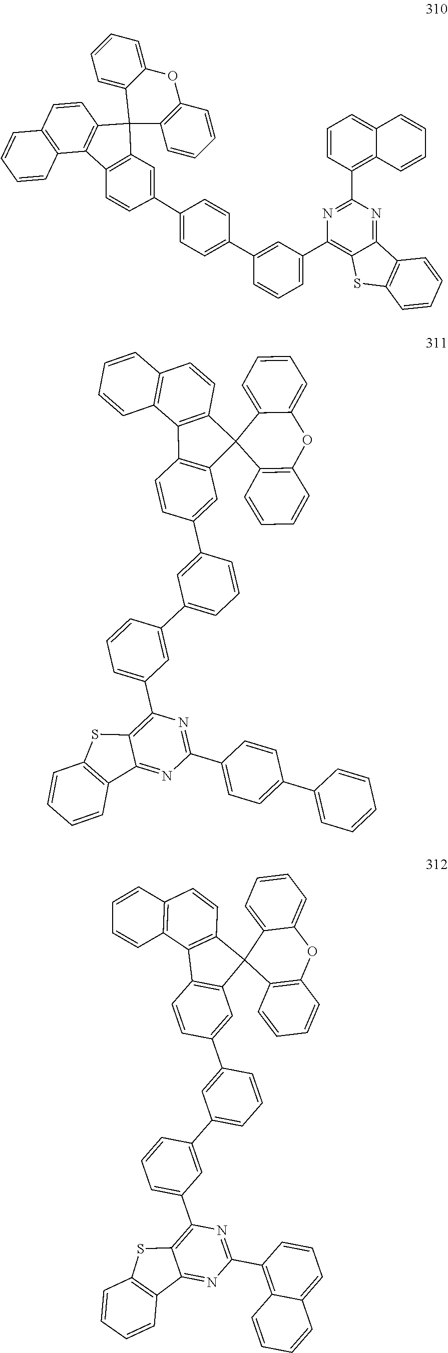 [SMILES: c1cc(-c2ccc(-c3ccc4c(c3)C3(c5ccccc5Oc5ccccc53)c3ccc5ccccc5c3-4)cc2)cc(-c2nc(-c3cccc4ccccc34)nc3c2sc2ccccc23)c1 ; c1ccc(-c2ccc(-c3nc(-c4cccc(-c5cccc(-c6ccc7c(c6)C6(c8ccccc8Oc8ccccc86)c6ccc8ccccc8c6-7)c5)c4)c4sc5ccccc5c4n3)cc2)cc1 ; c1cc(-c2cccc(-c3nc(-c4cccc5ccccc45)nc4c3sc3ccccc34)c2)cc(-c2ccc3c(c2)C2(c4ccccc4Oc4ccccc42)c2ccc4ccccc4c2-3)c1]

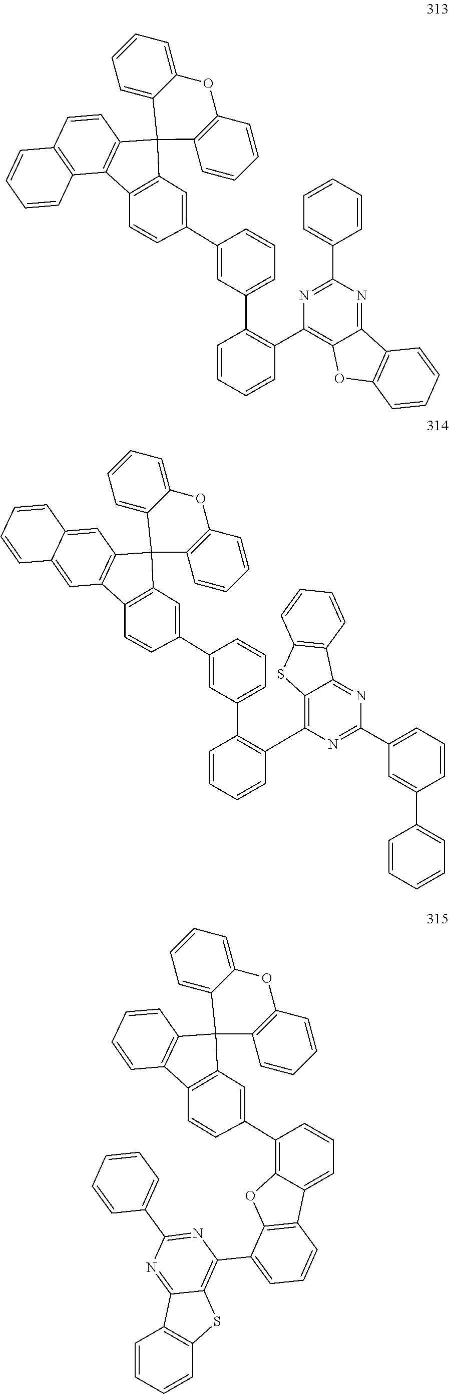 [SMILES: c1ccc(-c2nc(-c3ccccc3-c3cccc(-c4ccc5c(c4)C4(c6ccccc6Oc6ccccc64)c4ccc6ccccc6c4-5)c3)c3oc4ccccc4c3n2)cc1 ; c1ccc(-c2cccc(-c3nc(-c4ccccc4-c4cccc(-c5ccc6c(c5)C5(c7ccccc7Oc7ccccc75)c5cc7ccccc7cc5-6)c4)c4sc5ccccc5c4n3)c2)cc1 ; c1ccc(-c2nc(-c3cccc4c3oc3c(-c5ccc6c(c5)C5(c7ccccc7Oc7ccccc75)c5ccccc5-6)cccc34)c3sc4ccccc4c3n2)cc1]

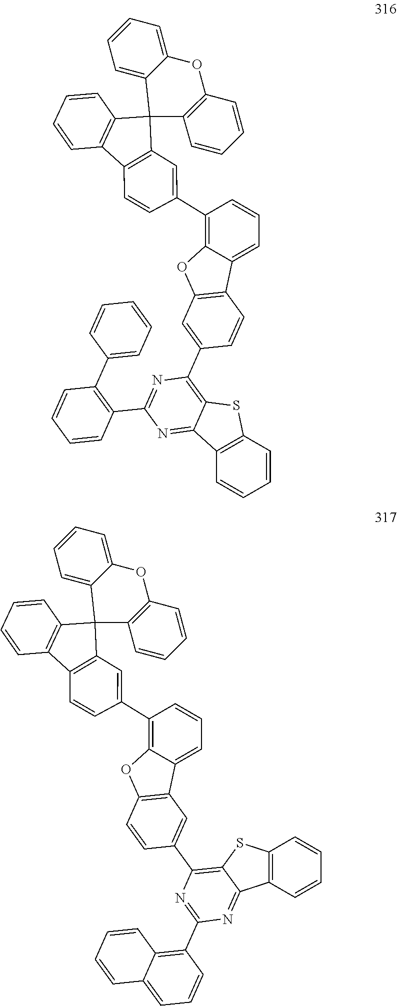 [SMILES: c1ccc(-c2ccccc2-c2nc(-c3ccc4c(c3)oc3c(-c5ccc6c(c5)C5(c7ccccc7Oc7ccccc75)c5ccccc5-6)cccc34)c3sc4ccccc4c3n2)cc1 ; c1ccc2c(c1)Oc1ccccc1C21c2ccccc2-c2ccc(-c3cccc4c3oc3ccc(-c5nc(-c6cccc7ccccc67)nc6c5sc5ccccc56)cc34)cc21]

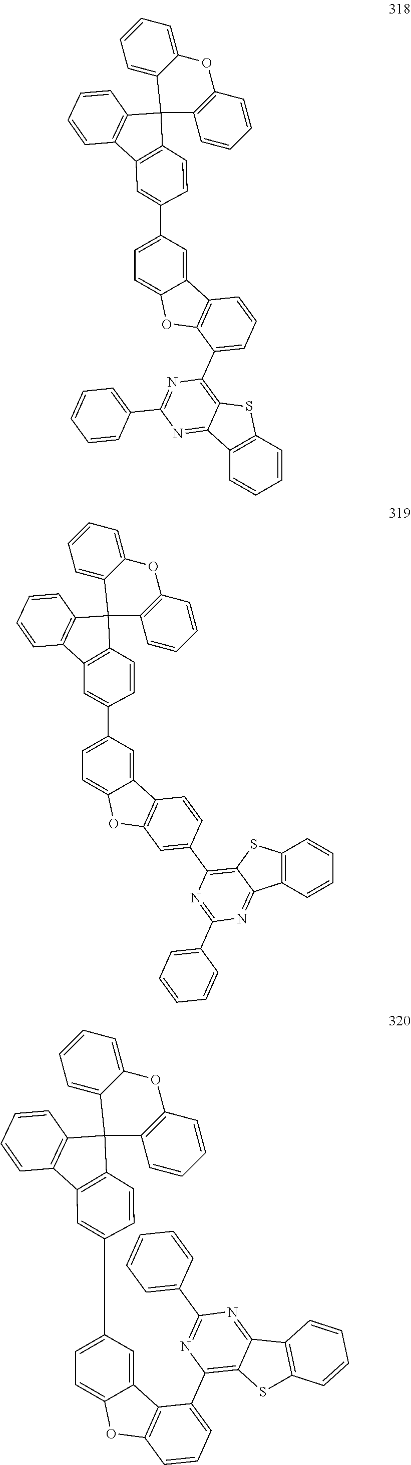 [SMILES: c1ccc(-c2nc(-c3cccc4c3oc3ccc(-c5ccc6c(c5)-c5ccccc5C65c6ccccc6Oc6ccccc65)cc34)c3sc4ccccc4c3n2)cc1 ; c1ccc(-c2nc(-c3ccc4c(c3)oc3ccc(-c5ccc6c(c5)-c5ccccc5C65c6ccccc6Oc6ccccc65)cc34)c3sc4ccccc4c3n2)cc1 ; c1ccc(-c2nc(-c3cccc4oc5ccc(-c6ccc7c(c6)-c6ccccc6C76c7ccccc7Oc7ccccc76)cc5c34)c3sc4ccccc4c3n2)cc1]

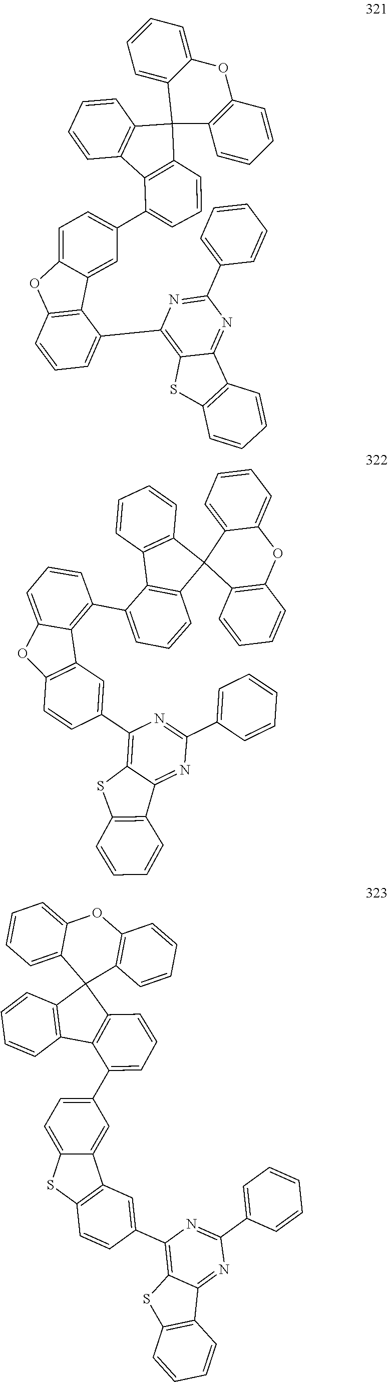

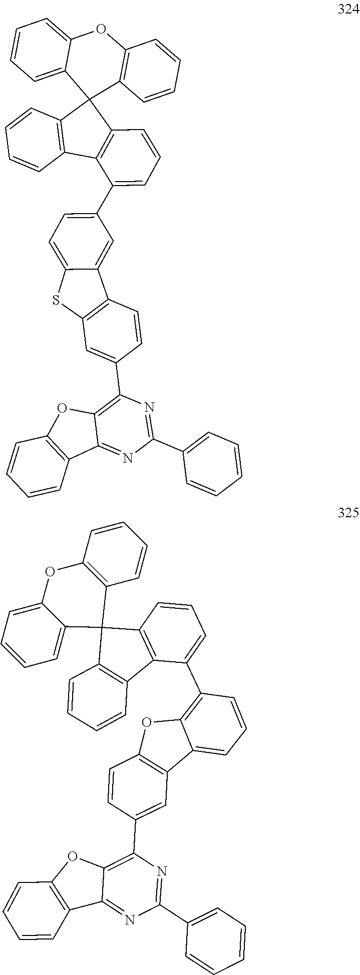 [SMILES: c1ccc(-c2nc(-c3ccc4c(c3)sc3ccc(-c5cccc6c5-c5ccccc5C65c6ccccc6Oc6ccccc65)cc34)c3oc4ccccc4c3n2)cc1 ; c1ccc(-c2nc(-c3ccc4oc5c(-c6cccc7c6-c6ccccc6C76c7ccccc7Oc7ccccc76)cccc5c4c3)c3oc4ccccc4c3n2)cc1]

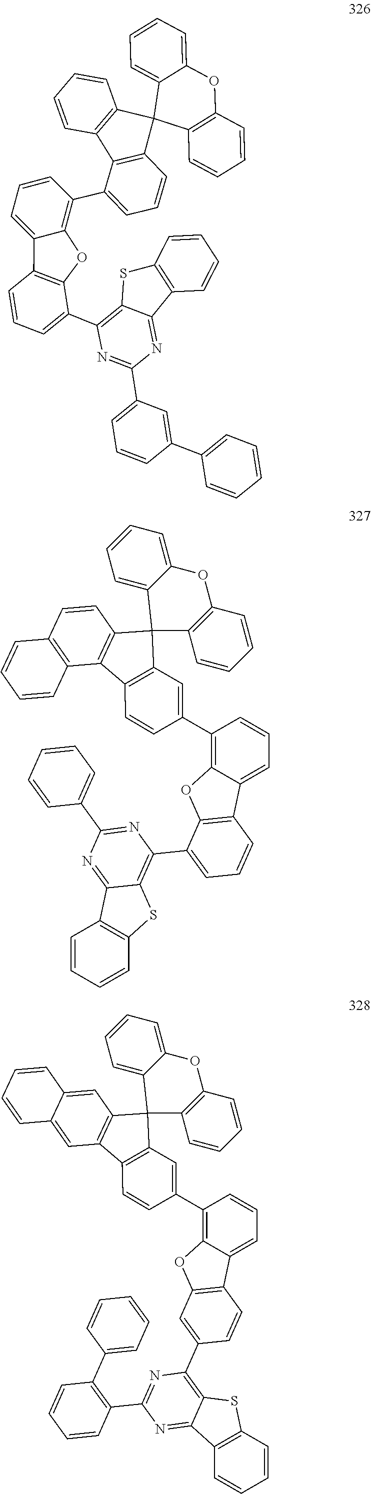

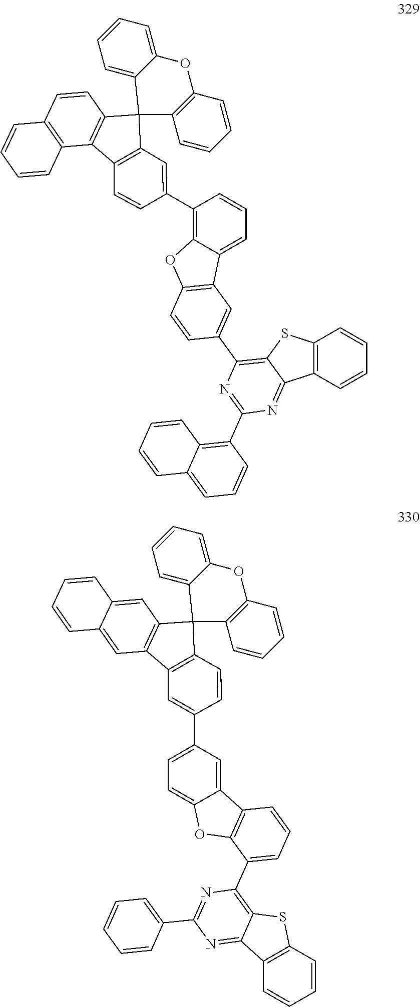

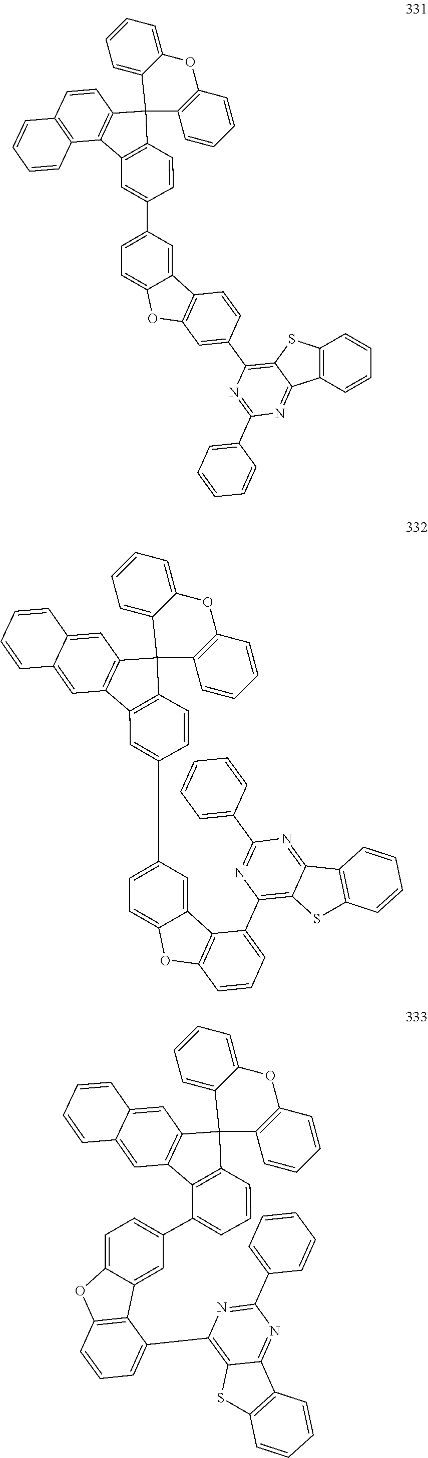

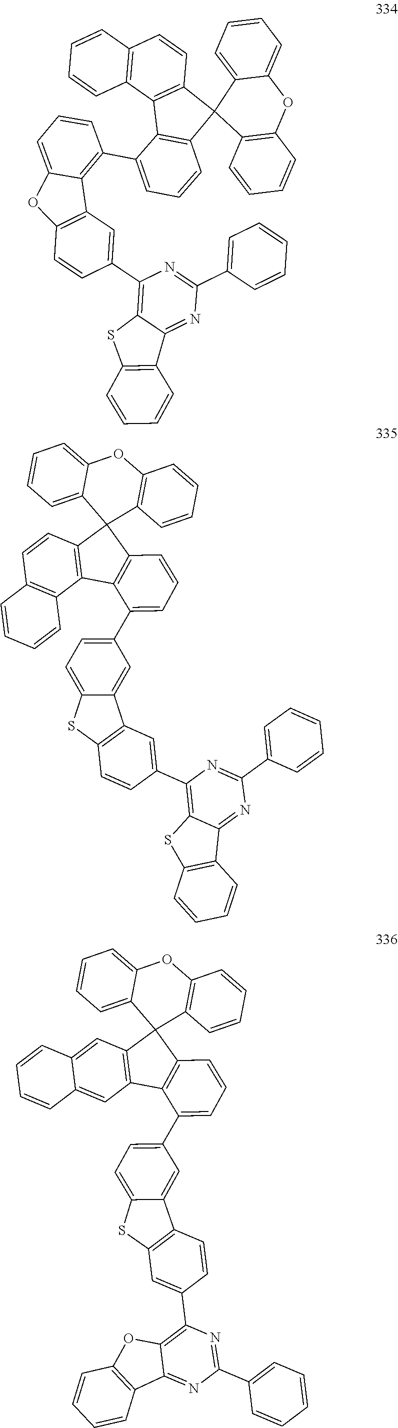

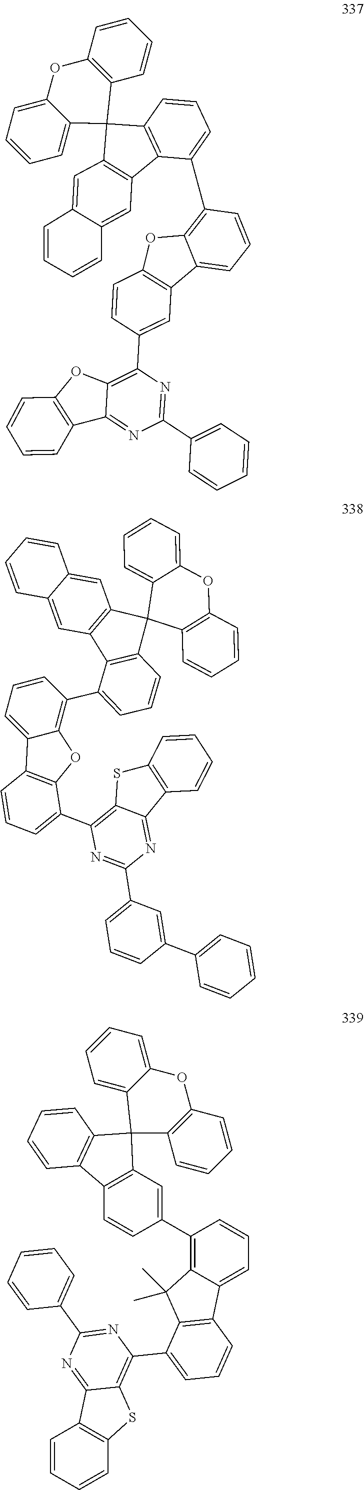 [SMILES: c1ccc(-c2nc(-c3ccc4oc5c(-c6cccc7c6-c6cc8ccccc8cc6C76c7ccccc7Oc7ccccc76)cccc5c4c3)c3oc4ccccc4c3n2)cc1 ; c1ccc(-c2cccc(-c3nc(-c4cccc5c4oc4c(-c6cccc7c6-c6cc8ccccc8cc6C76c7ccccc7Oc7ccccc76)cccc45)c4sc5ccccc5c4n3)c2)cc1 ; CC1(C)c2c(-c3ccc4c(c3)C3(c5ccccc5Oc5ccccc53)c3ccccc3-4)cccc2-c2cccc(-c3nc(-c4ccccc4)nc4c3sc3ccccc34)c21]

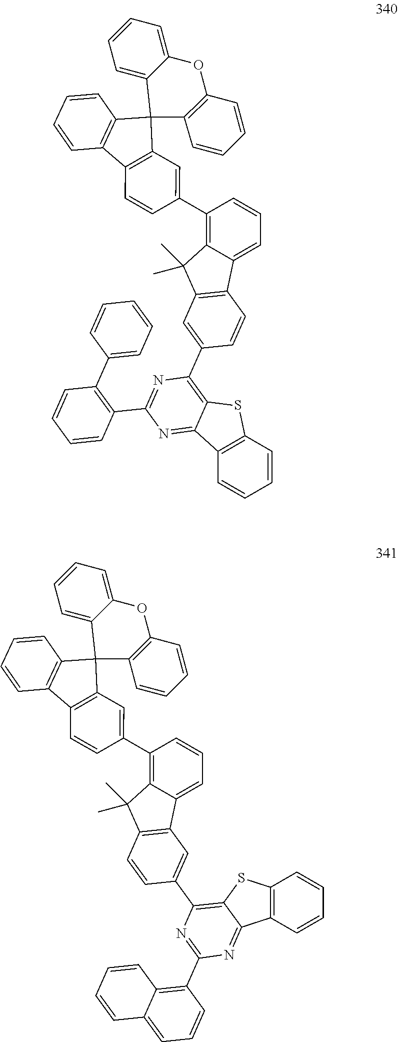 [SMILES: CC1(C)c2cc(-c3nc(-c4ccccc4-c4ccccc4)nc4c3sc3ccccc34)ccc2-c2cccc(-c3ccc4c(c3)C3(c5ccccc5Oc5ccccc53)c3ccccc3-4)c21 ; CC1(C)c2ccc(-c3nc(-c4cccc5ccccc45)nc4c3sc3ccccc34)cc2-c2cccc(-c3ccc4c(c3)C3(c5ccccc5Oc5ccccc53)c3ccccc3-4)c21]

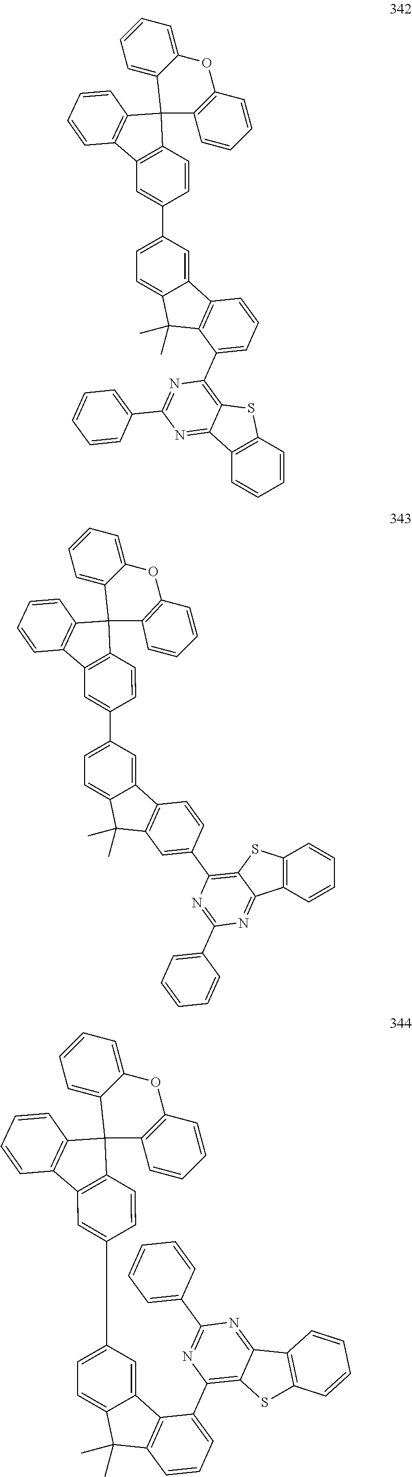 [SMILES: CC1(C)c2ccc(-c3ccc4c(c3)-c3ccccc3C43c4ccccc4Oc4ccccc43)cc2-c2cccc(-c3nc(-c4ccccc4)nc4c3sc3ccccc34)c21 ; CC1(C)c2ccc(-c3ccc4c(c3)-c3ccccc3C43c4ccccc4Oc4ccccc43)cc2-c2ccc(-c3nc(-c4ccccc4)nc4c3sc3ccccc34)cc21 ; CC1(C)c2ccc(-c3ccc4c(c3)-c3ccccc3C43c4ccccc4Oc4ccccc43)cc2-c2c(-c3nc(-c4ccccc4)nc4c3sc3ccccc34)cccc21]

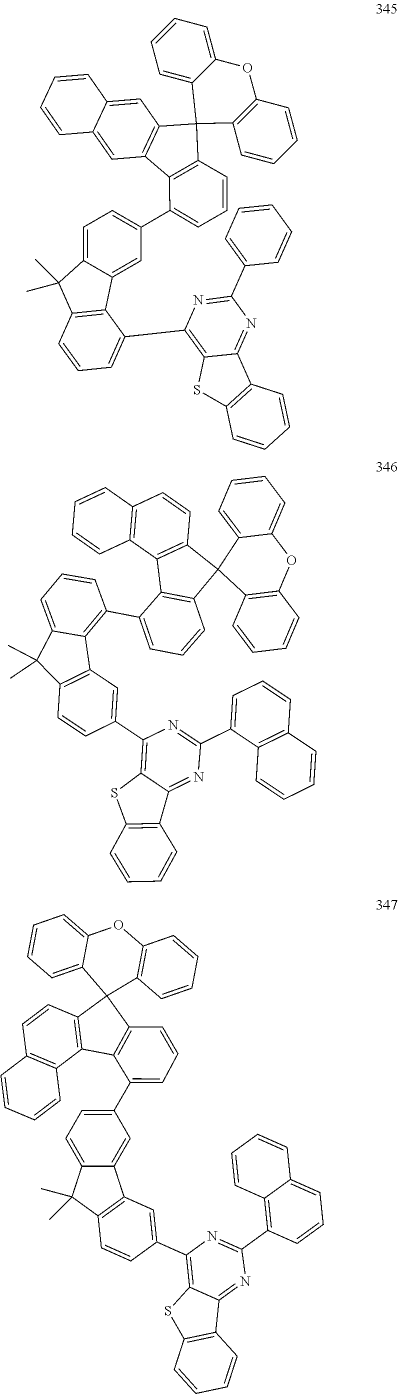

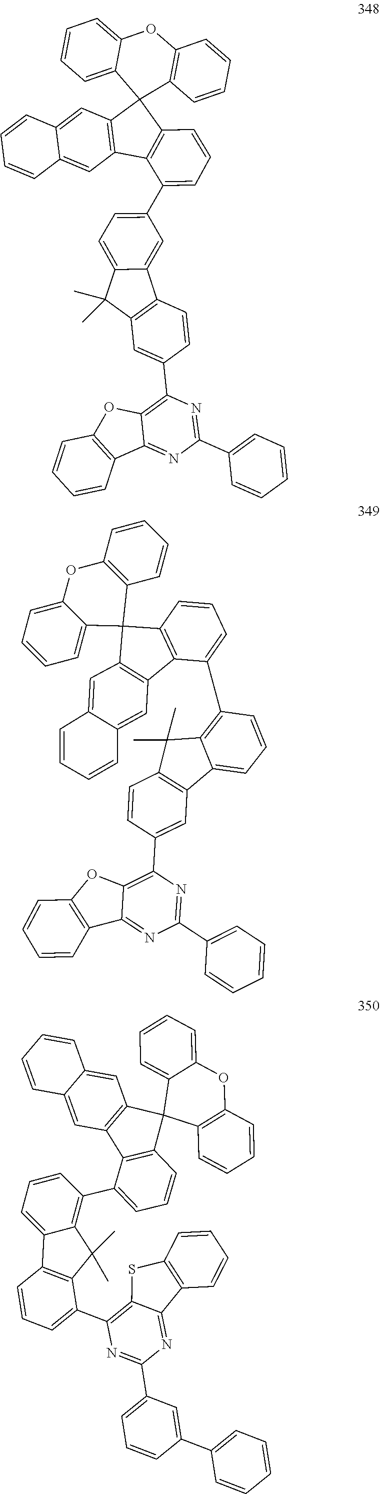

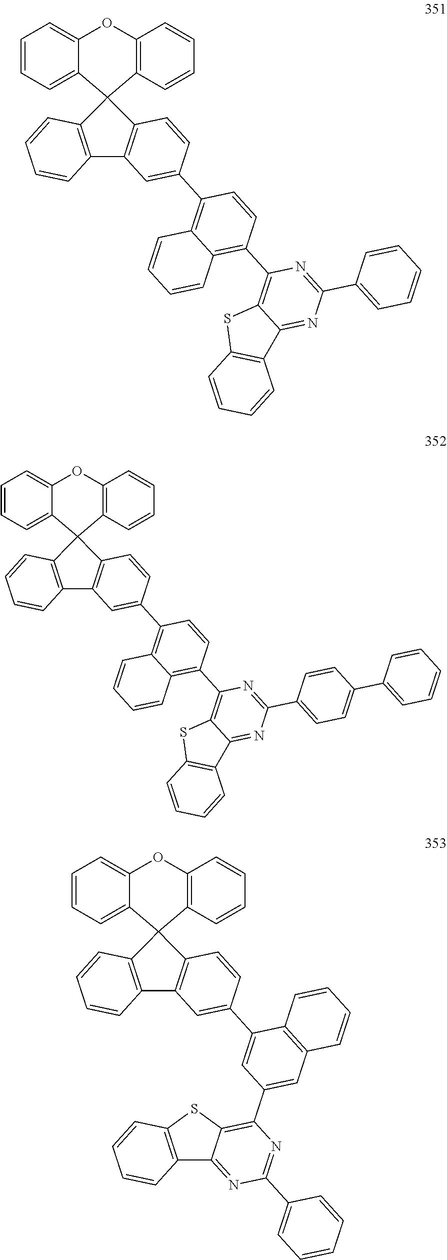

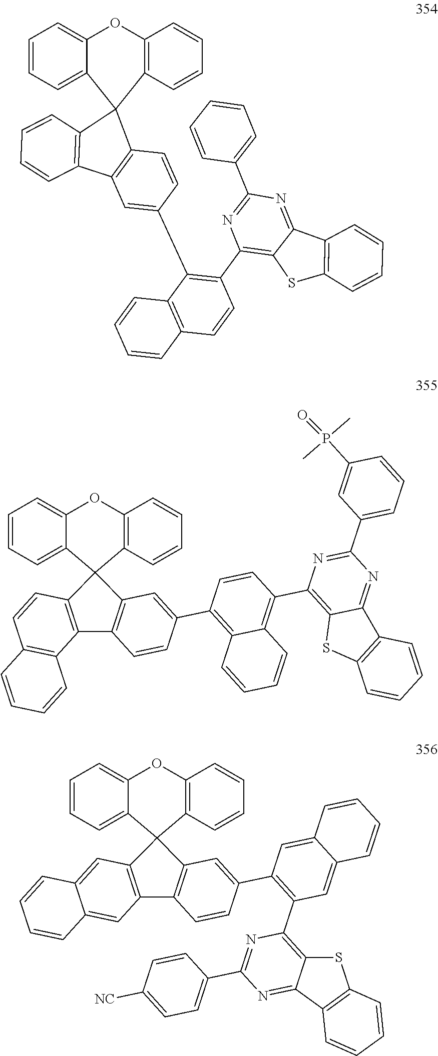 [SMILES: c1ccc(-c2nc(-c3ccc4ccccc4c3-c3ccc4c(c3)-c3ccccc3C43c4ccccc4Oc4ccccc43)c3sc4ccccc4c3n2)cc1 ; CP(C)(=O)c1cccc(-c2nc(-c3ccc(-c4ccc5c(c4)C4(c6ccccc6Oc6ccccc64)c4ccc6ccccc6c4-5)c4ccccc34)c3sc4ccccc4c3n2)c1 ; N#Cc1ccc(-c2nc(-c3cc4ccccc4cc3-c3ccc4c(c3)C3(c5ccccc5Oc5ccccc53)c3cc5ccccc5cc3-4)c3sc4ccccc4c3n2)cc1]

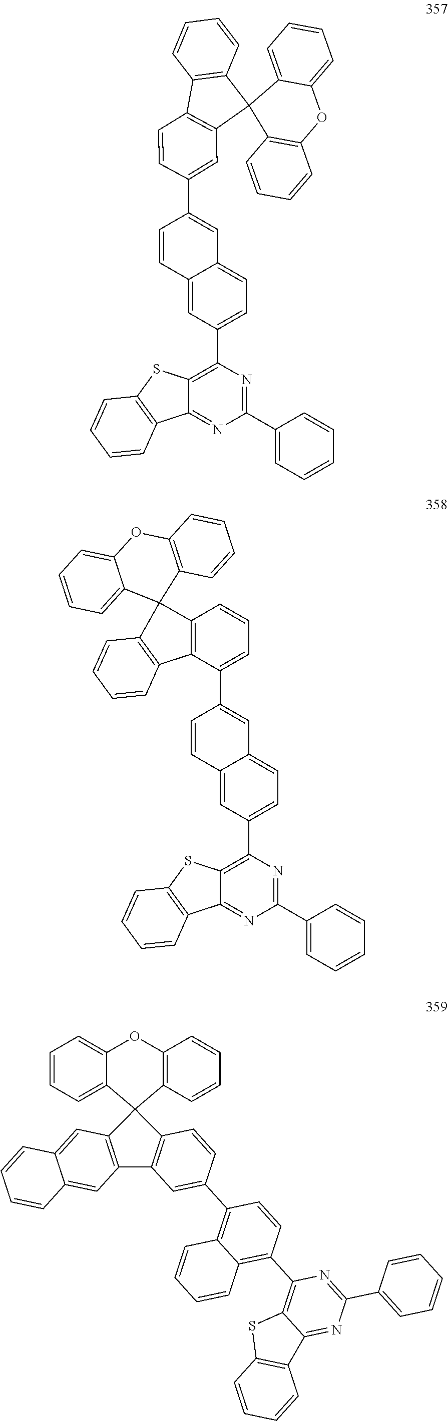 [SMILES: c1ccc(-c2nc(-c3ccc4cc(-c5ccc6c(c5)C5(c7ccccc7Oc7ccccc75)c5ccccc5-6)ccc4c3)c3sc4ccccc4c3n2)cc1 ; c1ccc(-c2nc(-c3ccc4cc(-c5cccc6c5-c5ccccc5C65c6ccccc6Oc6ccccc65)ccc4c3)c3sc4ccccc4c3n2)cc1 ; c1ccc(-c2nc(-c3ccc(-c4ccc5c(c4)-c4cc6ccccc6cc4C54c5ccccc5Oc5ccccc54)c4ccccc34)c3sc4ccccc4c3n2)cc1]

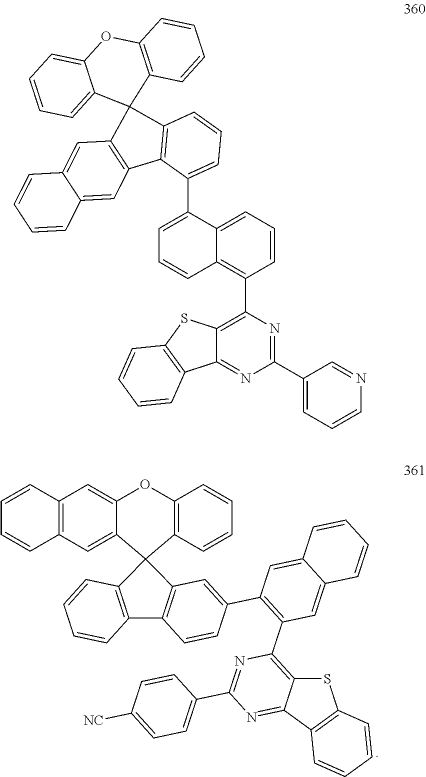 [SMILES: c1cncc(-c2nc(-c3cccc4c(-c5cccc6c5-c5cc7ccccc7cc5C65c6ccccc6Oc6ccccc65)cccc34)c3sc4ccccc4c3n2)c1 ; N#Cc1ccc(-c2nc(-c3cc4ccccc4cc3-c3ccc4c(c3)C3(c5ccccc5Oc5cc6ccccc6cc53)c3ccccc3-4)c3sc4ccccc4c3n2)cc1]

12. The compound of claim 1, wherein the compound represented by Chemical Formula 1 is an electron transport layer material or an electron transport auxiliary layer material.

13. An electroluminescent device comprising: an anode, a cathode, and one or more organic layers disposed between the anode and the cathode, wherein at least one of the one or more organic layers comprises the compound of claim 1.

14. The electroluminescent device of claim 13, wherein the organic layer comprising the compound is selected from a light emitting layer, a light emitting auxiliary layer, a hole injection layer, a hole transport layer, an electron injection layer, an electron transport layer, and an electron transport auxiliary layer.

15. The electroluminescent device of claim 13, wherein the organic layer comprising the compound is a light emitting layer, an electron transport layer or an electron transport auxiliary layer.
